SEQUENCE LISTING (10 sequences)

SEQ ID NO: 1 - protein, 27 aa, Artificial Sequence
  Mouse N-terminal CD8 alpha signal peptide
MASPLTRFLSLNLLLLGESIILGSGEA

SEQ ID NO: 2 - protein, 112 aa, Artificial Sequence
  anti-murine CD19 variable heavy chain derived from the 1D3 rat monoclonal anti- CD19 antibody
LQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVS

SEQ ID NO: 3 - protein, 105 aa, Artificial Sequence
  anti-murine CD19 variable light chain derived from the 1D3 rat monoclonal anti- CD19 antibody
IQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLEL

SEQ ID NO: 4 - protein, 16 aa, Artificial Sequence
  Murine IgG3 hinge region
PRIPKPSTPPGSSCPP

SEQ ID NO: 5 - protein, 27 aa, Artificial Sequence
  murine CD28 transmembrane region
FWALVVVAGVLFCYGLLVTVALCVIWT

SEQ ID NO: 6 - protein, 48 aa, Artificial Sequence
  intracellular signaling domain of murine 41BB
SVLKWIRKKFPHIFKQPFKKTTGAAQEEDACSCRCPQEEEGGGGGYEL

SEQ ID NO: 7 - protein, 113 aa, Artificial Sequence
  Intracellular signaling domain of murine CD3
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 8 - protein, 162 aa, Artificial Sequence
  Mouse Thy1.1
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 9 - protein, 120 aa, Artificial Sequence
  anti-human CD19 variable heavy chain derived from the FMC63 anti- CD19 antibody
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 10 - protein, 106 aa, Artificial Sequence
  anti-murine CD19 variable light chain derived from the FMC63 anti- CD19 antibody
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEI

(19) United States
(12) Patent Application Publication
(10) Pub. No.: US 2022/0225597 A1
(43) Pub. Date: Jul. 21, 2022

(54) MOUSE MODEL FOR ASSESSING TOXICITIES ASSOCIATED WITH IMMUNOTHERAPIES

(71) Applicant: Juno Therapeutics, Inc., Seattle, WA (US)

(72) Inventors: Eric Martin CHADWICK, Seattle, WA (US); Ronald James HAUSE, JR., Seattle, WA (US); Yue JIANG, Seattle, WA (US); Hyam I. LEVITSKY, Seattle, WA (US); Ruth Amanda SALMON, Seattle, WA (US); Rafael Angel PONCE, Seattle, WA (US); Neis Eric OLSON, Seattle, WA (US)

(73) Assignee: Juno Therapeutics, Inc., Seattle, WA (US)

(21) Appl. No.: 16/624,766

(22) PCT Filed: Jun. 29, 2018
(86) PCT No.: PCT/US2018/040497

§ 371 Date: Dec. 19, 2019

Related U.S. Application Data
(60) Provisional application No. 62/584,731, filed on Nov. 10, 2017.
(60) Provisional application No. 62/563,635, filed on Sep. 26, 2017.
(60) Provisional application No. 62/527,030, filed on Jun. 29, 2017.

(51) Int. Cl. A01K 67/027; G01N 33/50; A61K 31/675; A61K 35/17; A61K 49/00; C07K 16/28; C07K 14/705; C07K 14/725

(52) U.S. Cl. CPC A01K 67/0271; A61K 2039/5158; G01N 33/5088; A61K 31/675; A61K 35/17; A61K 49/0008; C07K 16/2803; C07K 14/70517; C07K 14/70578; C07K 14/70521; C07K 14/7051; A01K 2267/0331; A01K 2227/105; A01K 2207/20; A01K 2207/12; C07K 2319/30; C07K 2319/33; G01N 33/5014

(57) ABSTRACT

Provided herein is a model, in particular a mouse model, for assessing or evaluating toxicity to an immunotherapy, for example a therapeutic cell therapy, such as a cell therapy containing engineered cells, such as T cells, expressing a recombinant receptor, e.g. a chimeric antigen receptor (CAR). Also provided is a method for generating the mouse model. Also provided herein are methods of use for the mouse models of toxicity, such as to evaluate modified or alternative immunotherapies, and/or to evaluate test agents, including agents to assess as potential interventions to reduce, prevent, or ameliorate toxicity to immunotherapy in human subjects and/or for use in combination with an immunotherapy, e.g. CAR−T cell therapy.

### CROSS-REFERENCE TO RELATED APPLICATIONS

This application is a National Stage application under 35 U.S.C. § 371 of International Application No. PCT/US2018/040497 filed on Jun. 29, 2018, which claims the benefit of priority to U.S. provisional patent application 62/527,030, filed Jun. 29, 2017, entitled “TOXICITY MODEL AND RELATED METHODS,” U.S. provisional patent application 62/563,635, filed Sep. 26, 2017, entitled “MOUSE MODEL FOR ASSESSING TOXICITIES ASSOCIATED WITH IMMUNOTHERAPIES,” and U.S. provisional patent application 62/584,731, filed Nov. 10, 2017, entitled “MOUSE MODEL FOR ASSESSING TOXICITIES ASSOCIATED WITH IMMUNOTHERAPIES,” the contents of which are hereby incorporated by reference in their entirety for all purposes

### INCORPORATION BY REFERENCE OF SEQUENCE LISTING

The present application is being filed along with a Sequence Listing in electronic format. The Sequence Listing is provided as a file entitled 735042011800SeqList.TXT, created Dec. 18, 2019, which is 9,113 bytes in size. The information in the electronic format of the Sequence Listing is incorporated by reference in its entirety.

### FIELD

The present disclosure provides a model, in particular a mouse model, for assessing or evaluating toxicity to an immunotherapy, for example a therapeutic cell therapy, such as a cell therapy containing engineered cells, such as T cells, expressing a recombinant receptor, e.g. a chimeric antigen receptor (CAR). Also provided is a method for generating the mouse model. Also provided herein are methods of use for the mouse models of toxicity, such as to evaluate modified or alternative immunotherapies, and/or to evaluate test agents, including agents to assess as potential interventions to reduce, prevent, or ameliorate toxicity to immunotherapy in human subjects and/or for use in combination with an immunotherapy, e.g. CAR−T cell therapy.

### BACKGROUND

Immunotherapies such a chimeric antigen receptors (CAR) T cell therapies have shown great promise for treating subjects with cancers, including relapsed and refractory B-cell neoplasms, such as acute lymphoblastic leukemia, chronic lymphocytic leukemia, and non-Hodgkin lymphomas. However, despite their success, immunotherapies such as CAR−T cell therapies can be accompanied by adverse effects and toxicity, such as cytokine release syndrome and neurotoxicity. The mechanisms underlying these toxicities are not completely understood. Additional research tools, such as in vivo models of toxicity, are needed to further understand and treat toxicity associated with immunotherapy.

### SUMMARY

Provided herein are methods for generating a mouse model of an immunotherapy-associated toxicity or an immunotherapy-associated toxic outcome, comprising: i) administering a lymphodepleting agent or therapy to an immunocompetent mouse, wherein the lymphodepleting agent or therapy does not comprise total body radiation and/or does not comprise complete or substantially complete immune ablation; and ii) subsequently administering to the mouse an immunotherapy, wherein the immunotherapy binds to and/or recognizes an antigen that is expressed on or in a cell or tissue of the immunocompetent mouse.

In certain embodiments of any of the provided methods, the antigen is an antigen naturally expressed on murine cell and/or the antigen is a cell surface antigen and/or the immunotherapy binds to or recognizes an extracellular epitope of the antigen. In some embodiments of any of the provided methods, the cell is a murine cell. In particular embodiments of any of the provided methods, the antigen is expressed on the surface of a circulating cell or the cell is a circulating cell. In particular embodiments of any of the provided methods, the antigen is a B cell antigen or is expressed on the surface of a B cell or wherein the cell is a murine B cell.

In certain embodiments of any of the provided methods, the immunotherapy is an agent that stimulates or activates immune cells. In particular embodiments of any of the provided methods, the immunotherapy is a T cell-engaging therapy, optionally wherein the T-cell engaging therapy comprises a bispecific antibody, wherein at least one binding portion specifically binds to a T cell antigen, optionally CD3. In some embodiments of any of the provided methods, the amino acid sequence of the T cell-engaging therapy comprises a murine sequence and/or is not immunogenic to the mouse.

In particular embodiments of any of the provided methods, the immunotherapy comprises a cell therapy, said cell therapy optionally comprising a dose or composition of genetically engineered cells expressing a recombinant receptor. In particular embodiments of any of the provided methods, the engineered cells comprise cells obtained from a biological sample from the immunocompetent mouse or from a mouse that is of the same strain or substrain as the immunocompetent mouse. In certain embodiments of any of the provided methods, the biological sample comprises splenocytes. In some embodiments of any of the provided methods, the engineered cells comprise NK cells or T cells, optionally wherein the T cells are CD4+ and/or CD8+ T cells.

Provided herein is a method for generating a mouse model of an immunotherapy-associated toxicity or an immunotherapy-associated toxic outcome, comprising: i) administering a lymphodepleting agent or therapy to an immunocompetent mouse, wherein the lymphodepleting agent or therapy does not comprise total body radiation and/or does not comprise complete or substantially complete immune ablation; and ii) subsequently administering to the mouse a cell therapy comprising murine T cells expressing a recombinant receptor that binds to and/or recognizes a murine antigen that is expressed on a B cell of the immunocompetent mouse.

In particular embodiments of any of the provided methods, the recombinant receptor is a T cell receptor or a functional non-T cell receptor. In particular embodiments of any of the provided methods, the recombinant receptor is a chimeric receptor, optionally a chimeric antigen receptor (CAR). In certain embodiments of any of the provided methods, wherein: the amino acid sequence of the recombinant receptor is murine; and/or the individual regions or domains of the chimeric receptor comprise regions or domains of a natural murine protein and/or comprises a murine sequence; and/or the individual regions or domains of the chimeric receptor are not immunogenic to the mouse. In particular embodiments of any of the provided methods, the recombinant receptor is a chimeric antigen receptor (CAR) and the CAR comprises an extracellular antigen-binding domain that specifically binds to the antigen. In some embodiments of any of the provided methods, the antigen-binding domain is an antibody or an antigen-binding fragment, wherein the antigen-binding fragment is optionally a single chain fragment, optionally an scFv.

In particular embodiments of any of the provided methods, the CAR comprises an intracellular signaling domain comprising an ITAM, wherein optionally, the intracellular signaling domain comprises an intracellular domain of a CD3-zeta (CD3ζ) chain, optionally murine CD3-zeta. In particular embodiments of any of the provided methods, the intracellular signaling domain further comprises a costimulatory signaling region, which optionally comprises a signaling domain of CD28 or 4-1BB, optionally murine CD28 or murine 4-1BB.

In certain embodiments of any of the provided methods, the antigen is or comprises ROR1, B cell maturation antigen (BCMA), carbonic anhydrase 9 (CAIX), Her2/neu (receptor tyrosine kinase erbB2), CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen.

In some embodiments of any of the provided methods, the antigen is B cell maturation antigen (BCMA), CD19, CD20, CD22, CD24, CD30, and/or CD38. In particular embodiments of any of the provided methods, the antigen is CD19. In particular embodiments of any of the provided methods, wherein: the antigen is expressed on cells administered to the mouse; and/or the method comprises administering to the immunocompetent mouse one or more cells expressing the antigen, optionally wherein the antigen-expressing cells are administered prior to administering of the lymphodepleting agent or therapy.

In certain embodiments of any of the provided methods, the antigen is expressed on or in tumor and/or cancer cells and/or the antigen-expressing cells are tumor and/or cancer cells, and wherein: the immunocompetent mouse comprises the tumor and/or cancer cells; and/or the method further comprises administering to the immunocompetent mouse one or more cancer cells and/or a tumor or tumor tissue, optionally prior to the administering of the lymphodepleting agent or therapy. In particular embodiments of any of the provided methods, the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor.

In some embodiments of any of the provided methods, the one or more cancer cells and/or the tumor comprise cancerous B cells, optionally mouse B cells and/or are B cell-derived. In particular embodiments of any of the provided methods, the mouse contains and/or the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

In particular embodiments of any of the provided methods, the mouse contains and/or the one or more cancer cells or tumor cells comprise A20 cells. In certain embodiments of any of the provided methods, the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R. In some embodiments of any of the provided methods, the immunocompetent mouse is not a C57BL/6 mouse or a substrain thereof. In particular embodiments of any of the provided methods, the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

In particular embodiments of any of the provided methods, the immunocompetent mouse, following challenge with an antigen and optionally an adjuvant, has an increase in one or more cytokines compared to an immunocompetent C57BL/6 mouse administered the same antigen, optionally wherein the one or more cytokine is an inflammatory cytokine. In certain embodiments of any of the provided methods, the immunocompetent mouse is a BALB/c mouse or is of a substrain thereof. In particular embodiments of any of the provided methods, the BALB/c mouse or substrain thereof is a BALB/cJ mouse or a BALB/cByJ mouse.

In certain embodiments of any of the provided methods, at or about or within 24 hours after the administering the lymphodepleting agent or therapy the mouse comprises: i) a depletion of a percentage of total circulating lymphocytes of between 10% and 95%, between 30% and 85%, or between about 50% and 75% compared to prior to initiation of the lymphodepleting agent or therapy; and/or ii) a depletion of a percentage of circulating T cells of between 10% and 95%, between 30% and 85%, or between about 50% and 75% compared to prior to initiation of the lymphodepleting agent or therapy; and/or iii) a depletion of a percentage of circulating B cells of between 50% and 99%, 75% and 99%, or 75% and 95% compared to prior to initiation of the lymphodepleting agent or therapy. In particular embodiments of any of the provided methods, the lymphodepleting agent or therapy comprises a chemotherapeutic agent.

In particular embodiments of any of the provided methods, the chemotherapeutic agent comprises one or more a toxin, an alkylating agent, a DNA strand-breakage agent, a topoisomerase II inhibitors, a DNA minor groove binding agents, an antimetabolite, a tubulin interactive agent, a progestin, an adrenal corticosteroid, a luteinizing hormone releasing agent antagonist, a gonadotropin-releasing hormone antagonist, or an antihormonal antigen.

In certain embodiments of any of the provided methods, chemotherapeutic agent comprises one or more of cyclophosphamide, chlorambucil, bendamustine, ifosfamide, prednisone, dexamethasone, cisplatin, carboplatin, oxaliplatin, fludarabine, pentostatin, clardribine, cytarabine, gemcitabine, methotrexate, pralatrexate, vincristine, doxorubicin, mitoxantrone, etoposide, bleomycin or combinations thereof.

In particular embodiments of any of the provided methods, the chemotherapeutic agent is or comprises cyclophosphamide. In particular embodiments of any of the provided methods: the lymphodepleting agent or therapy comprises administering cyclophosphamide at a dose of at least or at least about 50 mg/kg, at least or at least about 100 mg/kg, at least or at least about 200 mg/kg, at least at least about 250 mg/kg, at least or at least about 300 mg/kg, at least or at least about 400 mg/kg, at least or at least about 500 mg/kg or at least or at least about 750 mg/kg or a range between any of the foregoing; or the lymphodepleting agent or therapy comprises administering cyclophosphamide at a dose between or between about 50 mg/kg and 750 mg/kg, 50 mg/kg and 500 mg/kg, 50 mg/kg and 250 mg/kg, 50 mg/kg and 100 mg/kg, 100 mg/kg and 750 mg/kg, 100 mg/kg and 500 mg/kg, 100 mg/kg and 250 mg/kg, 250 mg/kg and 750 mg/kg, 250 mg/kg and 500 mg/kg or 500 mg/kg and 750 mg/kg, each inclusive.

In some embodiments of any of the provided methods, the lymphodepleting agent or therapy comprises administering cyclophosphamide at a dose of 250 mg/kg or about 250 mg/kg. In particular embodiments of any of the provided methods, the dose of cyclophosphamide is administered one time prior to initiation of administration of the immunotherapy. In certain embodiments of any of the provided methods, the cyclophosphamide is administered intraperitoneally. In particular embodiments of any of the provided methods, the cell therapy has not previously been cryofrozen. In particular embodiments of any of the provided methods, initiation of administration of the immunotherapy is between 0.5 hours and 120 hours after administering the lymphodepleting agent or therapy. In some embodiments of any of the provided methods, initiation of administration of the immunotherapy is between 12 hours and 48 hours after administering the lymphodepleting agent or therapy.

In certain embodiments of any of the provided methods, initiation of administration of the immunotherapy is 24 hours or about 24 hours after administering the lymphodepleting agent or therapy. In particular embodiments of any of the provided methods, the cell therapy comprises the administration of from or from about 1×10⁶ to 1×10⁸ total recombinant receptor-expressing cells or total T cells. In particular embodiments of any of the provided methods, a the cell therapy comprises the administration of at least or about at least or at or about 5×10⁶ total recombinant receptor-expressing cells or total T cells, 1×10⁷ total recombinant receptor-expressing cells or total T cells, or 5×10⁷ total recombinant receptor-expressing cells or total T cells.

In some embodiments of any of the provided methods, the method results in a toxicity comprising one or more signs, symptoms or outcomes associated with or selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior. In particular embodiments of any of the provided methods, the one or more signs, symptoms or outcomes is or is associated with inflammation, wherein the inflammation comprises histiocytic granulomatous infiltration, optionally of the liver, lung, spleen, or brain. In certain embodiments of any of the provided methods, the one or more signs, symptoms or outcomes is or is associated with altered level, amount or expression or ratio thereof of one or more molecules in the serum, wherein the one or more molecules is a cytokine, chemokine or growth factor.

In particular embodiments of any of the provided methods, the altered level, amount or expression or ratio thereof of the molecule comprises an increased level, amount or expression compared to the level, amount or expression of the molecule in the mouse prior to administering the lymphodepleting therapy and/or immunotherapy and/or compared to the level, amount or expression of the molecule, on average, in a naïve mouse of the same strain and/or compared to the level, amount or expression of the molecule, on average, in a naïve mouse of the same strain and/or compared to the level, amount or expression of the molecule in a mouse administered a non-target immunotherapy. In particular embodiments of any of the provided methods, the level, amount or expression is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In some embodiments of any of the provided methods, the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2.

In some embodiments, the increased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In particular embodiments of any of the provided methods, the altered level, amount or expression or ratio thereof is or comprises an altered ratio of angiopoetin-2 to angiopoetin-1 (Ang2:Ang1 ratio) in the serum, optionally wherein the altered ratio is an increased ratio. In some embodiments, the Ang2:Ang1 ratio is increased by at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold compared to the Ang2:Ang1 ratio in the mouse prior to administering the lymphodepleting therapy and/or immunotherapy and/or compared to the Ang2:Ang1 ratio, on average, in a naïve mouse of the same strain and/or compared to the Ang2:Ang1 ratio in a mouse administered a non-target immunotherapy.

In some embodiments, the altered level, amount or expression or ratio thereof is or comprises a ratio of angiopoetin-2 to angiopoetin-1 (Ang2:Ang1 ratio) in the serum of at least 1, at least 2, at least 3, at least 4, at least 5, at least 10, at least 15, at least 20, at least 25, at least 30, at least 40, at least 50, at least 100, at least 500, at least 1,000, or at least 5,000 or higher. In some embodiments, the Ang2:Ang1 ratio is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In certain embodiments of any of the provided methods, the altered level, amount or expression or ratio thereof of the molecule comprises a decreased level, amount or expression compared to the level, amount or expression of the molecule in the mouse prior to administering the lymphodepleting therapy and/or immunotherapy and/or compared to the level, amount or expression of the molecule, on average, in a naïve mouse of the same strain and/or compared to the level, amount or expression of the molecule, on average, in a naïve mouse of the same strain and/or compared to the level, amount or expression of the molecule in a mouse administered a non-target immunotherapy. In particular embodiments of any of the provided methods, the level, amount or expression is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In particular embodiments of any of the provided methods, the one or more molecules is selected from among IL-9, VEGF, IL-17E/IL-25, IL-15, IL-22, MIP-3a and IL-12/IL-23p40.

In some embodiments, the decreased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In some embodiments of any of the provided methods, the one or more signs, symptoms or outcomes is or is associated with altered expression or ratio thereof of one or more gene products in a tissue, wherein the tissue is brain. In particular embodiments of any of the provided methods, the one or more gene products is or comprises a polynucleotide or portion thereof, optionally wherein the portion is a partial transcript of the polynucleotide. In certain embodiments of any of the provided methods, the polynucleotide is an RNA, optionally wherein the RNA is a messenger RNA (mRNA). In particular embodiments of any of the provided methods, expression of the one or more gene products or portions thereof is measured by polymerase chain reaction (PCR), northern blotting, Southern blotting, microarray, and/or a sequencing technique. In particular embodiments of any of the provided methods, expression of one or more gene products or portions thereof is assessed by reverse transcriptase PCR (rtPCR) and/or real-time or quantitative PCR (qPCR). In some embodiments of any of the provided methods, the expression of the one or more gene products or portions thereof is assessed by microarray.

In certain embodiments of any of the provided methods, the expression of the one or more gene products or portions thereof is assessed by a sequencing technique, optionally a non-Sanger sequencing technique and/or a next generation sequencing technique. In particular embodiments of any of the provided methods, the expression of the one or more gene products or portions thereof is assessed by massively parallel signature sequencing (MPSS), ion semiconductor sequencing, pyrosequencing, SOLiD sequencing, single molecule real time (SMRT) sequencing, and/or nanopore DNA sequencing. In particular embodiments of any of the provided methods, the expression of the one or more gene products or portions thereof is assessed by RNA sequencing (RNA-seq). In some embodiments of any of the provided methods, the expression of the one or more gene products is increased, optionally is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

In particular embodiments of any of the provided methods, the one or more gene products is associated with or involved in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, or a combination of any of the foregoing. In some embodiments, the one or more gene products are associated with a viral process, a multi-organism cellular process, a reactive oxygen species, a metabolic process, a negative regulation of protein modification process, a positive regulation of cell adhesion, an adhesion of symbiont to host, a cell-substrate adhesion, a chaperone-mediated protein folding, a peptidyl-tyrosine modification, taxis, a defense response to another organism, a sterol biosynthetic process, a cellular response to nitrogen compound, or a combination of any of the foregoing.

In certain embodiments, the one or more gene products is selected from among Acer2 (Alkaline ceramidase 2), Adipoq (Adiponectin), Aif1 (Allograft inflammatory factor 1), Angpt1 (angeopotein 1), Angpt14 (angiopoietin-like 4), Angpt2 (angiopotein 2), Aox1 (Aldehyde oxidase), Aqp4 (Aquaporin-4), Atf3 (cyclic AMP-dependent transcription factor ATF-3), Bnip3 (BCL2/adenovirus E1B 19 kDa protein-interacting protein 3), Ccl2 (C-C motif chemokine 2), CCL4 (MIP-1b, C-C motif chemokine 4), CD31 (PECAM-1), CD274, CD68, CIITA (class II transactivator), CXCL1 (KC, Growth-regulated alpha protein), CXCL10 (IP-10), CXCL11 (I-TAC, C-X-C motif chemokine 11), Edn1 (Endothelin-1), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), HIF3a (hypoxia inducible factor 3 alpha subunit), ICAM-1 (Intercellular adhesion molecule 1), IL2ra (Interleukin-2 receptor subunit alpha), IL-4, IL-6, IL-13, Lrg1 (leucine rich alpha-2-glycoprotien 1), Mgst3 (Microsomal glutathione S-transferase 3), Mmrn2 (Multimerin-2), Ncf1 (Neutrophil cytosol factor 1), NLRC5 (class I transactivator), Nos3 (Nitric oxide synthase, endothelial), Pdk4 (pyruvate dehydrogenase kinase, isozyme 4), Pla2g3 (group 3 secretory phospholipase A2 precursor), Ptgs2 (Prostaglandin G/H synthase 2), Pxdn (Peroxidasin homolog), Scara3 (Scavenger receptor class A member 3), Sele (E-selectin), Selp (P-selectin), IL2ra, IL-13, Serpine 1, Sult1a1 (Sulfotransferase 1A1), Tgfb1 (Transforming growth factor beta-1), Tgfb2 (transforming growth factor beta 2), Tgfb3 (transforming growth factor beta 3), Tgtp1 (T-cell-specific guanine nucleotide triphosphate-binding protein 1), Tlr2 (Toll-like receptor 2), Tlr4 (toll like receptor 4), TNF (tumor necrosis factor), VCAM-1 (Vascular cell adhesion protein 1), Vwf (von Willebrand factor), or Xdh (xanthine dehydrogenase).

In certain embodiments, the one or more gene product is selected from among Adipoq (Adiponectin), Aif1 (Allograft inflammatory factor 1), Aqp4 (Aquaporin-4), Ccl2 (C-C motif chemokine 2), CD68, Edn1 (Endothelin-1), Serpine 1, Tgfb1 (Transforming growth factor beta-1), Tgfb2 (transforming growth factor beta 2), Tgfb3 (transforming growth factor beta 3), Tlr2 (Toll-like receptor 2), Tlr4 (toll like receptor 4), IL2ra, IL-13, Gzmb (Granzyme B), TNF, CXCL10 (IP-10), CCL2 (MCP-1, C-C motif chemokine 2), CXCL11 (I-TAC, C-X-C motif chemokine 11), CXCL1 (KC, Growth-regulated alpha protein), CCL4 (MIP-1b, C-C motif chemokine 4), NLRC5 (class I transactivator), or CIITA (class II transactivator).

In certain embodiments of any of the provided methods, the one or more gene products is associated with or involved in immune response, angiogenesis, sterol metabolic processes, oxidative stress, antioxidant defense, nitric oxide signaling pathway, cell adhesion or a combination of any of the foregoing. In particular embodiments of any of the provided methods, the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31 (PECAM-1). In particular embodiments of any of the provided methods, the expression of the one or more gene products is decreased, optionally is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In some embodiments of any of the provided methods, one or more signs, symptoms or outcomes associated is or is associated with altered blood chemistry and the altered blood chemistry comprises a decrease in serum glucose, serum albumin, total serum protein and/or serum levels of calcium.

In certain embodiments of any of the provided methods, one or more signs, symptoms or outcomes is or is associated with tissue damage, optionally wherein the tissue damage comprises histiocytic granulomatous infiltration of the tissue, necrosis, vascular damage and/or vascular leakage. In particular embodiments of any of the provided methods, one or more signs, symptoms or outcomes is or is associated with altered behavior, optionally wherein the altered behavior comprises reduced food intake, reduced water intake, reduced grooming, and/or reduced locomotor activity.

Provided herein is a mouse model that is produced by the methods herein. Provided herein is a mouse model, the model comprising an immunocompetent mouse comprising: a partial depletion in number of one or more populations of lymphocytes compared to the number of the one or more populations of lymphocytes, on average, in a naïve mouse of the same strain; and an immunotherapy, wherein the immunotherapy binds to and/or recognizes an antigen that is expressed on or in a cell or tissue of the immunocompetent mouse, optionally wherein the immunotherapy is exogenous to the immunocompetent mouse, optionally wherein the immunotherapy is recombinant or chimeric.

In particular embodiments of any of the provided mouse models, the partial depletion is not permanent or is transient, optionally wherein the partial depletion is present for greater than 14 days, 28 days, 45 days, 60 days, 3 months, 6 months, 1 year or more following administration of a lymphodepleting therapy or agent, optionally wherein the lymphodepleting agent or therapy comprises cyclophosphamide.

In some embodiments of any of the provided mouse models, the mouse comprises: i) a depletion of a percentage of total circulating lymphocytes of between 10% and 95%, between 30% and 85%, or between about 50% and 75%; and/or ii) a depletion of a percentage of circulating T cells of between 10% and 95%, between 30% and 85%, or between about 50% and 75%; and/or iii) a depletion of a percentage of circulating B cells of between 50% and 99%, 75% and 99%, or 75% and 95%.

In particular embodiments of any of the provided mouse models, the number of the one or more populations of lymphocytes comprises: between or between about 0.1 and 1,000 lymphocytes per μl of blood; between 0.1 and 1,000 B cells per μl of blood; and/or between 0.1 and 100 T cells per μl of blood. In certain embodiments of any of the provided mouse models, the antigen is an antigen naturally expressed on murine cell and/or the antigen is a cell surface antigen and/or the immunotherapy binds to or recognizes an extracellular epitope of the antigen.

In particular embodiments of any of the provided mouse models, the cell is a murine cell. In particular embodiments of any of the provided mouse models, the antigen is expressed on the surface of a circulating cell or the cell is a circulating cell.

In some embodiments of any of the provided mouse models, the antigen is a B cell antigen or is expressed on the surface of a B cell or wherein the cell is a murine B cell. In certain embodiments of any of the provided mouse models, the immunotherapy is an agent that stimulates or activates immune cells. In particular embodiments of any of the provided mouse models, the immunotherapy is a T cell-engaging therapy, optionally wherein the T-cell engaging therapy comprises a bispecific antibody, wherein at least one binding portion specifically binds to a T cell antigen, optionally CD3. In particular embodiments of any of the provided mouse models, the amino acid sequence of the T cell-engaging therapy comprises a murine sequence and/or is not immunogenic to the mouse. In some embodiments of any of the provided mouse models, the immunotherapy comprises a cell therapy, said cell therapy optionally comprising a dose or composition of genetically engineered cells expressing a recombinant receptor.

In particular embodiments of any of the provided mouse models, the engineered cells comprise cells obtained from a biological sample from the immunocompetent mouse or from a mouse that is of the same strain or substrain as the immunocompetent mouse. In certain embodiments of any of the provided mouse models, the biological sample comprises splenocytes. In particular embodiments of any of the provided mouse models, the engineered cells comprise NK cells or T cells, optionally wherein the T cells are CD4+ and/or CD8+ T cells.

In particular embodiments of any of the provided mouse models, the recombinant receptor is a T cell receptor or a functional non-T cell receptor. In some embodiments of any of the provided mouse models, the recombinant receptor is a chimeric receptor, optionally a chimeric antigen receptor (CAR). In certain embodiments of any of the provided mouse models: the amino acid sequence of the recombinant receptor is murine; and/or the individual regions or domains of the chimeric receptor comprise regions or domains of a natural murine protein and/or comprises a murine sequence; and/or the individual regions or domains of the chimeric receptor are not immunogenic to the mouse.

In particular embodiments of any of the provided mouse models, the recombinant receptor is a chimeric antigen receptor (CAR) and the CAR comprises an extracellular antigen-binding domain that specifically binds to the antigen. In particular embodiments of any of the provided mouse models, the antigen-binding domain is an antibody or an antigen-binding fragment, wherein the antigen-binding fragment is optionally a single chain fragment, optionally an scFv. In some embodiments of any of the provided mouse models, the CAR comprises an intracellular signaling domain comprising an ITAM, wherein optionally, the intracellular signaling domain comprises an intracellular domain of a CD3-zeta (CD3ζ) chain, optionally murine CD3-zeta. In particular embodiments of any of the provided mouse models, the intracellular signaling domain further comprises a costimulatory signaling region, which optionally comprises a signaling domain of CD28 or 4-1BB, optionally murine CD28 or murine 4-1BB.

In certain embodiments of any of the provided mouse models, the antigen is or comprises ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen.

In particular embodiments, the antigen is or comprises αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is a human antigen. In certain embodiments, the antigen is a mouse antigen.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In particular embodiments of any of the provided mouse models, the antigen is B cell maturation antigen (BCMA), CD19, CD20, CD22, CD24, CD30, and/or CD38. In particular embodiments of any of the provided mouse models, the antigen is CD19. In some embodiments of any of the provided mouse models, the mouse comprises one or more exogenous cells expressing the antigen. In certain embodiments of any of the provided mouse models, the exogenous antigen-expressing cells comprise tumor and/or cancer cells. In particular embodiments of any of the provided mouse models, the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor.

In particular embodiments of any of the provided mouse models, the one or more cancer cells and/or the tumor cells comprise cancerous B cells, optionally mouse B cells and/or are B cell-derived. In some embodiments of any of the provided mouse models, the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells. In particular embodiments of any of the provided mouse models, the one or more cancer cells and/or tumor cells comprise A20 cells.

In certain embodiments of any of the provided mouse models, the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R. In particular embodiments of any of the provided mouse models, the immunocompetent mouse is not a C57BL/6 mouse or a substrain thereof. In particular embodiments of any of the provided mouse models, the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

In some embodiments of any of the provided mouse models, the immunocompetent mouse, following challenge with an antigen and optionally an adjuvant, has an increase in one or more cytokines compared to an immunocompetent C57BL/6 mouse administered the same antigen, optionally wherein the one or more cytokine is an inflammatory cytokine. In certain embodiments of any of the provided mouse models, the immunocompetent mouse is a BALB/c mouse or is of a substrain thereof. In particular embodiments of any of the provided mouse models, the BALB/c mouse or substrain thereof is a BALB/cJ mouse or a BALB/cByJ mouse.

In particular embodiments of any of the provided mouse models, the immunocompetent mouse exhibits one or more signs, symptoms or outcomes associated with a toxicity and/or selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior.

In some embodiments of any of the provided mouse models, the toxicity and/or the one or more signs, symptoms or outcomes is or is associated with inflammation, wherein the inflammation comprises histiocytic granulomatous infiltration, optionally of the liver, lung, spleen, or brain. In particular embodiments of any of the provided mouse models, the toxicity and/or the one or more signs, symptoms or outcomes is or is associated with altered level, amount or expression or ratio thereof of one or more molecules in the serum, wherein the one or more molecules is a cytokine, chemokine or growth factor. In certain embodiments of any of the provided mouse models, the altered level, amount or expression or ratio thereof of the molecule comprises an increased level, amount or expression compared to the level, amount or expression of the molecule, on average, in a naïve mouse of the same strain and/or compared to the level, amount or expression of the molecule in a mouse administered a non-target immunotherapy.

In particular embodiments of any of the provided mouse models, level, amount or expression is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In particular embodiments of any of the provided mouse models, the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2.

In some embodiments, the increased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In particular embodiments of any of the provided methods, the altered level, amount or expression or ratio thereof is or comprises an altered ratio of angiopoetin-2 to angiopoetin-1 (Ang2:Ang1 ratio) in the serum, optionally wherein the altered ratio is an increased ratio. In some embodiments, the Ang2:Ang1 ratio is increased by at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold compared to the Ang2:Ang1 ratio in the mouse prior to administering the lymphodepleting therapy and/or immunotherapy and/or compared to the Ang2:Ang1 ratio, on average, in a naïve mouse of the same strain and/or compared to the Ang2:Ang1 ratio in a mouse administered a non-target immunotherapy.

In some embodiments, the altered level, amount or expression or ratio thereof is or comprises a ratio of angiopoetin-2 to angiopoetin-1 (Ang2:Ang1 ratio) in the serum of at least 1, at least 2, at least 3, at least 4, at least 5, at least 10, at least 15, at least 20, at least 25, at least 30, at least 40, at least 50, at least 100, at least 500, at least 1,000, or at least 5,000 or higher. In some embodiments, the Ang2:Ang1 ratio is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In some embodiments of any of the provided mouse models, the altered level, amount or expression or ratio thereof of the molecule comprises a decreased level, amount or expression compared to the level, amount or expression of the molecule, on average, in a naïve mouse of the same strain and/or compared to the level, amount or expression of the molecule in a mouse administered a non-target immunotherapy.

In certain embodiments of any of the provided mouse models, the level, amount or expression is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In particular embodiments of any of the provided mouse models, the one or more molecules is selected from among IL-9, VEGF, IL-17E/IL-25, IL-15, IL-22, MIP-3a and IL-12/IL-23p40. In some embodiments, the decreased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

In particular embodiments of any of the provided mouse models, the toxicity and/or the one or more signs, symptoms or outcomes is or is associated with altered expression or ratio thereof of one or more gene products in a tissue, wherein the tissue is brain. In some embodiments of any of the provided mouse models, the one or more gene products is or comprises a polynucleotide or portion thereof, optionally wherein the portion is a partial transcript of the polynucleotide.

In particular embodiments of any of the provided mouse models, the polynucleotide is an RNA, optionally wherein the RNA is a messenger RNA (mRNA). In certain embodiments of any of the provided mouse models, expression of the one or more gene products or portions thereof is determined by polymerase chain reaction (PCR), northern blotting, Southern blotting, microarray, and/or a sequencing technique. In particular embodiments of any of the provided mouse models, expression of one or more gene products or portions thereof is determined by reverse transcriptase PCR (rtPCR) and/or real-time or quantitative PCR (qPCR). In particular embodiments of any of the provided mouse models, the expression of the one or more gene products or portions thereof is determined by microarray.

In some embodiments of any of the provided mouse models, the expression of the one or more gene products or portions thereof is determined by a sequencing technique, optionally a non-Sanger sequencing technique and/or a next generation sequencing technique. In certain embodiments of any of the provided mouse models, the expression of the one or more gene products or portions thereof is assessed by massively parallel signature sequencing (MPSS), ion semiconductor sequencing, pyrosequencing, SOLiD sequencing, single molecule real time (SMRT) sequencing, and/or nanopore DNA sequencing. In particular embodiments of any of the provided mouse models, the expression of the one or more gene products or portions thereof is assessed by RNA sequencing (RNA-seq). In particular embodiments of any of the provided mouse models, the expression of the one or more gene products is increased, optionally is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

In particular embodiments, the one or more gene products is associated with or involved in wherein the one or more gene products is associated with or involved in viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound.

In some embodiments of any of the provided mouse models, the one or more gene products is associated with or involved in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

In particular embodiments of any of the provided mouse models, the one or more gene products is associated with or involved in immune response, angiogenesis, sterol metabolic processes, oxidative stress, antioxidant defense, nitric oxide signaling pathway, cell adhesion or a combination of any of the foregoing.

In certain embodiments of any of the provided methods, the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31. In particular embodiments of any of the provided mouse models, the expression of the one or more gene products is decreased, optionally is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more. In particular embodiments of any of the provided mouse models, the toxicity and/or the one or more signs, symptoms or outcomes is or is associated with altered blood chemistry and the altered blood chemistry comprises a decrease in serum glucose, serum albumin, total serum protein and/or serum levels of calcium.

In some embodiments of any of the provided mouse models, the toxicity and/or the one or more signs, symptoms or outcomes is or is associated with tissue damage, optionally wherein the tissue damage comprises histiocytic granulomatous infiltration of the tissue, necrosis, vascular damage and/or vascular leakage. In certain embodiments of any of the provided mouse models, the toxicity and/or the one or more signs, symptoms or outcomes is or is associated with altered behavior, optionally wherein the altered behavior comprises reduced food intake, reduced water intake, reduced grooming, and/or reduced locomotor activity. In particular aspects a tissue sample is provided that is obtained from a mouse produced by the methods provided herein.

In particular embodiments of the provided tissues, the tissue sample is or comprises blood, serum, brain tissue, liver tissue, lung tissue, kidney tissue, and/or spleen tissue. In some embodiments of the provided tissues, the tissue sample is or comprises brain tissue.

Provided herein is a method of identifying and/or assessing one or more effects of an agent, the method comprising: i) administering a lymphodepleting agent or therapy and an immunotherapy to an immunocompetent mouse to generate a toxicity and/or one or more sign, symptom or outcome associated with or indicative of a toxic outcome or side effect; ii) administering a test agent, optionally at a test dosage regimen or frequency of the test agent, to the immunocompetent mouse; and iii) assessing the toxicity and/or one or more of the sign, symptom, or outcome in the mouse.

In particular embodiments of the provided methods, the test agent is administered prior to, subsequent to, or concurrently and/or or simultaneously with initiation of administration of the lymphodepleting agent or therapy and/or initiation of administration of the immunotherapy. In certain embodiments of the provided methods, the test agent is administered prior to initiation of administration of the lymphodepleting agent or therapy and/or initiation of administration of the immunotherapy.

In particular embodiments of the provided methods, the method further comprising: iv) comparing the toxicity and/or the one or more of the sign, symptom, or outcome to a control mouse, the control mouse having been administered the lymphodepleting agent or therapy and the immunotherapy but not the test agent, wherein the control mouse is immunocompetent.

Provided herein is a method of identifying and/or assessing one or more effects of an agent, the method comprising: i) administering a test agent, optionally at a test dosage regimen or frequency of the test agent, to an immunocompetent mouse, the immunocompetent mouse having been previously administered a lymphodepleting agent or therapy and an immunotherapy, wherein the immunocompetent mouse exhibits a toxicity and/or one or more sign, symptom or outcome associated with or indicative of a toxic outcome or side effect; and ii) assessing the toxicity and/or the one or more sign, symptom, or outcome in the mouse. In particular embodiments of the provided methods, the immunocompetent mouse is a mouse produced by the methods provided herein, or is any immunocompetent mouse or any mouse model provided herein.

In some embodiments of the provided methods, the method further comprises: iii) comparing the toxicity and/or the one or more sign, symptom, or outcome to a control mouse, the control mouse having been administered the lymphodepleting agent or therapy and the immunotherapy but not the test agent, wherein the control mouse is immunocompetent. In certain embodiments of the provided methods, the test agent is administered subsequent to the administration of the lymphodepleting agent or therapy and/or the immunotherapy.

In particular embodiments of the provided methods, the test dosage regimen of the test agent is for assessing if a particular or predetermined amount or concentration of the test agent for administration and/or the dosing frequency of the agent for administration alters the toxicity and/or one or more of the sign, symptom, or outcome in the mouse. In particular embodiments of the provided methods, the test agent comprises a small molecule, a small organic compound, a peptide, a polypeptide, an antibody or antigen binding fragment thereof, a non-peptide compounds, a synthetic compound, a fermentation product, a cell extract, a polynucleotide, an oligonucleotide, an RNAi, an siRNA, an shRNA, a multivalent siRNA, an miRNA, and/or a virus. In some embodiments of the provided methods, the test agent, optionally the test dosage regimen of the test agent, is a candidate for ameliorating the toxicity and/or the sign, symptom, or outcome.

In particular embodiments of the provided methods, if the comparison indicates the toxicity and/or the sign, symptom, or outcome is altered, optionally reduced, in the presence of the test agent, optionally the test dosage regimen of the test agent, the test agent is identified as an agent for ameliorating toxicity to the immunotherapy or likely to or predicted to ameliorate toxicity to the immunotherapy. In certain embodiments of the provided methods, the test agent, optionally the test dosage regimen of the test agent, is an agent for use in combination with the cell therapy, optionally wherein the agent is or is likely or is a candidate to improve the activity, efficacy, survival and/or persistence of the cell therapy. In particular embodiments of the provided methods, if the comparison indicates the toxicity and/or the sign, symptom, or outcome is altered, optionally increased, in the presence of the test agent, optionally the test dosage regimen of the test agent, the test agent or test dosage regimen is identified as exacerbating the toxicity to the immunotherapy or is likely to or predicted to exacerbate toxicity to the immunotherapy.

In particular embodiments of the provided methods, the toxicity comprises and/or the one or more signs, symptoms or outcomes associated with the toxicity is selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior. In certain embodiments of the provided methods, the toxicity and/or the one or more signs, symptoms or outcomes is or is associated with inflammation, wherein the inflammation comprises histiocytic granulomatous infiltration, optionally of the liver, lung, spleen, or brain.

In particular embodiments of the provided methods, the toxicity and/or the one or more signs, symptoms or outcomes is or is associated with altered level, amount or expression or ratio thereof of one or more molecules in the serum, wherein the one or more molecules is a cytokine, chemokine or growth factor. In particular embodiments of the provided methods, the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2. In some embodiments of the provided methods, the one or more molecules is selected from among IL-9, VEGF, IL-17E/IL-25, IL-15, IL-22, MIP-3a and IL-12/IL-23p40.

In certain embodiments of the provided mouse model, the toxicity and/or the one or more signs, symptoms or outcomes is or is associated with altered expression or ratio thereof of one or more gene products in a tissue, wherein the tissue is brain. In particular embodiments of the provided methods, the one or more gene products is or comprises a polynucleotide or portion thereof, optionally wherein the portion is a partial transcript of the polynucleotide. In some embodiments of the provided methods, the polynucleotide is an RNA, optionally wherein the RNA is a messenger RNA (mRNA). In certain embodiments of the provided methods, the one or more gene products is associated with or involved in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

In particular embodiments of the provided methods, the one or more gene products is associated with or involved in immune response, angiogenesis, sterol metabolic processes, oxidative stress, antioxidant defense, nitric oxide signaling pathway, cell adhesion or a combination of any of the foregoing. In some embodiments of the provided methods, the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31.

In certain embodiments of the provided methods, the toxicity and/or the one or more signs, symptoms or outcomes is or is associated with altered blood chemistry and the altered blood chemistry comprises a decrease in serum glucose, serum albumin, total serum protein and/or serum levels of calcium. In particular embodiments of the provided methods, the toxicity and/or the one or more signs, symptoms or outcomes is or is associated with tissue damage, optionally wherein the tissue damage comprises histiocytic granulomatous infiltration of the tissue, necrosis, vascular damage and/or vascular leakage. In some embodiments of the provided methods, the toxicity and/or the one or more signs, symptoms or outcomes is or is associated with altered behavior, optionally wherein the altered behavior comprises reduced food intake, reduced water intake, reduced grooming, and/or reduced locomotor activity.

In certain embodiments of the provided methods, assessing the toxicity and/or the one or more sign, symptom, or outcome in the mouse is determined by polymerase chain reaction (PCR), northern blotting, Southern blotting, microarray, a sequencing technique, an immunoassay, flow cytometry, histochemistry, monitoring weight, monitoring temperature, and/or observing physical, phenotypic and/or behavioral changes or features. In particular embodiments of the provided methods, the expression of the one or more gene products or portions thereof is assessed by RNA sequencing (RNA-seq). In some embodiments of the provided methods, the lymphodepleting agent or therapy does not comprise total body radiation and/or does not comprise complete or substantially complete immune ablation.

In certain embodiments of the provided methods, the immunotherapy binds to and/or recognizes an antigen that is expressed on or in a cell or tissue of the immunocompetent mouse. In particular embodiments of the provided methods, the antigen is an antigen naturally expressed on murine cell and/or the antigen is a cell surface antigen and/or the immunotherapy binds to or recognizes an extracellular epitope of the antigen. In some embodiments of the provided methods, the cell is a murine cell. In certain embodiments of the provided methods, the antigen is expressed on the surface of a circulating cell or the cell is a circulating cell.

In particular embodiments of the provided methods, the antigen is a B cell antigen or is expressed on the surface of a B cell or wherein the cell is a murine B cell. In some embodiments of the provided methods, the immunotherapy is an agent that stimulates or activates immune cells. In certain embodiments of the provided methods, the immunotherapy is a T cell-engaging therapy, optionally wherein the T-cell engaging therapy comprises a bispecific antibody, wherein at least one binding portion specifically binds to a T cell antigen, optionally CD3. In particular embodiments of the provided methods, the amino acid sequence of the T cell-engaging therapy comprises a murine sequence and/or is not immunogenic to the mouse.

In some embodiments of the provided methods, the immunotherapy comprises a cell therapy, said cell therapy optionally comprising a dose or composition of genetically engineered cells expressing a recombinant receptor. In certain embodiments of the provided methods, the engineered cells comprise cells obtained from a biological sample from the immunocompetent mouse or from a mouse that is of the same strain or substrain as the immunocompetent mouse.

In particular embodiments of the provided methods, the biological sample comprises splenocytes. In certain embodiments of the provided methods, the engineered cells comprise NK cells or T cells, optionally wherein the T cells are CD4+ and/or CD8+ T cells. In some embodiments of the provided methods, the recombinant receptor is a T cell receptor or a functional non-T cell receptor.

In particular embodiments of the provided methods, the recombinant receptor is a chimeric receptor, optionally a chimeric antigen receptor (CAR). In certain embodiments of the provided methods: the amino acid sequence of the recombinant receptor is murine; and/or the individual regions or domains of the chimeric receptor comprise regions or domains of a natural murine protein and/or comprises a murine sequence; and/or the individual regions or domains of the chimeric receptor are not immunogenic to the mouse. In some embodiments of the provided methods, the recombinant receptor is a chimeric antigen receptor (CAR) and the CAR comprises an extracellular antigen-binding domain that specifically binds to the antigen.

In particular embodiments of the provided methods, the antigen-binding domain is an antibody or an antigen-binding fragment, wherein the antigen-binding fragment is optionally a single chain fragment, optionally an scFv. In certain embodiments of the provided methods, the CAR comprises an intracellular signaling domain comprising an ITAM, wherein optionally, the intracellular signaling domain comprises an intracellular domain of a CD3-zeta (CD3ζ) chain, optionally murine CD3-zeta. In some embodiments of the provided methods, the intracellular signaling domain further comprises a costimulatory signaling region, which optionally comprises a signaling domain of CD28 or 4-1BB, optionally murine CD28 or murine 4-1BB.

In particular embodiments of the provided methods, the antigen is or comprises ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen.

In certain embodiments of the provided methods, the antigen is B cell maturation antigen (BCMA), CD19, CD20, CD22, CD24, CD30, and/or CD38. In some embodiments of the provided methods, the antigen is CD19. In particular embodiments of the provided methods: the antigen is expressed on cells administered to the mouse; and/or the method comprises administering to the immunocompetent mouse one or more cells expressing the antigen, optionally wherein the antigen-expressing cells are administered prior to administering of the lymphodepleting agent or therapy. In certain embodiments of the provided methods, the antigen is expressed on or in tumor and/or cancer cells and/or the antigen-expressing cells are tumor and/or cancer cells, and wherein: the immunocompetent mouse comprises the tumor and/or cancer cells; and/or the method further comprises administering to the immunocompetent mouse one or more cancer cells and/or a tumor or tumor tissue, optionally prior to the administering of the lymphodepleting agent or therapy.

In some embodiments of the provided methods, the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor. In particular embodiments of the provided methods, the one or more cancer cells and/or the tumor comprise cancerous B cells, optionally mouse B cells and/or are B cell-derived. In certain embodiments of the provided methods, the mouse contains and/or the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

In some embodiments of the provided methods, the mouse contains and/or the one or more cancer cells or tumor cells comprise A20 cells. In particular embodiments of the provided methods, the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R. In certain embodiments of the provided methods, the immunocompetent mouse is not a C57BL/6 mouse or a substrain thereof. In some embodiments of the provided methods, the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

In particular embodiments of the provided methods, the immunocompetent mouse, following challenge with an antigen and optionally an adjuvant, has an increase in one or more cytokines compared to an immunocompetent C57BL/6mouse administered the same antigen, optionally wherein the one or more cytokine is an inflammatory cytokine. In certain embodiments of the provided methods, the immunocompetent mouse is a BALB/c mouse or is of a substrain thereof. In some embodiments of the provided methods, the BALB/c mouse or substrain thereof is a BALB/cJ mouse or a BALB/cByJ mouse. In particular embodiments of the provided methods, the lymphodepleting agent or therapy comprises a chemotherapeutic agent. In certain embodiments of the provided methods, the chemotherapeutic agent comprises one or more a toxin, an alkylating agent, a DNA strand-breakage agent, a topoisomerase II inhibitors, a DNA minor groove binding agents, an antimetabolite, a tubulin interactive agent, a progestin, an adrenal corticosteroid, a luteinizing hormone releasing agent antagonist, a gonadotropin-releasing hormone antagonist, or an antihormonal antigen. In particular embodiments of the provided methods, chemotherapeutic agent comprises one or more of cyclophosphamide, chlorambucil, bendamustine, ifosfamide, prednisone, dexamethasone, cisplatin, carboplatin, oxaliplatin, fludarabine, pentostatin, clardribine, cytarabine, gemcitabine, methotrexate, pralatrexate, vincristine, doxorubicin, mitoxantrone, etoposide, bleomycin or combinations thereof. In certain embodiments of the provided methods, the chemotherapeutic agent is or comprises cyclophosphamide.

In some embodiments of the provided methods: the lymphodepleting agent or therapy comprises administering cyclophosphamide at a dose of at least or at least about 50 mg/kg, at least or at least about 100 mg/kg, at least or at least about 200 mg/kg, at least at least about 250 mg/kg, at least or at least about 300 mg/kg, at least or at least about 400 mg/kg, at least or at least about 500 mg/kg or at least or at least about 750 mg/kg or a range between any of the foregoing; or the lymphodepleting agent or therapy comprises administering cyclophosphamide at a dose between or between about 50 mg/kg and 750 mg/kg, 50 mg/kg and 500 mg/kg, 50 mg/kg and 250 mg/kg, 50 mg/kg and 100 mg/kg, 100 mg/kg and 750 mg/kg, 100 mg/kg and 500 mg/kg, 100 mg/kg and 250 mg/kg, 250 mg/kg and 750 mg/kg, 250 mg/kg and 500 mg/kg or 500 mg/kg and 750 mg/kg, each inclusive.

In particular embodiments of the provided methods, the lymphodepleting agent or therapy comprises administering cyclophosphamide at a dose of 250 mg/kg or about 250 mg/kg. In certain embodiments of the provided methods, the dose of cyclophosphamide is administered one time prior to initiation of administration of the immunotherapy. In some embodiments of the provided methods, the cyclophosphamide is administered intraperitoneally. In particular embodiments of the provided methods, initiation of administration of the immunotherapy is between 0.5 hours and 120 hours after administering the lymphodepleting agent or therapy. In certain embodiments of the provided methods, initiation of administration of the immunotherapy is between 12 hours and 48 hours after administering the lymphodepleting agent or therapy. In some embodiments of the provided methods, initiation of administration of the immunotherapy is 24 hours or about 24 hours after administering the lymphodepleting agent or therapy. In particular embodiments of the provided methods, the cell therapy comprises the administration of from or from about 1×10⁶ to 1×10⁸ total recombinant receptor-expressing cells or total T cells. In particular embodiments of the provided methods, a the cell therapy comprises the administration of at least or about at least or at or about 5×10⁶ total recombinant receptor-expressing cells or total T cells, 10×10⁶ total recombinant receptor-expressing cells or total T cells, or 50×10⁶ total recombinant receptor-expressing cells or total T cells.

In some embodiments, the antigen is a B cell antigen or is expressed on the surface of a B cell or wherein the cell is a murine B cell. In certain embodiments, the antigen is expressed on cells administered to the mouse. In various embodiments, the cells expressing the antigen are tumor cells. In some embodiments, the cells expressing the antigen are administered prior to initiating administration of the lymphodepleting agent or therapy or the immunotherapy.

Provided herein is a method for generating a mouse model of an immunotherapy-associated toxicity or an immunotherapy-associated toxic outcome, comprising: i) administering to an immunocompetent mouse tumor cells that express an antigen; ii) after administering the tumor cells, administering a lymphodepleting agent or therapy to the immunocompetent mouse, wherein the lymphodepleting agent or therapy does not comprise total body radiation and/or does not comprise complete or substantially complete immune ablation; and iii) subsequently administering to the mouse an immunotherapy, wherein the immunotherapy binds to and/or recognizes the antigen that is expressed on the tumor cells.

Provided herein is a method for generating a mouse model of an immunotherapy-associated toxicity or an immunotherapy-associated toxic outcome, comprising: i) administering a lymphodepleting agent or therapy to an immunocompetent mouse comprising tumor cells that express an antigen, optionally wherein the tumor cells had been administered to the mouse prior to initiation of administration of the lymphodepleting agent or therapy, wherein the lymphodepleting agent or therapy does not comprise total body radiation and/or does not comprise complete or substantially complete immune ablation; and ii) subsequently administering to the mouse an immunotherapy, wherein the immunotherapy binds to and/or recognizes the antigen that is expressed on the tumor cells.

In certain embodiments, the tumor cells are administered in an amount sufficient to form a tumor in the mouse. In various embodiments, the lymphodepleting agent or therapy and/or the immunotherapy is administered to the mouse at a time after tumor burden in the mouse comprises: a tumor size greater than or greater than about or about 5 mm, greater than or greater than about or about 10 mm, greater than or greater than about or about 15 mm, optionally 5 mm to 15 mm or 10 mm to 15 mm in diameter; and/or a tumor volume of greater than or greater than about or about 60 mm³, greater than or greater than about or about 70 mm³, greater than or greater than about or about 80 mm³, greater than or greater than about or about 90 mm³, or greater than or greater than about or about 100 mm³. In some embodiments, the tumor cells are administered between or between about 7 days and 28 days, 14 days and 21 days, or 17 days and 19 days, each inclusive, prior to initiation of administration of the lymphodepleting agent or therapy or the immunotherapy. In certain embodiments, the tumor cells are administered at or about 17 days, 18 days, or 19 days prior to administration of the immunotherapy. In various embodiments, the tumor cells are administered at or about 27 days prior to administration of the immunotherapy. tumor cell is a B cell cancer cell line.

In some embodiments, the B cell cancer cell line is selected from L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells or a combination thereof.

In certain embodiments, the B cell cancer cell line comprises A20 cells. In various embodiments, the cell therapy comprises murine T cells expressing a recombinant receptor that binds to and/or recognizes a murine antigen that is expressed on a B cell of the immunocompetent mouse In some embodiments, the cell therapy comprises the administration of between or between about 5×10⁶ and about 5×10⁷ total recombinant receptor-expressing cells or total T cells.

Provided herein is a mouse model, comprising an immunocompetent mouse comprising: a partial depletion in number of one or more populations of lymphocytes compared to the number of the one or more populations of lymphocytes, on average, in a naïve mouse of the same strain; an immunotherapy, wherein the immunotherapy binds to and/or recognizes an antigen, wherein the immunotherapy is exogenous to the immunocompetent mouse, optionally wherein the immunotherapy is recombinant or chimeric; and tumor cells comprising the antigen, optionally wherein the antigen is expressed on the tumor cell surface.

In various embodiments, the B cell cancer cell line is selected from L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells. In some embodiments, the B cell cancer cell line comprises A20 cells.

In certain embodiments, the immunotherapy comprises a cell therapy, said cell therapy comprising genetically engineered cells expressing a recombinant receptor. In various embodiments, the engineered cells comprise cells obtained from a biological sample from the immunocompetent mouse or from a mouse that is of the same strain or substrain as the immunocompetent mouse. In some embodiments, the biological sample comprises splenocytes. In certain embodiments, the cell therapy comprises murine T cells expressing a recombinant receptor that binds to and/or recognizes a murine antigen that is expressed on a B cell of the immunocompetent mouse. In various embodiments, the recombinant receptor is a T cell receptor or a functional non-T cell receptor. In some embodiments, the recombinant receptor is a chimeric receptor, optionally a chimeric antigen receptor (CAR). In certain embodiments, the antigen is B cell maturation antigen (BCMA), CD19, CD20, CD22, CD24, CD30, and/or CD38. In various embodiments, the antigen is CD19.

### BRIEF DESCRIPTION OF THE DRAWINGS

FIGS. 1A-1D show graphs displaying the amounts of circulating cells in mice that were administered 100 mg/kg mg/kg cyclophosphamide i.p. (100 mpk CPA) or 250 mg/kg cyclophosphamide i.p. (250 mpk CPA) and a cell composition containing cells expressing an anti-mouse CD19 chimeric antigen receptor (CD19 CAR−T) or a cell composition that did not contain CAR expressing cells (mock). FIG. 1A shows the level of circulating Thy1.1+ cells, indicating CAR expression) in 100 mpk CPA+CD19 CAR−T and 100 mpk CPA+CD19 CAR−T treatment groups. Circulating levels of B cells (FIG. 1B), T cells (FIG. 1C), and CD11b+ cells (FIG. 1D) of mice that received treatment with 100 mpk CPA; 100 mpk CPA+CD19 CAR−T; 250 mpk CPA; 250 mpk CPA+mock; and 250 mpk CPA+CD19 CAR are shown.

FIGS. 2A-2V show levels of circulating cytokines in naïve mice and mice that received treatment with 250 mg/kg CPA i.p. (250 mpk CPA), 250 mg/kg CPA i.p. and a mock cell composition (CPA+mock), and 250 mg/kg CPA i.p. and a T cell composition containing T cells expressing anti an anti-mouse CD19 chimeric antigen receptor (CPA+CAR−T). Circulating levels of IL-2 (FIG. 2A), IL-4 (FIG. 2B), IL-5 (FIG. 2C), GM-CSF (FIG. 2D), IFN-gamma (FIG. 2E), TNF-alpha (FIG. 2F), IL-10 (FIG. 2G), MIP-1b (FIG. 2H), MCP-1 (FIG. 2I), IL-6 (FIG. 2J), Angiopoietin-2 (FIG. 2K) EPO (FIG. 2L), IL-12p70 (FIG. 2M), IL-13 (FIG. 2N), IL-15 (FIG. 2O), IL-17E/IL-25 (FIG. 2P), IL-21 (FIG. 2Q), IL-23 (FIG. 2R), IL-30 (FIG. 2S), IP-10 (FIG. 2T), KC/GRO (FIG. 2U), and MIP-1a (FIG. 2V) are shown.

FIG. 2W shows the circulating IL-6 levels in naïve mice and mice that received treatment with 250 mg/kg cyclophosphamide (CPA) i.p, 250 mg/kg CPA i.p. and anti-mouse CD19 CAR-expressing T cells 24 hours later (CPA+muCD19 CAR−T) or 250 mg/kg CPA i.p. and non-target control anti-human CD19 CAR+ T cells 24 hours later (CPA+control CAR−T) cells. Serum IL-6 levels were determined on days 2, 5 and 6 after infusion of CAR T cells.

FIG. 2X shows the serum Angiopoietin-2: Angiopoietin-1 ratio (Ang2:Ang1 ratio) in naïve mice and mice that received treatment with 250 mg/kg cyclophosphamide (CPA) i.p, 250 mg/kg CPA i.p. and anti-mouse CD19 CAR-expressing T cells 24 hours later (CPA+muCD19 CAR−T) or 250 mg/kg CPA i.p. and non-target control anti-human CD19 CAR+ T cells 24 hours later (CPA+control CAR−T) cells. Serum Angiopoietin-2 and Angiopoietin-1 levels were determined on days 2, 5 and 6 after infusion of CAR T cells.

FIGS. 3A-3G shows graphs displaying the levels of circulating cells in blood as assessed by flow cytometry of naïve mice or mice that were treated with 250 mg/kg CPA i.p. (250 mpk CPA only), 250 mg/kg CPA i.p. and a mock cell composition (CPA+Mock), and 250 mg/kg CPA i.p. and 5×10⁶ cells of a T cell composition containing T cells expressing anti an anti-mouse CD19 chimeric antigen receptor (CPA+5e6 muCD19 CAR−T) at 24 hours, 48 hours, or 72 hours after treatment with the cells. Circulating levels of total live CD45+ cells (FIG. 3A), CD11b+ cells (FIG. 3B), T cells (FIG. 3C), B cells (FIG. 3D), and CAR−T (Thy1.1+; FIG. 3E) cells are shown. The ratio of circulating CD4+ to CD8+CAR−T cells (FIG. 3F) and total T cells (FIG. 3G) are also shown. Results of statistical analysis comparing CPA+Mock and CPA+CAR−T treated mice at 24 hour, 48 hour, and 72 hour time points by 2-way ANOVA are shown along the X-axis: ns=not significant; *=p<0.05; **=p<0.01; ***=p<0.001; ****=p<0.0001.

FIGS. 4A-4H displays levels of cells in mice that were naïve or treated with 250 mg/kg CPA i.p. (CPA), 250 mg/kg CPA i.p. and a mock cell composition (CPA+Mock), and 250 mg/kg CPA i.p. and a T cell composition containing T cells expressing anti an anti-mouse CD19 chimeric antigen receptor (CPA+CAR−T) at 24 hours, 48 hours, or 72 hours after treatment with the cells. FIG. 4A shows flow cytometry plots displaying Thy1.2+ gated T cells (Y axis) and Thy1.1 (CAR+) X axis. Results from cells isolated from blood (top row), spleen (middle row), and brain (bottom row) are shown for naïve mice (left column) and mice treated with CPA+Mock (middle column) and CPA+CAR−T (right column). Graphs displaying levels CAR−T (Thy1.1+) cells (FIG. 4B), T cells (FIG. 4D), B cells (FIG. 4F), total CD45+ live cells (FIG. 4G), and CD11b+ cells (FIG. 4H) isolated from brains of individual mice are shown. FIGS. 4C and 4E show graphs displaying the percentage of CAR−T cells (FIG. 4C) and total T cells (FIG. 4E) that are CD4 positive. Results of statistical analysis comparing CPA+Mock and CPA+CAR−T treated mice at 24 hour, 48 hour, and 72 hour time points by 2-way ANOVA are shown along the X-axis: ns=not significant; *=p<0.05; **=p<0.01; ***=p<0.001; ****=p<0.0001.

FIGS. 5A and 5B show results of RNA sequencing (RNA-seq) analysis brains of mice that were naïve (0h) or treated with 250 mg/kg CPA i.p. (CPA), 250 mg/kg CPA i.p. and a mock cell composition (CPA+mock T), and 250 mg/kg CPA i.p. and a an anti-mouse CD19 CAR−T cell composition (CPA+CAR−T). Brains were collected at 24 hours, 48 hours, or 72 hours after treatment with the cells. FIG. 5A shows alignments of transcripts that were detected by RNA-seq in brains that encode the scFv region of anti-mouse CD19 CAR. FIG. 5B shows a graph displaying the levels of transcripts that encode the scFv region of anti-mouse CD19 chimeric antigen receptor (CAR) expressed by transcripts per million (TPM).

FIGS. 6A and B show results of an RNA-Seq analysis. FIG. 6A shows heat map depicting gene expression in brains from naïve mice or mice treated with 250 mg/kg CPA i.p. (CPA alone), 250 mg/kg CPA i.p. and a mock cell composition (CPA+Mock), or 250 mg/kg CPA i.p. and an anti-mouse CD19 CAR T-cell composition (CY+CAR−T). The scale indicates the log 10 Q-value. FIG. 6B shows a summary of the ontological enrichment analysis performed on results from RNA-seq gene expression analysis. Thirty gene ontology categories with the largest amounts of differentially expressed genes detected in brain tissues from mice treated with 250 mg/kg CPA i.p. and a T cell composition containing T cells expressing anti an anti-mouse CD19 CAR are listed. The amount of differentially expressed genes detected in each category (out of 3,558 total differentially expressed genes), the amount of genes detected in in each category (out of 17,589 total genes detected), the enrichment fold, and the enrichment omega values are shown.

FIGS. 7A-7P show graphs displaying results from RNA sequencing (RNA-seq) analysis. The expression of individual genes from brains of mice treated with 250 mg/kg CPA i.p. (CPA), 250 mg/kg CPA i.p. and a mock cell composition (CPA+Mock), and 250 mg/kg CPA i.p. and an anti-mouse CD19 CAR T-cell composition (CPA+CAR−T) that were collected 24 hours, 48 hours, and 72 hours after cells were administered are shown. The gene expression was normalized to the expression of the genes in brains collected from naïve mice, and is displayed as transcripts per million (TPM). Exemplary genes from different categories are shown. FIGS. 7A and 7B show the expression of exemplary adhesion molecule genes VCAM-1, ICAM-1, Sele (E-Selectin), SELP (P-Selectin) and CD31. FIGS. 7C and 7D show the expression of exemplary immune response genes GBP2, GBP4, GBP5, and GBP9. FIGS. 7E-7G show the expression of exemplary angiogenesis genes Angpt2, Angp14, Hif3a, Lrg1, Mmrn2, and Xdh. FIGS. 7H-7J show the expression of exemplary sterol metabolic process genes Acer2, Atf3, Pdk4, Pla2g3, and Sult1a1. FIGS. 7K-7M show the expression of exemplary oxidative stress and antioxidant defense genes Ncf1, Aox1, Bnip3, Pxdn, Scara3, Mgst3, and Ptgs2. FIGS. 7N and 7O show the expression of exemplary nitric oxide signaling pathway genes Ncf1, Nos3, and Scara3. FIG. 7P shows expression levels of exemplary cytokine encoding genes IL-4, IL-6, and GM-CSF.

FIG. 8 shows a graph displaying results from RNA sequencing (RNA-seq) analysis. The expression of individual genes from brains of mice treated with 250 mg/kg CPA i.p. (CPA), 250 mg/kg CPA i.p. and a mock cell composition (CPA+Mock), and 250 mg/kg CPA i.p. and an anti-mouse CD19 CAR T-cell composition (CPA+CAR−T) that were collected 24 hours, 48 hours, and 72 hours after cells were administered are shown. The gene expression was normalized to the expression of the genes in brains collected from naïve mice and is displayed as TPM. The expression of CD274 (PD-L1), Tgtp1, and Vwf are shown.

FIGS. 9A-9D. show the graphs displaying the results of serum chemistry analysis of serum samples taken at 24 hours, 48 hours, 72 hours, and 5 days after administration of cells collected from naïve mice and mice treated with 250 mg/kg CPA i.p. (CPA), 250 mg/kg CPA i.p. and a mock cell composition (CPA+Mock), and 250 mg/kg CPA i.p. and a T cell composition containing T cells expressing anti an anti-mouse CD19 chimeric antigen receptor (CPA+CAR−T). FIGS. 11A-11D show levels of serum glucose (FIG. 9A), serum albumin (FIG. 9B) and serum calcium (FIG. 9D) as well as the serum ratio of albumin to globulin (FIG. 9C). Statistical comparisons are shown: *=p<0.05; **=p<0.01; ***=p<0.001; ****=p<0.0001.

FIG. 10 shows a graph displaying changes in body weight in naïve mice (No Tx) and mice treated with CPA (250 mg/kg CPA), CPA and a mock cell composition (250 mg/kg CPA+Mock CAR−T), and CPA and a T cell composition containing T cells expressing anti an anti-mouse CD19 CAR (250 mg/kg CPA+CAR−T). Weights were measured at the time of CPA treatment (day 0), and on days 1-4 following CPA treatment.

FIGS. 11A-11C display results of histopathology analysis of tissues from naïve mice and mice treated with 250 mg/kg CPA i.p. (CPA), 250 mg/kg CPA i.p. and a mock cell composition (CPA+Mock), and 250 mg/kg CPA i.p. and a T cell composition containing T cells expressing anti an anti-mouse CD19 chimeric antigen receptor (CPA+CAR−T). Scores of the severity of histiocytic granulomatous infiltration observed in liver (FIG. 11A), lung (FIG. 11B), and spleen (FIG. 11C) are shown.

FIG. 12 shows graphs displaying changes in body weight. The top graph displays changes in body weights of naïve mice and mice treated with CPA and a mock cell composition (CPA+Mock), and CPA and an anti-mouse CD19 CAR−T cell composition (CPA+CAR−T). The bottom graph shows changes in body weights of naïve mice, and mice that were administered A20 cells (A20), A20 cells and CPA (A20+CPA); A20 cells, CPA, and a mock cell composition (A20+CPA+Mock); and A20 cells, CPA and an anti-mouse CD19 CAR−T cell composition (A20+CPA+CAR−T).

FIGS. 13A-13D show graphs that display results of histopathology analysis of spleen collected 3 days and 6 days after administration of cells from naive mice and mice treated with CPA and a mock cell composition (CPA+Mock); CPA and an anti-mouse CD19 CAR−T cell composition (CPA+CAR−T), and mice that were administered A20 cells (A20); A20 cells and CPA (A20+CPA); A20 cells, CPA, and a mock cell composition (A20+CPA+Mock); and CPA and an anti-mouse CD19 CAR−T cell composition (A20+CPA+CAR−T). Spleens were rated for severity of lymphoid depletion (FIG. 13A), extramedullary hematopoiesis (FIG. 13B), fibrosis (FIG. 13C), and histiocytic granulomatous infiltration (FIG. 13D).

FIGS. 14A and 14B show graphs that display results of histopathology analysis of liver collected 3 days and 6 days after administration of cells from naive mice and mice treated with CPA and a mock cell composition (CPA+Mock); CPA and an anti-mouse CD19 CAR−T cell composition (CPA+CAR−T), and mice that were administered A20 cells (A20); A20 cells and CPA (A20+CPA); A20 cells, CPA, and a mock cell composition (A20+CPA+Mock); and CPA and an anti-mouse CD19 CAR−T cell composition (A20+CPA+CAR−T). Livers were rated for extramedullary hematopoiesis (FIG. 14A) and histiocytic/granulomatous infiltration (FIG. 14B).

FIGS. 15A and 15B show graphs displaying tumor mass of spleen (FIG. 15A) and liver (FIG. 15B) collected 3 days and 6 days after administration of cells from naive mice and mice treated with CPA and a mock cell composition (CPA+Mock); CPA and an anti-mouse CD19 CAR−T cell composition (CPA+CAR−T), and mice that were administered A20 cells (A20); A20 cells and CPA (A20+CPA); A20 cells, CPA, and a mock cell composition (A20+CPA+Mock); and CPA and an anti-mouse CD19 CAR−T cell composition (A20+CPA+CAR−T).

FIGS. 16A-16C show graphs displaying changes in body weight (FIG. 16A), body temperature (FIG. 16B), and brain water content (FIG. 16C) in naïve mice and mice treated with CPA, CPA and 10⁷ cells of a control anti-human CD19 CAR−T cell composition (CPA+10e6 control CAR−T), and CPA and 10⁷ cells of an anti-mouse CD19 CAR−T cell composition (CPA+10e6 muCD19 CAR−T). Weights were measured at the time of CPA treatment (day −1), at the time of administering cells (day 0), and on days 1-5 following cell treatment.

FIG. 17 shows graphs displaying amounts of IL-4, IL-6, and CM-CSF protein detected in brain tissue in A20 tumor cell bearing mice that received no treatment, treatment with cyclophosphamide (cy alone), treatment with cyclophosphamide and anti-human CD19CAR−T cells (cy+control CAR−T), or treatment with cyclophosphamide and anti-mouse CD19 CAR−T cells (cy+muCD19 CAR−T).

FIGS. 18A-18K provide graphs displaying concentrations of cytokines in serum collected at different time points following CAR−T cell injection in mice injected with A20 tumor cells that received no treatment (A20 tumor only), treatment with cyclophosphamide and anti-human CD 19CAR−T cells (+Cy+Control CAR−T), or treatment with cyclophosphamide and anti-mouse CD19 CAR−T cells (+Cy+muCD19 CAR−T). Concentrations of IFN-gamma (FIG. 18A), TNF-alpha (FIG. 18B), GM-CSF (FIG. 18C), IL-2 (FIG. 18D), IL4 (FIG. 18E), IL-5(FIG. 18F), IL-6 (FIG. 18G), IL-10 (FIG. 18H), MIP-1b (FIG. 18I), and MCP-1 (FIG. 18J) that were detected between 0 and 5 days after CAR−T cell injection are shown. The ratio of the concentrations of angiopoietin 2 to angiopoietin 1 detected in serum at 2 and 5 days following CAR−T cell injection are shown in FIG. 18K.

FIGS. 19A-19N display graphs depicting results of RNA-Seq analysis.

FIG. 19A provides a heat map displaying clustering of samples on all stably expressed genes (>5 TPM) of samples from perfused brain tissues of A20 tumor bearing mice that received no treatment (A20-tumor No Tx), treatment with cyclophosphamide and anti-human CD19 CAR−T cells (+Cy+control CAR−T), or treatment with cyclophosphamide and anti-mouse CD19 CAR−T cells (+Cy+mCD19 CAR−T). The scale indicates the log 10 Q-value.

FIG. 19B provides a table and a graph summarizing ontological enrichment analysis. Twenty gene GO categories with the largest amounts of differentially expressed genes detected in brain tissues from mice injected with A20 tumor cells and treated with 250 mg/kg cyclophosphamide i.p. and a T cell composition containing T cells expressing anti an anti-mouse CD19 CAR are listed. The amount of differentially expressed genes detected in each category (out of 1,822 total differentially expressed genes), the amount of genes detected in in each category (out of 17,783 total genes detected), and the enrichment Q-values are shown.

FIG. 19C shows a heat map depicting the expression of exemplary differentially expressed genes associated with inflammation and vascular changes in brains of A20 tumor bearing mice that received no treatment (A20-tumor No Tx), treatment with cyclophosphamide and anti-human CD19 CAR−T cells (+Cy+control CAR−T), or treatment with cyclophosphamide and anti-mouse CD19 CAR−T cells (+Cy+mCD19 CAR−T).

FIGS. 19D-19N show graphs displaying results of individual gene expression in brains collected 48 hours after CAR−T cell injection. For each graph, the expression of individual genes from brains of A20 tumor bearing mice that received no treatment (A20-tumor No Tx; left bar), treatment with cyclophosphamide and anti-human CD19 CAR−T cells (A20-tumor, +CPA+control CAR−T; middle bar), or treatment with cyclophosphamide and anti-mouse CD19 CAR−T cells (A20-tumor, +CPA+muCD19 CAR−T; right bar) are shown. The gene expression is displayed as transcripts per million (TPM). Exemplary genes associated with inflammation and vascular changes (FIG. 19D), immune response (FIG. 19E), angiogenesis (FIGS. 19F and 19G), sterol metabolic processes (FIGS. 19H and 191), adhesion molecules (FIGS. 19J and 19K), cytokines, chemokines, and MHC proteins (FIGS. 19L and 19M) and other exemplary genes (FIG. 19N) are shown. For statistical comparisons, NS=not significant, *=p<0.05, **=p<0.01, ***=p<0.001, ****=p<0.0001, by one-way ANOVA with Sidak's multiple comparison test.

### DETAILED DESCRIPTION

Provided herein is a mouse model of toxicity to an immunotherapy e.g., a cell therapy, and methods for generating and carrying out research with the same. In some aspects, the model is or includes a mouse with a reduced number of B cells, e.g., B cell aplasia, that contains an immunotherapy, such as an engineered cell that expresses a recombinant receptor, e.g., a CAR. In some embodiments, provided herein are methods of generating a mouse model of toxicity to an immunotherapy, for example by administering a lymphodepleting agent or therapy and an immunotherapy to a mouse, such as an immunocompetent mouse. In some embodiments, such methods result in signs symptoms or outcomes that resemble aspects of administering the immunotherapy in human subjects. In some embodiments, these aspects include adverse effects such as toxicity, such as toxicity associated with systemic and/or neuroinflammation. In some embodiments, the mouse models provided herein are useful as preclinical models to research, study, and/or investigate one or more aspects administering an immunotherapy, such as expansion, persistence, and activity of an immunotherapy, as well as mechanisms and potential interventions of toxicity associated with immunotherapy.

Immunotherapies, such as adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies), can be effective in treating cancer and other diseases and disorders. In certain contexts, available approaches to immunotherapy, such as adoptive cell therapy, may not always be entirely satisfactory. In some contexts, one or more desired outcomes, such as optimal efficacy, can depend on the ability of the administered cells (e.g., the ability of one or more subpopulations thereof) to carry out one or more activities or functions and/or to exhibit one or more particular properties. In some aspects, optimal efficacy depends upon the cells' ability to recognize and bind to a target, e.g., target antigen; in some aspects, it depends upon the ability of the cells to traffic, localize to and/or successfully enter and/or circulate through one or more appropriate sites within the subject, such as sites or tissues expressing target antigen or in which activity is desired. Exemplary of sites for entry are tumors, and environments thereof, e.g., microenvironments, vasculature, and/or lymphoid system or organs. Optimal efficacy typically depends upon the ability of the cells to become activated, expand, and/or to exert various effector functions, including cytotoxic killing and/or secretion of various factors such as cytokines. Optimal efficacy may depend upon the ability of the engineered cells to persist in desired locations or environments and/or for desired periods of time, such as long-term and/or within tumor or disease environments. In some aspects, optimal efficacy may depend upon at least a subset of the cells' ability to differentiate, transition or engage in reprogramming into one or more certain phenotypic states (such as effector, long-lived memory, less-differentiated, and effector states). In some embodiments, optimal efficacy may depend upon the cells' ability to effect recall responses, such as robust and effective recall responses, in contexts following clearance and re-exposure to target ligand or antigen, such as following clearance of disease (such as reexposure to antigen, such as in the context of relapse, in a subject having previously achieved complete remission, optionally minimal residual disease (MRD) negative remission); thus, in some aspects, optimal efficacy may depend on the ability of cells to and avoid adopting a less-optimal state or phenotype following initial or early exposure to antigen, such as the ability of the cells to avoid becoming exhausted or anergic or terminally differentiated (or to exhibit reduced degrees of exhaustion anergy terminal differentiation compared to a reference cell population). In some aspect, optimal efficacy may depend upon the cells' ability to avoid adopting or differentiating into a suppressive state.

In some aspects, the provided embodiments are based on observations that the efficacy of adoptive cell therapy may be limited in some context by the development of, or risk of developing, toxicity or one or more toxic outcomes in the subject to whom such cells are administered. In some cases, such toxicities can be severe. For example, in some cases, administering a dose of cells expressing a recombinant receptor, e.g. a CAR, can result in toxicity or risk thereof, such as CRS or neurotoxicity. In some cases, risk of one or more toxic outcomes may increase in a manner correlated with increases of properties associated with improved efficacy. For example, while in some contexts the administration of relatively higher doses of such cells and/or combination therapy with an additional agent to increase activity, efficacy and/or persistence of administered cells can increase efficacy, for example, by increasing exposure to the cells such as by promoting expansion and/or persistence, they may also result in an even greater risk of developing a toxicity or a more severe toxicity. Similarly, while the co-administration of one or more agents to promote immune function, may in some contexts promote desired activity and function such as secretion of cytokines and target-specific cytotoxicity, and/or reduce suppressive factors, it may in certain aspects also be associated with an increased risk of one or more factors associated with toxicity.

Certain available methods for treating or ameliorating toxicity may not always be entirely satisfactory. In some contexts, available methods for treating or ameliorating toxicity are limited or hindered by a lack of understanding in the cause of toxicity. For example, it has not been entirely understood how some therapies and/or particular cell therapy or therapies may cause or be at risk for leading to toxicity, such as CRS, neurotoxicity, and/or cerebral edema. Many available approaches focus, for example, on targeting downstream effects of toxicity, such as by cytokine blockade, and/or delivering agents such as high-dose steroids which can also eliminate or impair the function of administered cells. Additionally, such approaches often involve administration of such interventions only upon detection of physical signs or symptoms of toxicity and/or certain degrees or levels thereof, which in general involve signs or symptoms of moderate or severe toxicity (e.g. moderate or severe CRS), which in many cases may be associated with risk of inefficacy of the intervention and/or require administration of greater dosage or higher intensity intervention, which may be associated with one or more undesirable side effects and/or reduce efficacy of the therapy. In some embodiments, available approaches are not entirely satisfactory in their ability to reduce or prevent one or more of various forms of toxicity such as neurotoxicity.

In some cases, available agents and/or therapies aimed at reducing or ameliorating therapy-associated toxicity (e.g. steroids) are themselves associated with toxic side effects. The intensity of such side effects may be greater at higher dosages of the agents and/or therapies, such as at the relatively higher dose or frequency that may be required in order to treat or ameliorate the severity of the toxicity at the time administered, e.g., after the sign or symptom or level or degree thereof. In addition, in some aspects, the available agent or therapy for treating a toxicity may limit the efficacy of the cell therapy, such as the efficacy of the chimeric receptor (e.g. CAR) expressing cells provided as part of the cell therapy (Sentman Immunotherapy, 5:10 (2013)), e.g., by reducing activity or one or more desired downstream effects induced by such therapy.

Understanding the signs, symptoms or outcomes of administration of immunotherapy, e.g., cell therapy, is useful for evaluating current or potential therapies, particularly in the context of potential life-threatening side effects (e.g. cytokine release syndrome or severe neurotoxicity). In some aspects, such signs, symptoms or outcomes may involve systemic effects or tissue-specific, such as brain-specific, effects that can lead to undesirable outcomes of an immunotherapy, e.g. cell therapy, e.g. CAR−T cell therapy.

In some embodiments, provided herein are mouse models, and methods for generating mouse models, that are useful tools to study, investigate, and/or evaluate aspects of a immunotherapy, for example, such as mechanisms of toxicity to immunotherapies. In some embodiments, the mouse models provided herein may be used to assay potential interventions or agents that may reduce the toxicity, including potential interventions or agents that would treat or prevent the toxicity while minimizing any loss of the cell therapy's efficacy. For example, in some embodiments, mouse models provided herein are useful in order to evaluate and prioritize potential agents and or interventions among numerous agents that could potentially prevent or treat neurotoxicity. In some embodiments, the mouse models provided herein also can be useful to identify new intervening agents and/or identify new pathways to target for intervention. In some aspects, employment of a preclinical mouse model of toxicity enables more efficient and robust evaluation or prioritization of such agents and/or identification of the potential for use of such agents.

Certain embodiments contemplate that there is a lack of animal models, and in particular mouse models, that are suitable for studying toxicity to an immunotherapy. Without being bound by theory, in some embodiments, administering an immunotherapy to a mouse will not necessarily result in any signs, symptoms, or features associated with toxicity in the mouse. In some embodiments, whether any signs, symptoms, or features associated with toxicity will manifest following administration of an immunotherapy to the mouse may depend on previously unidentified factors, such as but not limited to genetic background of the mouse, the target or dose of the immunotherapy, and/or the manner and timing of lymphodepletion. In some embodiments, the methods provided herein demonstrate that mice can be used to generate an animal model of toxicity to an immunotherapy, and furthermore, provide the steps and conditions necessary to do so. The mouse models provided herein are contemplated to serve a role for, among other uses, preclinical studies, for example, to study and identify underlying mechanisms of toxicity, evaluating new immunotherapies, and for identifying potential interventions to prevent or reduce toxicity.

While the mouse model provided herein is useful for investigating toxicity to an immunotherapy, research applications of this model are not limited to studies of toxicity. For example, in some embodiments, the mouse model provided herein may be used to evaluate features of an immunotherapy that are not related to toxicity, such as in vivo expansion, persistence, and activity of an immunotherapy, such as a cell therapy, e.g. CAR−T cell therapy. For example, in some embodiments, the mouse model may be used to evaluate how administering a second therapeutic agent with the immunotherapy may affect the rate of expansion of the immunotherapy and/or the activity or ability of the immunotherapy to remove tumor or cancer cells in vivo and/or to effect or exacerbate a new or underlying toxicity of the immunotherapy or combined therapy. In some such embodiments, the second therapeutic agent may be administered prior to, concurrently with, or subsequent to the immunotherapy. In some embodiments, the timing of the administration of the immunotherapy and the second therapeutic agent is such that both agents are present in a subject at the same time and/or the second therapeutic agent is present at therapeutic levels during a time period at which the levels of the first therapeutic agent is present at, or is predicted or projected to be present at or within, levels within the subject or an organ or fluid or tissue thereof corresponding to a therapeutic window of the immunotherapy. Thus, in some embodiments, the mouse models provided herein are useful for evaluating or assessing any aspect or outcome of administering an immunotherapy.

The provided methods and mouse models offer particular advantages for the study of immunotherapies, e.g., researching toxicity to immunotherapy. Mice have many similarities to humans in terms of anatomy, physiology, and genetics; grow and breed quickly; are small; and have a relatively short lifespan. This allows for relatively complex biological questions to be addressed within a relatively short time period. With respect to the study of immunotherapies, developing a mouse model that reflects certain aspects of administering the immunotherapy in humans, e.g., toxicity that can, in some cases, develop from the immunotherapy, allows for the evaluation or assessment of various different conditions, adjustments, and variations of a given immunotherapy or procedure for predation or administration of the immunotherapy, that would not be possible in models developed in other animals. Thus, in some embodiments, the mouse models provided herein represent a useful tool for the study and understanding of many different aspects of immunotherapy treatments.

Particular embodiments of the methods provided herein utilize immunotherapies that target antigens expressed on circulating cells to generate toxicity in the mouse. For example, in some embodiments, the immunotherapy is a cell composition containing engineered cells, e.g., CAR expressing cells, that target an antigen expressed on or in B cells, e.g., CD19. Circulating cells provide a target that is dispersed throughout the body, as opposed to a localized target, e.g., an antigen expressed on a solid tumor. In some embodiments, the dispersed nature of the target can allow for rapid expansion of engineered cells, widespread inflammation, robust release of cytokines, and/or damage to multiple organs or tissues. Thus, in some embodiments, the methods for generating a mouse model of toxicity provided herein include one or more steps of administering an immunotherapy that targets an antigen expressed on a circulating cell.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. MOUSE MODEL OF TOXICITY TO AN IMMUNOTHERAPY

Provided herein are mouse models of toxicity to an immunotherapy. In some embodiments, the mouse models provided herein are or include mice that are administered an immunotherapy, e.g., a composition of cells expressing a chimeric antigen receptor (CAR), and a lymphodepleting agent or therapy, e.g., cyclophosphamide (CPA). In certain embodiments, the mouse models provided herein are or include mice that have a reduced level or amount of one or more populations of immune cells that are administered an immunotherapy. In particular embodiments, the mouse model provided herein is or includes a mouse that is administered an immunotherapy subsequent to the administration of a lymphodepleting agent or therapy. In certain embodiments, the mouse model provided herein is or includes a mouse that has a reduced level or amount of one or more populations of lymphocytes, e.g., T cells and/or B cells, and contains at least a portion of the immunotherapy. For example, in some embodiments, the immunotherapy is circulating in the mouse and/or is present in one or more organs and tissues. In certain embodiments, the level or amount of one or more populations of lymphocytes have been reduced by a prior treatment with a lymphodepleting agent or therapy. In some embodiments, the immunotherapy is a cell composition that contains one or more cells expressing a recombinant receptor, e.g., CAR. In some embodiments, the recombinant receptor binds to and/or recognizes a mouse antigen, such as a mouse B cell antigen or mouse CD19.

In certain embodiments, provided herein are methods for generating a mouse of the mouse model. In some embodiments, the methods include one or more steps of administering an immunotherapy and one or more steps of reducing one or more populations of lymphocytes in a mouse. In certain embodiments, the one or more populations of lymphocytes are reduced by administering a lymphodepleting agent or therapy. In some embodiments, the methods provided herein include one or more steps of administering an immunotherapy to a mouse with a reduced population of one or more lymphocytes. In certain embodiments, the methods include one or more steps of administering an immunotherapy to a mouse that has been administered a lymphodepleting agent or therapy. In some embodiments, the methods include detecting and/or measuring a sign, symptom, or outcome of the model. In certain embodiments, the one or more signs, symptoms, or outcomes is or is related to toxicity. In some embodiments, an immunotherapy that is a cell composition containing one or more cells expressing a recombinant receptor, e.g., a CAR, is administered. In some embodiments, an immunotherapy that is or includes cell composition containing one or more cells expressing a recombinant receptor, e.g., a CAR, that binds to and/or recognizes a mouse antigen, such as a mouse B cell antigen or mouse CD19.

In particular embodiments, the immunotherapy is administered to a mouse with a reduction of one or more populations of lymphocytes. In certain embodiments, the reduction of one or more populations of lymphocytes is not a complete reduction, removal, or ablation of the lymphocytes. Thus, in certain embodiments, the immunotherapy is administered to a mouse that has an amount of one or more populations of lymphocytes. In certain embodiments, administration of the lymphodepleting agent or therapy does not completely reduce, remove, or ablate one or more populations of lymphocytes. In some embodiments, the immunotherapy and/or the lymphodepleting agent are administered to an immunocompetent mouse, e.g., a mouse that is capable of having a normal or unimpaired immune response. In some embodiments, the mouse has a reduction of lymphocytes, where the reduction is not a complete reduction. In particular embodiments, the mouse is not an immunocompromised mouse, such as a mouse of an immunocompromised mouse strain and/or a mouse that is suitable for and/or capable of receiving a xenograft, e.g., human cells, without experiencing an immune response.

In some embodiments, the methods provided herein result in reduced or depleted levels of B cells in the mouse. In certain embodiments, the methods provided herein result in B cell aplasia. In particular embodiments, the mouse is administered a lymphodepleting agent and an immunotherapy that binds to and/or targets B cells. In certain embodiments, a sign, symptom, or outcome of the model is or includes B cell aplasia.

In some embodiments, the mice of the model provided herein may be administered one or more cells. In some embodiments, the one or more cells are antigen-expressing cells that express an antigen that is recognized and/or bound by the immunotherapy. In certain embodiments, the antigen-expressing cells provide additional targets for the immunotherapy, e.g., CAR-expressing cells. In some embodiments, the administration of the antigen-expressing cells may increase the expansion, persistence, and/or activity of the immunotherapy, such as by providing additional targets for the immunotherapy. In certain embodiments, the administration of the antigen-expressing cells may alter a sign, symptom, or outcome that is associated with the mouse model. For example, in some embodiments, antigen expressing cells may increase the degree or severity of one or more signs, symptoms, of outcomes or toxicity. In certain embodiments, antigen expressing cells are cancer and/or tumor cells, the clearance of which may be used to assess the activity of the immunotherapy. In certain embodiments, the effect of an additional agent or test agent on the activity of an immunotherapy may be evaluated in a mouse of the mouse model provided herein that contains and/or has been administered antigen expressing cells. In particular embodiments, the antigen-expressing cells are mouse cells and/or are of a stable cell line that is derived from mouse cells. In some embodiments, the antigen-expressing cells are syngeneic to the mouse.

In some embodiments, mouse models provided herein are useful for studying signs, symptoms, and/or outcomes that are associated with the mouse model. In some embodiments, the mouse model provided herein replicates and/or is a model for one or more signs, symptoms, and/or outcomes seen in human subjects. In some embodiments, the one or more signs, symptoms, or outcomes are or include the in vivo expansion, persistence, distribution and/or activity of the immunotherapy. In particular embodiments, the models provided herein are particularly useful to model toxicity, such as CRS or neurotoxicity, that can occur in humans. In some embodiments, the model provided herein is useful for the assessment, evaluation, and/or research of aspects, symptoms, characteristics, and/or features that may contribute to or may be associated with toxicity observed in humans to a cell therapy, e.g., severe neurotoxicity and/or CRS. In some embodiments, these features include but are not limited to elevated serum cytokines and alterations of blood chemistry indicative of systemic inflammatory responses, drops in serum albumin and glucose levels, changes in gene expression associated with cytokine and chemokine expression, microglial activation, endothelial inflammation, and oxidative stress, pathology observed in organs including spleen, liver, and lung, and reductions of body weight and body temperature.

In some embodiments, the mice of the toxicity model provided herein may have one or more cancerous cells, e.g., cancerous B cells. In certain embodiments, the cancer cells provide additional targets for the immunotherapy, e.g., CAR expressing cells. For example, in some embodiments, the mice are injected with cancerous and/or tumorigenic B cells that express CD19 and then are subsequently infused with cells expressing anti-CD19 CARs. In certain embodiments, the additional targets provided by the cancerous B cells lead to a rapid in vivo expansion of the CAR expressing cells. In some embodiments, the rapid in vivo expansion is accompanied by a high degree of severity or toxicity. Thus, in some embodiments, the degree or severity of the toxicity in the model is increased or enhanced in mice injected with or otherwise having cancerous and/or tumorigenic cells.

In some embodiments, mouse models provided herein are useful for studying toxicity associated with immunotherapy. In some embodiments, the mouse model provided herein replicates one or more signs, symptoms, and/or outcomes seen in human subjects with associated with immunotherapies such as T cell therapy. In certain embodiments, signs, symptoms, and/or symptoms are signs, symptoms, and/or symptoms of toxicity. In particular embodiments, the models provided herein are particularly useful to model toxicity, such as CRS or neurotoxicity, that can occur in humans. In certain embodiments, the models provided herein are useful as preclinical models of toxicity to immunotherapies that include therapeutic T cell therapies, such as with recombinant antigen receptor expressing cells, e.g., chimeric antigen receptors (CARs). In certain embodiments, the models provided herein model one or more aspects, symptoms, characteristics, and/or features that contribute to or are associated with toxicity observed in humans to a cell therapy, e.g., severe neurotoxicity and/or CRS. In certain embodiments, such symptoms, characteristics, and/or features include, but are not limited to, rapid in vivo expansion of engineered cells, in vivo expansion of CAR expressing cells into different tissues, including brain tissue. In some embodiments, these features include but are not limited to elevated serum cytokines and alterations of blood chemistry indicative of systemic inflammatory responses, drops in serum albumin and glucose levels, changes in gene expression associated with cytokine and chemokine expression, microglial activation, endothelial inflammation, and oxidative stress, pathology observed in organs including spleen, liver, and lung, and reductions of body weight and body temperature.

In some embodiments, the mouse models provided herein are useful for modeling a toxicity to an immunotherapy, e.g., a neurotoxicity to an immune cell therapy such as a CAR T cell therapy. In certain embodiments, the provided mouse models are generated by administering a lymphodepleting agent or therapy to an immunocompetent mouse, and then subsequently administering an immunotherapy. In particular embodiments, the immunocompetent mouse is not a C57BL/6 mouse or a strain or substrain thereof. In particular embodiments, the administration of the lymphodepleting agent or therapy does not result in complete immune ablation. In certain embodiments, the immunotherapy binds to or recognizes an antigen that is expressed by cell or tissue of or within the immunocompetent mouse.

In certain embodiments, the provided mouse models are generated by (i) injecting antigen-expressing cells, e.g., cancer cells, to an immunocompetent mouse, (ii) subsequently administering a lymphodepleting agent or therapy and then (iii) subsequently administering an immunotherapy that binds to or recognizes the antigen of the antigen expressing cells. In certain embodiments, the antigen-expressing cells do not trigger an immune response in the mouse. In particular embodiments, the immunocompetent mouse is not a C57BL/6 mouse or a strain or substrain thereof. In particular embodiments, the administration of the lymphodepleting agent or therapy does not result in complete immune ablation.

A. Mice

In some embodiments, a mouse model of toxicity is generated and/or produced by performing, implementing, and/or executing the methods provided herein on a mouse. In certain embodiments, the mouse is an adult mouse. In particular embodiments, the mouse is a male mouse. In some embodiments, the mouse is a female mouse. In particular embodiments, the mouse is about or at least about 3 weeks, 4 weeks, 5 weeks, 6 weeks, 7 weeks, 8 weeks, 9 weeks, 10 weeks, 11 weeks, 12 weeks, 14 weeks, 16 weeks, 18 weeks, 20 weeks, 24 weeks, 28 weeks, 32 weeks, 6 months, 9 months, 12 months, 15 months, 18 months, 21 months, or about or at least about 24 months old.

In some embodiments, the mouse is not an immunodeficient and/or an immunosuppressed mouse. In some embodiments, immunodeficient and/or immunosuppressed mice include strains and substrains of mice that can be used for a xenograft, e.g., a human tumor and/or cancer cell xenograft. In some embodiments, an immunodeficient and/or immunosuppressed mouse is a mouse than can be injected with a protein or a cell from another species, e.g., human, without rejecting and/or experiencing an immune response to the foreign protein or cell. Examples of immunodeficient and/or immunosuppressed mice include athymic nude mice, severely compromised immunodeficient (SCID) mice, and non-obese diabetic (NOD)/SCID humanized mice. In some embodiments, a mouse model of toxicity is generated and/or produced by performing, implementing, and/or executing the methods provided herein on a mouse that is not an immunocompromised and/or an immunosuppressed mouse.

In particular embodiments, the mouse expresses and/or is capable of expressing one or more humanized or chimeric proteins. In certain embodiments, the one or more humanized or chimeric proteins are proteins that are expressed and/or released by an immune cell. In certain embodiments, the immune cell is a lymphocyte. In particular embodiments, the lymphocyte is a T cell or a B cell. In particular embodiments, the one or more humanized or chimeric proteins are or include humanized or chimeric MHC proteins and/or humanized or chimeric TCR proteins. In certain embodiments, the one or more humanized and/or chimeric proteins are or include human or chimeric antibodies.

In some embodiments, the mouse is immunocompetent. In certain embodiments, an immunocompetent mouse is a mouse that is able to develop an immune response, for example, to an antigen. In some embodiments, an immunocompetent mouse is capable of rejecting and/or developing an immune response to a foreign cell or protein, e.g., a human cell or protein. In certain embodiments, a mouse model of toxicity is generated and/or produced by performing, implementing, and/or executing the methods provided herein on a mouse that is immunocompetent.

In some embodiments, the mouse is of an outbred strain. In certain embodiments, the outbred mouse strain is a closed population for at least four generations of genetically variable animals that are bred to maintain maximum heterozygosity. Outbred strains are available, including commercially and are described in detail by Chia et al. Nature Genetics 37(11): 1181-1186 (2005) and Festing ILAR Journal 55 (3): 399-404 (2014). In addition, The Institute for Laboratory Animal Research has a tool on its website (dels.nas.edu/ilar_n/ilarhome/) that searches the websites of suppliers of laboratory animals for named strains and stocks and their suppliers. The International Mouse Strain Resource (http://www.imsr.org/) is a database of strains and stocks that are available worldwide and has updates from contributing repositories.

In some embodiments, the mouse is of an inbred strain. Particular embodiments contemplate that an advantage of using an inbred mouse strain with the methods provided herein is that cells from an individual mouse of an inbred strain may be infused and/or administered to a different individual mouse of the same inbred strain without triggering an immune response to the cells without the need of any immunosuppressant interventions or treatments. In certain embodiments, the mouse model of toxicity to a cell therapy is generated from one or more mice of an inbred mouse strain. In particular embodiments, the model is generated from one or more mice of a substrain of an inbred mouse strain.

In certain embodiments, inbred mouse strains include, but are not limited to 129S1, 129T2, 129X1, 129P3, 129P1, A, AKR, BALB/c, C3H, C57BL/10, C57BLKS, C57BR/cd, C57L, CAST/Ei, CBA, DBA/1, DBA/2, FVB, MRL, NOD, SJL, MOLF/Ei, SWR, NOR, NZB, NZW, RBF, BUB, I, LP, NON, P, PL, RIIS, SM, C58, ALR, ALS, BPH, BPL, BPN, DDY, EL, KK, LG, MA, NH, NZM2410, NZO, RF, SB, SEA, SI, SOD1, SPRET/Ei, WSB/Ei, YBR, and all inbred substrains of each of these mouse strains.

In particular embodiments, the mouse is of an inbred substrain. In certain embodiments, a substrain is a colony and/or a population of mice within the same mouse strain, that are genetically different from other mice, colonies, and/or populations from the same mouse strain. For example, in some embodiments, a substrain may arise where two colonies of the same inbred strain have been separated for more than 10 generations, or, in some embodiments, the substrain may arise where there is known genetic difference between separate colonies of the same strain. In some embodiments, the genetic difference between different substrains may also be a result of residual heterozygosity in the ancestors at the time of separation which becomes fixed, and/or becomes a result of spontaneous mutation during subsequent generations (e.g., genetic drift).

In certain embodiments, suitable substrains include, but are not limited to, 129S1/SvImJ, 129T2/SvEmsJ, 129X1/SvJ, 129P3/J, A/J, AKR/J, BALB/cByJ, BALB/cJ, BTBR T⁺ tf/J, BUB/BnJ, C3H/HeJ, C3H/HeOuJ, C3HeB/FeJ, C57BL/10J, C57L/J, C58/J, C57BR/cdJ, CBA/CaHN-Btkxid/J, CBA/J, DBA/1J, CAST/EiJ, DBA/1LacJ, DBA/2J, DDY/Jc1SidSeyFrkJ, FVB/NJ, KK/H1J, MRL/MpJ, MOLF/EiJ, NONcNZO10/LtJ, NON/ShiLtJ, NOD/ShiLtJ, NZL/LtJ, PL/J, SM/J, SJL/J, SWR/J, NOR/LtJ, NZB/B1NJ, NZW/LacJ, PWD/PhJ, RBF/DnJ, WSB/EiJ, 12956/SvEvTac, AJTAC, BALB/cAnNTac, BALB/cJBomTac, BALB/cABomTac, C57BL/6NTac, C57BL/6JBomTac, C57BL/10SgAiTac, C3H/HeNTac, CBA/JBomTac, DBA/1JBomTac, DBA/2NTac, DBA/2JBomTac, FVB/NTac, NOD/MrkTac, NZM/AegTac, SJL/JcrNTac, BALB/cAnNCr1BR, C3H/HeNCr1BR, C57BL/6NCr1BR, DBA/2NCr1BR, FVB/NCr1BR, C.B-17/IcrCr1BR, 129/SvPasIcoCr1BR, SJL/Jor1IcoCr1BR, A/Jo1aHsd, BALB/cAnNHsd, C3H/HeNHsd, C57BL/10ScNHsd, C57BL/6NHsd, CBA/JCrHsd, DBA/2NHsd, FVB/NHsd, SAMP1/KaHsd, SAMP6/TaHsd, SAMP8/TaHsd, SAMP10/TaHsd, SJL/JCrHsd, AKR/O1aHsd, BiozziABII/RijIIsd, C57BL/6JO1aHsd, FVB/NhanIIsd, MRL/MpO1aIIsd, NZB/O1aIIsd, NZW/O1aHsd, SWR/O1aHsd, 129P2/O1aHsd, and 129S2/SvHsd. In certain embodiments, the inbred mouse strain is a strain produced by a transgenic, knockout, siRNA, and/or CRISPR technique or other genetic manipulation technologies that have bred brother with sister or parent-offspring for ten or more consecutive generations.

In particular embodiments, the mouse is not a C57BL/6 mouse. In certain embodiments, the mouse is not of a substrain of C57BL/6. In some embodiments, the mouse is not a C57BL/6J, C57BL/6JJcl, C57BL/6JJmsSlc, C57BL/6NJcl, C57BL/6NCrlCrlj, C57BL/6NTac, and/or a C57BL/6CrSlc mouse. In certain embodiments, the mouse has less than about a 100%, 90%, 80%, 75%, 70%, 60%, 50%, 40%, 34%, 30%, 25%, 20%, 12.5%, 10%, 6.25%, 5%, 4%, 3%, 2%, 1%, 0.1%, or 0.01% C57BL/6 background. In some embodiments, the mouse has less than about a 100%, 90%, 80%, 75%, 70%, 60%, 50%, 40%, 34%, 30%, 25%, 20%, 12.5%, 10%, 6.25%, 5%, 4%, 3%, 2%, 1%, 0.1%, or 0.01% C57BL/6J, C57BL/6JJcl, C57BL/6JJmsSlc, C57BL/6NJcl, C57BL/6NCrlCrlj, C57BL/6NTac, and/or C57BL/6CrSlc background.

In certain embodiments, the ability for a mouse model to serve as a model for toxicity to an immunotherapy relies in part on the mouse strain and/or genetic background of the mouse. For example, in some embodiments, the methods provided herein are not implemented, performed, and/or executed on a C57BL/6 mouse. In particular embodiments, C57BL/6 mice are not suitable to model toxicity to a cell therapy because the mice have reduced and/or delayed immune and/or inflammatory responses as compared to mice of other strains, e.g., BALB/c mice. For example, in some embodiments, the methods provided herein stimulate a lower expression of and/or stimulate fewer cytokines in C57BL/6 mice than in other strains. In certain embodiments, aspects, characteristics, and/or phenotypes of the toxicity that correspond to toxicity to cell therapy seen in humans are not replicated in C57BL/6 mice. Thus, in some embodiments, previous difficulties in developing and/or identifying a mouse model suitable for studying toxicity is due in part to the widespread use of C57BL/6 mice for research purposes.

In some embodiments, impaired immune responses of C57BL/6 mice are due, at least in part, to the fact that C57BL/6 mice can have a mutation in the Nod-like receptor pyrin domain containing 12 (NLRP12) gene. An NLRP12 missense mutation that inhibits immune responses in C57BL/6J mice was recently identified. Investigation revealed that the mutation occurred prior to 1971, potentially impacting decades of research conducted on affected mouse models. NLRP12 is a component of the innate immune system that regulates immune cell trafficking and cytokine production. The C57BL/6J mutation (G to A at position 3,222,537, Chr. 7) causes an amino acid substitution in NLRP12 (arginine to lysine at residue 1034) in a conserved leucine rich repeat (LRR) domain which is putatively important for protein-protein interactions. The affected LRR domain from NLRP12 is highly conserved among mammals. There are two important exceptions within the Mus genus: 1) lysine (K) to arginine (R) substitution at position 1034 and 2) methionine (M)/valine (V) to lysine (K) substitution at position 1035. Essentially, the lysine at position 1034 in this domain is shifted by one residue within the Mus genus. It is notable that the subsequent C57BL/6J mutation introduced a double lysine residue in this domain which is unique among all mammals. A double lysine within this highly-conserved domain may potentially affect protein-protein interactions or the post-translational regulation of NLRP12. It is unclear how either of the mouse NLRP12 variants functionally compare to human NLRP12; however, data indicates that the C57BL/6J NLRP12 variant is a loss-of-function mutation as compared to NLRP12 from other mouse strains (e.g., BALB/c). (See Ulland et al, Nature Communications 7: 13180 (2016)). Thus, in some embodiments, the methods provided herein are executed, implemented, and/or performed on a mouse with one or fewer copies of an NLRP12 gene encoding a mutant and/or variant NLRP polypeptide with an arginine to lysine at residue 1034.

In some embodiments, the mouse has fewer than two copies of an NLRP12 mutant and/or variant having one or more missense mutations. In certain embodiments, the mouse has one or fewer copies of an NLRP12 mutant and/or has one or fewer variants of NLRP having one or more missense mutations. In particular embodiments, the NLRP12 mutant and/or variant having one or more missense mutations results in defective neutrophil recruitment to a stimulus. In some embodiments, the NLRP12 mutant or variant results in at least a 10%, at least a 20%, at least a 30%, at least a 40%, at least a 50%, at least a 60%, at least a 70%, at least a 80%, at least a 90%, at least a 95%, at least a 97%, at least a 99%, at least a 99.9%, and/or at least a 99.99% reduction in neutrophil recruitment to a stimulus as compared to a mouse with no copies of the NLRP12 mutant and/or variant. In certain embodiments, NLRP12 mutant or variant encodes an NLRP polypeptide with an arginine to lysine substitution at residue 1034. In certain embodiments, the neutrophil recruitment to a stimulus can be assessed as a matter of routine, for example as described by Ulland et al, Nature Communications 7: 13180 (2016).

In particular embodiments, the mouse has a neutrophil recruitment in response to a stimulus that is at least at least a 50%, at least a 60%, at least a 70%, at least a 80%, at least a 90%, at least a 95%, at least a 97%, at least a 99% of the neutrophil recruitment in response to the same challenge in a mouse with no copies of an NLRP12 mutant or variant gene encoding a NLRP12 polypeptide with an arginine to lysine substitution at residue 1034. In some embodiments, the mouse has a neutrophil recruitment in response to a stimulus that is at least at least a 50%, at least a 60%, at least a 70%, at least a 80%, at least a 90%, at least a 95%, at least a 97%, at least a 99% of the neutrophil recruitment in response to the same challenge in a mouse with no copies of an NLRP12 mutant or variant gene encoding a NLRP12 polypeptide with an arginine to lysine substitution at residue 1034.

In particular embodiments, a C57BL/6 mouse contains one or more copies of an NLRP12 mutant and/or variant gene with one or more missense mutations. In certain embodiments, a C57BL/6 mouse contains one or more copies of an NLRP12 mutant and/or variant gene that encodes a NLRP12 polypeptide with an arginine to lysine substitution at residue 1034.

In some embodiments, the mouse has a greater increase in the amount or level of a circulating proinflammatory cytokine in response to an antigen than a C57BL/6 mouse. In certain embodiments, the mouse has an increase in the level or amount of one or more proinflammatory cytokines that are not increased in a C57BL/6 mouse that is exposed to the antigen, such as exposed to the antigen under the same or similar conditions. Proinflammatory cytokines include, but are not limited to, interleukins (IL), such as IL-2, IL-4, IL-5, IL-6, IL-10, and IL-18, and tumor necrosis factor (TNF), IFN-gamma, MCP-1, MIP-1a, MIP-1b, GM-CSF, and angiopoetin-2.

In some embodiments the genetic background of a mouse may be determined as a matter of routine, and include genetic techniques such as identifying SNPs and polymorphisms associated with specific mouse strains, for example SNPs and/or polymorphisms identified by publicly available databases, e.g., Mouse Genome Informatics maintained by Jackson Laboratories.

In particular embodiments, the mouse is a BALB/c mouse. In certain embodiments, the mouse is of a BALB/c substrain. In some embodiments, the mouse is a BALB/cJ, BALB/cAnNCr, BALB/cByJ, or a BALB/cCum mouse. In certain embodiments, the mouse has about at least a 10%, at least a 25%, at least a 30%, at least a 40%, at least a 50%, at least a 60%, at least a 70%, at least a 75%, at least an 80%, at least an 87.5%, at least a 90%, at least a 95%, at least a 97%, at least a 99%, or at least a 99.9% BALB/c background. In particular embodiments, the mouse has a 10%, at least a 25%, at least a 30%, at least a 40%, at least a 50%, at least a 60%, at least a 70%, at least a 75%, at least an 80%, at least an 87.5%, at least a 90%, at least a 95%, at least a 97%, at least a 99%, or at least a 99.9% BALB/cJ, BALB/cAnNCr, BALB/cByJ, or BALB/cCum background.

In some embodiments, the mouse has a reduced population of one or more populations of lymphocytes and/or immune cells. In certain embodiments, the mouse has been administered a lymphodepleting agent or therapy. In certain embodiments, the one or more populations of lymphocytes or immune cells are reduced in relation to an immunocompetent mouse, an immunocompetent mouse that has not been administered a lymphodepleting agent or therapy, and/or an immunocompetent mouse of any of the strains and/or substrains mentioned herein. In particular embodiments, the population of lymphocytes or immune cells are or include total lymphocytes, total immune cells, T cells, B cells, and/or natural killer cells. In some embodiments, the population of lymphocytes or immune cells are or include effector T cells, helper T cells, cytotoxic T cells, memory T cells, regulatory (suppressor) T cells, natural killer T cells, mucosal associated invariant T cells, gamma delta T cells, plasma cells, memory B cells, follicular B cells, marginal zone B cells, B1 cells, B2 cells, regulatory B cells, and/or natural killer cells. In some embodiments, the one or more lymphocytes are or include CD45⁺ cells, CD11b⁺ cells, CD45^{hi}; CD11b⁺ cells, B cells, T cells, CD4⁺ cells, and/or CD8⁺ cells. In some embodiments, the mouse has between or between about 0.0001 and 1,000 cells, 0.0001 and 0.1 cells, 0.001 and 1 cell, 0.01 and 10 cells, 0.1 and 10 cells, 0.1 and 100 cells, 0.1 and 50 cells, 1 and 10 cells, 1 and 100 cells, 10 and 1,000 cells, 10 and 500 cells, 2.5 and 250 cells, 5 and 1,000 cells, or 0.01 and 10 cells, each inclusive, of the population of lymphocytes or immune cells per 1 μl of blood.

In some embodiments, the mouse has one or more exogenous cells, such as cells from another mouse and/or from a cell line, that express an antigen that is bound by and/or recognized by an immunotherapy, e.g., antigen-expressing cells. In some embodiments, the exogenous cells, e.g., cells from another mouse and/or from a cell line, that express the antigen have been administered, injected, or infused to the mouse. In certain embodiments, the mouse has, or has been injected and/or infused with, between or between about 5×10⁴ and 1×10⁹ antigen expressing-cells, 1×10⁵ and 1×10⁸ CD4+ antigen expressing-cells, 1×10⁵ and 1×10⁶ antigen expressing-cells, 5×10⁵ and 1×10⁷ antigen expressing-cells, 2×10⁵ and 1×10⁷ antigen expressing-cells, 5×10⁵ and 5×10⁷ antigen expressing-cells, 1×10⁵ and 1×10⁷ antigen expressing-cells, 1×10⁷ and 1×10⁸ antigen expressing-cells, 5×10⁵ and 5×10⁷ antigen expressing-cells, 1×10⁶ and 1×10⁸ antigen expressing-cells, 1×10⁷ and 1×10⁹ antigen expressing-cells, 1×10⁵ and 1×10⁸ antigen-expressing cells, each inclusive. In particular embodiments, the mouse has, or has been injected and/or infused with, an amount of, of at least, or of about 1×10⁵, 2×10⁵, 2.5×10⁵, 3×10⁵, 4×10⁵, 5×10⁵, 6×10⁵, 7×10⁵, 7.5×10⁵, 8×10⁵, 9×10⁵, 1×10⁶, 2×10⁶, 2.5×10⁶, 3×10⁶, 4×10⁶, 5×10⁶, 6×10⁶, 7×10⁶, 8×10⁶, 9×10⁶, 1×10⁷, 1.1×10⁷, 1.2×10⁷, 1.25×10⁷, 1.3×10⁷, 1.4×10⁷, 1.5×10⁷, 1.6×10⁷, 1.7×10⁷, 1.75×10⁷, 1.8×10⁷, 1.9×10⁷, 2×10⁷, 2.5×10⁷, 5×10⁷, 7.5×10⁷, 3×10⁷, 3.5×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁷, 7×10⁸, 8×10⁸, 1×10⁸, 1×10⁹, 5×10⁹, or 1×10¹⁰ antigen expressing cells.

B. Lymphodepleting Agent or Therapy

In some embodiments, the methods provided herein contain one or more steps of administering a lymphodepleting agent or therapy to a mouse. In particular embodiments, the lymphodepleting agent or therapy reduces the level and/or amount of one or more lymphocytes in the mouse. In certain embodiments, the lymphodepleting agent or therapy reduces the level and/or amount of one or more circulating lymphocytes in the mouse. In some embodiments, the one or more populations of lymphocytes are or include T cells, B cells, and/or natural killer cells.

In some embodiments, the one or more lymphocytes are or include effector T cells, helper T cells, cytotoxic T cells, memory T cells, regulatory (suppressor) T cells, natural killer T cells, mucosal associated invariant T cells, gamma delta T cells, plasma cells, memory B cells, follicular B cells, marginal zone B cells, B1 cells, B2 cells, regulatory B cells, and/or natural killer cells. In some embodiments, the one or more lymphocytes are or include CD45⁺ cells, CD11b⁺ cells, CD45^{hi}; CD11b⁺ cells, B cells, T cells, CD4⁺ cells, and/or CD8⁺ cells. In particular embodiments, the lymphodepleting agent or therapy reduces the level and/or amount of one or more lymphocytes by at least 10%, at least 20%, at least 30%, at least 40%, at least 50%, at least 60%, at least 70%, at least 80%, at least 90%, at least 95%, at least 97%, at least 99%, at least 99.9%, or at least a 99.99%. In some embodiments, the lymphodepleting agent or therapy removes between or between about 10% and 99.99%, 30% and 99.9%, 30% and 70%, 40% and 80%, 50% and 90%, 40% and 60%, 50% and 70%, 60% and 80%, 70% and 90%, 75% and 99%, 60% and 90%, 80% and 99.9%, 90% and 99.9%, 95% and 99.99%, 50% and 60%, 55% and 65%, 60% and 70%, 65% and 75%, 70% and 80%, 75% and 85%, 80% and 90%, 85% and 95%, or 80% and 100%, each inclusive, or about 100% of the one or more lymphocytes. In certain embodiments, the reduction of the one or more populations of lymphocytes by the lymphodepleting agent or therapy is measured and/or determined at, at about, or at least 30 minutes, 1 hour, 2 hours, 3 hours, 4 hours, 6 hours, 8 hours, 12 hours, 24 hours, 36 hours, 48 hours, 60 hours, 72 hours, 5 days, 7 days, 1 week, 2 weeks, 3 weeks, or 4 weeks after the lymphodepleting agent or therapy is administered. In particular embodiments, the lymphodepleting agent or therapy reduces less than 100%, less than 95%, less than 90%, or less than 85% of the one or more lymphocytes.

In some embodiments, the lymphodepleting agent or therapy is not or does not include total body radiation, also referred to as total body irradiation or TBI. In certain embodiments, TBI is a radiation treatment that is delivered to the entire body.

In particular embodiments, the lymphodepleting agent or therapy does not completely remove all lycophytes or cause complete or substantially complete immune ablation. In some embodiments, at least 0.001%, at least 0.01%, at least 0.1%, at least 1%, at least 5%, at least 10%, at least %, at least 20%, at least 25%, at least 30%, at least 40%, or at least 50% of one or more populations are present after the lymphodepleting therapy is administered. In certain embodiments, at least or at least about 0.001%, at least 0.01%, at least 0.1%, at least 1%, at least 5%, at least 10%, at least %, at least 20%, at least 25%, at least 30%, at least 40%, or at least 50% of one or more populations are present 30 minutes, 1 hour, 2 hours, 3 hours, 4 hours, 6 hours, 8 hours, 12 hours, 24 hours, 36 hours, 48 hours, 60 hours, 72 hours, 5 days, 7 days, 1 week, 2 weeks, 3 weeks, or 4 weeks after the lymphodepleting agent or therapy is administered.

In particular embodiments, the lymphodepleting agent or therapy (i) is not or does not include total body irradiation and (ii) does not completely remove all lymphocytes or cause complete or substantially complete immune ablation.

In some embodiments, a lymphodepleting agent is administered to the mouse, e.g., the immunocompetent BALB/c mouse. In certain embodiments, a lymphodepleting therapy is administered to the mouse. In particular embodiments, the lymphodepleting therapy is or includes the administration of two or more doses of one or more lymphodepleting agents. In certain embodiments, the lymphodepleting therapy is or includes administration of two or more different lymphodepleting agents. In some embodiments, the lymphodepleting therapy is or includes administration of at least two, three, four, five, ten, twenty, or fifty different lymphodepleting agents.

In particular embodiments, the lymphodepleting agent is administered to a tumor-bearing mouse, such as a mouse that was previously injected with an antigen-expressing cell or a tumor cell, such as those described in Section I.D. In some embodiments, the lymphodepleting agent is administered to a mouse at a time when the tumor burden is or includes a tumor size greater than or greater than about or about 1 mm, 2 mm, 3 mm, 4 mm, 5 mm, 6 mm, 7 mm, 8 mm, 9 mm, 10 mm, 15 mm, 20 mm, or greater than 20 mm in diameter. In some embodiments, the tumor burden is between or between about 1 mm and 20 mm, 5 mm and 15 mm, 5 mm and 10 mm, or 10 mm and 15 mm in diameter, inclusive. In certain embodiments, the tumor burden is or includes a tumor size of, of about, or of at least 5 mm in diameter. In various embodiments, the tumor burden is or includes a tumor volume of greater than or greater than about or about 30 mm³, 40 mm³, 50 mm³, 60 mm³, 70 mm³, 80 mm³, 90 mm³, 100 mm³, 500 mm³, or 1,000 mm³. In particular embodiments, the tumor burden is or includes a tumor volume of greater than or greater than about or about 60 mm³, 70 mm³, 80 mm³, 90 mm³, or 100 mm³. Methods and techniques of measuring tumor burden are known, and include those described in Bendandi et al., J Vaccines Immunol: JVII-120. DOI: 10.29011/2575-789X. 000020.

In some embodiments, the lymphodepleting agent is or includes an antibody or an antigen binding fragment thereof that targets an antigen that is present on a lymphocyte and/or one or more populations of lymphocytes. In some embodiments lymphodepleting agent is or includes an antibody or an antigen binding fragment thereof that binds to a T cell antigen. In certain embodiments, the antigen is CD2, CD3, CD4, CD8, CD11a, CD18, and/or CD52.

In some embodiments, the lymphodepleting agent is or includes a chemotherapeutic agent. In some embodiments, the lymphodepleting agent is or includes one or more chemotherapeutic agents selected from alkylating agents, cisplatin and its analogues, antimetabolites, topoisomerase interactive agents, antimicrotubule agents, interferons, inteleukin-2, histone deacetylase inhibitors, monoclonal antibodies, estrogen modulators, megestrol, and/or aromatase inhibitors. In certain embodiments, the lymphodepleting agent is or includes a toxin (e.g., saporin, ricin, abrin, ethidium bromide, diptheria toxin, Pseudomonas exotoxin, and others listed above); an alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitrosoureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); luteinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

In some embodiments, the lymphodepleting agent is or includes alkylating agents. In certain embodiments, the alkylating agent is or includes nitrogen mustards. In particular embodiments, the alkylating agent is or includes chlorambucil, cyclophosphamide, isofamide, mechlorethamine, melphalan, or uracil mustard. In some embodiments, the alkylating agent is or includes an aziridine. In certain embodiments, the alkylating agent is or includes thiotepa. In particular embodiments, the alkylating agent is or includes a methanesulphonate ester. In some embodiments, the alkylating agent is or includes busulfan. In particular embodiments, the alkylating agent is or includes a nitrosourea. In particular embodiments, the alkylating agent is or includes carmustine, lomustine, or streptozocin. In particular embodiments, the alkylating agent is or includes a platinum complex. In particular embodiments, the alkylating agent is or includes cisplatin or carboplatin. In some embodiments, the alkylating agent is or includes a bioreductive alkylator. In some embodiments, the alkylating agent is or includes mitomycin, procarbazine, dacarbazine or altretamine.

In some embodiments, the immunotherapy is or includes one or more doses of one or more lymphodepleting agents. In some embodiments, a single dose of the lymphodepleting agent is administered to the mouse, e.g., the immunocompetent mouse. In particular embodiments, one dose, two doses, three doses, four doses, five doses, six doses, seven doses, eight doses, nine doses, ten doses, more than ten doses, more than twenty doses, more than thirty doses, more than forty doses, or more than fifty doses of the one or more lymphodepleting agents are administered to the mouse. In some embodiments, one or more lymphodepleting agents are administered once. In certain embodiments, more than one dose of the lymphodepleting agent is administered over a period of or about 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, or more than six weeks. In particular embodiments, more than one dose of the lymphodepleting agent is administered over a period of less than 24 hours, less than 48 hours, less than 72 hours, less than 4 days, less than 5 days, less than 6 days, less than 7 days, less than 8 days, less than 9 days, less than 10 days, less than 11 days, less than 12 days, less than 13 days, less than 14 days, less than 2 weeks, less than 3 weeks, less than 4 weeks, less than 5 weeks, or less than 6 weeks. In certain embodiments, the lymphodepleting agent is administered to the subject once daily, twice daily, three times daily, four times daily, five times daily, six times daily, eight times daily, ten times daily, or twelve times daily. In some embodiments, doses of the lymphodepleting agent are administered at, at about, or within 1 hour apart, 2 hours apart, 3 hours apart, 4 hours apart, or between 5 minutes and 1 hour apart, between 1 hour and 2 hours apart, between 2 and 4 hours apart, between 4 and 12 hours apart, or between 12 and 24 hours apart, each inclusive. In some embodiments, the lymphodepleting agent is administered once a day, once every 2 days, 3 days, 4 days, 5 days, 6 days, once a week, twice a week, three times a week, once a month, twice a month, three times a month, four times a month, or five times a month. In some embodiments, the lymphodepleting therapy is or includes two or more doses that are administered within a period of three days. In some embodiments, the lymphodepleting therapy is or includes administration of one or more doses or two or more lymphodepleting agents. In certain embodiments, the two or more lymphodepleting agents are or include fludarabine and cyclophosphamide. In some embodiments, the lymphodepleting therapy is or includes one or more doses of a single lymphodepleting agent. In some embodiments, the lymphodepleting therapy is or includes a single dose of a single lymphodepleting agent. In certain embodiments, the lymphodepleting agent is or includes cyclophosphamide.

In some embodiments, the one or more doses of the lymphodepleting agent are administered orally, intravenously, intraperitoneally, transdermally, intrathecally, intramuscularly, intranasally, transmucosally, subcutaneously, or rectally. In some embodiments, the dose of the lymphodepleting agent is or includes between or between about 1 μg/kg and 1,000 mg/kg, between 1 μg/kg and 100 μg/kg, between 100 μg/kg and 500 μg/kg, between 500 μg/kg and 1,000 μg/kg, between 1 mg/kg and 10 mg/kg, between 10 mg/kg and 100 mg/kg, between 100 mg/kg and 500 mg/kg, between 200 mg/kg and 300 mg/kg, between 100 mg/kg and 250 mg/kg, between 200 mg/kg and 400 mg/kg, between 250 mg/kg and 500 mg/kg, between 250 mg/kg and 750 mg/kg, between 50 mg/kg and 750 mg/kg, between 1 mg/kg and 10 mg/kg, or between 100 mg/kg and 1,000 mg/kg (amount of the lymphodepleting agent over body weight, each inclusive). In some embodiments, the dose of the lymphodepleting agent is, is at least, or is about 1 μg/kg, 5 μg/kg, 10 μg/kg, 50 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1 mg/kg, 5 mg/kg, 10 mg/kg, 25 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1 g/kg.

In certain embodiments, the lymphodepleting agent is or includes cyclophosphamide. In some embodiments, the cyclophosphamide is administered once. In particular embodiments, the cyclophosphamide is administered orally, intravenously, intraperitoneally, transdermally, intrathecally, intramuscularly, intranasally, transmucosally, subcutaneously, or rectally. In particular embodiments, the cyclophosphamide (CPA) is administered intraperitoneally. In particular embodiments, the dose of cyclophosphamide is between 1 mg/kg and 10 mg/kg, between 10 mg/kg and 100 mg/kg, between 100 mg/kg and 500 mg/kg, between 200 mg/kg and 300 mg/kg, between 100 mg/kg and 250 mg/kg, between 200 mg/kg and 400 mg/kg, between 250 mg/kg and 500 mg/kg, between 250 mg/kg and 750 mg/kg, between 50 mg/kg and 750 mg/kg, between 1 mg/kg and 10 mg/kg, or between 100 mg/kg and 1,000 mg/kg (amount of the cyclophosphamide over body weight, each inclusive). In some embodiments, the dose of the cyclophosphamide is or is about 10 mg/kg, 25 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1 g/kg.

In some embodiments, about, greater than, or greater than about 100 mg/kg cyclophosphamide is administered to a mouse, e.g., an immunocompetent mouse, intraperitoneally. In certain embodiments, 100 mg/kg cyclophosphamide is administered to a mouse, e.g., an immunocompetent mouse, intraperitoneally (i.p.) In various embodiments, about, at least, or at least about 250 mg/kg cyclophosphamide is administered to a mouse, e.g., an immunocompetent mouse, intraperitoneally. In certain embodiments, 250 mg/kg cyclophosphamide is administered to a mouse, e.g., an immunocompetent mouse, intraperitoneally.

C. Immunotherapy

Particular embodiments of the methods provided herein include one or more steps of administering an immunotherapy, such as a cell therapy, a T cell therapy (e.g. engineered T cell therapy, such as CAR-expressing T cells), and/or a T cell-engaging therapy. In some embodiments, the immunotherapy is administered to a mouse, e.g., an immunocompetent mouse.

In some embodiments, the methods provided herein contain one or more steps of administering the immunotherapy to a mouse described herein, e.g., a mouse described in Section I.A. In particular embodiments, the methods provided herein include one or more steps of administering the immunotherapy to a mouse with a reduced population of lymphocytes or immune cells. In certain embodiments, the methods provided herein include one or more steps of administering the immunotherapy, e.g., an immunotherapy as described herein such as in Section I.C, to a mouse that has been administered a lymphodepleting agent or therapy, e.g., a lymphodepleting agent or therapy that is described herein such as in Section I.B. In some embodiments, the methods provided herein include one or more steps of administering the immunotherapy to a mouse that has exogenous cells that express an antigen that is bound by and/or recognized by the immunotherapy. In some embodiments, the methods provided herein include one or more steps of administering the immunotherapy to a mouse that has been administered, injected, or infused with antigen-expressing cells, e.g., exogenous cells that express an antigen that is bound by and/or recognized by the immunotherapy. In certain embodiments, the exogenous cells and/or the antigen-expressing cells are antigen-expressing cells described herein, such as those described in Section I.D.

In certain embodiments, the immunotherapy is administered prior to, subsequent to, or during the administration of a lymphodepleting agent. In certain embodiments, the immunotherapy is administered during the administration of the lymphodepleting agent. In some embodiments, the lymphodepleting agent is administered subsequent to the administration of the lymphodepleting agent. In certain embodiments, the immunotherapy is administered within about 4 weeks, within 3 weeks, within 2 weeks, within 14 days, within 13 days, within 12 days, within 11 days, within 10 days, within 9 days, within 8 days, within 7 days, within 6 days, within 5 days, within 4 days, within 3 days, within 72 hours, within 60 hours, within 48 hours, within 42 hours, within 36 hours, within 30 hours, within 24 hours, within 18 hours, within 12 hours, within 6 hours, within 4 hours, within 3 hours, within 2 hours, or within 1 hour after the lymphodepleting agent is administered. In particular embodiments, the immunotherapy is administered at, at about, or within 4 weeks, 3 weeks, 2 weeks, 14 days, 13 days, 12 days, 11 days, 10 days, 9 days, 8 days, 7 days, 6 days, 5 days, 4 days, 3 days, 72 hours, 60 hours, 48 hours, 42 hours, 36 hours, 30 hours, 24 hours, 18 hours, 12 hours, 6 hours, 4 hours, 3 hours, 2 hours, or 1 hour after the lymphodepleting agent is administered.

In certain embodiments, the immunotherapy binds to an antigen. In particular embodiments, the immunotherapy binds to and/or recognizes an antigen that is expressed on or in a cell or tissue. In certain embodiments, the antigen is expressed on or in a cell or tissue. In some embodiments, the antigen is expressed on or in a mouse cell or tissue. In particular embodiments, the antigen is expressed on the surface of a cell. In some embodiments, the antigen is expressed on the surface of a mouse cell. In particular embodiments, the antigen is expressed in or on a circulating cell. In some embodiments, the antigen is expressed on the surface of a circulating cell. In particular embodiments, the antigen is expressed on the surface of a mouse cell circulating cell.

In certain embodiments, mice are injected with an immunotherapy that is or is a candidate immunotherapy administered to human subjects for the treatment of a disease. In particular embodiments, the immunotherapy binds to and/or recognizes an antigen associated with a disease. Among the diseases, conditions, and disorders that may be treated in human subjects with the immunotherapy are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the disease or condition is a tumor, e.g., a large tumor burden such as a large solid tumor or a large number or bulk of disease-associated, e.g., tumor, cells. In some aspects, disease or condition is or includes a high number of metastases and/or widespread localization of metastases. In some aspects, the tumor burden in the subject is low and the subject has few metastases.

In some embodiments, the antigen is associated with a disease or condition. In particular embodiments, the antigen is a mouse protein homolog of a human antigen that is associated with a disease or condition. In particular embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In certain embodiments, the antigen is a mouse protein or a portion thereof. In some embodiments, the antigen is selected from the group consisting of αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In particular embodiments, the antigen is a mouse antigen that is expressed on a B cell. Antigens expressed in or on a B cell include, but are not limited to, CD19, CD20, CD22, CD23, CD38, B220, CD40, CD43, CD138, CXCR4, BCMA, IL-6R, B220, CD21, CD35, CD24, CD23, and/or CD40. In certain embodiments, the antigen is B cell maturation antigen (BCMA), CD19, CD20, CD22, CD24, CD30, and/or CD38. In some embodiments, the antigen is CD19.

In some embodiments, the immunotherapy is of exogenous origin, such as a non-host cell, antibody, or protein. In certain embodiments, the immunotherapy is exogenous to the mouse. Hence, in some embodiments, the immunotherapy, e.g., a cell, antibody, or protein, is foreign, e.g., foreign to the mouse. In some embodiments, the immunotherapy is not normally produced by or is not derived from the mouse.

In particular embodiments, the immunotherapy is administered to a tumor-bearing mouse, such as a mouse that was previously injected with an antigen-expressing cell or a tumor cell, such as those described in Section I.D. In particular embodiments, the immunotherapy is administered to a mouse at a time when the tumor burden is or includes a tumor size greater than or greater than about or about 1 mm, 2 mm, 3 mm, 4 mm, 5 mm, 6 mm, 7 mm, 8 mm, 9 mm, 10 mm, 15 mm, 20 mm, or greater than 20 mm in diameter. In some embodiments, the tumor burden is between or between about 1 mm and 20 mm, 5 mm and 15 mm, 5 mm and 10 mm, or 10 mm and 15 mm in diameter, inclusive. In certain embodiments, the tumor burden is or includes a tumor size of, of about, or of at least 5 mm in diameter. In various embodiments, the tumor burden is or includes a tumor volume of greater than or greater than about or about 30 mm³, 40 mm³, 50 mm³, 60 mm³, 70 mm³, 80 mm³, 90 mm³, 100 mm³, 500 mm³, or 1,000 mm³. In particular embodiments, the tumor burden is or includes a tumor volume of greater than or greater than about or about 60 mm³, 70 mm³, 80 mm³, 90 mm³, or 100 mm³.

In particular embodiments, the mouse model is generated by (i) administering a lymphodepleting agent that does not result in complete immune ablation to an immunocompetent mouse, and (ii) administering an immunotherapy. In some embodiments, the immunotherapy is administered at, at about, or within 7 days, 6 days, 5 days, 4 days, 3 days, 72 hours, 60 hours, 48 hours, 36 hours, 24 hours, 18 hours, 12 hours, or 6 hours after the lymphodepleting agent. In particular embodiments, the mouse model is generated by administering a lymphodepleting agent, e.g., cyclophosphamide (CPA), at a dose that does not result in complete immune ablation and then administering an immunotherapy, e.g., an immune cell therapy, to an immunocompetent mouse, e.g., a BALB/c mouse, at, at about, or within 72 hours, 60 hours, 48 hours, 36 hours, 30 hours, 24 hours, 18 hours, 12 hours, or 6 hours, or between or between about 6 hours and 72 hours, 12 hours and 48 hours, or 18 hours and 30 hours after administration of the lymphodepleting agent, each inclusive. In some embodiments, the immunotherapy is an immune system stimulator or a cell therapy, e.g., a CAR T cell therapy. In certain embodiments, the immunocompetent mouse was previously administered or injected with antigen-expressing cells that express the antigen that is bound by or recognized by the immunotherapy.

In certain embodiments, the mouse model is generated by administering between or between about 1 mg/kg and 1,000 mg/kg, between 10 mg/kg and 750 mg/kg, or between 50 mg/kg and 500 mg/kg (amount of the agent over body weight) i.p. of a lymphodepleting agent to an immunocompetent BALB/c mouse (or strain or substrain thereof) and then administering an immunotherapy, e.g., an immune cell therapy, between or between about 6 hours and 72 hours, 12 hours and 48 hours, or 18 hours and 30 hours after administration of the lymphodepleting agent. In particular embodiments, the lymphodepleting agent is CPA. In particular embodiments, the immunocompetent BALB/c mouse was previously administered or injected with antigen-expressing cells that express the antigen that is bound by or recognized by the immunotherapy.

1. Immune System Stimulators

In certain embodiments, the immunotherapy is or contains an immune system activator or stimulator. In certain embodiments, the immune system stimulator is an agent or therapy that activates at least one immune cell. In some embodiments, the immune cell is a T cell. In certain embodiments, the immune cell activator is IL-2, e.g., Proleukin; rhu-IFN-alpha-2a and/or rhu-IFN-alpha-2b, e.g., Pegasys, Roferon-A, Intron-A, and PEG intron; Anti-CD3 monoclonal antibody, e.g., Muromonab-CD3 and/or Orthoclone OKT 3; TGN-1412; and/or Blinatumomab, e.g., anti-CD3×CD19 BiTE.

In some embodiments, the immunotherapy is or contains a T cell-engaging therapy that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the T cell-engaging therapy is or comprises an antibody or antigen-binding fragment. In some embodiments, the T cell-engaging therapy is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. CD19. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). In some embodiments, the T-cell engaging therapy is blinatumomab or AMG 330. Any of such T cell-engagers can be used in used in the provided methods, compositions or combinations.

The immune system stimulator and/or the T cell engaging therapy can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the immunotherapy is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In certain embodiments, one or more doses of a T cell engaging therapy and/or an immune system stimulator are administered. In particular embodiments, between or between about 0.001 μg and about 5,000 μg, inclusive, of the T cell engaging therapy and/or an immune system stimulator are administered. In particular embodiments, between or between about 0.001 μg and 1,000 μg, 0.001 μg to 1 μg, 0.01 μg to 1 μg, 0.1 μg to 10 μg, 0.01 μg to 1 μg, 0.1 μg and 5 μg, 0.1 μg and 50 μg, 1 μg and 100 μg, 10 μg and 100 μg, 50 μg and 500 μg, 100 μg and 1,000 μg, 1,000 μg and 2,000 μg, or 2,000 μg and 5,000 μg of the T cell engaging therapy is administered. In some embodiments, the dose of the T cell engaging therapy is or includes between or between about 0.01 μg/kg and 100 mg/kg, 0.1 μg/kg and 10 μg/kg, 10 μg/kg and 50 μg/kg, 50 μg/kg and 100 μg/kg, 0.1 mg/kg and 1 mg/kg, 1 mg/kg and 10 mg/kg, 10 mg/kg and 100 mg/kg, 100 mg/kg and 500 mg/kg, 200 mg/kg and 300 mg/kg, 100 mg/kg and 250 mg/kg, 200 mg/kg and 400 mg/kg, 250 mg/kg and 500 mg/kg, 250 mg/kg and 750 mg/kg, 50 mg/kg and 750 mg/kg, 1 mg/kg and 10 mg/kg, or 100 mg/kg and 1,000 mg/kg (amount of the lymphodepleting agent over body weight), each inclusive. In some embodiments, the dose of the T cell engaging therapy is at least or at least about or is or is about 0.1 μg/kg, 0.5 μg/kg, 1 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1,000 mg/kg. In particular embodiments, the T cell engaging therapy is administered orally, intravenously, intraperitoneally, transdermally, intrathecally, intramuscularly, intranasally, transmucosally, subcutaneously, or rectally.

2. Cell Therapy

In some embodiments, the methods provided herein contain one or more steps of administering and/or infusing an immunotherapy. In certain embodiments, the immunotherapy is a cell composition that contains one or more engineered cells. In some embodiments, the engineered cells express a recombinant receptor. In particular embodiments, the recombinant receptor is a chimeric antigen receptor (CAR). In particular embodiments, the recombinant receptor is a T cell receptor (TCR), e.g., a recombinant TCR. In some embodiments, the cell composition includes or contains cells that express a recombinant receptor. In particular embodiments, the cell composition includes or contains cells that express a CAR. In certain embodiments, the cell composition is or includes cells that express a recombinant TCR. In particular embodiments, the cell composition includes and/or is composed of mouse cells.

In some embodiments, the cells for use in or administered in connection with the immunotherapy provided herein contain or are engineered to contain an engineered receptor, e.g., an engineered antigen receptor, such as a chimeric antigen receptor (CAR), or a T cell receptor (TCR). Also provided are populations of such cells, e.g., immune cells, compositions containing such cells and/or enriched for such cells, such as in which cells of a certain type such as T cells or CD8+ or CD4+ cells are enriched or selected. In some embodiments, the cells are or include immune cells, such as immune cells that are isolated, enriched, or selected from splenocytes, e.g., mouse splenocytes. Among the compositions are pharmaceutical compositions and formulations for administration, such as for adoptive cell therapy. Also provided are therapeutic methods for administering the cells and compositions to subjects, e.g., patients, in accord with the provided methods.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In certain embodiments, the immunotherapy is or contains cells that generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In certain embodiments, the mouse model is generated by administering between or between about 1 mg/kg and 1,000 mg/kg, between 10 mg/kg and 750 mg/kg, or between 50 mg/kg and 500 mg/kg (amount of the agent over body weight) i.p. of a lymphodepleting agent to an immunocompetent BALB/c mouse (or strain or substrain thereof) and then administering an immunotherapy, e.g., an immune cell therapy, between or between about 6 hours and 72 hours, 12 hours and 48 hours, or 18 hours and 30 hours after administration of the lymphodepleting agent. In particular embodiments, the lymphodepleting agent is CPA. In some embodiments, the immunotherapy is or includes a composition of cells, e.g., mouse cells, that express a recombinant receptor. In some embodiments, between or between about 1×10⁶ and 50×10⁶ cells are administered. In certain embodiments, between or between about 1×10⁶ and 50×10⁶ cells expressing a recombinant receptor, e.g., a TCR or CAR, are administered. In some embodiments, the immunocompetent BALB/c mouse was previously administered or injected with antigen-expressing cells that express the antigen that is bound by or recognized by the immunotherapy.

a. Chimeric Antigen Receptors (CARs)

In some embodiments, chimeric receptors, such as chimeric antigen receptors (CARs), contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the chimeric receptors, such as CARs, include an extracellular antigen binding domain, such as a portion of an antibody molecule, generally a variable heavy (VH) chain region and/or variable light (VL) chain region of the antibody, e.g., an scFv antibody fragment that is derived from an antibody that binds to and/or recognizes a mouse B cell antigen. In certain embodiments, the extracellular antigen binding domain recognizes and/or binds to a murine CD19. In certain embodiments, the chimeric receptors, such as CARs, include an extracellular antigen binding domain, such as a portion of an antibody molecule, generally a variable heavy (VH) chain region and/or variable light (VL) chain region of the antibody, e.g., an scFv antibody fragment that is derived from the 1D3 rat monoclonal anti-mouse CD19 antibody. In some embodiments, the extracellular antigen binding domain contains a variable heavy (VH) chain region that is at least 85%, 90%, or 95% identical to the variable heavy (VH) chain region set forth in SEQ ID NO: 2. In certain embodiments, the extracellular antigen binding domain contains a variable heavy (VH) chain region that is set forth in SEQ ID NO: 2. In certain embodiments, the extracellular antigen binding domain contains a variable light (VL) chain region that is at least 85%, 90%, or 95% identical to the variable light (VL) chain region set forth in SEQ ID NO: 3. In certain embodiments, the extracellular antigen binding domain contains a variable light (VL) chain region that is set forth in SEQ ID NO: 3.

In some embodiments, a control receptor is designed that recognizes or binds to an antigen in humans but in mice. Thus, in some embodiments, the receptor contains an extracellular binding domain that binds to and/or recognizes a human antigen but not the mouse antigen. In certain embodiments, the extracellular antigen binding domain of a control receptor recognizes and/or binds to a human CD19 but not mouse CD19. In certain embodiments, the chimeric receptors, such as CARs, include an extracellular antigen binding domain, such as a portion of an antibody molecule, generally a variable heavy (VH) chain region and/or variable light (VL) chain region of the antibody, e.g., an scFv antibody fragment that is derived from the monoclonal FMC63 anti-human CD19 antibody. In some embodiments, the extracellular antigen binding domain contains a variable heavy (VH) chain region that is at least 85%, 90%, or 95% identical to the variable heavy (VH) chain region set forth in SEQ ID NO: 9. In certain embodiments, the extracellular antigen binding domain contains a variable heavy (VH) chain region that is set forth in SEQ ID NO: 9. In certain embodiments, the extracellular antigen binding domain contains a variable light (VL) chain region that is at least 85%, 90%, or 95% identical to the variable light (VL) chain region set forth in SEQ ID NO: 10. In certain embodiments, the extracellular antigen binding domain contains a variable light (VL) chain region that is set forth in SEQ ID NO: 10.

In some embodiments, the antigen targeted by the receptor is a polypeptide, e.g., a mouse polypeptide. In some embodiments, it is a carbohydrate or other molecule, e.g., a carbohydrate or other molecule that is endogenous to a mouse. In some embodiments, the antigen is selectively expressed or overexpressed on antigen expressing cells, e.g., antigen expressing cells described herein, such as in Section I.D.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30. In certain embodiments, the antigens targeted by the receptors mouse antigens that are expressed on B cells and/or are associated with a B cell malignancy, such as any of a number of known mouse B cell marker. In some embodiments, the antigen is mouse CD20, CD19, CD22, ROR1, CD45, CD21, CD5, CD33, Igkappa, Iglambda, CD79a, CD79b or CD30. In particular embodiments, the antigen is mouse CD19.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor, e.g., CAR, further includes at least a portion of an immunoglobulin constant region, such as a hinge region, e.g., an IgG3 hinge region, and/or a CH1/CL and/or Fc region. In some embodiments, the constant region or portion is of a mouse IgG, such as IgG3 or IgG1. In some embodiments, the constant region or portion of mouse IgG is or includes mouse IgG3. In certain embodiments, the mouse IgG is or is a portion of the IgG set forth in SEQ ID NO: 4. In particular embodiments, the mouse IgG is or is a portion of an IgG sequence with at least 85%, 90%, of 95% sequence identity to all or a portion of the IgG set forth in SEQ ID NO: 4. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex. In particular embodiments, the transmembrane domain is derived from a mouse protein.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the transmembrane domain is derived from murine CD28. In particular embodiments, the transmembrane domain is set forth in SEQ ID NO: 5. In particular embodiments, the transmembrane domain has at least 85%, 90%, 95% sequence identity to all or a portion of the transmembrane domain set forth in SEQ ID NO: 5.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In certain embodiments, the extracellular portion is derived from a mouse protein, e.g., mouse CD28.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. In certain embodiments, the one or both signaling components are derived from mouse proteins.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta. In certain embodiments, the ITAM is a mouse ITAM and/or is derived from mouse protein.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, the receptor includes an intracellular component of a murine TCR complex, such as a murine TCR CD3 chain that mediates T-cell activation and cytotoxicity, e.g., murine CD3 zeta chain. In certain embodiments, the TCR complex is a murine TCR complex. In some embodiments, cell signaling modules include a murine CD3 transmembrane domain, a murine CD3 intracellular signaling domain, and/or other murine CD transmembrane domains. In some embodiments, the receptor, e.g., CAR, further includes a portion of one or more additional molecules such as a murine Fc receptor γ, murine CD8, murine CD4, murine CD25, or murine CD16. For example, in some aspects, the murine CAR or other chimeric receptor includes a chimeric molecule between murine CD3-zeta (CD3-ζ) or murine Fc receptor γ and murine CD8, murine CD4, murine CD25 or murine CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the intracellular domain to a T cell costimulatory molecule is derived from a mouse T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB. In certain embodiments, the T cell costimulatory molecule is mouse CD28, 4-1BB, OX40, DAP10, or ICOS.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain. In some embodiments, the intracellular signaling domain comprises a chimeric CD28 and CD137 (4-1BB, TNFRSF9) co-stimulatory domains, linked to a CD3 zeta intracellular domain. In some embodiments, the CAR contains a transmembrane region set forth in SEQ ID NO: 5 and an intracellular signaling domain set forth in SEQ ID NO: 6 and/or a signaling domain set forth in SEQ ID NO: 7.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB. In some embodiments, the intracellular components are derived from mouse CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the CAR or other antigen receptor further includes a marker, such as a cell surface marker, which may be used to confirm transduction or engineering of the cell to express the receptor. In some embodiments, the marker is a peptide, protein, or portion thereof that does not induce an immune response in the subject, e.g., mouse administered the cell composition, but is one that is not endogenous to and/or expressed by the subject. In some embodiments, the peptide is an isoform of mouse Thy1, Ly45, or CD45.

An exemplary polypeptide for a Thy1.1 comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of mouse CD28 (or variant thereof, such as a transmembrane domain that comprises the sequence of amino acids set forth in SEQ ID NO: 5 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 5.

In some embodiments, the intracellular signaling domain of the recombinant receptor, e.g. the CAR, comprises a mouse CD3 zeta stimulatory signaling domain or functional variant thereof, such as a cytoplasmic domain mouse CD3 (Accession No.: P20963.2) or a CD3 zeta signaling domain. For example, in some embodiments, the intracellular signaling domain comprises the sequence of amino acids as set forth in SEQ ID NO: 7 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

In some aspects, the spacer contains only a hinge region of an IgG, such as only a hinge of mouse IgG3 or IgG1. In other embodiments, the spacer is or contains an Ig hinge, e.g., a mouse IgG3-derived hinge, optionally linked to a CH2 and/or CH3 domains. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a Thy1.1 sequence, e.g., downstream of the sequence encoding the CAR. In some embodiments, T cells expressing an antigen receptor (e.g. CAR) can also be generated to express a Thy1.1 as a non-immunogenic selection epitope (e.g. by introduction of a construct encoding the CAR and Thy1.1 separated by a T2A ribosome switch to express two proteins from the same construct), which then can be used as a marker to detect such cells (see e.g. U.S. Pat. No. 8,802,374). In some embodiments, the sequence encodes a Thy1.1 sequence set forth in SEQ ID NO: 8, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

In particular embodiments, the mouse model is generated by administering CPA, e.g., between or about between 50 mg/kg and 500 mg/kg i.p. of CPA, to an immunocompetent BALB/c mouse (or strain or substrain thereof) and then administering between or between about 1×10⁶ and 50×10⁶ cells expressing a CAR between or between about 18 and 30 hours after administration of the CPA. In certain embodiments, the CAR binds to or recognizes an antigen expressed by a cell within the mouse, e.g., an antigen expressed by a mouse cell or by a cell that has been injected into the mouse. In some embodiments, the antigen is associated with a cancer. In particular embodiments, the CAR recognizes or binds to a B cell antigen, such as a mouse B cell antigen or B cell marker. In some embodiments, the CAR binds to or recognizes mouse CD20, CD19, CD22, ROR1, CD45, CD21, CD5, CD33, Igkappa, Iglambda, CD79a, CD79b or CD30. In certain embodiments, the CAR binds to or recognizes mouse CD19. In particular embodiments, the immunocompetent BALB/c mouse was previously injected with antigen-expressing cells prior to the injection of the CPA and the immunotherapy.

In some embodiments, the mouse model is generated by (i) administering a dose of or of about 100 mg/kg or 250 mg/kg i.p. to an immunocompetent BALB/c mouse (or strain or substrain thereof) and then (ii) administering a dose of or of about 5×10⁶, 10×10⁶, or 20×10⁶ total CAR expressing cells at, at about, or within 24 hours after the CPA injection. In some embodiments, the CAR is an anti-mouse CD19 CAR.

b. TCRs

In some embodiments, the immunotherapy is or includes engineered cells, such as T cells, are provided that express a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In certain embodiments, the TCR is a mouse TCR.

In some embodiments, a TCR includes a full TCR or an antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In certain embodiments, the transmembrane domain is derived from a mouse TCR. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex. In some embodiments, the CD3 is mouse CD3 and/or is derived from mouse CD3. In particular embodiments, the intracellular tails of CD3 signaling subunits (e.g. CD3γ, CD3δ, CD3ε and CD3ζ chains) are mouse CD3 signaling subunits and/or are derived from mouse CD3 proteins.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, the sequence is a mouse sequence, e.g., a sequence of a mouse TCR. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a mouse T cell (e.g. cytotoxic T cell), a mouse T-cell hybridomas or other publicly available source. In some embodiments, the mouse T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a mouse TCR identified or selected from screening a library of candidate mouse TCRs against a target polypeptide antigen, or target T cell epitope thereof. TCR libraries can be generated by amplification of the repertoire of Vα and Vβ from T cells isolated from a mouse, e.g., a donor mouse, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an MHC-restricted motif in a target polypeptide of interest, such as a target polypeptide described below. In some embodiments, peptides are identified using available computer prediction models. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell. In certain embodiments, the TCR is derived from mouse.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane. In some embodiments, the dTCR is derived from one or more mouse proteins.

In some embodiments, a dTCR contains a TCR α chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129). In particular embodiments, the scTCR is derived from one or more mouse proteins.

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by αβ chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids.

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

3. Genetically Engineered Cells and Methods of Producing Cells

In some embodiments, the provided methods involve administering to a subject, e.g., a subject having a disease or condition cells and/or a mouse, expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

a. Vectors and Methods for Genetic Engineering

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., mouse T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the anti-CD3/anti-CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

b. Cells and Preparation of Cells for Genetic Engineering

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject or donor, e.g., a donor mouse of the same strain, substrain, or genetic makeup as the mouse administered the cell therapy.

Accordingly, the cells in some embodiments are primary cells, e.g., primary mouse cells. The samples include tissue, fluid, and other samples taken directly from the subject, e.g., mouse, or donor, e.g., donor mouse, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. In particular embodiments, the samples are or include splenocytes, e.g., mouse splenocytes.

In some embodiments, the cells are derived from the blood, bone marrow, lymph, spleen, or lymphoid organs, are cells of the immune system, such as cells of the innate or adaptive immunity, e.g., myeloid or lymphoid cells, including lymphocytes, typically T cells and/or NK cells. In particular embodiments, the cells are derived from spleen, e.g., mouse spleen, and/or are isolated, selected, or enriched from splenocytes, e.g., mouse splenocytes. Other exemplary cells include stem cells, such as multipotent and pluripotent stem cells, including induced pluripotent stem cells (iPSCs). The cells typically are primary cells, e.g., primary mouse cells, such as those isolated directly from a subject e.g., a mouse such as a donor mouse, and/or isolated from a subject, e.g., a mouse subject, and frozen, e.g., cryofrozen, cryogenically frozen, or cryopreserved In some embodiments, the cells include one or more subsets of mouse T cells or other cell types, such as whole mouse T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In particular embodiments, the one or more subsets of mouse T cells are isolated, selected, or enriched from mouse splenocytes. With reference to the subject, e.g., the individual mouse subject, to be treated, the cells may be allogeneic and/or autologous. In particular embodiments, the allogenic cells may be derived from a mouse of the same strain or substrain as the subject, such as a mouse having the same or similar genetic makeup, e.g., at least 80%, 85%, 90%, 95%, 97%, 98%, 99%, 99.9%, 99.99%, or 99.999% identity to the subject's genome. In some embodiments, the allogenic cells do not induce or induce a minimal immune response when administered to a subject that is not lymphodepleted or when the cells have not been engineered to express a recombinant receptor.

Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of mouse T cells and/or of mouse CD4+ and/or of mouse CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (T_{IL}), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are mouse natural killer (NK) cells. In some embodiments, the cells are mouse monocytes or mouse granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, e.g., a mouse such as a donor mouse, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In particular embodiment, the cells are derived or isolated from mouse spleen and/or mouse lymph node. In particular embodiments, the cells are derived or isolated from single cells suspensions derived from and/or produced from mouse spleen and/or mouse lymph node.

In some embodiments, the cells are derived from cell lines, e.g., mouse T cell lines. The cells in some embodiments are obtained from a xenogeneic source, for example, from, rat, non-human primate, human, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject, e.g., mouse subject, are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of mouse T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, mouse CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, mouse T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, mouse CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample, or a single cell suspension prepared from and/or derived from mouse spleen and/or mouse lymph node is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, and CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6,000 to 10,000 rads. The LCL feeder cells in some aspects is provided in any suitable amount, such as a ratio of LCL feeder cells to initial T lymphocytes of at least about 10:1.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

c. Administering the Cell Composition

In certain embodiments, the cell composition can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the immunotherapy is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In particular embodiments, the cell composition is administered intravenously. In certain embodiment, the cell composition is administered intravenously into the lateral tail vein.

In some embodiments, the cell composition contains cells that have not been previously cryofrozen, e.g., cryopreserved, cryoprotected, or cryogenically frozen. In particular embodiments, the cells or at least a portion of the cells of the cell composition have been cryofrozen at one or more times during the process of generating, manufacturing and/or producing the cell composition. In some embodiments, the cells or at least a portion of the cells of the cell composition have and/or between the steps of collecting, isolating, activating, transducing, and/or expanding the cells.

In certain embodiments, between or between about 1×10⁵ and 1×10¹⁰ cells, 1×10⁵ and 1×10⁷ cells, 1×10⁶ and 1×10⁸ cells, 5×10⁵ and 5×10⁸ cells, 1×10⁷ and 5×10⁹ cells, 5×10⁶ and 2×10⁷ cells, 5×10⁶ and 1×10⁸ cells, 5×10⁶ and 2×10⁷ cells, 1×10⁶ and 2×10⁷ cells, 1×10⁷ and 5×10⁷ cells, 5×10⁶ and 2×10⁷ cells, 2×10⁷ and 3×10⁷ cells, 3×10⁷ and 4×10⁷ cells, or 5×10⁷ and 1×10⁸ cells, e.g., total cells, of the composition are administered each inclusive. In particular embodiments, an amount of at least or at last about or that is or is about 1×10⁵, 2.5×10⁵, 5×10⁵, 6×10⁵, 7×10⁵, 8×10⁵, 9×10⁵, 1×10⁶, 1.5×10⁶, 2×10⁶, 2.5×10⁶, 5×10⁶, 7.5×10⁶, 1×10⁷, 2×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 1×10⁹, 2×10⁹, 5×10⁹, or 1×10¹⁰ cells, e.g., total cells, of the composition are administered.

In particular embodiments, between or between about 1×10⁵ and 1×10⁹ cells, 1×10⁵ and 1×10⁷ cells, 1×10⁶ and 1×10⁸ cells, 1×10⁶ and 5×10⁷ cells, 5×10⁶ and 2×10⁷ cells, 5×10⁶ and 2×10⁷ cells, 5×10⁶ and 1×10⁸ cells, 5×10⁶ and 2×10⁷ cells, 1×10⁷ and 2×10⁸ cells, 1×10⁷ and 5×10⁷ cells, 5×10⁶ and 1×10⁸ cells, 5×10⁶ and 3×10⁷ cells, 3×10⁶ and 4×10⁶ cells, or 5×10⁶ and 5×10⁷ cells expressing a recombinant receptor, e.g., a CAR, are administered, each inclusive. In particular embodiments, an amount of at least or at last about or that is or is about 1×10⁵, 2.5×10⁵, 5×10⁵, 7.5×10⁵, 1×10⁶, 1×10⁶, 2.5×10⁶, 5×10⁶, 6×10⁶, 7×10⁶, 8×10⁶, 9×10⁶, 1×10⁷, 1.1×10⁷, 1.2×10⁷, 1.25×10⁷, 1.3×10⁷, 1.4×10⁷, 1.5×10⁷, 1.6×10⁷, 1.7×10⁷, 1.75×10⁷, 1.8×10⁷, 1.9×10⁷, 2×10⁷, 2.5×10⁷, 5×10⁷, 7.5×10⁷, 3×10⁷, 3.5×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁷, 7×10⁸, 8×10⁸, 1×10⁸, 1×10⁹, 5×10⁹, or 1×10¹⁰ cells expressing a recombinant receptor, e.g., a CAR, are administered.

In particular embodiments, between or between about 1×10⁵ and 1×10⁹ CD4+ cells, 1×10⁵ and 1×10⁷ CD4+ cells, 1×10⁶ and 1×10⁸ CD4+ cells, 1×10⁶ and 5×10⁶ CD4+ cells, 2.5×10⁶ and 1×10⁷ CD4+ cells, 2.5×10⁶ and 1×10⁷ CD4+ cells, 5×10⁶ and 2×10⁷ CD4+ cells, 5×10⁶ and 2×10⁷ CD4+ cells, 5×10⁶ and 1×10⁸ CD4+ cells, 5×10⁶ and 2×10⁷ CD4+ cells, 1×10⁷ and 2×10⁸ CD4+ cells, 1×10⁷ and CD4+5×10⁷ cells, 5×10⁶ and 1×10⁸ CD4+ cells, 5×10⁶ and 3×10⁷ CD4+ cells, 3×10⁶ and 4×10⁶ CD4+ cells, or 5×10⁶ and 5×10⁷ CD4+ cells expressing a recombinant receptor, e.g., a CAR, are administered, each inclusive. In particular embodiments, an amount of at least or at last about or that is or is about 1×10⁵, 2.5×10⁵, 5×10⁵, 7.5×10⁵, 1×10⁶, 1×10⁶, 2.5×10⁶, 5×10⁶, 6×10⁶, 7×10⁶, 8×10⁶, 9×10⁶, 1×10⁷, 1.1×10⁷, 1.2×10⁷, 1.25×10⁷, 1.3×10⁷, 1.4×10⁷, 1.5×10⁷, 1.6×10⁷, 1.7×10⁷, 1.75×10⁷, 1.8×10⁷, 1.9×10⁷, 2×10⁷, 2.5×10⁷, 5×10⁷, 7.5×10⁷, 3×10⁷, 3.5×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁷, 7×10⁸, 8×10⁸, 1×10⁸, 1×10⁹, 5×10⁹, or 1×10¹⁰ CD4+ cells expressing a recombinant receptor, e.g., a CD4+CAR, are administered.

In particular embodiments, between or between about 1×10⁵ and about 1×10⁹ CD8+ cells, between 1×10⁵ and 1×10⁷ CD8+ cells, between 1×10⁶ and 1×10⁸ CD8+ cells, between 1×10⁶ and 5×10⁶ CD8+ cells, between 2.5×10⁶ and 1×10⁷ CD8+ cells, between 2.5×10⁶ and 1×10⁷ CD8+ cells, between 5×10⁶ and 2×10⁷ CD8+ cells, between 5×10⁶ and 2×10⁷ CD8+ cells, between 5×10⁶ and 1×10⁸ CD8+ cells, between 5×10⁶ and 2×10⁷ CD8+ cells, between 1×10⁷ and 2×10⁸ CD8+ cells, between 1×10⁷ and CD8+5×10⁷ cells, between 5×10⁶ and 1×10⁸ CD8+ cells, between 5×10⁶ and 3×10⁷ CD8+ cells, between 3×10⁶ and 4×10⁶ CD8+ cells, or between 5×10⁶ and 5×10⁷ CD8+ cells expressing a recombinant receptor, e.g., a CAR, are administered, each inclusive. In particular embodiments, an amount of at least or at last about or that is or is about 1×10⁵, 2.5×10⁵, 5×10⁵, 7.5×10⁵, 1×10⁶, 1×10⁶, 2.5×10⁶, 5×10⁶, 6×10⁶, 7×10⁶, 8×10⁶, 9×10⁶, 1×10⁷, 1.1×10⁷, 1.2×10⁷, 1.25×10⁷, 1.3×10⁷, 1.4×10⁷, 1.5×10⁷, 1.6×10⁷, 1.7×10⁷, 1.75×10⁷, 1.8×10⁷, 1.9×10⁷, 2×10⁷, 2.5×10⁷, 5×10⁷, 7.5×10⁷, 3×10⁷, 3.5×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁷, 7×10⁸, 8×10⁸, 1×10⁸, 1×10⁹, 5×10⁹, or 1×10¹⁰ CD8+ cells expressing a recombinant receptor, e.g., a CD8+ CAR, are administered.

In particular embodiments, an immunotherapy is administered to a mouse that has previously been administered a lymphodepleting agent or therapy, thereby producing a mouse model of toxicity, e.g., toxicity to an immunotherapy. In certain embodiments, the lymphodepleting agent or therapy is described herein, such as in Section I.B. In particular embodiments, the lymphodepleting agent or therapy is CPA. In some embodiments, the CPA is administered i.p. at a dose that is greater than 100 mg/kg. In some embodiments, the immunotherapy is a cell composition that contains cells that express a recombinant receptor, e.g., a CAR, that binds to and/or recognizes a mouse antigen. In certain embodiments, the immunotherapy binds to and/or recognizes mouse CD19. In particular embodiments, the immunotherapy is administered at or about 24 hours after the lymphodepleting agent or therapy is administered. In some embodiments, the mouse is an immunocompetent BALB/c mouse.

In particular embodiments, an immunocompetent BALB/c mouse is administered CPA at a dose that is, is about, or is greater than 100 mg/kg i.p. or 250 mg/kg i.p., and is then administered a T cell composition containing T cells that express a CAR that binds to and/or recognizes a mouse antigen, thereby producing a mouse model of toxicity, e.g., toxicity to an immunotherapy. In certain embodiments, an immunocompetent BALB/c mouse is administered between 100 and 500 mg/kg CPA i.p., and then 24 hour or about 24 hours later, the mouse is administered a between 5×10⁶ cells 2×10⁷ cells of a cell composition that contains anti-mouse CD19 CAR expressing T cells.

In particular embodiments, the mouse model is generated by administering between or about between 50 mg/kg and 500 mg/kg i.p. of CPA to an immunocompetent BALB/c mouse (or strain or substrain thereof) and then administering between or between about 1×10⁶ and 50×10⁶ cells expressing a CAR between or between about 18 and 30 hours after administration of the CPA. In certain embodiments, the CAR binds to or recognizes an antigen expressed by a cell within the mouse, e.g., an antigen expressed by a mouse cell or by a cell that has been injected into the mouse. In some embodiments, the antigen is associated with a cancer. In particular embodiments, the CAR recognizes or binds to a B cell antigen, such as a mouse B cell antigen or B cell marker. In some embodiments, the CAR binds to or recognizes mouse CD20, CD19, CD22, ROR1, CD45, CD21, CD5, CD33, Igkappa, Iglambda, CD79a, CD79b or CD30. In certain embodiments, the CAR binds to or recognizes mouse CD19. In some embodiments, the CPA is administered at a dose of or of about 100 mg/kg or 250 mg/kg i.p. In certain embodiments, the cells expressing the CAR are administered at a dose of or of about 5×10⁶, 10×10⁶, or 20×10⁶ total CAR expressing cells. In particular embodiments, the cells expressing the CAR are administered at, at about, or within 24 hours after the CPA is administered.

D. Antigen-Expressing Cells

In particular embodiments, the methods provided herein contain one or more steps of injecting cells, e.g., antigen-expressing cells, into a mouse. e.g., a mouse described herein such as in Section I.A. In certain embodiments, the cells, e.g., antigen-expressing cells are exogenous, heterologous, and/or autologous to the mouse. In some embodiments, the cells are exogenous to the individual mouse. In certain embodiments, the cells, e.g., the antigen-expressing cells, express an antigen that is bound by and/or recognized by the immunotherapy. In some embodiments, when the immunotherapy is or includes a cell composition, the antigen-expressing cells are separate and/or different from some or all of the cells of the immunotherapy. In certain embodiments, the antigen-expressing cells are administered during or subsequent to administration of the immunotherapy. In particular embodiments; the antigen-expressing cells are administered prior to administering the immunotherapy. In some embodiments, the antigen-expressing cells are tumor cells.

In some embodiments, the antigen-expressing cells are cells that do not trigger an immune response in immunocompetent mice. In certain embodiments, the antigen-expressing cells are mouse cells. In particular embodiments, the antigen-expressing cells are primary cells. In some embodiments, the cell line is an immortal cell line. In some embodiments, the antigen-expressing cells are cells of a cell line that is derived from or has originated from mouse cells or mouse tissue. In particular embodiments, the cells are derived from or have originated from BALB/c mouse cells or tissue. In particular embodiments, the antigen expressing cells are cancerous cells and/or tumor cells. In some embodiments, the antigen-expressing cells are derived from a cancer cell and/or a tumor cells, e.g., mouse cancer cells and/or mouse tumor cells.

In particular embodiments, the antigen expressing cells are tumor cells. In some embodiments, the antigen-expressing cells are circulating tumor cells, e.g., neoplastic immune cells such as neoplastic B cells (or cells derived from neoplastic B cells).

In particular embodiments, the antigen-expressing cells express αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-AIA1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome, tautomerase, dopachrome delta-isomerase or DCT), vascular endothelial growth factor receptor (VEGFR), vascular endothelial growth factor receptor 2 (VEGFR2), Wilms Tumor 1 (WT-1), or a combination thereof. In some embodiments, the antigen-expressing cells express a pathogen-specific or pathogen-expressed antigen, or an antigen associated with a universal tag, and/or biotinylated molecules, and/or molecules expressed by HIV, HCV, HBV or other pathogens. In particular embodiments, the antigen expressing cells express one or more antigens associated with a B cell malignancy, such as any of a number of known B cell markers. In certain embodiments, the antigen-expressing cells express CD20, CD19, CD22, ROR1, CD45, CD21, CD5, CD33, Igkappa, Iglambda, CD79a, CD79b, CD30 or a combination thereof. In some embodiments, the antigen expression-cells express CD19, e.g., mouse CD19.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In certain embodiments, the antigen-expressing cells are, or are derived from, a tumor cell. In some embodiments, the tumor cell is cancerous. In particular embodiments the tumor cells is non-cancerous. In some embodiments, the tumor cell is or is derived a circulating B cell, such as a circulating B cell capable of forming a tumor in vivo. In some embodiments, the tumor cell is or is derived from a circulating B cell that is a neoplastic, tumorigenic, or cancerous B cell.

In certain embodiments, the tumor cell is, or is derived from, a mouse cancer cell. In some embodiments, the tumor cell is derived from a cell of a(n) AIDS-related cancer, a breast cancer, a cancer of the digestive/gastrointestinal tract, an anal cancer, an appendix cancer, a bile duct cancer, a colon cancer, a colorectal cancer, an esophageal cancer, a gallbladder cancer, islet cell tumors, pancreatic neuroendocrine tumors, a liver cancer, a pancreatic cancer, a rectal cancer, a small intestine cancer, a stomach (gastric) cancer, an endocrine system cancer, an adrenocortical carcinoma, a parathyroid cancer, a pheochromocytoma, a pituitary tumor, a thyroid cancer, an eye cancer, an intraocular melanoma, a retinoblastoma, a bladder cancer, a kidney (renal cell) cancer, a penile cancer, a prostate cancer, a transitional cell renal pelvis and ureter cancer, a testicular cancer, a urethral cancer, a Wilms' tumor or other childhood kidney tumor, a germ cell cancer, a central nervous system cancer, an extracranial germ cell tumor, an extragonadal germ cell tumor, an ovarian germ cell tumor, a gynecologic cancer, a cervical cancer, an endometrial cancer, a gestational trophoblastic tumor, an ovarian epithelial cancer, a uterine sarcoma, a vaginal cancer, a vulvar cancer, a head and neck cancer, a hypopharyngeal cancer, a laryngeal cancer, a lip and oral cavity cancer, a metastatic squamous neck cancer, a nasopharyngeal cancer, an oropharyngeal cancer, a paranasal sinus and nasal cavity cancer, a pharyngeal cancer, a salivary gland cancer, a throat cancer, a musculoskeletal cancer, a bone cancer, a Ewing's sarcoma, a gastrointestinal stromal tumors (GIST), an osteosarcoma, a malignant fibrous histiocytoma of bone, a rhabdomyosarcoma, a soft tissue sarcoma, a uterine sarcoma, a neurologic cancer, a brain tumor, an astrocytoma, a brain stem glioma, a central nervous system atypical teratoid/rhabdoid tumor, a central nervous system embryonal tumors, a central nervous system germ cell tumor, a craniopharyngioma, an ependymoma, a medulloblastoma, a spinal cord tumor, a supratentorial primitive neuroectodermal tumors and pineoblastoma, a neuroblastoma, a respiratory cancer, a thoracic cancer, a non-small cell a lung cancer, a small cell lung cancer, a malignant mesothelioma, a thymoma, a thymic carcinoma, a skin cancer, a Kaposi's sarcoma, a melanoma, or a Merkel cell carcinoma, or any equivalent mouse cancer thereof, e.g., a cancer in a mouse model of a human cancer.

In particular embodiments, the tumor cell is derived from a non-hematologic cancer, e.g., a solid tumor. In certain embodiments, the tumor cell is derived from a hematologic cancer. In certain embodiments, the tumor cell is derived from a cancer that is a B cell malignancy or a hematological malignancy. In particular embodiments, the tumor cell is derived from a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM), or any equivalent mouse cancer thereof e.g., a cancer in a mouse model of a human cancer. In some embodiments, the antigen-expressing cell is a neoplastic, cancerous, and/or tumorigenic B cell.

In certain embodiments, the antigen-expressing cell is or includes a cell of a B cell cancer line. In particular embodiments, the B cell cancer line is a cell line that originates from or is derived from a neoplastic, cancerous and/or tumorigenic B cell, e.g., a mouse B cell. In certain embodiments, the antigen-expressing cells are or include L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells. In certain embodiments, the antigen-expressing cells are A20 cells. A20 cells are known, and are described for example in Kim et al., (1979) Establishment and characterization of BALB/c lymphoma lines with B cell properties. Journal of Immunology, 122(2): 549-554 and Graner et al., Immunoprotective activities of multiple chaperone proteins isolated from murine B-cell leukemia/lymphoma. Clin Cancer Res 2000; 6(3):909-915.

In certain embodiments, the antigen-expressing cells are administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the immunotherapy is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In particular embodiments, the antigen-expressing cells are administered intravenously. In certain embodiment, the antigen expressing cells are administered intravenously into the lateral tail vein. In some embodiments, the antigen expressing cells are administered subcutaneously.

In certain embodiments, between 5×10³ to 1×10⁹ antigen expressing-cells, between 1×10⁵ to 1×10⁸ CD4+antigen expressing-cells, between 1×10⁴ to 1×10⁶ antigen expressing-cells, between 5×10⁴ to 1×10⁷ antigen expressing-cells, between 2×10⁴ to 1×10⁶ antigen expressing-cells, between 5×10⁴ to 5×10⁶ antigen expressing-cells, between 1×10⁴ to 1×10⁶ antigen expressing-cells, between 1×10⁶ to 1×10⁷ antigen expressing-cells, between 5×10⁶ to 5×10⁸ antigen expressing-cells, between 1×10⁵ to 1×10⁷ antigen expressing-cells, between 1×10⁶ to 1×10⁸ antigen expressing-cells, or between 1×10⁵ to 1×10⁶ antigen expressing-cells are administered, injected, or infused. In particular embodiments, an amount of, of about, or of at least 1×10⁴, 2×10⁴, 2.5×10⁴, 3×10⁴, 4×10⁴, 5×10⁴, 6×10⁴, 7×10⁴, 7.5×10⁴, 8×10⁴, 9×10⁴, 1×10⁵, 2×10⁵, 2.5×10⁵, 3×10⁵, 4×10⁵, 5×10⁵, 6×10⁵, 7×10⁵, 7.5×10⁵, 8×10⁵, 9×10⁵, 1×10⁶, 2×10⁶, 2.5×10⁶, 3×10⁶, 4×10⁶, 5×10⁶, 6×10⁶, 7×10⁶, 8×10⁶, 9×10⁶, 1×10⁷, 1.1×10⁷, 1.2×10⁷, 1.25×10⁷, 1.3×10⁷, 1.4×10⁷, 1.5×10⁷, 1.6×10⁷, 1.7×10⁷, 1.75×10⁷, 1.8×10⁷, 1.9×10⁷, 2×10⁷, 2.5×10⁷, 5×10⁷, 7.5×10⁷, 3×10⁷, 3.5×10⁷, 4×10⁷, 5×10⁷, 7×10⁷, 8×10⁷, 9×10⁷, 1×10⁸, 2×10⁸, 3×10⁸, 4×10⁸, 5×10⁸, 6×10⁷, 7×10⁸, 8×10⁸, 1×10⁸, 1×10⁹, 5×10⁹, or 1×10¹⁰ antigen expressing cells are injected and/or infused. In particular embodiments, an amount of, of about, or of at least, 5×10⁴, 1×10⁵, 2×10⁵, 5×10⁵, or 1×10⁶ cells are administered, injected, or infused. In some embodiments, an amount of, of about, or of at least 2×10⁵ cells are administered, injected, or infused. In certain embodiments, the antigen-expressing cells are cells of a B cell cancer line, e.g., A20 cells.

In particular embodiments, the antigen-expressing cells are administered, injected, or infused prior to, during, or subsequent to administering the lymphodepleting agent or therapy and/or the immunotherapy. In particular embodiments, the antigen-expressing cells are administered, injected, or infused prior to administering the lymphodepleting agent or therapy and/or the immunotherapy. In some embodiments, the antigen-expressing cells are administered and or infused between 20 weeks and 1 hour, between 20 weeks and 10 weeks, between 15 weeks and 5 weeks, between 10 weeks and 1 week, between 10 weeks and 5 weeks, between 6 weeks and 1 week, between 6 weeks and 4 weeks, between 5 weeks and 1 week, between about 3 weeks and about 2 weeks, between 3 weeks and 1 day, between 28 days and 14 days, between 21 days and 7 days, between 21 days and 14 days, between 18 days and 10 days, between 20 days and 10 days, or between 17 days and 1 day prior to administering the lymphodepleting agent or therapy and/or the immunotherapy, each inclusive. In particular embodiments, the antigen expressing cells are administered at, at about, or within 20 weeks, 16 weeks, 12 weeks, 10 weeks, 8 weeks, 6 weeks, 5 weeks, 4 weeks, 28 days, 24 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 48 hours, 36 house, 24 hours, 12 hours, 8 hours, 6 hours, 4 hours, 2 hours, or 1 hour prior to administering the lymphodepleting agent or therapy and/or the immunotherapy. In certain embodiments, the antigen expressing cells are administered at, at about, or within 20 weeks, 16 weeks, 12 weeks, 10 weeks, 8 weeks, 6 weeks, 5 weeks, 4 weeks, 28 days, 24 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 48 hours, 36 house, 24 hours, 12 hours, 8 hours, 6 hours, 4 hours, 2 hours, or 1 hour prior to administering the lymphodepleting agent or therapy and/or the immunotherapy. In particular embodiments, the antigen-expressing cells are administered and or infused at or about 2 weeks and 4 weeks or 2 weeks and 3 weeks, each inclusive, prior to administration of the immunotherapy. In various embodiments, the antigen-expressing cells are administered and or infused at or about 17 days, 19 days, or 27 days prior to the administration of the immunotherapy.

In certain embodiments, between 1×10⁴ to 1×10⁶ A20 cells are administered, injected, and/or infused subcutaneously. In particular embodiments, between 1×10⁴ to 1×10⁶ A20 cells are intravenously injected or infused into the lateral tail vein. In certain embodiments, an amount of or about 2×10⁵ A20 cells are injected or infused into the lateral tail vein.

In particular embodiments, the mouse model is generated by administering a lymphodepleting agent and an immunotherapy to a mouse that contains antigen-expressing cells. In particular embodiments, the immunotherapy binds to or recognizes the antigen. In some embodiments, the antigen-expressing cell is a cancer or tumor cell. In particular embodiments, the antigen is a B cell antigen, e.g., an antigen that is expressed by B cells such as endogenously or naturally expressed by B cells. In particular embodiments, the antigen-expressing cell expresses a B cell antigen, such as a mouse B cell antigen or B cell marker. In some embodiments, the antigen-expressing cell expresses mouse CD20, CD19, CD22, ROR1, CD45, CD21, CD5, CD33, Igkappa, Iglambda, CD79a, CD79b or CD30. In particular embodiments, the antigen-binding cells expresses mouse CD19. In particular embodiments, the mouse model is generated by administering a lymphodepleting agent and an immunotherapy that binds to or recognizes mouse CD19 to a mouse that contains cells that express mouse CD19. In certain embodiments, the cells expressing mouse CD19 are or include A20 cells.

In particular embodiments, the provided mouse models are generated by (i) injecting antigen-expressing cells, e.g., cancer cells, to an immunocompetent mouse and then (ii) subsequently administering a lymphodepleting agent or therapy at, at about, or within 6 weeks, 5 weeks, 4 weeks, 3 weeks, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day after injecting the antigen-expressing cells and then (iii) administering an immunotherapy that binds to or recognizes the antigen of the antigen expressing cells at, at about, or within 30 hours, 24 hours, or 18 hours after the administration of the lymphodepleting agent. In certain embodiments, the immunotherapy is administered at about, or within 6 weeks, 5 weeks, 4 weeks, 3 weeks, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day after injecting the antigen-expressing cells. In some embodiments, the lymphodepleting agent or the immunotherapy is administered between or between about 1 week and 6 weeks, 2 weeks and 4 weeks, or 2 weeks and 3 weeks after the antigen expressing cells are injected, each inclusive.

In certain embodiments, the mouse model is generated by (i) injecting between or between about 1×10⁴ to 1×10⁶ antigen expressing-cells, inclusive, and then (ii) administering between or about between 1 mg/kg and 1,000 mg/kg of a lymphodepleting agent or therapy within between 1 week and 4 weeks, inclusive, after the antigen-expressing cells are injected, and then (iii) administering an immunotherapy, e.g., an immune cell therapy, that binds to or recognizes the antigen expressed by the antigen-expressing cells within between 18 hours and 30 hours, inclusive, after administration of the lymphodepleting agent.

In particular embodiments, the mouse model is generated by (i) injecting between or between about 5×10⁴ to 5×10⁵ antigen expressing-cells that express a B cell antigen or marker, inclusive, into an immunocompetent BALB/c mouse (or strain or substrain thereof) and then (ii) administering between or about between 50 mg/kg and 500 mg/kg i.p. of CPA within between 1 week and 4 weeks, inclusive, after the antigen-expressing cells are injected, and then (iii) administering CAR−T cells that bind to or recognize the B cell antigen or marker within between 18 hours and 30 hours, inclusive, after injection of the CPA.

In some embodiments, the mouse model is generated by (i) injecting between or between about 5×10⁴ to 5×10⁵ A20 cells, inclusive, into the tail vein of an immunocompetent BALB/c mouse (or strain or substrain thereof) and then (ii) administering between or about between 50 mg/kg and 500 mg/kg i.p. of CPA within between 2 weeks and 4 weeks, inclusive, after the antigen expressing cells are injected, and then (iii) administering between or between about 5×10⁶ and 50×10⁶ anti-mouse CD19 CAR−T cells within between 18 hours and 30 hours, inclusive, after injection of the CPA. In particular embodiments, the mouse model is generated by (i) injecting an amount of or of about 2×10⁶ A20 cells into the tail vein of an immunocompetent BALB/c mouse (or strain or substrain thereof) and then (ii) administering a dose of or about 100 mg/kg or 250 mg/kg i.p. of CPA within between 2 weeks and 4 weeks, inclusive, after the antigen expressing cells are injected, or infused, and then (iii) administering a dose of or of about 5×10⁶, 10×10⁶, or 20×10⁶ anti-mouse CD19 CAR−T cells at or about 24 hours after the injection of CPA. In some embodiments, the CPA or the CAR−T cells are administered at or about 17 days, 19 days, or 27 days after the A20 cells are injected.

### II. ATTRIBUTES AND PHENOTYPES OF THE MOUSE MODEL

In some embodiments, a mouse that is a mouse model of toxicity has been administered an immunotherapy. In certain embodiments, the mouse is a mouse produced by any of the methods described herein. In particular embodiments, the mouse that is a mouse model of toxicity has been administered an immunotherapy that is described herein, e.g., in Section I.C. In certain embodiments, the mouse that is a mouse model of toxicity has been administered an immunotherapy after being administered a lymphodepleting agent or therapy. In particular embodiment, the lymphodepleting agent or therapy is a lymphodepleting agent or therapy that is described herein, e.g., in Section I.B. In some embodiments, the immunotherapy was a T-cell engaging immunotherapy. In some embodiments, the immunotherapy was a cell therapy, e.g., a cell composition containing cells expressing a recombinant receptor. In certain embodiments, the recombinant receptor is a CAR. In certain embodiments, the mouse is a mouse model of toxicity that had been administered, injected, or infused with cells that express an antigen, e.g., exogenous cells that express an antigen that is bound by and/or recognized by the immunotherapy. In certain embodiments, the mouse has been administered, injected, or infused with antigen-expressing cells described herein, e.g., in Section I.D.

In particular embodiments, a sign, symptom, or outcome of the mouse model described herein is assessed, measured, detected, and/or quantified in relation to the individual mouse at a time prior to or at the same time as administration of the immunotherapy. Thus, in certain embodiments, an appearance, increase, or decrease of one or more phenotypes or attributes of the mouse model is in relation to the one or more phenotypes of the mouse prior to or at the time of the administration of the immunotherapy. In some embodiments, an attribute or phenotype of the mouse model described herein is assessed, measured, detected, and/or quantified in relation to a mouse that did not receive the immunotherapy or in relation to a naive mouse. In certain embodiments, an appearance, increase, or decrease of one or more phenotypes or attributes of the mouse model is in relation to the one or more phenotypes of the mouse that was not administered the immunotherapy. In some embodiments, a sign, symptom, or outcome of the mouse model, e.g., altered level, amount or expression an attribute, e.g., expression of a molecule, comprises an increased level, amount or expression compared to the level, amount or expression of the molecule in the mouse prior to administering the lymphodepleting therapy and/or immunotherapy and/or compared to the level, amount or expression of the molecule, on average, in a naïve mouse of the same strain and/or compared to the level, amount or expression of the molecule, on average, in a naïve mouse of the same strain.

In some embodiments, an appearance, increase, or decrease of one or more phenotypes or attributes of the mouse model is assessed, measured, detected, and/or quantified after immunotherapy is administered, e.g., about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy. In some embodiments, the appearance, increase, or decrease of one or more phenotypes or attributes of the mouse model is assessed, measured, detected, and/or quantified prior to administration of immunotherapy. In some embodiments, the an appearance, increase, or decrease of one or more phenotypes or attributes of the mouse model is assessed, measured, detected, and/or quantified at the time of administration of immunotherapy.

In certain embodiments, the appearance, increase, or decrease of one or more phenotypes or attributes of the mouse model is detectable within 4 weeks, within 3 weeks, within 2 weeks, within 1 week, within 10 days, within 9 days, within 8 days, within 7 days, within 6 days, within 5 days, within 4 days, within 3 days, within 2 days, of within 1 day after the immunotherapy is administered. In some embodiments, the appearance, increase, or decrease of one or more phenotypes or attributes of the mouse model is detectable between 1 day and 4 weeks, between 1 day and 21 days, between 1 day and 14 days, between 1 and 7 days, between 1 and 3 days, or between 2 days and 5 days after administration of the immunotherapy. In certain embodiments, the appearance, increase, or decrease of one or more phenotypes or attributes of the mouse model is detectable at, at about, or within 1 day, 2 days, 3 days, 4 days, 5 days, 6 days, or 7 days after administration of the immunotherapy.

In particular embodiments, the mouse contains a portion, aspect, or component of an immunotherapy, e.g., a circulating antibody or cell of an immunotherapy. In certain embodiments, the mouse that is a mouse model of toxicity contains a portion, aspect, or component of an immunotherapy that is described herein, e.g., in Section I.C. In some embodiments, the mouse that is a mouse model of toxicity contains a T-cell engaging therapy or an aspect or portion thereof, e.g., contains circulating levels of an agent and/or an antibody associated with a T-cell engaging therapy. In certain embodiments, the mouse contains one or more cells associated with an immunotherapy. In some embodiments, the mouse contains one or more cells that express a recombinant receptor. In certain embodiments, the recombinant receptor is a CAR. In certain embodiments, the mouse contains cells that express an antigen, e.g., exogenous cells that express an antigen that is bound by and/or recognized by the immunotherapy. In certain embodiments, the mouse contains antigen-expressing cells described herein, e.g., in Section I.D.

In some embodiments, the immunotherapy, or a portion, aspect, or component thereof, undergoes in vivo expansion in the mouse. In some embodiments, one or more cells of the immunotherapy undergo and/or have undergone in vivo expansion in the mouse. In some embodiments, the cells that undergo and/or have undergone expansion reach a peak level of circulating cells between about 1 day and about 7 days, between 3 days and 5 days, between 2 days and 4 days, between 2 days and 5 days, between 3 days and 10 days, between 1 day and 5 days, between 5 days and 10 days, between 2 days and 7 days, between 3 days and 8 days, between 4 days and 9 days, between 7 days and 9 days, between 6 days and 10 days, between 1 day and 20 days, between 10 days and 14 days, between 14 days and 21 days, and/or between 7 days and 28 days after the immunotherapy is administered, each inclusive. In particular embodiments, the peak level is reached between 7 days and 14 days after administration of the immunotherapy. In certain embodiments, the peak level is reached at or at about 10 days after administration of the immunotherapy.

In some embodiments, the peak level of cells, e.g., cells of the immunotherapy, is, is at least, or is about 0.01 cells/μl blood, 0.1 cells/μl blood, 0.2 cells/μl blood, 0.3 cells/μl blood, 0.4 cells/μl blood, 0.5 cells/μl blood, 06 cells/μl blood, 0.7 cells/μl blood, 0.8 cells/μl blood, 0.9 cells/μl blood, 1 cells/μl blood, 1.2 cells/μl blood, 1.4 cells/μl blood, 1.6 cells/μl blood, 1.8 cells/μl blood, 2 cells/μl blood, 2.5 cells/μl blood, 3 cells/μl blood, 4 cells/μl blood, 5 cells/μl blood, 6 cells/μl blood, 7 cells/μl blood, 8 cells/μl blood, 9 cells/μl blood, or greater than 10 cells/μl blood, 15 cells/μl blood, 20 cells/μl blood, 25 cells/μl blood, 50 cells/μl blood, 100 cells/μl blood, 200 cells/μl blood, 200 cells/μl blood, 200 cells/μl blood, 300 cells/μl blood, 400 cells/μl blood, 500 cells/μl blood, 600 cells/μl blood, 700 cells/μl blood, 800 cells/μl blood, 900 cells/μl blood, 1,000 cells/μl blood, 2,000 cells/μl blood, 3,000 cells/μl blood, 4,000 cells/μl blood, or 5,000 cells/μl blood. In particular embodiments, the peak level is between 1 and 10 cells/μl blood, inclusive. In certain embodiments, the peak level is between 10 and 200 cells/μl blood, inclusive. In particular embodiments, the peak level is between 130 and 170 cells/μl blood. In some embodiments, the cells express a recombinant receptor, e.g., a CAR.

In certain embodiments, the immunotherapy of the mouse model persists over an amount of time. In particular embodiments, the immunotherapy is detectable in vivo for an amount of time after the immunotherapy is administered. In certain embodiments, the immunotherapy is persists in vivo for at least or at least about 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after the immunotherapy is administered. In some embodiments, the immunotherapy persists in vivo for at least or at least about 42 days. In some embodiments, the immunotherapy is or contains a T cell composition containing cells that express a recombinant receptor or a CAR. In certain embodiments, CAR expressing cells persist in vivo for at least or at least about 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after the immunotherapy is administered. In particular embodiments, the CAR expressing cells persist in vivo for at least or at least about 42 days.

In certain embodiments, the immunotherapy is active and/or possesses an activity. In some embodiments, the immunotherapy is active and/or possesses an activity in vivo. In particular embodiments, the activity is the removal of cancer cells and/or tumor cells. In certain embodiments, the activity is the removal of cells that express an antigen that is recognized and/or bound by the immunotherapy. In particular embodiments, the activity is the removal of antigen-expressing cells, e.g., any one or more antigen-expressing cells that are described herein, such as in Section I.D. In some embodiments, the antigen-expressing cells are A20 cells. In certain embodiments, the mouse administered the immunotherapy has at least about 10%, 20%, 30%, 40%, 50%, 60%, 70%, 80%, 90%, 95%, 99%, 99.9%, 99.99%, 99.999%, or 99.9999% less antigen-expressing cells, e.g., A20 cells, than a mouse that was administered the same amount of antigen-expressing cells and was not administered the immunotherapy.

In some embodiments, the immunotherapy, e.g., a cell expressing the recombinant receptor and/or CAR, binds to and/or recognizes an antigen that is expressed on a B cell. In certain embodiments, the mouse has previously been administered a lymphodepleting agent or therapy. In some embodiments, the mouse has B cell aplasia. In certain embodiments, the mouse has a reduced amount and/or level of B cells. In certain embodiments, the level and/or amount of B cells is reduced as compared to the level and/or amount of B cells in a mouse that does not contain the cells expressing a recombinant receptor and/or that has not been administered a lymphodepleting agent or therapy. In some embodiments, the mouse has a reduction of at least 25%, at least 33%, at least 50%, at least 60%, at least 70%, at least 80%, at least 90%, at least 95%, at least 97%, at least 98%, at least 99%, at least 99.5%, at least 99.9%, at least 99.95%, at least 99.99%, or at least 99.999% B cells. In particular embodiments, the mouse has a reduction of at least 25%, at least 33%, at least 50%, at least 60%, at least 70%, at least 80%, at least 90%, at least 95%, at least 97%, at least 98%, at least 99%, at least 99.5%, at least 99.9%, at least 99.95%, at least 99.99%, or at least 99.999% circulating B cells. In some embodiments, the mouse has an amount of B cells less than or equal or about equal to 100 B cells/μl blood, 50 B cells/μl blood, 40 B cells/μl blood, 30 B cells/μl blood, 25 B cells/μl blood, 20 B cells/μl blood, 15 B cells/μl blood, 14 B cells/μl blood, 13 B cells/μl blood, 12 B blood, 11 B cells/μl blood, 10 B cells/μl blood, 9 B cells/μl blood, 8 B cells/μl blood, 7 B cells/μl blood, 6 B cells/μl blood, 5 B cells/μl blood, 4 B cells/μl blood, 3 B cells/μl blood, 2 B cells/μl blood, 1 B cells/μl blood, 0.5 B cells/μl blood, 0.1B cells/μl blood, 0.05 B cells/μl blood, or 0.01 B cells/μl blood.

In certain embodiments, the level and/or amount of B cells or circulating B cells is reduced for at least about 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after the immunotherapy is administered.

In certain embodiments, the immunotherapy, e.g., one or more cells expressing a recombinant receptor and/or a CAR, infiltrates one or more tissues. In certain embodiments, the immunotherapy infiltrates one or more of connective tissue, muscle tissue, nervous tissue, or epithelial tissue. In certain embodiments, the immunotherapy and/or a cell of the immunotherapy infiltrates one or more tissues of heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons. In certain embodiments, the one or more tissues are brain tissue, liver tissue, spleen tissue, lung tissue, or kidney tissue. In some embodiments, the immunotherapy infiltrates brain tissue.

In some embodiments, the immunotherapy, e.g., one or more cells expressing a recombinant receptor and/or a CAR, infiltrates a tissue in an amount of or about 0.001 cells, 0.005 cells, 0.01 cells, 0.05 cells, 0.1 cells, 0.5 cells, 1.0 cells, 1.5 cells, 2.0 cells, 2.5 cells, 3.0 cells, 3.5 cells, 4.0 cells, 4.5 cells, 5.0 cells, 5.5 cells, 6.0 cells, 6.5 cells, 7.0 cells, 7.5 cells, 8.0 cells, 8.5 cells, 9.0 cells, 9.5 cells, 10 cells, 15 cells, 20 cells, 25 cells, 30 cells, 35 cells, 40 cells, 45 cells, 50 cells, 100 cells, 200 cells, 300 cell, 400 cells, 500 cells, 600 cell, 700 cells, 800 cells, 900 cells, 1,000 cells, 1,200 cells, 1,400 cells, 1,600 cells, 1,800 cell, 2,000 cells, 3,000 cells, 4,000 cells, 5,000 cells, or greater than or greater than about 5,000 cells the immunotherapy, e.g., one or more cells expressing a recombinant receptor and/or a CAR, per mg of tissue. In particular embodiments, the immunotherapy the immunotherapy, e.g., one or more cells expressing a recombinant receptor and/or a CAR, infiltrates the tissue in an amount that is at least about 0.001 cells, 0.005 cells, 0.01 cells, 0.05 cells, 0.1 cells, 0.5 cells, 1.0 cells, 1.5 cells, 2.0 cells, 2.5 cells, 3.0 cells, 3.5 cells, 4.0 cells, 4.5 cells, 5.0 cells, 5.5 cells, 6.0 cells, 6.5 cells, 7.0 cells, 7.5 cells, 8.0 cells, 8.5 cells, 9.0 cells, 9.5 cells, 10 cells, 15 cells, 20 cells, 25 cells, 30 cells, 35 cells, 40 cells, 45 cells, 50 cells, 100 cells, 200 cells, 300 cell, 400 cells, 500 cells, 600 cell, 700 cells, 800 cells, 900 cells, 1,000 cells, 1,200 cells, 1,400 cells, 1,600 cells, 1,800 cell, 2,000 cells, 3,000 cells, 4,000 cells, or 5,000 of the immunotherapy, e.g., one or more cells expressing a recombinant receptor and/or a CAR, per mg of tissue.

In some embodiments, the immunotherapy, e.g., one or more cells expressing a recombinant receptor and/or a CAR, infiltrates brain tissue in an amount of or about 0.001 cells, 0.005 cells, 0.01 cells, 0.05 cells, 0.1 cells, 0.5 cells, 1.0 cells, 1.5 cells, 2.0 cells, 2.5 cells, 3.0 cells, 3.5 cells, 4.0 cells, 4.5 cells, 5.0 cells, 5.5 cells, 6.0 cells, 6.5 cells, 7.0 cells, 7.5 cells, 8.0 cells, 8.5 cells, 9.0 cells, 9.5 cells, 10 cells, 15 cells, 20 cells, 25 cells, 30 cells, 35 cells, 40 cells, 45 cells, 50 cells, or greater than or greater than about 50 cells the immunotherapy, e.g., one or more cells expressing a recombinant receptor and/or a CAR, per mg of tissue. In particular embodiments, the immunotherapy the immunotherapy, e.g., one or more cells expressing a recombinant receptor and/or a CAR, infiltrates brain tissue in an amount that is at least about 0.001 cells, 0.005 cells, 0.01 cells, 0.05 cells, 0.1 cells, 0.5 cells, 1.0 cells, 1.5 cells, 2.0 cells, 2.5 cells, 3.0 cells, 3.5 cells, 4.0 cells, 4.5 cells, 5.0 cells, 5.5 cells, 6.0 cells, 6.5 cells, 7.0 cells, 7.5 cells, 8.0 cells, 8.5 cells, 9.0 cells, 9.5 cells, 10 cells, 15 cells, 20 cells, 25 cells, 30 cells, 35 cells, 40 cells, 45 cells, or 50 cells of the immunotherapy, e.g., one or more cells expressing a recombinant receptor and/or a CAR, per mg of tissue.

In particular embodiments, the sign, symptom, and/or outcome resembles, is equivalent to, and/or is similar to a sign, symptom, and/or outcome associated with toxicity in a human subject. In particular embodiments, the sign, symptom, and/or outcome of the mouse resembles, is equivalent to, and/or is similar to a sign, symptom, or outcome associated with toxicity to an immunotherapy in a human subject. In some embodiments, the toxicity is a toxicity to an immune system stimulator in a human subject. In certain embodiments, the toxicity is a toxicity to a T-cell engaging therapy in a human subject. In particular embodiments, the toxicity is a toxicity to a cell therapy in a human subject. In certain embodiments, the cell therapy is or includes administration of a cell composition that contains one or more engineered cells. In certain embodiments, the cell composition contains one or more cells that express a recombinant receptor. In certain embodiments, the composition contains one or more cells expressing a CAR. In particular embodiments, the cell composition contains one or more cells that binds to and/or recognizes an antigen expressed by a B cell. In some embodiments, the antigen is CD19. In particular embodiments, the toxicity to the immunotherapy experienced by the human subject is or includes cytokine release syndrome. In certain embodiments, the toxicity to the immunotherapy experienced by the human subject is or includes neurotoxicity.

In some embodiments, the mouse has one or more signs, symptoms, or outcomes associated with the mouse model that are caused by, manifested as, and/or associated with increased inflammation, changes in gene expression, altered blood chemistry, tissue damage, brain edema, weight loss, reduced body temperature, and/or altered behavior. In particular embodiments, the mouse have one or more signs, symptoms, or outcomes, e.g., symptoms of toxicity, as compared to a mouse that has not been administered the immunotherapy. In certain embodiments, the mouse has one or more signs, symptoms, and/or outcomes as compared to a mouse that has not been administered the lymphodepleting agent or therapy. In certain embodiments, the mouse has one or more signs, symptoms, and/or outcomes, e.g., symptoms of toxicity, as compared to a naïve mouse, i.e., a mouse that has not been administered the lymphodepleting agent or therapy and has not been administered the immunotherapy, nor any mock or control immunotherapy thereof. In some embodiments, the mouse that has not been administered the immunotherapy has been administered a mock immunotherapy. In certain embodiments, the mock immunotherapy is does not recognize and/or bind to the antigen that is recognized and/or bound by the immunotherapy. In some embodiments, the mock immunotherapy is or includes a cell composition that does not contain any cells that express a recombinant receptor. In some embodiments, the mouse that has not been administered the immunotherapy has been administered a control immunotherapy. In some embodiments, the control immunotherapy does not bind to the antigen. In particular embodiments, the control immunotherapy binds to and/or recognizes a human antigen, but not a mouse antigen. In certain embodiments, the control immunotherapy is or includes a cell composition that contains cells expressing a recombinant receptor, e.g., a CAR, that binds to and/or recognizes a human antigen but not a mouse antigen.

In certain embodiments, the one or more signs, symptoms, and/or outcomes of the mouse model is inflammation and/or an increase in inflammation. Inflammatory markers include, but are not limited to, cytokines or other inflammatory mediators that promote the attraction of white blood cells or inflammatory cells. Inflammatory markers can be, but are not necessarily, released from inflammatory cells. Inflammatory markers include, but are not limited to, 8-isoprostane, myeloperoxidase, IL-6, and C-reactive protein. Oxidative stress markers indicate cell damage caused by oxidants or free-radicals. Oxidative stress markers include the radicals and oxidants that reach their respective targets, such as lipids, protein, or DNA, as well as indirect markers of the damage caused by radicals and oxidants. Oxidative stress markers include, but are not limited to, free iron, 8-isoprostane, superoxide dismutase, glutathione peroxidase, lipid hydroperoxidase, dityrosine, and 8-hydroxyguanine. For example, 8-isoprostane can be classified as both an inflammatory marker and an oxidative stress marker.

In certain embodiments, the one or more signs, symptoms, and/or outcomes of the mouse model is or includes an increase in one or more cytokines and/or chemokines. In some embodiments, the increase is an increase of one or more circulating or serum cytokines and/or chemokines. In some embodiments, the one or more cytokines and/or chemokines are proinflammatory cytokines and/or chemokines. In some embodiments, the one or more cytokines and/or chemokines are or include of one or more of IL-2, IL-4, IL-5, GM-CSF, IFN-gamma, TNF-alpha, IL-10, MIP-1b, MCP-1, IL-6, Angiopoietin-2, EPO, IL-12p70, IL-13, IL-15, IL-17E/IL25, IL-21, IL-23, IL-30, IP-10, KC/GRO, and MIP-1a.

In certain embodiments, the one or more cytokines and/or chemokines are increased by at least 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold. In certain embodiments, one or more of IL-2, IL-4, IL-5, GM-CSF, IFN-gamma, TNF-alpha, IL-10, MIP-1b, MCP-1, IL-6, Angiopoietin-2, EPO, IL-12p70, IL-13, IL-15, IL-17E/IL25, IL-21, IL-23, IL-30, IP-10, KC/GRO, and MIP-1a are increased by at least 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold.

In some embodiments, the increase in one or more cytokines and/or chemokines is in comparison with the level of one or more cytokines and/or chemokines in control mice. In some embodiments, the increase in one or more cytokines and/or chemokines is in comparison with the level of one or more cytokines and/or chemokines in a mouse that has not been administered the immunotherapy, a mouse that has been administered a control (non-target) immunotherapy or in naïve mice. In some embodiments, the increase in one or more cytokines and/or chemokines is in comparison with the level of one or more cytokines and/or chemokines in the same mouse prior to or at the time of administering immunotherapy. In some embodiments, the increase in one or more cytokines and/or chemokines is in comparison with increase in one or more cytokines and/or chemokines in the mouse prior to administering the lymphodepleting therapy and/or immunotherapy and/or compared to the increase in one or more cytokines and/or chemokines, on average, in a naïve mouse of the same strain.

In some embodiments, the increase in one or more cytokines and/or chemokines is observed after immunotherapy is administered, e.g., about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy. In some embodiments, the increase in one or more cytokines and/or chemokines is observed prior to administration of immunotherapy. In some embodiments, the increase in one or more cytokines and/or chemokines is observed at the time of administration of immunotherapy.

In some embodiments, the one or more cytokines or chemokines, e.g., one or more of IL-2, IL-4, IL-5, GM-CSF, IFN-gamma, TNF-alpha, IL-10, MIP-1b, MCP-1, IL-6, Angiopoietin-2, EPO, IL-12p70, IL-13, IL-15, IL-17E/IL25, IL-21, IL-23, IL-30, IP-10, KC/GRO, and MIP-1a, are present in serum at a concentration of, of about, or at least 5 pg/μl, 10 pg/μl, 25 pg/μl, 50 pg/μl, 75 pg/μl, 100 pg/μl, 200 pg/μl, 250 pg/μl, 300 pg/μl, 400 pg/μl, 500 pg/μl, 600 pg/μl, 700 pg/μl, 750 pg/μl, 800 pg/μl, 900 pg/μl, or 1,000 pg/μl, such as at a time point within 14 days, 10 days, 7 days, 5 days, 4 days, 3 days, 72 hours, 60 hours, 48 hours, 36 hours, 24 hours, 18 hours, or 12 hours after administration of the immunotherapy. In some embodiments, the time point is at or at about 72 hours after administration of the immunotherapy. In particular embodiments, the one or more signs, symptoms, or outcomes of the model is or includes a serum concertation of at least 10 pg/μl of one or more of IL-2, GM-CSF, IFN-gamma, TNF-alpha, IL-10, EPO, IL-19p70, IL-15, IL-30, IL-23, or MIP-1a at, at about, or within 3 days after the administration of the immunotherapy. In various embodiments, the one or more signs, symptoms, or outcomes of the model is or includes a serum concertation of at least 100 pg/μl of one or more of IL-4, IL-5, MIP-1b, MCP1, IL-6, Angiopoietin-2, IL-13, IP-10, or KC/GRO at, at about, or within 3 days after the administration of the immunotherapy.

In particular embodiments, the one or more signs, symptoms, and/or outcomes of the mouse model is or includes an increase in ratio of angiopoetin-2 to angiopoetin-1 (Ang2:Ang1 ratio). In some embodiments, the Ang2:Ang1 ratio is the ratio of angiopoetin-2 to angiopoetin-1 present in the serum or circulation. In some embodiments, the ratio of angiopoetin-2 to angiopoetin-1 (Ang2:Ang1 ratio) is increased by at least 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold, e.g., compared to the Ang2:Ang1 ratio in the mouse prior to administering the lymphodepleting therapy and/or immunotherapy and/or compared to the Ang2:Ang1 ratio, on average, in a naïve mouse of the same strain.

In some embodiments, the ratio of angiopoetin-2 to angiopoetin-1 (Ang2:Ang1 ratio) exhibited by the mouse at any time point before or after administration of the immunotherapy is high, e.g., at least 1 or higher, e.g., at least 2, at least 3, at least 4, at least 5, at least 10, at least 15, at least 20, at least 25, at least 30, at least 40, at least 50, at least 100, at least 500, at least 1,000, or at least 5,000 or higher. In some embodiments, the ratio of angiopoetin-2 to angiopoetin-1 is greater than 1, e.g., between about 2 and 100 such as at or about 32, within 2, 3, or 4 days after the administration of the immunotherapy.

In some embodiments, the Ang2:Ang1 ratio is increased compared to control mice. In some embodiments, the Ang2:Ang1 ratio is increased compared to the ratio in a mouse that has not been administered the immunotherapy, a mouse that has been administered a control (non-target) immunotherapy, a mouse that has been administered a control (non-target) immunotherapy or in naïve mice. In some embodiments, the Ang2:Ang1 ratio is increased compared to the ratio in the same mouse prior to or at the time of administering immunotherapy.

In some embodiments, the increase in Ang2:Ang1 ratio or a high Ang2:Ang1 ratio, e.g., Ang2:Ang1 ratio of at least 1 or higher, is observed after immunotherapy is administered, e.g., about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy. In some embodiments, the increase in or high Ang2:Ang1 ratio is observed prior to administration of immunotherapy. In some embodiments, the increase in or high Ang2:Ang1 ratio is observed at the time of administration of immunotherapy.

The provided mouse model of toxicity exhibits one or more features of toxicity observed in human subjects. In some contexts, a higher ratio of angiopoetin-2 to angiopoetin-1 is observed in human subjects, e.g., human patients, that exhibit severe CRS, e.g., grade 4 or higher CRS, compared to subjects that do not exhibit CRS. In some contexts, a higher Ang2:Ang1 ratio is observed in human subjects, e.g., human patients, that exhibit severe CRS, including prior to the start of lymphodepletion chemotherapy, before CAR−T cell infusion (pre-infusion), and on day 1 after CAR−T cell infusion (see Hay et al. Blood 2017: blood-2017-06-793141). In some contexts, the mouse model exhibits similar signs, symptoms, and/or outcomes, including an Ang2:Ang1 ratio of at least 1 and/or a higher Ang2:Ang1 ratio compared to control mice or naïve mice.

In particular embodiments, the one or more signs, symptoms, and/or outcomes of the mouse model is or includes a change in gene expression of one or more genes in an organ, tissue, or cell type. In particular embodiments, the change in gene expression is an increase in gene expression, such as compared to a control described herein, e.g., a naïve mouse or a mouse that is not administered the immunotherapy. In some embodiments, the change in gene expression is a decrease in gene expression. In some embodiments, the change in gene expression is at least above a log 2 fold change of 0.1, 0.2, 0.3, 0.4, 0.5, 0.6, 0.7, 0.8, 0.9, 1.0, 1.2, 1.4, 1.6, 1.8, or 2.0, or is at least below a log 2 fold change of −0.1, −0.2, −0.3, −0.4, −0.5, −0.6, −0.7, −0.8, −0.9, −1.0, −1.2, −1.4, −1.6, −1.8, or −2.0, as compared to a control. In various embodiments, the change in gene expression is at least above a log 2 fold change of 0.5, 1.0, 1.4, or 2.0, or is at least below a log 2 fold change of −0.1, −0.2, −0.3, −0.4, −0.5, −0.6, −0.7, −0.8, −0.9, −1.0, −1.2, −1.4, −1.6, −1.8, or −2.0, as compared to a control, e.g., a naive mouse or a mouse that did not receive the immunotherapy.

In certain embodiments, one or more signs, symptoms, and/or outcomes of the mouse model is or includes a change in the expression of one or more genes. In certain embodiments, the one or more signs, symptoms, and/or outcomes is a change in the expression of one or more genes within a cell type, a cell from a tissue or organ, or within a tissue or organ, e.g., in brain, brain tissue, a brain cell, and/or a portion of the brain. In certain embodiments, the change in gene expression is an increase or decrease in the expression of the gene as compared to a control mouse, e.g., a that did not receive the immunotherapy. In some embodiments, the control mouse did not receive the immunotherapy or received an inactive variation of the immunotherapy, such as an immunotherapy that does not bind or recognize antigen present in the mouse. In some embodiments, the control mouse is a naïve mouse. In particular embodiments, the change in expression or one or more genes is observed in a cell or tissue of the mouse at, at about, or within 1 day, 2 days, 3 days, 4 days, 5 days, 6 days, 7 days, 10 days, 14 days, 18 days, 21 days, 25 days, or 28 days after the administration of the immunotherapy. In some embodiments, the change in expression of one or more genes is detectable in a cell, tissue, or organ, e.g., In certain embodiments, the change in gene expression is or includes a change in the expression of one or more genes in tissue or a cell found within tissue of the mouse, e.g., the brain.

In various embodiments, the one or more genes are or include one or more of Acer2 (Alkaline ceramidase 2), Adipoq (Adiponectin), Aif1 (Allograft inflammatory factor 1), Angpt1 (angeopotein 1), Angpt14 (angiopoietin-like 4), Angpt2 (angiopotein 2), Aox1 (Aldehyde oxidase), Aqp4 (Aquaporin-4), Atf3 (cyclic AMP-dependent transcription factor ATF-3), Bnip3 (BCL2/adenovirus E1B 19 kDa protein-interacting protein 3), Ccl2 (C-C motif chemokine 2), CCL4 (MIP-1b, C-C motif chemokine 4), CD31 (PECAM-1), CD274, CD68, CIITA (class II transactivator), CXCL1 (KC, Growth-regulated alpha protein), CXCL10 (IP-10), CXCL11 (I-TAC, C-X-C motif chemokine 11), Edn1 (Endothelin-1), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), HIF3a (hypoxia inducible factor 3 alpha subunit), ICAM-1 (Intercellular adhesion molecule 1), IL2ra (Interleukin-2 receptor subunit alpha), IL-4, IL-6, IL-13, Lrg1 (leucine rich alpha-2-glycoprotien 1), Mgst3 (Microsomal glutathione S-transferase 3), Mmrn2, (Multimerin-2), Ncf1 (Neutrophil cytosol factor 1), NLRC5 (class I transactivator), Nos3 (Nitric oxide synthase, endothelial), Pdk4 (pyruvate dehydrogenase kinase, isozyme 4), Pla2g3 (group 3 secretory phospholipase A2 precursor), Ptgs2 (Prostaglandin G/H synthase 2), Pxdn (Peroxidasin homolog), Scara3 (Scavenger receptor class A member 3), Sele (E-selectin), Selp (P-selectin), IL2ra, IL-13, Serpine 1, Sult1a1 (Sulfotransferase 1A1), Tgfb1 (Transforming growth factor beta-1), Tgfb2 (transforming growth factor beta 2), Tgfb3 (transforming growth factor beta 3), Tgtp1 (T-cell-specific guanine nucleotide triphosphate-binding protein 1), Tlr2 (Toll-like receptor 2), Tlr4 (toll like receptor 4), TNF (tumor necrosis factor), VCAM-1 (Vascular cell adhesion protein 1), Vwf (von Willebrand factor), and Xdh (xanthine dehydrogenase).

In certain embodiments, the one or more signs, symptoms, and/or outcomes of the mouse model, e.g., a mouse administered a lymphodepleting agent or therapy and an immunotherapy, is or includes a change in the expression of one or more of Acer2, Angpt14, Angpt2, Aox1, Atf3, Bnip3, CD274 (also known as PD-L1), CD31(PECAM-1), E-Selectin, Gbp2, Gbp4, Gbp5 and Gbp9, GM-CSF, Hif3a, ICAM-1, IL-4, IL-6, Lrg1, Mgst3, Mmrn2, Ncf1, Nos3, Pdk4, Pla2g3, P-Selectin, Ptgs2, Pxdn, Scara3, Scara3, Sult1a1, Ncf1, Tgtp1, Vwf, VCAM-1, and Xdh, such as compared to the expression in a control mouse. In some embodiments, the mouse model is or includes a mouse that was administered CPA and CAR−T cells.

In various embodiments, the one or more signs, symptoms, and/or outcomes of the mouse model, e.g., a mouse administered a lymphodepleting agent and an immunotherapy, is or includes a change in the expression of one or more of Acer2 (Alkaline ceramidase 2), Aif1 (Allograft inflammatory factor 1), Angpt14 (angiopoietin-like 4), Angpt2 (angiopotein 2), CD31 (PECAM-1), CXCL10 (IP-10), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), ICAM-1 (Intercellular adhesion molecule 1), IL2ra (Interleukin-2 receptor subunit alpha), IL-4, NLRC5 (class I transactivator), Pdk4 (pyruvate dehydrogenase kinase, isozyme 4), Sele (E-selectin), IL2ra, IL-13, Serpine 1, Sult1a1 (Sulfotransferase 1A1), Tgfb1 (Transforming growth factor beta-1), TNF (tumor necrosis factor), VCAM-1 (Vascular cell adhesion protein 1), Vwf (von Willebrand factor), and Xdh (xanthine dehydrogenase), such as compared to the expression in a control mouse. In some embodiments, the mouse model is or includes a mouse that was administered CPA and CAR−T cells.

In various embodiments, the one or more signs, symptoms, and/or outcomes of the mouse model, e.g., a mouse administered antigen-expressing cells, a lymphodepleting agent, and an immunotherapy, is or includes a change in the expression of one or more of one or more of Acer2, Adipoq (Adiponectin), Aif1 (Allograft inflammatory factor 1), Xdh (xanthine dehydrogenase, Angpt1, Angpt14 (angiopoietin-like 4), Angpt2 (angiopotein 2), Aqp4 (Aquaporin-4), Atf3 (cyclic AMP-dependent transcription factor ATF-3), Ccl2 (C-C motif chemokine 2), CD68, Edn1 (Endothelin-1), Gbp2 (guanylate-binding protein 2), Gbp4(guanylate-binding protein 4), Gdp5 (guanylate-binding protein 5), Gdp9 (guanylate-binding protein 9, HIF3a (hypoxia inducible factor 3 alpha subunit), isozyme 4), Lrg1 (leucine rich alpha-2-glycoprotien 1), Mmrn2, Pdk4 (pyruvate dehydrogenase kinase, Pla2g3 (group 3 secretory phospholipase A2 precursor), Sele (E-selectin), Serpine 1, Sult1a1 (Sulfotransferase 1A1, Tgfb1 (Transforming growth factor beta-1), Tgfb2 (transforming growth factor beta 2), Tgfb3 (transforming growth factor beta 3), Tlr2 (Toll-like receptor 2), Tlr4 (toll like receptor 4), VCAM-1 (Vascular cell adhesion protein 1), Vwf (von Willebrand factor), ICAM-1 (Intercellular adhesion molecule 1), Selp (P-selectin), IL2ra, IL-13, Gzmb (Granzyme B), TNF, CXCL10 (IP-10), CCL2 (MCP-1, C-C motif chemokine 2), CXCL11 (I-TAC, C-X-C motif chemokine 11), CXCL1 (KC, Growth-regulated alpha protein), CCL4 (MIP-1b, C-C motif chemokine 4), NLRC5 (class I transactivator), CIITA (class II transactivator),CD274, and Tgtp (T-cell-specific guanine nucleotide triphosphate-binding protein 1), such as compared to the expression in a control mouse. In certain embodiments, the mouse model is or includes a mouse that was administered A20 cells, CPA, and CAR−T cells.

In certain embodiments, the one or more genes are or include one or more of Adipoq (Adiponectin), Aif1 (Allograft inflammatory factor 1), Aqp4 (Aquaporin-4), Ccl2 (C-C motif chemokine 2), CD68, Edn1 (Endothelin-1), Serpine 1, Tgfb1 (Transforming growth factor beta-1), Tgfb2 (transforming growth factor beta 2), Tgfb3 (transforming growth factor beta 3), Tlr2 (Toll-like receptor 2), Tlr4 (toll like receptor 4), IL2ra, IL-13, Gzmb (Granzyme B), TNF, CXCL10 (IP-10), CCL2 (MCP-1, C-C motif chemokine 2), CXCL11 (I-TAC, C-X-C motif chemokine 11), CXCL1 (KC, Growth-regulated alpha protein), CCL4 (MIP-1b, C-C motif chemokine 4), NLRC5 (class I transactivator), CIITA (class II transactivator), such as compared to the expression in a control mouse. In certain embodiments, the mouse model is or includes a mouse that was administered A20 cells, CPA, and CAR−T cells.

In particular embodiments, the one or more genes are associated with a particular activity or function and/or encode a polypeptide that is associated with a particular activity or function. In particular embodiments, the expression is an increase in expression. In some embodiments, the activity is a decrease in expression. In some embodiments, the particular activity or function is or is associated with a gene ontology category. In some embodiments, the gene ontology category relates to a biological process, a molecular function, and/or a cellular component. In some embodiments, the gene ontology category is defined by a consortium, database, and/or a society. For example, in some embodiments, the gene ontology category is a category as defined by Gene Ontology Consortium and/or data bases or research tools or programs associated with the Gene Ontology Consortium. Examples of such resources include, but are not limited to, those as described by: The Gene Ontology Consortium (2008) Nucleic Acids Research. 36 (Database issue): D440-4; Smith et al., Nature Biotechnology. 25 (11): 1251-5 (2007); Dessimoz, C; Skunca, N. The Gene Ontology Handbook. Methods in Molecular Biology. 1446. Springer (New York); Carbon et al. Bioinformatics. 25 (2): 288-9 (2009); and Götz et al, Nucleic Acids Research. 36 (10): 3420-35 (2008).

In some embodiments, the one or more genes that are differentially expressed, e.g., differentially expressed in brain, are genes associated with an activity or function, e.g., a gene ontology category, that are or include, a response to cytokines, response to interferon-beta, cellular response to interferon-beta, antigen processing and presentation of peptide antigen via MHC class I, regulation of cell morphogenesis, cellular response to cytokine stimulus, antigen processing and presentation of peptide antigen, innate immune response, response to interferon-gamma, antigen processing and presentation, cell junction assembly, angiogenesis, positive regulation of cell projection organization, regulation of neuron projection development, blood vessel morphogenesis, negative regulation of protein modification processes, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, negative regulation of phosphorylation, antigen processing and presentation of endogenous peptide antigen, response to peptide hormone, positive regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

In certain embodiments, the one or more genes are related to immune response. In some embodiments, the genes related to immune response are or include GBP2 (Guanylate-binding protein 2), GBP4 (Guanylate-binding protein 4), GBP5 Guanylate-binding protein 5), and/or GBP9 (Guanylate-binding protein 9). In some embodiments, the one or more genes are related to angiogenesis. In particular embodiments, the one or more genes related to angiogenesis are or include ANGPT1 (Angiopoietin-1), ANGPT2 (Angiopoietin-2), ANGPTL4 (Angiopoietin-related protein 4), HIF3A (Hypoxia-inducible factor 3-alpha), LRG1 (Leucine-rich alpha-2-glycoprotein), MMRN2 (Multimerin-2), and/or XDH (Xanthine dehydrogenase/oxidase). In particular embodiments, the one or more genes are related to sterol metabolic process. In some embodiments, the genes related to sterol metabolic process are or include ACER2 (Alkaline ceramidase 2), ATF3 (Cyclic AMP-dependent transcription factor ATF-3), PDK4 (Pyruvate dehydrogenase (acetyl-transferring)] kinase isozyme 4), PLA2G3 (Group 3 secretory phospholipase A2), and/or SULT1A1 (Sulfotransferase 1A1). In some embodiments, the genes from two or more categories or clusters are combined into a single category or cluster. In particular embodiments, the genes are CD274 (Programmed cell death 1 ligand 1; PD-L1), TGTP1 (T-cell-specific guanine nucleotide triphosphate-binding protein 1), and/or VWF (von Willebrand factor).

In some embodiments, the one or more genes are related to cell adhesion. In particular embodiments, the one or more genes associated with cell adhesion are or include VCAM-1(Vascular cell adhesion protein 1), ICAM-1 (Intercellular adhesion molecule 1), SELE (E-selectin), SELP (P-selectin), CD31 (PECAM-1), IL2ra (Interleukin-2 receptor subunit alpha), and Aqp4 (Aquaporin-4). In some embodiments, the one or more genes associated with cell adhesion are or include platelet endothelial cell adhesion molecule (PECAM-1), also known as cluster of differentiation 31 (CD31). In some embodiments, upregulation of these genes in tissue contribute to infiltration of the immunotherapy, e.g., one or more cells expressing a recombinant receptor and/or a CAR. In certain embodiments, the one or more genes are related to oxidative stress and antioxidant defense. In some embodiments, the genes related to oxidative stress and oxidative defense are or include NCF1 (Neutrophil cytosol factor 1), AOX1 (Aldehyde oxidase), BNIP3 (BCL2/adenovirus E1B 19 kDa protein-interacting protein 3), PXDN (Peroxidasin), SCARA3 (Scavenger receptor class A member 3), MGST3 (Microsomal glutathione S-transferase 3), and PTGS2 (Prostaglandin G/H synthase 2). In certain embodiments, the one or more genes are related to the nitric oxide signaling pathway. In certain embodiments, the one or more gene related to the nitric oxide signaling pathway are or include NCF1 (Neutrophil cytosol factor 1), NOS3 (Nitric oxide synthase, endothelial), and/or SCARA3 (Scavenger receptor class A member 3).

In some embodiments, the one or more genes are genes encoding cytokines, chemokines, and MHC proteins. In particular embodiments, the one or more genes are genes encoding cytokines, chemokines, and MHC proteins are or include CCL4, C-C motif chemokine 4, CIITA (class II transactivator), CXCL1, CXCL10 (IP-10), CXCL11 (I-TAC, GM-CSF, IL-13, IL-4, IL-6, TNF (tumor necrosis factor), Ccl2 (C-C motif chemokine 2), and CCL4 (MIP-1b). In particular embodiments, the one or more genes are genes involved in inflammation and vascular changes. In certain embodiments, the one or more genes involved in inflammation and vascular changes is or includes Adipoq (Adiponectin), Aif1 (Allograft inflammatory factor 1), CD68, Edn1 (Endothelin-1), Serpine 1, Tgfb1 (Transforming growth factor beta-1), Tgfb2 (transforming growth factor beta 2), Tgfb3 (transforming growth factor beta 3), Tlr2 (Toll-like receptor 2), and Tlr4 (toll like receptor 4).

In certain embodiments, the one or more genes are markers of endothelial activation. In particular embodiments, the one or more markers of endothelial activation are or include Gbp5, Selp, or vwf. In certain embodiments, similar or the same markers of endothelial activation are elevated in human cases of neurotoxicity, e.g., severe neurotoxicity, associated with an immunotherapy, e.g., a CAR−T cell therapy. In various embodiments, the expression of one or more of Gbp5, Selp, and vwf are elevated in brain endothelial cells in human cases of neurotoxicity, e.g., severe neurotoxicity, associated with an immunotherapy, e.g., a CAR−T cell therapy. In some embodiments, the one or more markers of endothelial activation are elevated in brain endothelial cells in the mouse model are elevated in human brain endothelial cells in human cases of neurotoxicity associated with an immunotherapy. Markers of endothelial activation that are elevated in brain endothelial cells in human cases of neurotoxicity are described in Gust et al., Cancer Discov; 7(12); 1-16 (2017).

In certain embodiments, the one or more signs, symptoms, and/or outcomes of the mouse model is or includes a change in the expression of one or more of any of the genes listed herein, including in any of the examples, figures, and or tables provided herein. In some embodiments, the one or more signs, symptoms, and/or outcomes is or includes a change in the expression of a gene that encodes one or more of any of the proteins listed herein, e.g., cytokines, including in any of the examples, figures, and or tables provided herein. In particular embodiments, the one or more genes are a gene that is that is involved in and/or encodes a protein that is involved in or is involved in a response to, any of the signs, symptoms, and/or outcomes of the mouse model described herein.

In some embodiments, the change in gene expression is an increase in gene expression. In certain embodiments, the increase is at least a 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold increase in gene expression.

In certain embodiments, the change in gene expression is a decrease or reduction in gene expression. In some embodiments, the decrease or reduction in gene expression is at least a 10%, at least 20%, at least 30%, at least 40%, at least 50%, at least 60%, at least 70%, at least 75%, at least 80%, at least 85%, at least 90%, at least 95%, at least 97%, at least 98%, at least 99%, at least 99.9% or at least 99.99%. In some embodiments, the decrease or reduction is or is about a 100% decrease or reduction. In some embodiments, the decrease or reduction in gene expression is at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold decrease or reduction in gene expression.

In some embodiments, the change in gene expression, e.g., increase or decrease in gene expression, is in comparison with the gene expression in control mice. In some embodiments, the change in gene expression, e.g., increase or decrease in gene expression, is in comparison with the gene expression in a mouse that has not been administered the immunotherapy or in naïve mice. In some embodiments, the change in gene expression, e.g., increase or decrease in gene expression, is in comparison with the gene expression in the same mouse prior to or at the time of administering immunotherapy.

In some embodiments, the change in gene expression, e.g., increase or decrease in gene expression, is observed after immunotherapy is administered, e.g., about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy. In some embodiments, the change in gene expression, e.g., increase or decrease in gene expression, is observed prior to administration of immunotherapy. In some embodiments, the change in gene expression, e.g., increase or decrease in gene expression, is observed at the time of administration of immunotherapy.

In certain embodiments, the one or more signs, symptoms, and/or outcomes of the mouse model is or includes a change in the expression of one or more genes in a tissue. In some embodiments, the one or more signs, symptoms, and/or outcomes of the mouse model is or includes a change in the expression of one or more genes in connective tissue, muscle tissue, nervous tissue, or epithelial tissue. In particular embodiments, the one or more signs, symptoms, and/or outcomes of the mouse model is or includes a change in the expression of one or more genes in heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, or tendons. In certain embodiments, the one or more tissues are brain tissue, liver tissue, spleen tissue, lung tissue, or kidney tissue. In some embodiments, the expression of one or more genes is changed in brain tissue. In particular embodiments, the change in expression is an increase in expression.

In some embodiments, the one or more signs, symptoms, and/or outcomes of the mouse model is or includes an alteration or change in one or more parameters and/or aspects of blood and/or blood chemistry. In particular embodiments, the one or more parameters and/or aspects of blood and/or blood chemistry are or include levels, amounts, or concentrations of electrolytes, such as levels of sodium, potassium, chloride, calcium, and/or measurements of plasma osmolality or renal function, e.g. creatinine urea BUN-to-creatinine ratio. In some embodiments, the one or more parameters and/or aspects of blood and/or blood chemistry are or include acid and base levels, e.g., levels of anions, arterial blood gas, base excess, bicarbonate content, and carbon dioxide content. In some embodiments, the one or more parameters and/or aspects of blood and/or blood chemistry relate to blood iron content, such as levels or amounts of ferritin, serum iron, transferrin saturation, total iron binding capacity, and/or transferrin receptor. In some embodiments, the one or more parameters and/or aspects of blood and/or blood chemistry are levels or amounts of hormones, e.g., thyroid stimulating hormone. In particular embodiments, the one or more parameters and/or aspects of blood and/or blood chemistry relate to markers of cardiovascular function such as amounts or levels of troponin, lactate dehydrogenase, myoglobin, and/or glycogen phosphorylase isoenzyme BB. In some embodiments, the one or more parameters and/or aspects of blood and/or blood chemistry are or include levels, concentrations, or amounts of proteins, e.g., serum albumin, total serum protein, ALP, ALT, AST, bilirubin and/or unconjugated bilirubin. In certain embodiments, the one or more signs, symptoms, and/or outcomes of the mouse model is or includes a reduction in the amount, level, or concentration of serum or blood calcium.

In certain embodiments, the one or more parameters and/or aspects of blood and/or blood chemistry are or include one or more changes to an amount or level of serum glucose, serum albumin, and/or total serum protein. In some embodiments, the parameters or aspects of blood chemistry are or include levels, amounts, or concentrations of serum or blood sodium, potassium, calcium, urea, creatinine, glucose, high density lipoprotein, low density lipoprotein, C-reactive protein, thyroid stimulating hormone, albumin, alkaline phosphatase, ALT (alanine aminotransferase), AST (aspartate aminotransferase), BUN (blood urea nitrogen), chloride, carbon dioxide, and/or bilirubin.

In some embodiments, the one or more signs, symptoms, and/or outcomes of the mouse model is or includes a reduction of the amount or level of serum glucose. In particular embodiments, the one or more signs, symptoms, and/or outcomes of the mouse model is or includes a reduction of the amount or level of serum albumin. In certain embodiments, the one or more signs, symptoms, and/or outcomes of the mouse model is or includes a reduction of the ratio of serum Albumin to globulin.

In particular embodiments, the one or more signs, symptoms, and/or outcomes of the mouse model is or includes damage and/or injury to one or more tissues. In some embodiments, the tissue damage and/or injury is associated, caused by, and/or has the appearance of tissue damage that is caused by or associated with inflammation. In particular embodiments, the inflammation is acute inflammation. In particular embodiments, the inflammation is chronic inflammation. In certain embodiments, connective tissue, muscle tissue, nervous tissue, and/or epithelial tissue are injured or damaged. In certain embodiments, one or more tissues of heart, vasculature, salivary glands, esophagus, stomach, liver, gallbladder, pancreas, intestines, colon, rectum, hypothalamus, pituitary gland, pineal gland, thyroid, parathyroid, adrenal gland, kidney, ureter, bladder, urethra, lymphatic system, skin, muscle, brain, spinal cord, nerves, ovaries, uterus, testes, prostate, pharynx, larynx, trachea, bronchi, lungs, diaphragm, bone, cartilage, ligaments, and/or tendons are injured or damaged. In certain embodiments, liver, spleen, and/or lung is damaged or injured.

In particular embodiments, the damage is or includes the presence or formation of one or more granulomas. In some embodiments, the granulomas are or include immune cells. In particular embodiments, the granulomas are or include macrophages. In some embodiments, the granulomas are or include histiocytes. In particular embodiments, the granulomas include one or more dead or necrotic cells. In particular embodiments, the granulomas, are the histiocytic granulomas.

In some embodiments, the damage is or includes necrosis. In particular embodiments, the necrosis is or includes coagulative necrosis, liquefactive necrosis, gangrenous necrosis, caseous necrosis, fat necrosis, and/or fibroid necrosis. In some embodiments, the necrosis is or includes fibrosis. In particular embodiments, the necrosis is or includes necrosis that is formed by and/or associated with vascular damage, e.g., immune-mediated vascular damage. In some embodiments, the damaged tissue contains one or more necrotic and/or dead cells.

In particular embodiments, the one or more signs, symptoms, and/or outcomes of the mouse model is or includes brain edema. In particular embodiments, the edema is vascular edema. In some embodiments, the brain edema is or includes brain water content that is or is greater than 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% brain water content. In certain embodiments, the brain water content is increased by or by at least 2%, 3%, 4%, 5%, 6%, 7%, 8%, 9%, 10%, 11%, 12%, 13%, 14%, 15%, 20%, 25% or 30%.

In certain embodiments, the one or more signs, symptoms, and/or outcomes of the mouse model is or includes brain tissue damage. In some embodiments, the brain tissue damage is or includes one or more hemorrhages, such acute hemorrhages. In some embodiments, hemorrhages, e.g., acute hemorrhages, may be present in any region of the brain, including but not limited to the diencephalon and the cerebellum. Hemorrhages in brain tissue can be identified and characterized as a matter of routine, such as with histological staining techniques. In some embodiments, the one or more signs, symptoms, and/or outcomes of the mouse model are hemorrhages, e.g., acute hemorrhages, that display extravascular red blood cells. In some embodiments, the hemorrhaging occurs at, at about, or within 14 days, 12 days, 10 days, 8 days, 7 days, 6 days, 5 days, 4 days, 3 days, or 2 days after the immunotherapy is administered. In particular embodiments, the hemorrhaging occurs at, at about, or within 5 days after the immunotherapy is administered.

Particular embodiments contemplate that the tumor burden, e.g., tumor size or tumor volume, of the mouse at the time of the administration of the lymphodepleting agent or therapy or the immunotherapy, correlates to the chance, probability, or likelihood that the mouse will develop hemorrhaging, e.g., acute hemorrhaging, within the brain. Without wishing to be bound by theory, particular aspects contemplate that increases to tumor burden at the time of administration of the lymphodepleting agent or therapy or immunotherapy increases or enhances the chance, probability, or likelihood that the mouse will develop brain hemorrhages. In some embodiments, an administrating an increased number of antigen-expressing cells, e.g., tumor cells such as A20 cells, increases or enhances the chance, probability, or likelihood that the mouse will develop brain hemorrhages.

Certain embodiments contemplate that the characteristics of the immunotherapy correlate to the chance, probability, or likelihood that the mouse will develop hemorrhaging, e.g., acute hemorrhaging, within the brain. Without wishing to be bound by theory, particular aspects contemplate that increases in transduction efficiency, or ratios of CD8 to CD4 cells (e.g., higher ratios of CD8 cells), increases or enhances the chance, probability, or likelihood that the mouse will develop brain hemorrhages.

In some embodiments, the one or more signs, symptoms, and/or outcomes of the mouse model is or includes altered appearance or behavior. In some embodiments, the one or more signs, symptoms, and/or outcomes of stress are or include signs of stressed behavior. In some embodiments, the one or more signs, symptoms, and/or outcomes of the mouse model is or includes reduced food intake. In particular embodiments, the signs of stress are or include reduced food intake, reduced water intake, reduced grooming, and/or reduced locomotor activity.

In particular embodiments, the one or more signs, symptoms, and/or outcomes of the mouse model is or includes weight loss. In certain embodiments, the weight loss is, is about, or is at least a loss of 3%, a loss of 5%, a loss of 6%, a loss of 7%, a loss of 8%, a loss of 9%, a loss of 10%, a loss of 11%, a loss of 12%, a loss of 13%, a loss of 14%, a loss of 15%, a loss of 16%, a loss of 17%, a loss of 18%, a loss of 19%, a loss of 20%, a loss of 25%, a loss of 30%, a loss of 35%, a loss of 40%, a loss of 45%, a loss of 50% of body weight. In certain embodiments, the weight loss is, is about, or is at least a loss of 0.5 grams, 1 gram, 1.5 grams, 2 grams, 2.5 grams, 3 grams, 3.5 grams, 4 grams, 4.5 grams, 5 grams, 6 grams, 7.5 grams, 10 grams, or 15 grams of body weight.

In certain embodiments, the one or more signs, symptoms, and/or outcomes of the mouse model is or includes a reduction in body temperature. In particular embodiments, the reduction in temperature is a reduction of, of about, or of at least a 2.5% reduction, a 3% reduction, a 4% reduction, a 5% reduction, a 6% reduction, a 7% reduction, a 8% reduction, a 9% reduction, a 10% reduction, a 15% reduction, a 20% reduction, a 25% reduction of body temperature. In certain embodiments, the reduction is temperature is a reduction of, of about, or of at least a 0.5° C. reduction, a 1.0° C. reduction, a 1.5° C. reduction, a 2.0° C. reduction, a 2.5° C. reduction, a 3.0° C. reduction, a 3.5° C. reduction, a 4.0° C. reduction, a 4.5° C. reduction, a 5.0° C. reduction, a 6.0° C. reduction, a 7.5° C. reduction, or a 10° C. reduction in body weight.

In certain embodiments, the one or more signs, symptoms, and/or outcomes of the mouse model is or includes morbidity or death. In some embodiments, the one or more signs, symptoms, and/or outcomes of the mouse model is or includes an increased probability of morbidity or death. In certain embodiments, the probability of morbidity or death within 1 hour, 6 hours, 12 hours, 24 hours, 48 hours, 72 hours, 3 days, 4 days, 5 days, 6 days, 7 days, 1 week, 2 weeks, 3 weeks, 4 weeks, 6 weeks, 8 weeks, 10 week, 12 weeks, 16 weeks, or 20 weeks after treatment with the immunotherapy is increased by at least 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold. In some embodiments, the one or more signs, symptoms, and/or outcomes of the mouse model is or includes an increased probability of requiring a treatment to prevent death. In some embodiments, of requiring a treatment to prevent death within 1 hour, 6 hours, 12 hours, 24 hours, 48 hours, 72 hours, 3 days, 4 days, 5 days, 6 days, 7 days, 1 week, 2 weeks, 3 weeks, 4 weeks, 6 weeks, 8 weeks, 10 week, 12 weeks, 16 weeks, or 20 weeks after treatment with the immunotherapy is increased by at least 10%, at least 20%, at least 30%, at least 40%, at least 50%, at least 60%, at least 70%, at least 80%, at least 90%, at least 100% at least 150%, at least 200%, at least 2-fold, at least 3-fold, at least 4-fold, at least 5-fold, at least 10-fold, at least 15-fold, at least 20-fold, at least 25-fold, at least 30-fold, at least 40-fold, at least 50-fold.

Certain provided embodiments are based on observations herein regarding various mechanisms and pathways involving events and factors that may contribute to the development of toxicities following the administration of certain immunotherapies such as CAR T therapies. For example, the mechanisms and pathways include those that may contribute to development of toxicities involving neurological symptoms or disturbances, e.g., neurotoxicity, including severe neurotoxicity, and/or cerebral edema. Among the provided embodiments are interventions (such as therapeutic compositions and methods) that target such events or pathways or component thereof. Also among the provided embodiments are methods and models for investigating and elucidating components of such pathways and/or testing such interventions.

In some embodiments, the pathways and steps targeted or investigated via the animal models may include aspects of or factors associated with local or systemic inflammation, such as local or systemic release or accumulation of cytokines, including neuroinflammation.

In some aspects, expansion of engineered T cells or T cells activated in response to an immunomodulatory agent can lead to inflammatory effects such as peripheral inflammation and increased production of cytokines and other factors. Such inflammation can in some contexts lead to or increase the risk of one or more events that may lead to neurotoxicity outcomes.

In some aspects, neuroinflammation such as increased accumulation of certain inflammatory cytokines in the brain and in some contexts may lead to or involve the activation of inflammatory cells in CNS or brain, such as activation of microglia cells.

In some aspects, cytokines and other factors such as blood-borne or systemic cytokines enter the brain via a circumventricular organs (CV or CVO). CVOs generally may be described as structures in the brain characterized by extensive vasculature and lack of a normal blood-brain barrier, composed of specialized tissue and located in the midline ventricular system. In some aspects, circulating PAMPs and cytokines may interact with the CVO and choroid plexus and stimulate cytokine production. In some aspects, such cytokines may directly interact with and/or enter the CNS. The CVO can express components of the immune system, such as TLRs, receptors for IL-113, IL-6, and TNF-α. See Dantzer et al. (2008) Nat Rev Neurosci 9:46. CVOs may be sensory organs (such as area postrema (AP), the subcortical organ (SFO) and the vascular organ of lamina terminalis) or secretory organs (such as subcommissural organ (SCO), the posterior pituitary, the pineal gland, the median eminence and the intermediate lobe of the pituitary gland).

In some embodiments, systemic or peripheral inflammation can lead to changes to the blood-brain barrier (BBB), which may be disruptive or non-disruptive. Varatharaj and Galea (2017) Brain, Behavior, and Immunity 60 (2017) 1-12. The pathophysiology of neurotoxicity in subjects treated with an immunotherapy, e.g. CAR−T cells, can involve disruptive and/or non-disruptive changes of the CNS environment. Blood brain barrier breakdown may be involved in pathophysiology, but may not necessarily be required.

In some aspects, pathophysiology and changes related to neurotoxicity and/or cerebral edema may not be associated with or caused by infiltration, e.g. perivascular infiltration, of engineered cells of a cell therapy, such as CAR+ T cells, into the CNS or brain.

In some aspects, systemic inflammation may contribute to underlying pathophysiological changes in the brain. In some cases, T cells and/or engineered cells administered for adoptive cell therapy may prompt the production of systemic cytokines, which, in some cases, could lead to adverse outcomes through various pathways, alone or in combination. In certain cases, the systemic cytokines may directly damage endothelial cells of the brain vasculature. In some cases, the systemic cytokines enter the brain and cause adverse outcomes through effects on brain cells or tissue, such as microglial cells. Thus, in some cases, adverse effects such as toxicity may result from multiple actions of cytokines that are produced systemically and/or locally in the brain.

In some cases cytokine activity in the brain may trigger, directly or indirectly, activation of microglia. In certain cases, the microglia are positioned within a close proximity to the neurovasculature and cells that maintain the blood brain barrier. Thus, in some cases, activation of the microglia may damage these cells, including by altering astrocyte morphology and damaging the astrocytic processes that align the blood brain barrier. Furthermore, in some cases cytokines that are produced peripherally, systemically or locally in the brain, may disrupt cell-to-cell adhesion of endothelial cells. In some cases, these events can lead to vascular damage and leakage of the blood brain barrier, which may in turn lead to cerebral edema or other adverse effects.

### III. ASSAYS AND METHODS OF USE OF THE MOUSE MODEL

In particular embodiments, the mouse models provided herein are useful for investigating and/or evaluating hypotheses, mechanisms, and modifiers of a sign, symptom, or outcome, e.g., a sign, symptom, or outcome of toxicity to an immunotherapy. In certain embodiments, the mouse model is generated by any of the methods described herein, such as those described in Section I, and/or is a mouse with an attribute or phenotype as described herein, such as in Section II.

A. Test Immunotherapies

In certain embodiments, the mouse models provided herein are useful for investigating alternative and/or modified immunotherapies or techniques for administering an immunotherapy. For example, in some embodiments, a mouse model is useful for evaluating a new or alternative lymphodepleting agent or therapy. In certain embodiments, the mouse model is useful for evaluating an alternative or next generation immunotherapy, e.g., a CAR−T cell composition with modifications such as kill switches.

In some embodiments, the method includes one or more steps administering a test immunotherapy to a mouse. In particular embodiments, a mouse that is described herein, e.g., in Section LA is administered the test immunotherapy. In particular embodiments, the mouse is administered the test immunotherapy prior to, during, or after the mouse is administered a lymphodepleting agent or therapy. In certain embodiments, the lymphodepleting agent or therapy is a lymphodepleting agent or therapy that is described herein, e.g., in Section I.B. In certain embodiments, the test immunotherapy prior to, during, or after, the mouse has been administered antigen-expressing cells. In particular embodiments, the antigen expressing cells are antigen-expressing cells that are described herein, e.g., antigen-expressing cells described in Section I.D.

In some embodiments, the methods provided herein include one or more steps of detecting, measuring, and/or assessing one or more signs, symptoms, or outcomes in a mouse that was administered the test immunotherapy. In particular embodiments, the one or more signs, symptoms, and/or outcomes are one or more of a sign, symptom, and/or outcome that is described herein, e.g., in Section II. In certain embodiments, the detection, measurement, and/or the assessment is compared to a detection, measurement, and/or the assessment of a sign, symptom, and/or outcome of the mouse model in a mouse that did not receive the test immunotherapy. In some embodiments, the sign, symptom, or outcome is activity, expansion, and/or persistence of the immunotherapy. In certain embodiments, the sign, symptom, and/or outcome is a sign, symptom, or outcome of toxicity.

In particular embodiments, the mouse that did not receive the test immunotherapy did not receive any prior treatments of antigen-expressing cells or a lymphodepleting agent or therapy. In particular embodiments, the mouse that did not receive the test immunotherapy was a naïve mouse. In certain embodiments, the mouse that did not receive the test immunotherapy was administered a lymphodepleting agent or therapy. In certain embodiments, the lymphodepleting agent or therapy was a lymphodepleting agent or therapy as described herein, e.g., in Section I.B. In particular embodiments, the lymphodepleting agent or therapy was the same lymphodepleting agent or therapy that was administered to the mouse that received the test immunotherapy. In certain embodiments, the mouse that did not receive the test immunotherapy was administered antigen-expressing cells. In certain embodiments, the antigen expressing cells were antigen-expressing cells that are described herein, e.g., in Section I.D. In particular embodiments, the antigen expressing cells were the same cells that were administered to the mouse that received the test immunotherapy. In particular embodiments, the mouse that did not receive the test immunotherapy did not receive an immunotherapy. In particular embodiments, the mouse that did not receive the test immunotherapy did receive an immunotherapy. In some embodiments, the immunotherapy was an immunotherapy that is described herein, e.g., in Section I.C.

In some embodiments, any one or more conditions or agents in connection with culturing, processing, or generating the immunotherapy can be altered to generate a test immunotherapy. For example, in particular embodiments, the test immunotherapy is generated from a population of cells that are isolated and/or enriched from a different source or population of donor cells than what are used to generate the immunotherapy. In certain embodiments, the test immunotherapy is generated from a population of cells that are isolated and/or enriched from the donor cells, activated, transduced, and/or expanded in the presence of one or more reagents that are different form the reagents used to generate the immunotherapy.

In some embodiments, the test immunotherapy is generated from a different population of cells than the immunotherapy. In some embodiments, the different population of immune cells may include a sub-type or subpopulation of T cells, including but not limited to subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (T_{IL}), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the test immunotherapy is generated from cells that are cultured in the presence of one or more reagents than are different from the reagents used culture cells for the immunotherapy. In particular embodiments, the cells used to generate the test immunotherapy are cultured with one or more different reagents from the immunotherapy prior to and/or subsequent to transduction. In certain embodiments, the cells used to generate the test immunotherapy are transfected in the presence of one or more reagents that are different from the reagents that are used to generate the immunotherapy. In some embodiments, the test immunotherapy is generated from cells that are incubated, cultured, and/or treated with one or more different reagents than the immunotherapy during one or more steps of isolating, processing, culturing, activating, transducing, engineering, expanding, and/or formulating.

In certain embodiments, the test immunotherapy is generated from cells that are processed in a different apparatus than the immunotherapy. In some embodiments, test immunotherapy is generated from cells that were processed in a different apparatus than the immunology during one or more steps of isolating, processing, culturing, activating, transducing, engineering, expanding, and/or formulating.

In certain embodiment, the test immunotherapy and the immunotherapy bind to and/or recognize the same antigen. In certain embodiments, the test immunotherapy expresses a different recombinant receptor than the immunotherapy.

In particular embodiments, the test immunotherapy expresses the same recombinant receptor as the immunotherapy. In some embodiments, the test immunotherapy is generated from cells that are transduced with a different virus than the immunotherapy. In particular embodiment, the virus is a retro virus. In certain embodiments, the virus is a lentivirus. In some embodiments, the cells of the test immunotherapy are transduced with non-viral technique.

In some embodiments, the test immunotherapy has a recombinant receptor or CAR with one or more different domains that the recombinant receptor or CAR of the immunotherapy. In certain embodiments, the recombinant receptor or CAR of the test immunotherapy and the recombinant receptor or CAR of the immunotherapy have different antigen recognition domains. In particular embodiments, the test immunotherapy has a different scFv than the immunotherapy. In some embodiments, the scFv of the test immunotherapy binds to and/or recognizes a different antigen than the scFv of the immunotherapy. In particular embodiments, the scFv of the test immunotherapy and the scFv of the immunotherapy bind to and/or recognize the same antigen. In particular embodiments, the recombinant receptor or CAR of the test immunotherapy and the recombinant receptor or CAR of the immunotherapy have a different transmembrane domain. In certain embodiments, the recombinant receptor or CAR of the test immunotherapy and the recombinant receptor or CAR of the immunotherapy have a different transmembrane domain. In some embodiments, the recombinant receptor or CAR of the test immunotherapy and the recombinant receptor or CAR of the immunotherapy have different IgG hinge regions. In some embodiments, the recombinant receptor or CAR of the test immunotherapy and the recombinant receptor or CAR of the immunotherapy have one or more different spacers. In particular embodiments, the recombinant receptor or CAR of the test immunotherapy and the recombinant receptor or CAR of the immunotherapy have one or more different intracellular signaling domains.

In particular embodiments, the test immunotherapy is or includes an immune system stimulator. In some embodiments, the test immunotherapy is or includes a T-cell engaging therapy. In certain embodiments, the test immunotherapy is a cell composition, e.g., a therapeutic cell composition. In some embodiments, the test immunotherapy is a cell composition that contains cells that express a recombinant receptor. In particular embodiments, the recombinant receptor is a CAR.

In certain embodiments, the test immunotherapy is a modified and/or second generation CAR−T cell therapy. In some embodiments, the test immunotherapy is or includes a T cell composition containing TRUCKs (T cells redirected for universal cytokine killing). In some embodiments, TRUCKs co-express a chimeric antigen receptor (CAR) and an anti-tumor cytokine. In particular embodiments, the cytokine expression may be constitutive or induced by T cell activation (for example, interleukin-12 (IL-12)). In some embodiments, the localized production of pro-inflammatory cytokines are focused and/or targeted by CAR's specificity recruits endogenous immune cells to tumor sites and to coordinate and/or potentiate an anti-tumor response. In some embodiments, the test immunotherapy is or includes universal, allogeneic CAR−T cells. In some embodiments, universal CAR−T cells are engineered to no longer express endogenous T cell receptor (TCR) and/or major histocompatibility complex (MHC) molecules.

In certain embodiments, the test immunotherapy is or includes a cell composition that contains cells expressing self-driving CARs. In some embodiments, self-driving CARs co-express a CAR and a chemokine receptor. In particular embodiments, the self-driving CAR−T cells binds to a tumor ligand, for example, to enhance tumor homing. In certain embodiments, the test immunotherapy is a cell composition containing CAR−T cells that are engineered to be resistant to immunosuppression, e.g., armored CARs. In some embodiments, the armored CAR may be genetically modified to have reduced expression of or to no longer express one or more immune checkpoint molecules e.g., cytotoxic T lymphocyte-associated antigen 4 (CTLA4) or programmed cell death protein 1 (PD1)). In some embodiments, an armored CAR is administered with an immune checkpoint switch receptor and/or a monoclonal antibody that blocks immune checkpoint signaling.

In certain embodiments, the test immunotherapy is or includes a cell composition containing cells that express a self-destruct CAR. In certain embodiments, a self-destruct CAR is designed, engineered, and/or transfected using RNA delivered by electroporation to encode the CAR. In certain embodiments, ganciclovir binding to thymidine kinase in T cells expressing the self-destruct CAR induce apoptosis of the T cell. In some embodiments, activation of human caspase 9 by a small-molecule dimerizer in the T cell expressing the self-destruct CAR induces apoptosis in the T cell. In particular embodiments, the test immunotherapy is or includes a cell composition containing cells that express a conditional CAR. In certain embodiments, the conditional CAR−T cell is by default unresponsive, or switched ‘off’, until the addition of a small molecule is added to allow for full activation of the CAR. In some embodiments, conditional CAR−T cells are engineered to express an adaptor-specific receptor with affinity for subsequently administered secondary antibodies directed at target antigen. In certain embodiments, the test immunotherapy is or includes a cell composition containing cells that express a marked CAR. In certain embodiments, marked CAR−T cells express a CAR plus a tumor epitope to which an existing monoclonal antibody agent binds. In some embodiments, the marked CAR is designed so that in the event of toxicity, e.g., severe neurotoxicity or CRS, administration of the monoclonal antibody clears the CAR T cells and alleviates symptoms with no additional off-tumor effects. In particular embodiments, the test immunotherapy is or includes a cell composition containing tandem CAR−T cells (TanCAR). In certain embodiments, TanCAR−T cell expresses a single CAR consisting of two linked single-chain variable fragments (scFvs) that have different affinities fused to intracellular co-stimulatory domain(s) and a CD3 domain. In some embodiments, TanCAR T cell activation is achieved only when target cells co-express both targets. In certain embodiments, the test immunotherapy is or includes a cell composition containing dual CAR−T cells. In some embodiments, a dual CAR−T expresses two separate CARs with different ligand binding targets; one CAR includes only the CD3 domain and the other CAR includes only the co-stimulatory domain(s). In certain embodiments, dual CAR T cell activation requires co-expression of both targets on the tumor. In certain embodiments, the test immunotherapy is or includes a cell composition containing cells that express a safety CAR (sCAR). In particular embodiments, the sCAR consists of an extracellular scFv fused to an intracellular inhibitory domain (for example, CTLA4 or PD1). In particular embodiments, sCAR−T cells co-expressing a standard CAR become activated only when encountering target cells that possess the standard CAR target but lack the sCAR target.

In some embodiments, if the detection, measurement, and/or the assessment of the toxicity in the mouse that received the test immunotherapy is greater than, more severe, and/or shows a higher degree of toxicity than the detection, measurement, and/or assessment of the toxicity in the mouse that did not receive the test immunotherapy, then the test immunotherapy is associated with a high, elevated, or increased risk of toxicity in a human subject. In particular embodiments, if the detection, measurement, and/or the assessment of the toxicity in the mouse that received the test immunotherapy is lower than, less severe, and/or shows a lower degree of toxicity than the detection, measurement, and/or assessment of the toxicity in the mouse that did received an immunotherapy, e.g., an immunotherapy described in Section I.C, then the test immunotherapy is associated with a low, reduced, decreased risk of toxicity in a human subject. In certain embodiments, the toxicity is neurotoxicity. In particular embodiments, the toxicity is severe neurotoxicity. In particular embodiments, the neurotoxicity is grade 3 or prolonged grade 3, grade 4, or grade 5. In some embodiments, the toxicity is cytokine release syndrome (CRS). In particular embodiments, the CRS is severe CRS. In particular embodiments, the CRS is grade 3, grade 4, or grade 5.

In some embodiments, a high, elevated, or increased risk of toxicity is, is about, or is at least a 5%, 10%, 15%, 20%, 25%, 30%, 35%, 40%, 45%, 50%, 55%, 60%, 70%, 75%, 80%, 85%, 90%, 95%, 97%, 98%, or 99% probability of developing toxicity after the immunotherapy, e.g., the test immunotherapy, is administered. In particular embodiments, a low, reduced, decreased risk of toxicity is, is about, or is less than a 50%, 45%, 40%, 35%, 30%, 25%, 20%, 15%, 10%, 5%, 1%, 0.1%, 0.01%, 0.001%, or 0.0001% probability of developing toxicity after the immunotherapy is administered.

B. Test Lymphodepleting Agents or Therapies

In certain embodiments, the method includes one or more steps administering a test lymphodepleting agent or therapy to a mouse. In particular embodiments, a mouse that is described herein, e.g., in Section I.A, is administered the lymphodepleting agent or therapy, e.g., a lymphodepleting agent or therapy such as those described in Section I.B. In particular embodiments, the mouse is administered the test lymphodepleting agent or therapy prior to, during, or after the mouse is administered an immunotherapy. In certain embodiments, the immunotherapy is one that is described herein, e.g., in Section I.C. In certain embodiments, the test lymphodepleting agent or therapy prior to, during, or after, the mouse has been administered antigen-expressing cells. In particular embodiments, the antigen expressing cells are cells that are described herein, e.g., in Section I.D.

In some embodiments, the methods provided herein include one or more steps of detecting, measuring, and/or assessing one or more signs, symptoms, or outcomes in a mouse that was administered the test lymphodepleting agent or therapy. In particular embodiments, the one or more signs, symptoms, and/or outcomes are one or more of a sign, symptom, and/or outcome that is described herein, e.g., in Section II. In certain embodiments, the detection, measurement, and/or the assessment is compared to a detection, measurement, and/or the assessment of a sign, symptom, and/or outcome in a mouse that received a lymphodepleting agent or therapy that was not the test lymphodepleting agent or therapy. In certain embodiments, the detection, measurement, and/or the assessment is compared to a detection, measurement, and/or the assessment of a sign, symptom, and/or outcome in a mouse that did not receive a lymphodepleting agent or therapy. In some embodiments, the sign, symptom, or outcome is activity, expansion, and/or persistence of the immunotherapy. In certain embodiments, the sign, symptom, and/or outcome is a sign, symptom, or outcome of toxicity.

In particular embodiments, the mouse that did not receive the test lymphodepleting agent or therapy did not receive any prior treatments of antigen-expressing cells or immunotherapy. In particular embodiments, the mouse that did not receive the test lymphodepleting agent or therapy was a naïve mouse. In certain embodiments, the mouse that did not receive the test lymphodepleting agent or therapy was administered a different lymphodepleting agent or therapy. In certain embodiments, the lymphodepleting agent or therapy was a lymphodepleting agent or therapy as described in Section I.B. In certain embodiments, the mouse that did not receive the test lymphodepleting agent or therapy was administered antigen-expressing cells. In certain embodiments, the antigen expressing cells were cells that are described in Section I.D. In particular embodiments, the antigen expressing cells were the same cells that were administered to the mouse that received the test lymphodepleting agent or therapy. In particular embodiments, the mouse that did not receive the test lymphodepleting agent or therapy received an immunotherapy. In particular embodiments, the immunotherapy was an immunotherapy that is described herein, e.g., in Section I.C. In some embodiments, the same immunotherapy was the administered to the mouse that received the test lymphodepleting agent or therapy and to the mouse that did not receive the test lymphodepleting agent or therapy.

In some embodiments, if the detection, measurement, and/or the assessment of the toxicity in the mouse that received the test lymphodepleting agent or therapy is greater than, more severe, and/or shows a higher degree of toxicity than the detection, measurement, and/or assessment of the toxicity in the mouse that did not receive the test lymphodepleting agent or therapy, then the test lymphodepleting agent or therapy is associated with a high, elevated, or increased risk of toxicity in a human subject. In particular embodiments, if the detection, measurement, and/or the assessment of the toxicity in the mouse that received the test lymphodepleting agent or therapy is lower than, less severe, and/or shows a lower degree of toxicity than the detection, measurement, and/or assessment of the toxicity in the mouse that did received a lymphodepleting agent or therapy that was not the test agent or therapy, e.g., an lymphodepleting agent or therapy described herein such as in Section I.C, then the test lymphodepleting agent or therapy is associated with a low, reduced, decreased risk of toxicity in a human subject. In certain embodiments, the toxicity is neurotoxicity. In particular embodiments, the toxicity is severe neurotoxicity. In particular embodiments, the neurotoxicity is grade 3 or prolonged grade 3, grade 4, or grade 5. In some embodiments, the toxicity is cytokine release syndrome (CRS). In particular embodiments, the CRS is severe CRS. In particular embodiments, the CRS is grade 3, grade 4, or grade 5.

C. Test Agents

In some embodiments, the mouse model of toxicity provided herein is useful for evaluating an agent, such as an agent that may inhibit or exacerbate one or more signs, symptoms, or outcomes of toxicity. Such agents may be used evaluate interventions to reduce toxicity, and/or, identify potential therapeutic targets for treating or ameliorating toxicity in human subjects. In some embodiments, provided herein is method of identifying and/or assessing one or more effects of an agent, e.g., a test agent. In particular an embodiment, the test agent is administered prior to, subsequent to, or during the administration of the lymphodepleting agent or therapy and/or the immunotherapy.

In some embodiments, the test agent is or includes a small molecule, a small organic compound, a peptide, a polypeptide, an antibody or antigen binding fragment thereof, a non-peptide compounds, a synthetic compound, a fermentation product, a cell extract, a polynucleotide, an oligonucleotide, an RNAi, an siRNA, an shRNA, a multivalent siRNA, an miRNA, and/or a virus.

In certain embodiments, the test agent is administered at, at about, or at least 5 minutes, 10 minutes, 15 minutes, 30 minutes, 60 minutes, 1 hour, 2 hours, 4 hours, 6 hours, 12 hours, 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, or more than six weeks prior to administering and/or initiating the lymphodepleting agent or therapy. In particular embodiments, the test agent is administered at, at about, or at least 5 minutes, 10 minutes, 15 minutes, 30 minutes, 60 minutes, 1 hour, 2 hours, 4 hours, 6 hours, 12 hours, 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, or more than six weeks prior to administering and/or initiating the immunotherapy.

In some embodiments, the test agent is administered during and/or in conjunction with the lymphodepleting agent or therapy and/or the immunotherapy. In certain embodiments, the test agent is administered within 24 hours, within 18 hours, within 12 hours, within 8 hours, within 6 hours, within 4 hours, within 2 hours, within 1 hour, within 30 minutes, within 15 minutes, within 10 minutes, within 5 minutes, within 3 minutes, or within 1 minute of administering the lymphodepleting agent or therapy. In particular embodiments, the test agent is administered within 24 hours, within 18 hours, within 12 hours, within 8 hours, within 6 hours, within 4 hours, within 2 hours, within 1 hour, within 30 minutes, within 15 minutes, within 10 minutes, within 5 minutes, within 3 minutes, or within 1 minute of administering the immunotherapy. In certain embodiments, the test agent is administered at the same time as the immunotherapy and/or the lymphodepleting agent or therapy. In some embodiments, the administration of the test agent overlaps the administration of the immunotherapy and/or the lymphodepleting agent or therapy.

In certain embodiments, the test agent is administered at, at about, or at least 5 minutes, 10 minutes, 15 minutes, 30 minutes, 60 minutes, 1 hour, 2 hours, 4 hours, 6 hours, 12 hours, 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, or more than six weeks after the administration and/or after the completion of the administration of the lymphodepleting agent or therapy. In particular embodiments, the test agent is administered at, at about, or within 5 minutes, 10 minutes, 15 minutes, 30 minutes, 60 minutes, 1 hour, 2 hours, 4 hours, 6 hours, 12 hours, 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, or 6 weeks after the administration and/or after the completion of the administration of the immunotherapy.

In some embodiments, the one or more doses of the test agent are administered. In some embodiments, a single dose of the test agent is administered. In particular embodiments, one dose, two doses, three doses, four doses, five doses, six doses, seven doses, eight doses, nine doses, ten doses, more than ten doses, more than twenty doses, more than thirty doses, more than forty doses, or more than fifty doses of the test agent are administered. In some embodiments, the test agent is administered once. In certain embodiments, more than one dose of the test agent is administered over a period of or about 24 hours, 48 hours, 72 hours, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, or more than six weeks. In particular embodiments, more than one dose of the test agent is administered over a period of less than 24 hours, less than 48 hours, less than 72 hours, less than 4 days, less than 5 days, less than 6 days, less than 7 days, less than 8 days, less than 9 days, less than 10 days, less than 11 days, less than 12 days, less than 13 days, less than 14 days, less than 2 weeks, less than 3 weeks, less than 4 weeks, less than 5 weeks, or less than 6 weeks. In certain embodiments, the test agent is administered once daily, twice daily, three times daily, four times daily, five times daily, six times daily, eight times daily, ten times daily, or twelve times daily. In some embodiments, doses of the test agent are administered at, at about, or within 1 hour apart, 2 hours apart, 3 hours apart, 4 hours apart, or between 5 minutes and 1 hour apart, between 1 hour and 2 hours apart, between 2 and 4 hours apart, between 4 and 12 hours apart, or between 12 and 24 hours apart, each inclusive. In some embodiments, the test agent is administered once a day, once every 2 days, 3 days, 4 days, 5 days, 6 days, once a week, twice a week, three times a week, once a month, twice a month, three times a month, four times a month, or five times a month.

In some embodiments, the one or more doses of the test agent are administered orally, intravenously, intraperitoneally, transdermally, intrathecally, intramuscularly, intranasally, transmucosally, subcutaneously, or rectally. In some embodiments, the dose of the test agent is or includes between or between about 1 μg/kg and 1,000 mg/kg, 1 μg/kg and 100 μg/kg, 100 μg/kg and 500 μg/kg, 500 μg/kg and 1,000 μg/kg, 1 mg/kg and 10 mg/kg, 10 mg/kg and 100 mg/kg, 100 mg/kg and 500 mg/kg, 200 mg/kg and 300 mg/kg, 100 mg/kg and 250 mg/kg, 200 mg/kg and 400 mg/kg, 250 mg/kg and 500 mg/kg, 250 mg/kg and 750 mg/kg, 50 mg/kg and 750 mg/kg, 1 mg/kg and 10 mg/kg, or 100 mg/kg and 1,000 mg/kg (amount of the test agent over body weight; each inclusive). In some embodiments, the dose of the test agent is or is about 1 μg/kg, 5 μg/kg, 10 μg/kg, 50 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1 mg/kg, 5 mg/kg, 10 mg/kg, 25 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1 g/kg. In some embodiments, the test agent is administered as part of a composition or formulation, such as a pharmaceutical composition or formulation as described herein. Thus, in some cases, the composition comprising the agent is administered as described herein. In other aspects, the agent is administered alone and may be administered by any known acceptable route of administration or by one described herein, such as with respect to compositions and pharmaceutical formulations.

In some embodiments, the test agent is administered ad libitum, for example added to and/or mixed into food, e.g., mouse chow, or drinking water.

In some embodiments, the methods provided herein include one or more steps of detecting, measuring, and/or assessing one or more signs, symptoms, or outcomes in a mouse that was administered the test agent. In particular embodiments, the one or more signs, symptoms, and/or outcomes are one or more of a sign, symptom, and/or outcome that is described herein, e.g., in Section II. In certain embodiments, the detection, measurement, and/or the assessment is compared to a detection, measurement, and/or the assessment of a sign, symptom, and/or outcome in a mouse that did not receive the test agent. In particular embodiments, the mouse that did not receive the test agent was administered an immunotherapy. In certain embodiments, the mouse that did not receive the test agent was administered the same immunotherapy as the mouse that received the test agent. In particular embodiments, the mouse that did not receive the test agent was administered a lymphodepleting agent or therapy. In certain embodiments, the mouse that did not receive the test agent was administered the same lymphodepleting agent or therapy as the mouse that received the test agent. In some embodiments, the mouse that did not receive the test agent was administered antigen-expressing cells. In certain embodiments, the mouse that did not receive the test agent was administered the same antigen-expressing cells as the mouse that received the test agent. In some embodiments, the sign, symptom, or outcome is activity, expansion, and/or persistence of the immunotherapy. In certain embodiments, the sign, symptom, and/or outcome is a sign, symptom, or outcome of toxicity.

In some embodiments, the test agent is administered to the mouse to assess and/or determine if a target of the test agent contributes to and/or is associated with one or more mechanisms of toxicity, e.g., toxicity to an immunotherapy. In some embodiments, the test agent, inhibits and/or antagonizes the target. In certain embodiments, the test agent activates and/or agonizes the target. In particular embodiments, the target is a polynucleotide, a DNA polynucleotide, e.g., genomic DNA, an RNA polynucleotide, e.g., mRNA, a polypeptide, e.g., an enzyme, a kinase, a phosphates, and/or receptor.

In some embodiments, if the comparison of the detection, measurement, and/or the assessment of toxicity indicate that the test agent alters one or more signs, symptoms, or outcomes of toxicity, then the target is identified as having a putative role in toxicity to the immunotherapy. For example, in some embodiments, if the test agent inhibits and/or antagonizes the target and the comparison indicates that the test agent reduces the toxicity, the target is identified as having an activity that putatively contributes to the toxicity. Likewise, in certain embodiments, if the test agent activates and/or agonizes the target and the comparison indicates that the test agent increases the toxicity, then target is identified as having an activity that putatively contributes to the toxicity. Such a target may be further considered to be a putative target for a therapeutic intervention of toxicity.

In certain embodiments, the test agent is a second therapy or a test therapy, e.g., an immunotherapy, that may be administered in conjunction with an immunotherapy, for example, to treat the same disease as the immunotherapy, and/or to treat a condition, such as a secondary condition that presents or manifests along with, or has the potential to present or manifest along with, a disease or condition that is treated by the immunotherapy.

1. Candidate and Test Interventions

In some embodiments, test agents are administered to a mouse that models toxicity described herein to determine if the test agent is a candidate agent for an intervention. In some embodiments, the candidate agent is a potential agent or therapy that prevents, reduces, and/or ameliorates toxicity, e.g., CRS or neurotoxicity, in a subject, e.g., a human subject.

In some embodiments, if the comparison of the detection, measurement, and/or the assessment of toxicity indicate that the test agent alters one or more signs, symptoms, or outcomes of toxicity, then the test agent is a candidate agent to reduce toxicity in a subject, e.g., a human subject. In some embodiments, the comparison indicates if the test agent is a candidate agent for reducing toxicity to be administered prior to, subsequent to, or during the administration of the lymphodepleting agent or therapy and/or the immunotherapy. For example, in some embodiments, the test agent is administered prior to the lymphodepleting agent or therapy and/or the immunotherapy and the test agent reduces, prevents, and/or ameliorates one or more signs, symptoms of toxicity, and the agent is therefore deemed to be a candidate agent to be administered prior to the lymphodepleting agent or therapy and/or the immunotherapy in a subject, e.g., a human. In certain embodiments, the test agent is administered during the administration of the lymphodepleting agent or therapy and/or the immunotherapy and the test agent reduces, prevents, and/or ameliorates one or more signs, symptoms of toxicity, and the agent is therefore deemed to be a candidate agent to be administered during to the immunotherapy in a subject, e.g., a human subject. In particular embodiments, the test agent is administered after the administration of the lymphodepleting agent or therapy and/or the immunotherapy and the test agent reduces, prevents, and/or ameliorates one or more signs, symptoms of toxicity, and the agent is therefore deemed to be a candidate agent to be administered after the immunotherapy in a subject, e.g., a human subject.

In certain embodiments, the test agent is administered during or at the appearance of one or more signs, symptoms, and/or outcomes of toxicity, and the and the test agent reduces, prevents, and/or ameliorates one or more of the signs, symptoms of toxicity. In some embodiments, the agent is therefore deemed to be a candidate agent to be administered at or during the appearance of one or more signs, symptoms, and/or outcomes of toxicity in a subject, e.g., a human subject.

In some embodiments, the test agent is administered to a mouse with one or more tumor and/or cancer cells. In particular embodiments, the one or more tumor and/or cancer cells are antigen expressing cells that express an antigen that is bound by and/or recognized by the immunotherapy. In certain embodiments, the antigen expressing cell is a cell described herein, e.g., in Section I.D. In some embodiments, administration of the immunotherapy prevents or reduces the formation of tumors that are composed of or include the antigen-expressing cells. In particular embodiments, if administration of the test agent prior to, during, and/or after administration of the immunotherapy results in a presence of one or more tumors that are composed of or include the antigen-expressing cells, that the test agent is not a candidate agent to treat, prevent, and/or ameliorate toxicity. In certain embodiments, if administration of the test agent results in an increase of one or more tumors that are composed of or include the antigen-expressing cells as compared to a mouse that received the immunotherapy but not the test agent, then the test agent is not a candidate agent.

In some embodiments, the test agent is a steroid, is an antagonist or inhibitor of a cytokine receptor, such as IL-6 receptor, CD122 receptor (IL-2R/IL-15Rbeta receptor), or CCR2, or is an inhibitor of a cytokine, such as IL-6, IL-15, MCP-1, IL-10, IFN-γ, IL-8, or IL-18. In some embodiments, the test agent is an agonist of a cytokine receptor and/or cytokine, such as TGF-β. In some embodiments, the test agent, e.g., agonist, antagonist or inhibitor, is an antibody or antigen-binding fragment, a small molecule, a protein or peptide, or a nucleic acid. In some embodiments, the test agent is an anti-histamine.

In some embodiments, the test agent is a steroid, e.g., corticosteroid. Corticosteroids typically include glucocorticoids and mineralocorticoids.

In some embodiments, the test agent is a glucocorticoid. In some embodiments, glucocorticoids include synthetic and non-synthetic glucocorticoids. Exemplary glucocorticoids include, but are not limited to: alclomethasones, algestones, beclomethasones (e.g. beclomethasone dipropionate), betamethasones (e.g. betamethasone 17-valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, fluprednisolones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylprednisolones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21-palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980).

In some examples, the glucocorticoid is selected from among cortisones, dexamethasones, hydrocortisones, methylprednisolones, prednisolones and prednisones. In a particular example, the glucocorticoid is dexamethasone.

In some embodiments, the test agent is an agent that targets a cytokine, e.g., is an antagonist or inhibitor of a cytokine, such as transforming growth factor beta (TGF-beta), interleukin 6 (IL-6), interleukin 10 (IL-10), interleukin 15 (IL-15), interferon gamma (IFN-gamma), or monocyte chemoattractant protein-1 (MCP-1). In some embodiments, the test agent targets (e.g. inhibits or is an antagonist of) a cytokine receptor, such as IL-6 receptor (IL-6R), CD122 receptor (IL-2R/IL-15Rbeta), MCP-1 (CCL2) receptor (CCR2 or CCR4), a TGF-beta receptor (TGF-beta I, II, or II), IFN-gamma receptor (IFNGR), IL-1 receptor (IL-1R) or IL-10 receptor (IL-10R). In some embodiments, the test agent is a blocker or inhibitor of a tumor necrosis factor. In some embodiments, the test agent is a JAK/STAT inhibitor. In some embodiments, the test agent is a kinase inhibitor, e.g., an inhibitor of Bruton's tyrosine kinase (BTK). In some embodiments, the test agent is a device used to reduce cytokines, such as a physical cytokine absorber.

In some embodiments, the test agent is an antibody or antigen binding fragment. In some embodiments, the test agent is tocilizumab, siltuximab, sarilumab, clazakizumab, olokizumab (CDP6038), elsilimomab, ALD518/BMS-945429, sirukumab (CNTO 136), CPSI-2634, ARGX-109, FE301, FM101, Hu-Mik-β-1, tofacitinib, ruxolitinib, CCX140-B, RO523444, BMS CCR2 22, INCB 3284 dimesylate, JNJ27141491, RS 504393, adalimumab, certolizumab pegol or golimumab. In some embodiments, the agent is infliximab, etanercept, or anakinra or an antigen-binding fragment or variant thereof. In some embodiments, the test agent is siltuximab or an antigen-binding fragment or variant thereof.

In some embodiments, the agent that treats or ameliorates symptoms of neurotoxicity and/or CRS is a small molecule. In some embodiments, the agent is ibrutinib, ruxolitinib, or an antigen-binding fragment thereof.

In some embodiments, the test agent is an antagonist or inhibitor of IL-6 or the IL-6 receptor (IL-6R). In some aspects, the test agent is an antibody that neutralizes IL-6 activity, such as an antibody or antigen-binding fragment that binds to IL-6 or IL-6R. For example, in some embodiments, the test agent is or comprises tocilizumab (atlizumab) or sarilumab, anti-IL-6R antibodies. In some embodiments, the test agent is an anti-IL-6R antibody described in U.S. Pat. No. 8,562,991. In some cases, the test agent that targets IL-6 is an anti-IL-6 antibody, such as siltuximab, sarilumab, clazakizumab, elsilimomab, ALD518/BMS-945429, sirukumab (CNTO 136), CPSI-2634, ARGX-109, FE301, FM101, or olokizumab (CDP6038), or an antigen-binding fragment or variant thereof. In some aspects, the test agent may neutralize IL-6 activity by inhibiting the ligand-receptor interactions. The feasibility of this general type of approach has been demonstrated with a natural occurring receptor antagonist for interleukin-1. See Harmurn, C. H. et al., Nature (1990) 343:336-340. In some aspects, the IL-6/IL-6R antagonist or inhibitor is an IL-6 mutein, such as one described in U.S. Pat. No. 5,591,827. In some embodiments, the test agent that is an antagonist or inhibitor of IL-6/IL-6R is a small molecule, a protein or peptide, or a nucleic acid.

In some embodiments, the test agent is an antagonist or inhibitor of IL-15 or the IL-15 receptor (CD122). In some aspects, the test agent is an antibody that neutralizes IL-15 activity, such as an antibody or antigen-binding fragment that binds to IL-15 or its receptor CD122. For example, in some instances, the test agent is Hu-Mik-β-1, a humanized monoclonal antibody directed to the IL-2/IL-15R-β subunit (CD122) that blocks IL-15 action. In some aspects, the IL-15 antagonist or inhibitor is an IL-15 mutein, such as one described in U.S. Pat. No. 7,235,240. In some embodiments, the agent that is an antagonist or inhibitor of IL-15/CD122 is a small molecule, a protein or peptide, or a nucleic acid.

In some embodiments, the test agent is an agonist or stimulator of TGF-β or a TGF-β receptor (e.g., TGF-β receptor I, II, or III). In some aspects, the test agent is an antibody that increases TGF-β activity, such as an antibody or antigen-binding fragment that binds to TGF-β or one of its receptors. In some embodiments, the agent that is an agonist or stimulator of TGF-β and/or its receptor is a small molecule, a protein or peptide, or a nucleic acid.

In some embodiments, the test agent is an antagonist or inhibitor of MCP-1 (CCL2) or a MCP-1 receptor (e.g., MCP-1 receptor CCR2 or CCR4). In some aspects, the test agent is an antibody that neutralizes MCP-1 activity, such as an antibody or antigen-binding fragment that binds to MCP-1 or one of its receptors (CCR2 or CCR4). In some embodiments, the MCP-1 antagonist or inhibitor is any described in Gong et al. J Exp Med. 1997 Jul. 7; 186(1): 131-137 or Shahrara et al. J Immunol 2008; 180:3447-3456. In some embodiments, the agent that is an antagonist or inhibitor of MCP-1 and/or its receptor (CCR2 or CCR4) is a small molecule, a protein or peptide, or a nucleic acid.

In some embodiments, the agent is an antagonist or inhibitor of IFN-γ or an IFN-γ receptor (IFNGR). In some aspects, the agent is an antibody that neutralizes IFN-γ activity, such as an antibody or antigen-binding fragment that binds to IFN-γ or its receptor (IFNGR). In some aspects, the IFN-gamma neutralizing antibody is any described in Dobber et al. Cell Immunol. 1995 February; 160(2):185-92 or Ozmen et al. J Immunol. 1993 Apr. 1; 150(7):2698-705. In some embodiments, the agent that is an antagonist or inhibitor of IFN-γ/IFNGR is a small molecule, a protein or peptide, or a nucleic acid.

In some embodiments, the agent is an antagonist or inhibitor of IL-10 or the IL-10 receptor (IL-10R). In some aspects, the agent is an antibody that neutralizes IL-10 activity, such as an antibody or antigen-binding fragment that binds to IL-10 or IL-10R. In some aspects, the IL-10 neutralizing antibody is any described in Dobber et al. Cell Immunol. 1995 February; 160(2):185-92 or Hunter et al. J Immunol. 2005 Jun. 1; 174(11):7368-75. In some embodiments, the agent that is an antagonist or inhibitor of IL-10/IL-10R is a small molecule, a protein or peptide, or a nucleic acid.

In some embodiments, the agent is an antagonist or inhibitor of IL-1 or the IL-1 receptor (IL-1R). In some aspects, the agent is an IL-1 receptor antagonist, which is a modified form of IL-1R, such as anakinra (see, e.g., Fleischmann et al., (2006) Annals of the rheumatic diseases. 65(8):1006-12). In some aspects, the agent is an antibody that neutralizes IL-1 activity, such as an antibody or antigen-binding fragment that binds to IL-1 or IL-1R, such as canakinumab (see also EP 2277543). In some embodiments, the agent that is an antagonist or inhibitor of IL-1/IL-1R is a small molecule, a protein or peptide, or a nucleic acid.

In some embodiments, the agent is an antagonist or inhibitor of a tumor necrosis factor (TNF) or a tumor necrosis factor receptor (TNFR). In some aspects, the agent is an antibody that blocks TNF activity, such as an antibody or antigen-binding fragment that binds to a TNF, such as TNFα, or its receptor (TNFR, e.g., TNFRp55 or TNFRp75). In some aspects, the agent is selected from among infliximab, adalimumab, certolizumab pegol, golimumab, and etanercept. In some embodiments, the agent that is an antagonist or inhibitor of TNF/TNFR is a small molecule, a protein or peptide, or a nucleic acid. In some embodiments, the agent is a small molecule that affects TNF, such as lenalidomide (see, e.g., Muller et al. (1999) Bioorganic & Medicinal Chemistry Letters. 9 (11):1625).

In some embodiments, the agent is an antagonist or inhibitor of signaling through the Janus kinase (JAK) and two Signal Transducer and Activator of Transcription (STAT) signaling cascade. JAK/STAT proteins are common components of cytokine and cytokine receptor signaling. In some embodiments, the agent that is an antagonist or inhibitor of JAK/STAT, such as ruxolitinib (see, e.g., Mesa et al. (2012) Nature Reviews Drug Discovery. 11(2):103-104), tofacitinib (also known as Xeljanz, Jakvinus tasocitinib and CP-690550), Baricitinib (also known as LY-3009104, INCB-28050), Filgotinib (G-146034, GLPG-0634), Gandotinib (LY-2784544), Lestaurtinib (CEP-701), Momelotinib (GS-0387, CYT-387), Pacritinib (SB1518), and Upadacitinib (ABT-494). In some embodiments, the agent is a small molecule, a protein or peptide, or a nucleic acid.

In some embodiments, the agent is a kinase inhibitor. In some embodiments, the agent is an inhibitor of Bruton's tyrosine kinase (BTK). In some embodiments, the inhibitor is or comprises ibrutinib or acalabrutinib (see, e.g., Barrett et al., ASH 58^{th} Annual Meeting San Diego, Calif. Dec. 3-6, 2016, Abstract 654; Ruella et al., ASH 58th Annual Meeting San Diego, Calif. Dec. 3-6, 2016, Abstract 2159). In some embodiments, the agent is an inhibitor as described in U.S. Pat. Nos. 7,514,444; 8,008,309; 8,476,284; 8,497,277; 8,697,711; 8,703,780; 8,735,403; 8,754,090; 8,754,091; 8,957,079; 8,999,999; 9,125,889; 9,181,257; or 9,296,753.

In some embodiments, the test agent is an inhibitor of a microglial cell activity. In some embodiments, the inhibitor is an antagonist that inhibits the activity of a signaling pathway in microglia. In some embodiments, the microglia inhibitor affects microglial homeostasis, survival, and/or proliferation. In some embodiments, the inhibitor targets the CSF1R signaling pathway. In some embodiments, the inhibitor is an inhibitor of CSF1R. In some embodiments, the inhibitor is a small molecule. In some cases, the inhibitor is an antibody.

In some embodiments, administration of the test agent results in an alteration of microglial homeostasis and viability, a decrease or blockade of microglial cell proliferation, a reduction or elimination of microglial cells, a reduction in microglial activation, a reduction in nitric oxide production from microglia, a reduction in nitric oxide synthase activity in microglia, or protection of motor neurons affected by microglial activation. In some embodiments, the test agent alters the level of a serum or blood biomarker of CSF1R inhibition, or a decrease in the level of urinary collagen type 1 cross-linked N-telopeptide (NTX) compared to at a time just prior to initiation of the administration of the inhibitor. In some embodiments, the administration of the test agent transiently inhibits the activity of microglia activity and/or wherein the inhibition of microglia activity is not permanent. In some embodiments, the administration of the test agent transiently inhibits the activity of CSF1R and/or wherein the inhibition of CSF1R activity is not permanent.

In some embodiments, the test agent is an antagonist that inhibits the activity of a signaling pathway in microglia. In some embodiments, the test agent reduces microglial cell activity affects microglial homeostasis, survival, and/or proliferation.

In some embodiments, the test agent is selected from an anti-inflammatory agent, an inhibitor of NADPH oxidase (NOX2), a calcium channel blocker, a sodium channel blocker, inhibits GM-CSF, inhibits CSF1R, specifically binds CSF-1, specifically binds IL-34, inhibits the activation of nuclear factor kappa B (NF-κB), activates a CB2 receptor and/or is a CB2 agonist, a phosphodiesterase inhibitor, inhibits microRNA-155 (miR-155), upregulates microRNA-124 (miR-124), inhibits nitric oxide production in microglia, inhibits nitric oxide synthase, or activates the transcription factor NRF2 (also called nuclear factor (erythroid-derived 2)-like 2, or NFE2L2).

In some embodiments, the test agent targets CSF1 (also called macrophage colony-stimulating factor MCSF). In some embodiments, the test agent affects MCSF-stimulated phosphorylation of the M-CSF receptor (Pryer et al. Proc Am Assoc Cancer Res, AACR Abstract nr DDT02-2 (2009)). In some cases, the test agent is MCS110 (international patent application publication number WO2014001802; Clinical Trial Study Record Nos.:A1 NCT00757757; NCT02807844; NCT02435680; NCT01643850).

In some embodiments, the test agent a small molecule that targets the CSF1 pathway. In some embodiments, the test agent is a small molecule that binds CSF1R. In some embodiments, the test agent is a small molecule which inhibits CSF1R kinase activity by competing with ATP binding to CSF1R kinase. In some embodiments, the test agent is a small molecule which inhibits the activation of the CFS1R receptor. In some cases, the test agent inhibits binding of the CSF-1 ligand. In some embodiments, the test agent that is any of the inhibitors described in US Patent Application Publication Number US20160032248.

In some embodiments, the test agent is a small molecule inhibitor selected from PLX-3397, PLX7486, JNJ-40346527, JNJ28312141, ARRY-382, PLX73086 (AC-708), DCC-3014, AZD6495, GW2580, Ki20227, BLZ945, PLX647, PLX5622. In some embodiments, the agent is any of the inhibitors described in Conway et al., Proc Natl Acad Sci USA, 102(44):16078-83 (2005); Dagher et al., Journal of Neuroinflammation, 12:139 (2015); Ohno et al., Mol Cancer Ther. 5(11):2634-43 (2006); von Tresckow et al., Clin Cancer Res., 21(8) (2015); Manthey et al. Mol Cancer Ther. (8(11):3151-61 (2009); Pyonteck et al., Nat Med. 19(10): 1264-1272 (2013); Haegel et al., Cancer Res AACR Abstract nr 288 (2015); Smith et al., Cancer Res AACR Abstract nr 4889 (2016); Clinical Trial Study Record Nos.: NCT01525602; NCT02734433; NCT02777710; NCT01804530; NCT01597739; NCT01572519; NCT01054014; NCT01316822; NCT02880371; NCT02673736; international patent application publication numbers WO2008063888A2, WO2006009755A2, US patent application publication numbers US20110044998, US 2014/0065141, and US 2015/0119267.

In some embodiments, the test agent is 4-((2-(((1R,2R)-2-hydroxycyclohexyl)amino)benzo[d]thiazol-6-yl)oxy)-N-methylpicolinamide (BLZ945) or a pharmaceutically acceptable salt thereof or derivatives thereof. In some embodiments, the agent is the following compound:

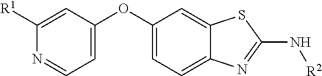

wherein R1 is an alkyl pyrazole or an alkyl carboxamide, and R2 is a hydroxycycloalkyl or a pharmaceutically acceptable salt thereof.

In some embodiments, the test agent is 5-((5-chloro-1H-pyrrolo[2,3-b]pyridin-3-yl)methyl)-N-((6-(trifluoromethyl)pyridin-3-yl)methyl)pyridin-2-amine, N-[5-[(5-Chloro-1H-pyrrolo[2,3-b]pyridin-3-yl)methyl]-2-pyridinyl]-6-(trifluoromethyl)-3-pyridinemethanamine) (PLX 3397) or a pharmaceutically acceptable salt thereof or derivatives thereof. In some embodiments, the agent is 5-(1H-Pyrrolo[2,3-b]pyridin-3-ylmethyl)-N-[[4-(trifluoromethyl)phenyl]methyl]-2-pyridinamine dihydrochloride (PLX647) or a pharmaceutically acceptable salt thereof or derivatives thereof. In some embodiments, the test agent is the following compound:

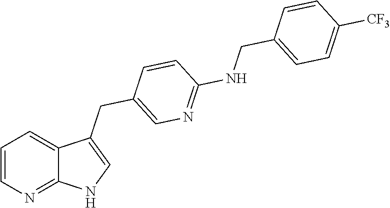 [SMILES: FC(F)(F)c1ccc(CNc2ccc(Cc3c[nH]c4ncccc34)cn2)cc1]

or a pharmaceutically acceptable salt thereof. In some embodiments, the test agent is the following compound:

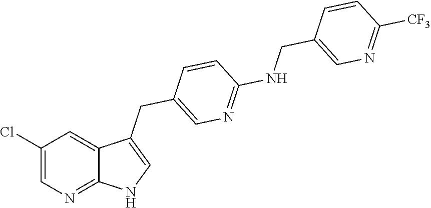 [SMILES: FC(F)(F)c1ccc(CNc2ccc(Cc3c[nH]c4ncc(Cl)cc34)cn2)cn1]

or a pharmaceutically acceptable salt thereof. In some embodiments, the agent is any of the inhibitors described in U.S. Pat. No. 7,893,075.

In some embodiments, the test agent is 4-cyano-N-[2-(1-cyclohexen-1-yl)-4-[1-[(dimethylamino)acetyl]-4-piperidinyl]phenyl]-1H-imidazole-2-carboxamide monohydrochloride (JNJ28312141) or a pharmaceutically acceptable salt thereof or derivatives thereof. In some embodiments, the agent is the following compound:

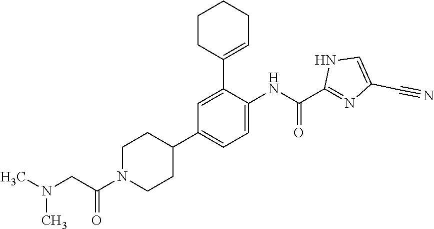 [SMILES: CN(C)CC(=O)N1CCC(c2ccc(NC(=O)c3nc(C#N)c[nH]3)c(C3=CCCCC3)c2)CC1]

or a pharmaceutically acceptable salt thereof. In some embodiments, the agent is any of the inhibitors described in U.S. Pat. No. 7,645,755.

In some embodiments, the test agent is 1H-Imidazole-2-carboxamide, 5-cyano-N-(2-(4,4-dimethyl-1-cyclohexen-1-yl)-6-(tetrahydro-2,2,6,6-tetramethyl-2H-pyran-4-yl)-3-pyridinyl)-, 4-Cyano-1H-imidazole-2-carboxylic acid N-(2-(4,4-dimethylcyclohex-1-enyl)-6-(2,2,6,6-tetramethyltetrahydropyran-4-yl)pyridin-3-yl)amide, 4-Cyano-N-(2-(4,4-dimethylcyclohex-1-en-1-yl)-6-(2,2,6,6-tetramethyl-tetrahy dro-2H-pyran-4-yl)pyridin-3-yl)-1H-imidazole-2-carboxamide (JNJ-40346527) or a pharmaceutically acceptable salt thereof or derivatives thereof. In some embodiments, the agent is the following compound:

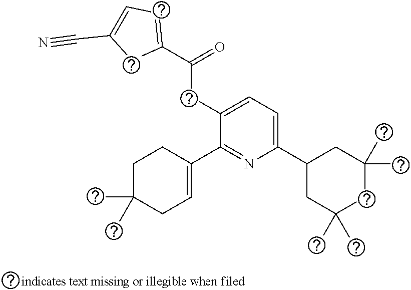 [SMILES: CC1(C)CC=C(c2nc(C3CC(C)(C)CC(C)(C)C3)ccc2CC(=O)C2=CC=C(C#N)C2)CC1]

or a pharmaceutically acceptable salt thereof.

In another embodiment, the test agent is 5-(3-Methoxy-4-((4-methoxybenzyl)oxy)benzyl)pyrimidine-2,4-diamine (GW2580) or a pharmaceutically acceptable salt thereof or derivatives thereof. In some embodiments, the agent is the following compound:

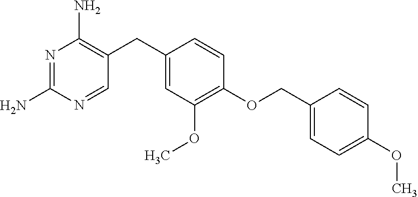 [SMILES: COc1ccc(COc2ccc(Cc3cnc(N)nc3N)cc2OC)cc1]

or a pharmaceutically acceptable salt thereof (international patent application publication number WO2009099553).

In some embodiments, the test agent is 4-(2,4-difluoroanilino)-7-ethoxy-6-(4-methylpiperazin-1-yl)quinoline-3-carboxamide (AZD6495) or a pharmaceutically acceptable salt thereof or derivatives thereof. In some embodiments, the agent is the following compound:

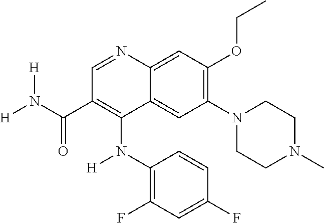 [SMILES: CCOc1cc2ncc(C(N)=O)c(Nc3ccc(F)cc3F)c2cc1N1CCN(C)CC1]

or a pharmaceutically acceptable salt thereof.

In some embodiments, the test agent is N-{4-[(6,7-dimethoxy-4-quinolyl)oxy]-2-methoxyphenyl}-N0-[1-(1,3-thiazole-2-yl)ethyl]urea (Ki20227) or a pharmaceutically acceptable salt thereof or derivatives thereof. In some embodiments, the agent is the following compound:

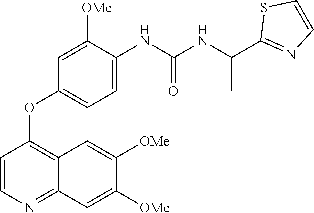 [SMILES: COc1cc(Oc2ccnc3cc(OC)c(OC)cc23)ccc1NC(=O)NC(C)c1nccs1]

or a pharmaceutically acceptable salt thereof.

In some embodiments, the agent that reduces microglial cell activation is an antibody that targets the CSF1 pathway. In some embodiments, the agent is an antibody that binds CSF1R. In some embodiments, the anti-CSF1R antibody blocks CSF1R dimerization. In some embodiments, the anti-CSF1R antibody blocks the CSF1R dimerization interface that is formed by domains D4 and D5 (Ries et al. Cancer Cell 25(6):846-59 (2014)). In some cases, the agent is selected from emactuzumab (RG7155; RO5509554), Cabiralizumab (FPA-008), LY-3022855 (IMC-CS4), AMG-820, TG-3003, MCS110, H27K15, 12-2D6, 2-4A5 (Rovida and Sbarba, J Clin Cell Immunol. 6:6 (2015); Clinical Trial Study Record Nos.: NCT02760797; NCT01494688; NCT02323191; NCT01962337; NCT02471716; NCT02526017; NCT01346358; NCT02265536; NCT01444404; NCT02713529, NCT00757757; NCT02807844; NCT02435680; NCT01643850).

In some embodiments, the agent that reduces microglial cell activation is a tetracycline antibiotic. For example, the agent affects IL-1b, IL-6, TNF-α, or iNOS concentration in microglia cells (Yrjänheikki et al. PNAS 95(26): 15769-15774 (1998); Clinical Trial Study Record No: NCT01120899). In some embodiments, the agent is an opioid antagonist (Younger et al. Pain Med. 10(4):663-672 (2009.) In some embodiments, the agent reduces glutamatergic neurotransmission (U.S. Pat. No. 5,527,814). In some embodiments, the agent modulates NFkB signaling (Valera et al J. Neuroinflammation 12:93 (2015); Clinical Trial Study Record No: NCT00231140). In some embodiments, the agent targets cannabinoid receptors (Ramirez et al. J. Neurosci 25(8):1904-13(2005)). In some embodiments, the agent is selected from minocycline, naloxone, riluzole, lenalidomide, and a cannabinoid (optionally WIN55 or 212-2).

Nitric oxide production from microglia is believed, in some cases, to result in or increase neurotoxicity. In some embodiments, the agent modulates or inhibits nitric oxide production from microglia. In some embodiments, the agent inhibits nitric oxide synthase (NOS). In some embodiments, the NOS inhibitor is Ronopterin (VAS-203), also known as 4-amino-tetrahydrobiopterin (4-ABH4). In some embodiments, the NOS inhibitor is cindunistat, A-84643, ONO-1714, L-NOARG, NCX-456, VAS-2381, GW-273629, NXN-462, CKD-712, KD-7040, or guanidinoethyldisulfide. In some embodiments, the agent is any of the inhibitors described in Ming et al., Cell Stem Cell. 2012 Nov. 2; 11(5):620-32.

In certain embodiments, the test agent is an agent capable of preventing, blocking or reducing microglial cell activation or function. In certain embodiments, the test agent is a small molecule, peptide, protein, antibody or antigen-binding fragment thereof, an antibody mimetic, an aptamer, or a nucleic acid molecule that is capable of blocking or reducing microglial activation or function. In some embodiments, the test agent is or includes minocycline, naloxone, nimodipine, Riluzole, MOR103, lenalidomide, a cannabinoid (optionally WIN55 or 212-2), intravenous immunoglobulin (IVIg), ibudilast, anti-miR-155 locked nucleic acid (LNA), MCS110, PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945, emactuzumab, IMC-CS4, FPA008, LY-3022855, AMG-820 and TG-3003, or any derivatives thereof.

In particular embodiments, the test agent is an inhibitor of colony stimulating factor 1 receptor (CSF1R). In certain embodiments, the inhibitor is or includes PLX-3397, PLX647, PLX108-D1, PLX7486, JNJ-40346527, JNJ28312141, ARRY-382, AC-708, DCC-3014, 5-(3-methoxy-4-((4-methoxybenzyl)oxy)benzyl)pyrimidine-2,4-diamine (GW2580), AZD6495, Ki20227, BLZ945 or a pharmaceutical salt or prodrug thereof; emactuzumab, IMC-CS4, FPA008, LY-3022855, AMG-820 and TG-3003 or is an antigen-binding fragment thereof; or a combination of any of the foregoing.

In some embodiments, a device, such as absorbent resin technology with blood or plasma filtration, can be used to reduce cytokine levels. In some embodiments, the device used to reduce cytokine levels is a physical cytokine absorber, such as an extracorporeal cytokine absorber. In some embodiments, a physical cytokine absorber can be used to eliminate cytokines from the bloodstream in an ex vivo, extracorporeal manner. In some embodiments, the agent is a porous polymer. In some embodiments, the agent is CytoSorb (see, e.g., Basu et al. Indian J Crit Care Med. (2014) 18(12): 822-824).

2. Test Agents for Combination Therapies

In some embodiments, an immunotherapy such as a cell therapy, e.g. dose of T cells (e.g. CAR+ T cells) is administered to subjects in combination with an additional therapeutic agent or therapy, generally other than the cell therapy or another cell therapy, such as other than a CAR+ T cell therapy. In some embodiments, the immunotherapy, e.g. dose of genetically engineered T cells, such as CAR+ T cells, is administered as part of a combination treatment or combination therapy, such as simultaneously with, sequentially with or intermittently with, in any order, one or more additional therapeutic intervention. In some embodiments, the one or more additional therapeutic intervention includes any agent or treatment for treating or preventing the disease or condition, such as the B cell malignancy, e.g. NHL, and/or any agent or treatment to increase the efficacy, persistence, and/or activity of the engineered cell therapy.

In some embodiments, an additional therapeutic agent or therapy is administered to subjects who are or are likely to be or who are predicted to be poor responders and/or who do not, are likely not to and/or who are predicted not to respond or do not respond within a certain time and/or to a certain extent to treatment with the cell therapy, e.g. dose of T cells (e.g. CAR+ T cells). In some embodiments, the additional therapeutic agent is administered to subjects who do not or are not likely to or are not predicted to exhibit a complete response or overall response, such as within 1 month, within two months or within three months after initiation of administration of the cell therapy. In some embodiments, the additional therapeutic agent is administered to subjects who exhibit or are likely to exhibit or who are predicted to exhibit progressive disease (PD), such as within 1 month, two months or three months, following administration of the cell therapy. In some embodiments, a subject is likely or predicted not to exhibit a response or a certain response based on a plurality of similarly situated subjects so treated or previously treated with the cell therapy.

In some contexts, optimal efficacy of a cell therapy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some aspects, the efficacy of the immunotherapy, e.g., T cell therapy, may be limited by the immunosuppressive activity or factors present in the local microenvironment of the disease or disorder, e.g., the TME. In some aspects, the TME contains or produces factors or conditions that can suppress the activity, function, proliferation, survival and/or persistence of T cells administered for T cell therapy.

In some embodiments, a test agent is administered to a mouse of the mouse model provided herein to evaluate and/or access a combination therapy. In some embodiments, the combination therapy is or includes the immunotherapy and/or an additional an additional therapeutic agent or therapy. In particular embodiments, the a test agent is administered to a mouse of the mouse model provided herein to assess the effects of the additional agent or therapy on one or more aspects of the immunotherapy. For example, in some embodiments, the test agent is administered to the mouse to evaluate the effects of an additional agent or therapy on the activity, expansion, and/or persistence of the immunotherapy. In certain embodiments, the test agent is administered to assess the effects of the additional agent or therapy and/or the combination therapy on or more signs, symptoms, or outcomes of the model. In some embodiments, the one or more signs, symptoms, or outcomes are one or more signs, symptoms, or outcomes of toxicity.

In some embodiments, a test agent is administered to a mouse of the model provided herein to evaluate or assess if administration of an additional agent or therapy, prior to, concomitantly with or at the same time and/or subsequently to initiation of administration of the immunotherapy, e.g. dose of T cells (e.g. CAR+ T cells) can result in improved activity, efficacy and/or persistence of the immunotherapy and/or improve responses of the treated subject. In some embodiments, a test agent is administered to a mouse of the model provided herein to evaluate or assess if the additional agent for combination treatment or combination therapy enhances, boosts and/or promotes the efficacy and/or safety of the therapeutic effect of the immunotherapy, e.g. engineered T cell therapy, such as CAR+ T cells. In some embodiments, the additional agent enhances or improves the efficacy, survival or persistence of the administered cells, e.g., cells expressing the recombinant receptor, e.g. CAR. In certain embodiments, the test agent is administered to the mouse to assess if an additional agent or therapy results in in improved activity, efficacy and/or persistence of the immunotherapy.

In some embodiments, the test agent, such as an additional agent or therapy, is an antibody or a cytotoxic or therapeutic agent, e.g., a chemotherapeutic agent. In some embodiments, test agent, such as the one or more additional agents for treatment or therapy, is an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy.

In some embodiments, the test agent, such as an additional agent, is selected from among a protein phosphatase inhibitor, a kinase inhibitor, a cytokine, an immunomodulator, or an agent that decreases the level or activity of a regulatory T (Treg) cell. In some embodiments, the test agent, e.g., the additional agent, enhances safety, by virtue of reducing or ameliorating adverse effects of the administered cell therapy. In some embodiments, the additional agent, can treat the same disease, condition or a comorbidity. In some embodiments, the additional agent can ameliorate, reduce or eliminate one or more toxicities, adverse effects or side effects that are associated with administration of the cells, e.g., CAR-expressing cells.

In some embodiments, the test agent, such as the additional therapy, treatment or agent, includes chemotherapy, radiation therapy, surgery, transplantation, adoptive cell therapy, antibodies, cytotoxic agents, chemotherapeutic agents, cytokines, growth inhibitory agents, antihormonal agents, kinase inhibitors, anti-angiogenic agents, cardioprotectants, immunostimulatory agents, immunosuppressive agents, immune checkpoint inhibitors, antibiotics, angiogenesis inhibitors, metabolic modulators or other therapeutic agents or any combination thereof. In some embodiments, the test agent, e.g., an additional agent, is a protein, a peptide, a nucleic acid, a small molecule agent, a cell, a toxin, a lipid, a carbohydrate or combinations thereof, or any other type of therapeutic agent, e.g. radiation. In some embodiments, the test agent, such as an additional therapy, agent or treatment, includes surgery, chemotherapy, radiation therapy, transplantation, administration of cells expressing a recombinant receptor, e.g., CAR, kinase inhibitor, immune checkpoint inhibitor, mTOR pathway inhibitor, immunosuppressive agents, immunomodulators, antibodies, immunoablative agents, antibodies and/or antigen binding fragments thereof, antibody conjugates, other antibody therapies, cytotoxins, steroids, cytokines, peptide vaccines, hormone therapy, antimetabolites, metabolic modulators, drugs that inhibit either the calcium dependent phosphatase calcineurin or the p70S6 kinase FK506) or inhibit the p70S6 kinase, alkylating agents, anthracyclines, vinca alkaloids, proteasome inhibitors, GITR agonists, protein tyrosine phosphatase inhibitors, protein kinase inhibitors, an oncolytic virus, and/or other types of immunotherapy. In some embodiments, the test agent, such as the additional agent or treatment, is bone marrow transplantation, T cell ablative therapy using chemotherapy agents such as, fludarabine, external-beam radiation therapy (XRT), cyclophosphamide, and/or antibody therapy.

In some embodiments, the test agent, e.g., the additional agent, is a kinase inhibitor, e.g., an inhibitor of Bruton's tyrosine kinase (BTK), e.g., ibrutinib. In some embodiments, the additional agent is an adenosine pathway or adenosine receptor antagonist or agonist. In some embodiments, the test agent, e.g., the additional agent, is an immunomodulator such as thalidomide or a thalidomide derivative (e.g., lenalidomide). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, the test agent, such as an additional agent, is a chemotherapeutic agent. Exemplary chemotherapeutic agents include an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzamab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); an immunomodulatory such as thalidomide or a thalidomide derivative (e.g., lenalidomide).

In some embodiments, the test agent, such as an additional agent, is an immunomodulatory agent. In some embodiments, the combination therapy includes an immunomodulatory agent that can stimulate, amplify and/or otherwise enhance an anti-tumor immune response, e.g. anti-tumor immune response from the administered engineered cells, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein. In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide. In some embodiments, the binding molecules, recombinant receptors, cells and/or compositions are administered in combination with a test agent, e.g., an additional agent, that is an antibody or an antigen-binding fragment thereof, such as a monoclonal antibody.

In some embodiments, the immunomodulatory agent blocks, inhibits or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens (Pardoll (2012) Nature Reviews Cancer 12:252-264), e.g., engineered cells such as CAR-expressing cells. Because many such immune checkpoints are initiated by ligand-receptor interactions, they can be readily blocked by antibodies against the ligands and/or their receptors. In contrast to the majority of anti-cancer agents, checkpoint inhibitors do not necessarily target tumor cells directly, but rather target lymphocyte receptors or their ligands in order to enhance the endogenous antitumor activity of the immune system.

In some embodiments, the test agent, e.g., the additional agent, is an immunomodulatory agent that is an antagonist molecule or is an immune checkpoint inhibitor capable of inhibiting or blocking a function of a molecule, or signaling pathway, involving an immune checkpoint molecule. In some embodiments, the immune checkpoint molecule or pathway is PD-1, PD-L1, PD-L2, CTLA-4, LAG-3, TIM3, VISTA, adenosine 2A Receptor (A2AR), or adenosine or a pathway involving any of the foregoing. In certain embodiments, antagonistic molecules blocking an immune checkpoint pathway, such as small molecules, nucleic acid inhibitors (e.g., RNAi) or antibody molecules, are becoming promising avenues of immunotherapy for cancer and other diseases.

In some embodiments, the immune checkpoint inhibitor is a molecule that totally or partially reduces, inhibits, interferes with or modulate one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta). Immune checkpoint inhibitors include antibodies, or antigen binding fragments thereof, or other binding proteins, that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary of immunomodulatory antibodies include, but are not limited to, Daclizumab (Zenapax), Bevacizumab (Avastin 0), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon gamma, CAS 951209-71-5, available from IRX Therapeutics).

In some embodiments, the test agent, such as an additional agent, is an agent that binds to and/or inhibits Programmed cell death 1 (PD-1). PD-1 is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1.

In some embodiments, the test agent, such as the additional agent, is an agent that binds to or inhibits PD-L1 (also known as CD274 and B7-H1) and/or PD-L2 (also known as CD273 and B7-DC). PD-L1 and PD-L2 are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MEDI4736 (Medimmune) MPDL3280A (Genentech), BMS-935559 (Bristol-Myers Squibb) and MSB0010718C. MEDI4736 (Medimmune) is a human monoclonal antibody that binds to PD-L1, and inhibits interaction of the ligand with PD-1. MDPL3280A (Genentech/Roche) is a human Fc optimized IgG1 monoclonal antibody that binds to PD-L1. MDPL3280A and other human monoclonal antibodies to PD-L1 are described in U.S. Pat. No. 7,943,743 and U.S Publication No. 20120039906. Other anti-PD-L1 binding agents include YW243.55.S70 (see WO2010/077634) and MDX-1105 (also referred to as BMS-936559, and, e.g., anti-PD-L1 binding agents described in WO2007/005874).

In some embodiments, the test agent, e.g., the additional agent, is an agent that is an inhibitor of Cytotoxic T-lymphocyte-associated antigen (CTLA-4), also known as CD152, or binds to CTLA-4. CTLA-4 is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

In some embodiments, the test agent, such as an additional agent, is an agent that bind to and/or inhibits Lymphocyte activation gene-3 (LAG-3), also known as CD223. LAG-3 is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include BMS-986016 (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420.

In some embodiments, the test agent, e.g., the additional agent, is an agent that bins to and/or inhibits T-cell immunoglobulin domain and mucin domain-3 (TIM-3). TIM-3 was initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the test agent, e.g., the additional agent, is an agent that is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross-reacts with CEACAM-1 and CEACAM-5 as described in, e.g., WO 2013/054331 and US 2014/0271618.

In some embodiments, the test agent, e.g., additional agent, is an agent that binds to and/or inhibits 4-1BB, also known as CD137. 4-1BB is a transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

In some embodiments, the test agent, e.g., the additional agent, is an agent that binds to and/or inhibits Tumor necrosis factor receptor superfamily, member 4 (TNFRSF4), also known as OX40 and CD134. TNFRSF4 is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXR0916 (RG7888, Genentech).

In some embodiments, the test agent, such as the additional agent, tis an agent or a molecule that decreases the regulatory T cell (Treg) population. Methods that decrease the number of (e.g., deplete) Treg cells are known in the art and include, e.g., CD25 depletion, cyclophosphamide administration, and modulating Glucocorticoid-induced TNFR family related gene (GITR) function. GITR is a member of the TNFR superfamily that is upregulated on activated T cells, which enhances the immune system. Reducing the number of Treg cells in a subject prior to apheresis or prior to administration of engineered cells, e.g., CAR-expressing cells, can reduce the number of unwanted immune cells (e.g., Tregs) in the tumor microenvironment and reduces the subject's risk of relapse. In some embodiments, the additional agent includes a molecule targeting GITR and/or modulating GITR functions, such as a GITR agonist and/or a GITR antibody that depletes regulatory T cells (Tregs). In some embodiments, the additional agent includes cyclophosphamide. In some embodiments, the GITR binding molecule and/or molecule modulating GITR function (e.g., GITR agonist and/or Treg depleting GITR antibodies) is administered prior to the engineered cells, e.g., CAR-expressing cells. For example, in some embodiments, the GITR agonist can be administered prior to apheresis of the cells. In some embodiments, cyclophosphamide is administered to the subject prior to administration (e.g., infusion or re-infusion) of the engineered cells, e.g., CAR-expressing cells or prior to apheresis of the cells. In some embodiments, cyclophosphamide and an anti-GITR antibody are administered to the subject prior to administration (e.g., infusion or re-infusion) of the engineered cells, e.g., CAR-expressing cells or prior to apheresis of the cells.

In some embodiments, the test agent, such as the additional agent, is an agent that is a GITR agonist. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No. 090505B 1, U.S. Pat. No. 8,586,023, PCT Publication Nos.: WO 2010/003118 and 2011/090754, or an anti-GITR antibody described, e.g., in U.S. Pat. No. 7,025,962, European Patent No. 1947183B 1, U.S. Pat. Nos. 7,812,135, 8,388,967, 8,591,886, European Patent No. EP 1866339, PCT Publication No. WO 2011/028683, PCT Publication No. WO 2013/039954, PCT Publication No. WO2005/007190, PCT Publication No. WO 2007/133822, PCT Publication No. WO2005/055808, PCT Publication No. WO 99/40196, PCT Publication No. WO 2001/03720, PCT Publication No. WO99/20758, PCT Publication No. WO2006/083289, PCT Publication No. WO 2005/115451, U.S. Pat. No. 7,618,632, and PCT Publication No. WO 2011/051726. An exemplary anti-GITR antibody is TRX518.

In some embodiments, the test agent, e.g., the additional agent, enhances tumor infiltration or transmigration of the administered cells, e.g., CAR-expressing cells. For example, in some embodiments, the additional agent stimulates CD40, such as CD40L, e.g., recombinant human CD40L. Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893. In some embodiments, the additional agent that enhances tumor infiltration includes tyrosine kinase inhibitor sunitnib, heparanase, and/or chemokine receptors such as CCR2, CCR4, and CCR7.

In some embodiments, the test agent, e.g., the additional agent, is an immunomodulatory agent that is a structural or functional analog or derivative of thalidomide and/or an inhibitor of E3 ubiquitin ligase. In some embodiments, the immunomodulatory agent binds to cereblon (CRBN). In some embodiments, the immunomodulatory agent binds to the CRBN E3 ubiquitin-ligase complex. In some embodiments, the immunomodulatory agent binds to CRBN and the CRBN E3 ubiquitin-ligase complex. In some embodiments, the immunomodulatory agent up-regulates the protein or gene expression of CRBN. In some aspects, CRBN is the substrate adaptor for the CRL4^{CRBN} E3 ubiquitin ligase, and modulates the specificity of the enzyme. In some embodiments, binding to CRB or the CRBN E3 ubiquitin ligase complex inhibits E3 ubiquitin ligase activity. In some embodiments, the immunomodulatory agent induces the ubiquitination of KZF1 (Ikaros) and IKZF3 (Aiolos) and/or induces degradation of IKZF1 (Ikaros) and IKZF3 (Aiolos). In some embodiments, the immunomodulatory agent induces the ubiquitination of casein kinase 1A1 (CK1α) by the CRL4cRBN E3 ubiquitin ligase. In some embodiments, the ubiquitination of CK1α results in CK1a degradation.

In some embodiments, the immunomodulatory agent is an inhibitor of the Ikaros (IKZF1) transcription factor. In some embodiments, the immunomodulatory agent enhances ubiquitination of Ikaros. In some embodiments, the immunomodulatory agent enhances the degradation of Ikaros. In some embodiments, the immunomodulatory agent down-regulates the protein or gene expression of Ikaros. In some embodiments, administration of the immunomodulatory agent causes a decrease in Ikaros protein levels.

In some embodiments, the immunomodulatory agent is an inhibitor of the Aiolos (IKZF3) transcription factor. In some embodiments, the immunomodulatory agent enhances ubiquitination of Aiolos. In some embodiments, the immunomodulatory agent enhances the degradation of Aiolos. In some embodiments, the immunomodulatory agent down-regulates the protein or gene expression of Aiolos. In some embodiments, administration of the immunomodulatory agent causes a decrease in Aiolos protein levels.

In some embodiments, the immunomodulatory agent is an inhibitor of both the Ikaros (IKZF1) and Aiolos (IKZF3) transcription factors. In some embodiments, the immunomodulatory agent enhances ubiquitination of both Ikaros and Aiolos. In some embodiments, the immunomodulatory agent enhances the degradation of both Ikaros and Aiolos. In some embodiments, the immunomodulatory agent enhances ubiquitination and degradation of both Ikaros and Aiolos. In some embodiments, administration of the immunomodulatory agent causes both Aiolos protein levels and Ikaros protein levels to decrease.

In some embodiments, the immunomodulatory agent is a selective cytokine inhibitory drug (SelCID). In some embodiments, the immunomodulatory agent inhibits the activity of phosphodiesterase-4 (PDE4). In some embodiments, the immunomodulatory agent suppresses the enzymatic activity of the CDCl25 phosphatases. In some embodiments, the immunomodulatory agent alters the intracellular trafficking of CDCl25 phosphatases.

In some embodiments, the immunomodulatory agent is thalidomide (2-(2,6-dioxopiperidin-3-yl)-1H-isoindole-1,3(2H)-dione) or an analog or derivative of thalidomide. In certain embodiments, a thalidomide derivative includes structural variants of thalidomide that have a similar biological activity. Exemplary thalidomide derivatives include, but are not limited to lenalidomide (REVLIMMUNOMODULATORY COMPOUND™; Celgene Corporation), pomalidomide (also known as ACTIMMUNOMODULATORY COMPOUND™ or POMALYST™ (Celgene Corporation)), CC-1088, CDC-501, and CDC-801, and the compounds disclosed in U.S. Pat. Nos. 5,712,291; 7,320,991; and 8,716,315; U.S. Appl. No. 2016/0313300; and PCT Pub. Nos. WO 2002/068414 and WO 2008/154252.

In some embodiments, the immunomodulatory agent is 1-oxo- and 1,3 dioxo-2-(2,6-dioxopiperldin-3-yl) isoindolines substituted with amino in the benzo ring as described in U.S. Pat. No. 5,635,517 which is incorporated herein by reference.

In some embodiments, the immunomodulatory agent is a compound of the following formula:

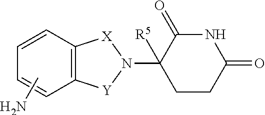

wherein one of X and Y is —C(O)— and the other of X and Y is —C(O)— or —CH₂—, and R⁵ is hydrogen or lower alkyl, or a pharmaceutically acceptable salt thereof. In some embodiments, X is —C(O)— and Y is —CH₂—. In some embodiments, both X and Y are —C(O)—. In some embodiments, R⁵ is hydrogen. In other embodiments, R⁵ is methyl.

In some embodiments, the immunomodulatory compound is a compound that belongs to a class of substituted 2-(2,6-dioxopiperidin-3-yl)phthalimmunomodulatory compounds and substituted 2-(2,6-dioxopiperldin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference.

In some embodiments, the immunomodulatory agent is a compound of the following formula:

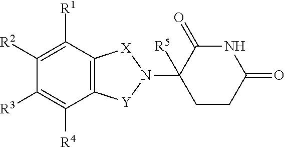

wherein

one of X and Y is —C(O)— and the other of X and Y is —C(O)— or —CH₂—;

(1) each of R¹, R², R³, and R⁴ are independently halo, alkyl of 1 to 4 carbon atoms, or alkoxy or 1 to 4 carbon atoms, or

(2) one of R¹, R³, R⁴, and R⁵ is —NHR^{a} and the remaining of R¹, R², R³, and R⁴ is are hydrogen, wherein R^{a} is hydrogen or alkyl of 1 to 8 carbon atoms;

R⁵ is hydrogen or alkyl of 1 to 8 carbon atoms, benzyl, or halo;

provided that R⁵ is other than hydrogen if X and Y are —C(O)— and (i) each of R¹, R², R³, and R⁴ is fluoro; or (ii) one of R¹, R², R³, and R⁴ is amino;

or a pharmaceutically acceptable salt thereof.

In some embodiments, the immunomodulatory agent is a compound that belongs to a class of isoindole-immunomodulatory compounds disclosed in U.S. Pat. No. 7,091,353, U.S. Patent Publication No. 2003/0045552, and International Application No. PCT/USOI/50401 (International Publication No. WO02/059106), each of which are incorporated herein by reference. For example, in some embodiments, the immunomodulatory agent is [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl)-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl)-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; or N-{[2-(2,6-dioxo(3-piperidyl))-1,3-dioxoisoindolin-4-yl]methyl}(benzylamino)carboxamide.

In some embodiments, the immunomodulatory agent is a compound that belongs to a class of isoindole-immunomodulatory compounds disclosed in U.S. Patent Application Publication Nos. 2002/0045643, International Publication No. WO 98/54170, and U.S. Pat. No. 6,395,754, each of which is incorporated herein by reference. In some embodiments, the immunomodulatory agent is a tetra substituted 2-(2,6-dioxopiperdin-3-yl)-1-oxoisoindolines described in U.S. Pat. No. 5,798,368, which is incorporated herein by reference. In some embodiments, the immunomodulatory agent is 1-oxo and 1,3-dioxo-2-(2,6-dioxopiperidin-3-yl) isoindolines disclosed in U.S. Pat. No. 6,403,613, which is incorporated herein by reference. In some embodiments the immunomodulatory agent is a 1-oxo or 1,3-dioxoisoindoline substituted in the 4- or 5-position of the indoline ring as described in U.S. Pat. Nos. 6,380,239 and 7,244,759, both of which are incorporated herein by reference.

In some embodiments, the immunomodulatory agent is 2-(4-amino-1-oxo-1,3-dihydro-isoindol-2-yl)-4-carbamoyl-butyric acid or 4-(4-amino-1-oxo-1,3-dihydro-isoindol-2-yl)-4-carbamoyl-butyric acid. In some embodiments, the immunomodulatory compound is 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, or 2-{4-[(furan-2-yl-methyl)-amino]-1,3-dioxo-1,3-dihydro-isoindol-2-yl}-pentanedioic acid.

In some embodiments, the immunomodulatory agent is a isoindoline-1-one or isoindoline-1,3-dione substituted in the 2-position with 2,6-dioxo-3-hydroxypiperidin-5-yl as described in U.S. Pat. No. 6,458,810, which is incorporated herein by reference. In some embodiments, the immunomodulatory compound is 3-(5-amino-2-methyl-4-oxo-4H-quinazolin-3-yl)-piperidine-2,6-dione, or an enantiomer or a mixture of enantiomers thereof; or a pharmaceutically acceptable salt, solvate, hydrate, co-crystal, clathrate, or polymorph thereof. In some embodiments, the immunomodulatory compound is 3-[4-(4-morpholin-4-ylmethyl-benzyloxy)-1-oxo-1,3-dihydro-isoindol-2-yl]-piperidine-2,6-dione.

In some embodiments, the immunomodulatory agent is as described in Oshima, K. et al., Nihon Rinsho., 72(6):1130-5 (2014); Millrine, D. et al., Trends Mol Med., 23(4):348-364 (2017); and Collins, et al., Biochem J., 474(7):1127-1147 (2017).

In some embodiments, the immunomodulatory agent is lenalidomide, pomalidomide, avadomide, a stereoisomer of lenalidomide, pomalidomide, avadomide or a pharmaceutically acceptable salt, solvate, hydrate, co-crystal, clathrate, or polymorph thereof. In some embodiments, the immunomodulatory compound is lenalidomide, a stereoisomer of lenalidomide or a pharmaceutically acceptable salt, solvate, hydrate, co-crystal, clathrate, or polymorph thereof. In some embodiments, the immunomodulatory compound is lenalidomide, or ((RS)-3-(4-Amino-1-oxo-1,3-dihydro-2H-isoindol-2-yl)piperidine-2,6-dione).

In some embodiments, the test agent, e.g., the additional agent, includes thalidomide drugs or analogs thereof and/or derivatives thereof, such as lenalidomide, pomalidomide or apremilast. See, e.g., Bertilaccio et al., Blood (2013) 122:4171, Otahal et al., Oncoimmunology (2016) 5(4):e1115940; Fecteau et al., Blood (2014) 124(10):1637-1644 and Kuramitsu et al., Cancer Gene Therapy (2015) 22:487-495). Lenalidomide ((RS)-3-(4-Amino-1-oxo-1,3-dihydro-2H-isoindol-2-yl)piperidine-2,6-dione; also known as Revlimid) is a synthetic derivative of thalidomide, and has multiple immunomodulatory effects, including enforcement of immune synapse formation between T cell and antigen presenting cells (APCs). For example, in some cases, lenalidomide modulates T cell responses and results in increased interleukin (IL)-2 production in CD4+ and CD8+ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4):e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. Lenalidomide also can enhance T-cell proliferation and interferon-γ production in response to activation of T cells via CD3 ligation or dendritic cell-mediated activation. Lenalidomide can also induce malignant B cells to express higher levels of immunostimulatory molecules such as CD80, CD86, HLA-DR, CD95, and CD40 (Fecteau et al., Blood (2014) 124(10):1637-1644). In some embodiments, lenalidomide is administered at a dosage of from about 1 mg to about 20 mg daily, e.g., from about 1 mg to about 10 mg, from about 2.5 mg to about 7.5 mg, from about 5 mg to about 15 mg, such as about 5 mg, 10 mg, 15 mg or 20 mg daily. In some embodiments, lenalidomide is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg.

In some embodiments, the test agent, e.g., the additional agent, is a B-cell inhibitor. In some embodiments, the test agent is one or more B-cell inhibitors selected from among inhibitors of CD10, CD19, CD20, CD22, CD34, CD123, CD79a, CD79b, CD179b, FLT-3, or ROR1, or a combination thereof. In some embodiments, the B-cell inhibitor is an antibody (e.g., a mono- or bispecific antibody) or an antigen binding fragment thereof. In some embodiments, the test agent, e.g., the additional agent, is an engineered cell expressing recombinant receptors that target B-cell targets, e.g., CD10, CD19, CD20, CD22, CD34, CD123, CD79a, CD79b, CD179b, FLT-3, or ROR1.

In some embodiments, the test agent, e.g., the additional agent, is a CD20 inhibitor, e.g., an anti-CD20 antibody (e.g., an anti-CD20 mono- or bi-specific antibody) or a fragment thereof. Exemplary anti-CD20 antibodies include but are not limited to rituximab, ofatumumab, ocrelizumab (also known as GA101 or RO5072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1):135-43. In some embodiments, the anti-CD20 antibody comprises rituximab. Rituximab is a chimeric mouse/human monoclonal antibody IgG1 kappa that binds to CD20 and causes cytolysis of a CD20 expressing cell. In some embodiments, the test agent includes rituximab. In some embodiments, the CD20 inhibitor is a small molecule.

In some embodiments, the test agent, such as the additional agent, is a CD22 inhibitor, e.g., an anti-CD22 antibody (e.g., an anti-CD22 mono- or bi-specific antibody) or a fragment thereof. Exemplary anti-CD22 antibodies include epratuzumab and RFB4. In some embodiments, the CD22 inhibitor is a small molecule. In some embodiments, the antibody is a monospecific antibody, optionally conjugated to a second agent such as a chemotherapeutic agent. For instance, in some embodiments, the antibody is an anti-CD22 monoclonal antibody-MMAE conjugate (e.g., DCDT2980S). In some embodiments, the antibody is an scFv of an anti-CD22 antibody, e.g., an scFv of antibody RFB4. In some embodiments, the scFv is fused to all of or a fragment of Pseudomonas exotoxin-A (e.g., BL22). In some embodiments, the scFv is fused to all of or a fragment of (e.g., a 38 kDa fragment of) Pseudomonas exotoxin-A (e.g., moxetumomab pasudotox). In some embodiments, the anti-CD22 antibody is an anti-CD19/CD22 bispecific antibody, optionally conjugated to a toxin. For instance, in some embodiments, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In some embodiments, the bispecific portion (e.g., anti-CD 19/anti-CD22) is linked to a toxin such as deglycosylated ricin A chain (e.g., Combotox).

In some embodiments, the test agent, such as the additional agent, is a cytokine or is an agent that induces increased expression of a cytokine in the tumor microenvironment. Cytokines have important functions related to T cell expansion, differentiation, survival, and homeostasis. Cytokines that can be administered to the subject receiving the combination therapy in the provided methods or uses, recombinant receptors, cells and/or compositions provided herein include one or more of IL-2, IL-4, IL-7, IL-9, IL-15, IL-18, and IL-21. In some embodiments, the cytokine administered is IL-7, IL-15, or IL-21, or a combination thereof. In some embodiments, administration of the cytokine to the subject that has sub-optimal response to the administration of the engineered cells, e.g., CAR-expressing cells improves efficacy and/or anti-tumor activity of the administered cells, e.g., CAR-expressing cells.

In some embodiments, the test agent, e.g., the additional agent, such as a protein that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2.

In some embodiments, the test agent, such as the additional agent, includes an interleukin-15 (IL-15) polypeptide, an interleukin-15 receptor alpha (IL-15Rα) polypeptide, or combination thereof, e.g., hetIL-15 (Admune Therapeutics, LLC). hetIL-15 is a heterodimeric non-covalent complex of IL-15 and IL-15Rα. hetIL-15 is described in, e.g., U.S. Pat. No. 8,124,084, U.S. 2012/0177598, U.S. 2009/0082299, U.S. 2012/0141413, and U.S. 2011/0081311. In some embodiments, the immunomodulatory agent can contain one or more cytokines. For example, the interleukin can include leukocyte interleukin injection (Multikine), which is a combination of natural cytokines.

In some embodiments, the test agent, such as an additional agent, is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the test agent, e.g., the additional agent, can reduce or reverse immunosuppressive effects of adenosine, adenosine analogs and/or adenosine signaling. In some embodiments, the test agent, e.g., the additional agent, can reduce or reverse hypoxia-driven A2-adenosinergic T cell immunosuppression. In some embodiments, the test agent, e.g., the additional agent, is selected from among antagonists of adenosine receptors, extracellular adenosine-degrading agents, inhibitors of adenosine generation by CD39/CD73 ectoenzymes, and inhibitors of hypoxia-HIF-la signaling. In some embodiments, the test agent is an adenosine receptor antagonist or agonist.

In some embodiments, the test agent, such as the additional agent, is an agent that inhibits the activity and/or an amount of an adenosine receptor. Particular embodiments contemplate that inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the test agent, e.g., the additional agent, is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the test agent, such as an additional agent, inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

In some embodiments, the test agent, such as the additional agent, is an adenosine receptor antagonist. In some embodiments, the antagonist is small molecule or chemical compound of an adenosine receptor, such as the A2a, A2b, or A3 receptor. In some embodiments, the antagonist is a peptide, or a peptidomimetic, that binds the adenosine receptor but does not trigger a Gi protein dependent intracellular pathway. Examples of such antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, the test agent, such as the additional agent, is an A2 receptor (A2R) antagonist, such as an A2a antagonist. Exemplary A2R antagonists include, but are not limited to, KW6002 (istradefyline), SCH58261, caffeine, paraxanthine, 3,7-dimethyl-l-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the test agent, such as the additional agent, is an A2R antagonist described in, e.g., Ohta et al., Proc Natl Acad Sci USA (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7):598-605; U.S. Pat. Nos. 8,080,554; 8,716,301; US 20140056922; WO2008/147482; U.S. Pat. No. 8,883,500; US 20140377240; WO02/055083; U.S. Pat. Nos. 7,141,575; 7,405,219; 8,883,500; 8,450,329 and 8,987,279).

In particular embodiments, an adenosine receptor antagonist that is an antisense molecule, inhibitory nucleic acid molecule (e.g., small inhibitory RNA (siRNA)) or catalytic nucleic acid molecule (e.g. a ribozyme) that specifically binds mRNA encoding an adenosine receptor. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gs protein- or Gi protein-dependent intracellular pathway. In some embodiments, the test agent, such as the additional agent, includes dominant negative mutant form of an adenosine receptor, such as A2a, A2b, or A3.

In some embodiments, the test agent, such as the additional agent, is an agent that inhibits extracellular adenosine. Agents that inhibit extracellular adenosine include agents that render extracellular adenosine non-functional (or decrease such function), such as a substance that modifies the structure of adenosine to inhibit the ability of adenosine to signal through adenosine receptors. In some embodiments, the test agent, such as the additional agent, is an extracellular adenosine-generating or adenosine-degrading enzyme, a modified form thereof or a modulator thereof. For example, in some embodiments, the test agent, such as the additional agent, is an enzyme (e.g. adenosine deaminase) or another catalytic molecule that selectively binds and destroys the adenosine, thereby abolishing or significantly decreasing the ability of endogenously formed adenosine to signal through adenosine receptors and terminate inflammation.

In some embodiments, the test agent, such as the additional agent, is an adenosine deaminase (ADA) or a modified form thereof, e.g., recombinant ADA and/or polyethylene glycol-modified ADA (ADA-PEG), which can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine includes agents that prevent or decrease formation of extracellular adenosine, and/or prevent or decrease the accumulation of extracellular adenosine, thereby abolishing, or substantially decreasing, the immunosuppressive effects of adenosine. In some embodiments, the test agent, such as the additional agent, specifically inhibits enzymes and proteins that are involved in regulation of synthesis and/or secretion of pro-inflammatory molecules, including modulators of nuclear transcription factors. Suppression of adenosine receptor expression or expression of the Gs protein- or Gi protein-dependent intracellular pathway, or the cAMP dependent intracellular pathway, can result in an increase/enhancement of immune response.

In some embodiments, the test agent, such as the additional agent, can target ectoenzymes that generate or produce extracellular adenosine. In some embodiments, the test agent, such as the additional agent, targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs within the tumor microenvironment (TME), in or around solid tumors. In some embodiments, the test agent, such as the additional agent, is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

In some embodiments, the test agent such as the additional agent is a chemotherapeutic agent (sometimes referred to as a cytotoxic agent). In particular embodiments, the chemotherapeutic agent is any agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. In particular embodiments, chemotherapeutic drugs include alkylating agents, anthracyclines, cytoskeletal disruptors (taxanes), epothilones, histone deacetylase inhibitors, topoisomerase inhibitors, topoisomerase II inhibitors, kinase inhibitors, nucleotide analogs and precursor analogs, peptide antibiotics, platinum-based agents, and vinca alkaloids and derivatives.

Chemotherapeutic agents may include, but are not limited to, abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

In some embodiments, the test agent, such as the additional agent, is an inhibitor of hypoxia inducible factor 1 alpha (HIF-1α) signaling. Exemplary inhibitors of HIF-1α include digoxin, acriflavine, sirtuin-7 and ganetespib.

In some embodiments, the test agent, such as the additional agent, includes a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor, e.g., an SHP-2 inhibitor described herein.

In some embodiments, the test agent, such as the additional agent, is a kinase inhibitor. Kinase inhibitors, such as a CDK4 kinase inhibitor, a BTK kinase inhibitor, a MNK kinase inhibitor, or a DGK kinase inhibitor, can regulate the constitutively active survival pathways that exist in tumor cells and/or modulate the function of immune cells. In some embodiments, the kinase inhibitor is a Bruton's tyrosine kinase (BTK) inhibitor, e.g., ibrutinib. In some embodiments, the kinase inhibitor is a phosphatidylinositol-4,5-bisphosphate 3-kinase (PI3K) inhibitor. In some embodiments, the kinase inhibitor is a CDK4 inhibitor, e.g., a CDK4/6 inhibitor. In some embodiments, the kinase inhibitor is an mTOR inhibitor, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor. In some embodiments, the kinase inhibitor is an MNK inhibitor, or a dual PI3K/mTOR inhibitor. In some embodiments, other exemplary kinase inhibitors include the AKT inhibitor perifosine, the mTOR inhibitor temsirolimus, the Src kinase inhibitors dasatinib and fostamatinib, the JAK2 inhibitors pacritinib and ruxolitinib, the PKCβ inhibitors enzastaurin and bryostatin, and the AAK inhibitor alisertib.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (1-[(3R)-3-[4-Amino-3-(4-phenoxyphenyl)-1H-pyrazolo[3,4-d]pyrimidin-1-yl]piperidin-1-yl]prop-2-en-1-one; also known as PCI-32765). In some embodiments, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered. In some embodiments, the BTK inhibitor is a BTK inhibitor described in International Application WO 2015/079417.

In some embodiments, the kinase inhibitor is a PI3K inhibitor. PI3K is central to the PI3K/Akt/mTOR pathway involved in cell cycle regulation and lymphoma survival. Exemplary PI3K inhibitor includes idelalisib (PI3K6 inhibitor). In some embodiments, the test agent, such as the additional agent, is idelalisib and rituximab.

In some embodiments, the test agent, such as the additional agent, is an inhibitor of mammalian target of rapamycin (mTOR). In some embodiments, the kinase inhibitor is an mTOR inhibitor selected from temsirolimus; ridaforolimus (also known as AP23573 and MK8669); everolimus (RAD001); rapamycin (AY22989); simapimod; AZD8055; PF04691502; SF1126; and XL765. In some embodiments, the test agent, such as the additional agent, is an inhibitor of mitogen-activated protein kinase (MAPK), such as vemurafenib, dabrafenib, and trametinib.

In some embodiments, the test agent, such as the additional agent, is an agent that regulates pro- or anti-apoptotic proteins. In some embodiments, the test agent, such as the additional agent, includes a B-cell lymphoma 2 (BCL-2) inhibitor (e.g., venetoclax, also called ABT-199 or GDC-0199; or ABT-737). Venetoclax is a small molecule (4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) that inhibits the anti-apoptotic protein, BCL-2. Other agents that modulate pro- or anti-apoptotic protein include BCL-2 inhibitor ABT-737, navitoclax (ABT-263); Mcl-1 siRNA or Mcl-1 inhibitor retinoid N-(4-hydroxyphenyl) retinamide (4-HPR) for maximal efficacy. In some embodiments, the test agent, such as the additional agent, provides a pro-apoptotic stimuli, such as recombinant tumor necrosis factor-related apoptosis-inducing ligand (TRAIL), which can activate the apoptosis pathway by binding to TRAIL death receptors DR-4 and DR-5 on tumor cell surface, or TRAIL-R2 agonistic antibodies.

In some embodiments, the test agent, such as the additional agent, includes a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the test agent, such as the additional agent, includes a hypomethylating agent, e.g., a DNA methyltransferase inhibitor, e.g., azacitidine or decitabine.

In another embodiment, the additional therapy is a transplantation, e.g., allogeneic stem cell transplant.

In some embodiments, the additional therapy is a lymphodepleting therapy. In some embodiments, lymphodepletion is performed on a subject, e.g., prior to administering engineered cells, e.g., CAR-expressing cells. In some embodiments, the lymphodepletion comprises administering one or more of melphalan, Cytoxan, cyclophosphamide, and fludarabine. In some embodiments, a lymphodepleting chemotherapy is administered to the subject prior to, concurrently with, or after administration (e.g., infusion) of engineered cells, e.g., CAR-expressing cells. In an example, the lymphodepleting chemotherapy is administered to the subject prior to administration of engineered cells, e.g., CAR-expressing cells.

In some embodiments, the test agent, such as the additional agent, is an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Other exemplary combination therapy, treatment and/or agents include anti-allergenic agents, anti-emetics, analgesics and adjunct therapies. In some embodiments, the test agent, such as the additional agent, includes cytoprotective agents, such as neuroprotectants, free-radical scavengers, cardioprotectors, anthracycline extravasation neutralizers and nutrients.

In some embodiments, an antibody used as an test agent, such as the additional agent, is conjugated or otherwise bound to a therapeutic agent, e.g., a chemotherapeutic agent (e.g., Cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein. In some embodiments, the test agent, such as the additional agent, is an antibody-drug conjugate.

In some embodiments, one or more test agents can be used to assess or evaluate any of the additional agents described herein that can be prepared and administered as a combination therapy, such as in pharmaceutical compositions comprising one or more agents of the combination therapy and a pharmaceutically acceptable carrier, such as any described herein. In some embodiments, the test agent is administered to evaluate and or assess a combination therapy that can be administered simultaneously, concurrently or sequentially, in any order with the additional agents, therapy or treatment, wherein such administration provides therapeutically effective levels each of the agents in the body of the subject. In some embodiments, the test agent is administered to evaluate or assess an additional agent that can be co-administered with the combination therapy in the provided methods or uses, for example, as part of the same pharmaceutical composition or using the same method of delivery. In some embodiments, the test agent is administered to assess or evaluate an additional agent that is administered simultaneously with the cell therapy, e.g. dose of engineered T cells (e.g. CAR+ T cells), but in separate compositions. In some embodiments, the additional agent is incubated with the engineered cell, e.g., CAR-expressing cells, prior to administration of the cells.

In some examples, the test agent is administered to evaluate one or more additional agents that are administered subsequent to or prior to the administration of the cell therapy, e.g. dose of engineered T cells (e.g. CAR+ T cells), separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times. In some embodiments, the additional agent is administered prior to the cell therapy, e.g. dose of engineered T cells (CAR+ T cells) in the provided methods or uses, e.g., two weeks, 12 days, 10 days, 8 days, one week, 6 days, 5 days, 4 days, 3 days, 2 days or 1 day before the administration. In some embodiments, the additional agent is administered after the cell therapy, e.g. dose of engineered T cells (e.g. CAR+ T cells) in the provided methods or uses, e.g., two weeks, 12 days, 10 days, 8 days, one week, 6 days, 5 days, 4 days, 3 days, 2 days or 1 day after the administration.

In some embodiments, the test agent is administered to assess or evaluate a dose of the additional agent that can be any therapeutically effective amount, e.g., any dose amount described herein, and the appropriate dosage of the additional agent may depend on the type of disease to be treated, the type, dose and/or frequency of the binding molecule, recombinant receptor, cell and/or composition administered, the severity and course of the disease, previous therapy, the patient's clinical history and response to cell therapy, e.g. dose of engineered T cells (CAR+ T cells), and the discretion of the attending physician.

D. Measuring Signs, Symptoms, and Outcomes of the Mouse Model

In some embodiments, the methods provided herein include one or more steps for assessing, measuring, determining, and/or quantifying one or more signs, symptoms, and/or outcomes of toxicity, for example to compare assessments, measurements, and/or quantifications of toxicity in a mouse that receives a test agent, a test immunotherapy, and/or a test lymphodepleting agent or therapy with assessments, measurements, and/or quantifications of toxicity in a mouse that did not receive the test agent, test immunotherapy, and/or test lymphodepleting agent or therapy.

In particular embodiments, the any phenotype, attribute, quality, sign, symptom, or outcome that can be measured, assessed, quantified, or detected in a mouse can be used to compare a mouse of the mouse model provided herein with another mouse, for example a mouse that has received a test agent, a test lymphodepleting agent, and/or a test immunotherapy. In some embodiments, the mouse of the mouse model provided herein is compared to a mouse that does not receive any treatments, e.g., a naïve mouse, or a mouse that has not been administered an immunotherapy, a mouse that has not been administered a lymphodepleting agent or therapy, e.g., a lymphodepleting agent or therapy as described herein such as in Section I.B, a mouse that has not been administered antigen expressing cells, and/or a mouse that has not been administered an immunotherapy, e.g., an immunotherapy described herein such as in Section I. C, but has been administered a mock or off target immunotherapy.

In some embodiments, assessing, measuring, and/or quantifying one or more signs, symptoms, and/or outcomes of the mouse model is or includes assessing, measuring, and/or quantifying in vivo expansion of an immunotherapy. In certain embodiments, the assessment, measurement, and/or quantification of the in vivo expansion is compared to another mouse, e.g., a mouse that receives a test agent and/or immunotherapy and/or a mouse that did not receive the immunotherapy. In some embodiments, the in vivo expansion of the immunotherapy is assessed, measured, and/or quantified, by collecting one or more samples at multiple time points following the administration of the immunotherapy and measuring or quantifying the amounts or levels of the immunotherapy in the sample. In certain embodiments, the immunotherapy is a cell composition, e.g., a CAR−T cell composition, and the amount or level of CAR−T cells are measured or quantified in the samples to determine the in vivo expansion of the immunotherapy. In some embodiments, the samples are blood samples. In particular embodiments, the embodiments are tissue samples.

In some embodiments, assessing, measuring, and/or quantifying one or more signs, symptoms, and/or outcomes of the mouse model is or includes assessing, measuring, and/or quantifying in vivo persistence of the immunotherapy. In certain embodiments, the assessment, measurement, and/or quantification of the in vivo persistence is compared to another mouse, e.g., a mouse that receives a test agent and/or immunotherapy and/or a mouse that did not receive the immunotherapy. In certain embodiments, the in vivo persistence of the immunotherapy is assessed, measured, and/or quantified, by collecting one or more samples at multiple time points following the administration of the immunotherapy and measuring or quantifying the amounts or levels of the immunotherapy in the sample. In certain embodiments, the immunotherapy is a cell composition, e.g., a CAR−T cell composition, and the amount or level of CAR−T cells are measured or quantified in the samples to determine the persistence of the immunotherapy. In some embodiments, the samples are blood samples. In particular embodiments, the embodiments are tissue samples.

In some embodiments, the degree or extent of the in vivo expansion and/or persistence of an administered immunotherapy can be detected or quantified after administration. For example, in some aspects, quantitative PCR (qPCR) or RNA sequencing (RNA-seq) is used to assess the quantity of the immunotherapy, e.g., cells expressing the recombinant receptor or CAR-expressing cells, in the blood or serum or organ or tissue. In some aspects, expansion and/or persistence is quantified as copies of DNA or plasmid encoded by, expressed by, and/or contained by the immunotherapy, such receptor, e.g., CAR or surrogate marker, e.g., Thy1.1, per microgram of DNA, or as the number of receptor-expressing, e.g., CAR-expressing, cells per microliter of the sample, e.g., of blood or serum, or per total number of blood cells or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor.

In some embodiments, assessing, measuring, and/or quantifying one or more signs, symptoms, and/or outcomes of the mouse model is or includes assessing, measuring, and/or quantifying tissue infiltration of the immunotherapy. In certain embodiments, the assessment, measurement, and/or quantification of tissue infiltration is compared to another mouse, e.g., a mouse that receives a test agent and/or immunotherapy and/or a mouse that did not receive the immunotherapy. In some embodiments, the infiltration is assessed, measured, and/or quantified by detecting a level or amount of the immunotherapy in a tissue. In certain embodiments, the immunotherapy is a cell composition, e.g., a CAR−T cell composition, and the amount or level of CAR−T cells are measured or quantified in tissue to determine the infiltration of the immunotherapy.

In some embodiments, the degree or extent of the infiltration of an administered immunotherapy can be detected or quantified after administration. For example, in some aspects, quantitative PCR (qPCR) or RNA sequencing (RNA-seq) is used to assess the quantity of the immunotherapy, e.g., cells expressing the recombinant receptor or CAR-expressing cells, in the organ or tissue. In certain embodiments, antibody staining techniques such as immunofluorescence, immunohistochemistry, and/or immunohistochemistry can be employed to detect and/or identify the immunotherapy, e.g., CAR expressing cells, in tissue or organ, for example in sections of the tissue or organ. In some embodiments, a detectable antibody that recognizes and/or binds to a recombinant receptor or CAR is expressed by the immunotherapy is used to assess infiltration in an organ or tissue. In particular embodiments, the antibody does not bind to or recognize any antigen that is endogenous to the mouse. In some embodiments, the antibodies recognize or bind to a molecule, such as a surrogate marker, that is not expressed by endogenous cells of the mouse.

In some embodiments, assessing, measuring, and/or quantifying one or more signs, symptoms, and/or outcomes of the mouse model is or includes assessing, measuring, and/or quantifying in vivo activity of an immunotherapy. In certain embodiments, the assessment, measurement, and/or quantification of the in vivo activity is compared to another mouse, e.g., a mouse that receives a test agent and/or immunotherapy and/or a mouse that did not receive the immunotherapy. In some embodiments, the in vivo activity of the immunotherapy is determined by measuring the amount of a target, e.g., cells that express an antigen bound by and/or recognized by the immunotherapy, that remains after the immunotherapy has been administered. In particular embodiments, the target is or includes cells are cancer cells and/or tumor cells. In some embodiments, the cells are antigen-expressing cells, e.g., any one or more antigen-expressing cells that are described herein such as in Section I.D. In some embodiments, the antigen-expressing cells are A20 cells. In certain embodiments, cells that are targeted by the immunotherapy may be quantified by any suitable technique, including but not limited to histology, e.g., to identify and measure lesions and/or tumors, flow cytometry analysis, antibody staining techniques such as immunofluorescence, immunohistochemistry, and/or immunohistochemistry, western blot analysis, and/or qPCR.

In particular embodiments, assessing, measuring, and/or quantifying one or more signs, symptoms, and/or outcomes of the mouse model is or includes assessing, measuring, and/or quantifying inflammation and/or an immune response, and/or one or more biomarkers associated with inflammation and/or an immune response. In certain embodiments, the assessment, measurement, and/or quantification of the inflammation and/or immune response is compared to another mouse, e.g., a mouse that receives a test agent and/or immunotherapy and/or a mouse that did not receive the immunotherapy. In certain embodiments, assessing, measuring, and/or quantifying inflammation and/or an immune response, and/or one or more biomarkers associated with inflammation and/or an immune response can be performed by any suitable methods, including any methods or techniques that are described herein.

In some embodiments, a biomarker associated with inflammation and/or immune response includes any molecule that is increased or decreased in association with an immune or inflammatory response. In some embodiments, the biomarker is a protein, a polynucleotide, e.g., an mRNA, a lipid, a saccharide. In some embodiments, the biomarker is protein. In some embodiments, the biomarker is a protein in a particular state, e.g., that has or contains one or more post-translational modifications. In some embodiments, post-translational modification refers to any modification of a polypeptide during or after the protein is synthesized. Post translational modifications include, but are not limited to, phosphorylation, acetylation, methylation, glycosylation, lipidation, myristoylation, palmitoylation, farnesylation, geranylgeranylation, formylation, amidation, glypiation, lipoylation, acylation, butyrylation, malonylation, hydroxylation, S-nitrosylation, succinylation, sumoylation, ubiquitination, and neddylation.

In some embodiments, assessing, measuring, and/or quantifying one or more signs, symptoms, and/or outcomes of the mouse model is or includes assessing, measuring, and/or quantifying the level or amount of one or more cytokines in the mouse. In particular embodiments, the assessment, measurement, and/or quantification of one or more cytokines is compared to another mouse, e.g., a mouse that receives a test agent and/or immunotherapy and/or a mouse that did not receive the immunotherapy.

In some embodiments, the one or more cytokines are assessed, measured, and/or quantified from a blood or serum sample. In certain embodiments, the one or more cytokines are assessed, measured, and/or quantified from a tissue sample. In certain embodiments, one or more cytokines are measured by any suitable means. For example, in particular embodiments, cytokines may be detected by ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), RIA (radioimmunoassay), SPR (surface plasmon resonance), nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays).

In certain embodiments, the cytokines may be assessed, measured and/or quantified by measuring and/or quantifying the expression of a gene that encodes a cytokine, and or by measuring and/or quantifying a gene that is upregulated or downregulated in response to a cytokine.

In certain embodiments, assessing, measuring, and/or quantifying one or more signs, symptoms, and/or outcomes of the mouse model is or includes assessing, measuring, and/or quantifying the expression of one or more genes. In particular embodiments, the assessment, measurement, and/or quantification of the expression of one or more genes is compared to another mouse, e.g., a mouse that receives a test agent and/or immunotherapy and/or a mouse that did not receive the immunotherapy. In certain embodiments, assessing, measuring, determining, and/or quantifying the expression of a gene is or includes assessing, measuring, determining, and/or quantifying an amount or level of a gene product that is encoded by the gene. In certain embodiments, the gene product is a polynucleotide that is expressed or encoded by the gene. In some embodiments, the gene product is a protein. In particular embodiments, the gene expression is compared to and/or normalized to the gene expression of a mouse that did not receive the immunotherapy, e.g., a naïve mouse.

In certain embodiments, assessing, measuring, and/or quantifying one or more signs, symptoms, and/or outcomes of the mouse model is or includes assessing, measuring, and/or quantifying the expression of one or more genes in the brain. In certain embodiments, the expression of one or more genes relating to or belonging to a category, e.g., a gene ontology category, defined by a response to cytokines, response to interferon-beta, cellular response to interferon-beta, antigen processing and presentation of peptide antigen via MHC class I, regulation of cell morphogenesis, cellular response to cytokine stimulus, antigen processing and presentation of peptide antigen, innate immune response, response to interferon-gamma, antigen processing and presentation, cell junction assembly, angiogenesis, positive regulation of cell projection organization, regulation of neuron projection development, blood vessel morphogenesis, negative regulation of protein modification processes, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, negative regulation of phosphorylation, antigen processing and presentation of endogenous peptide antigen, response to peptide hormone, positive regulation of cellular component biogenesis, and/or positive regulation of cell migration is measured in brain.

In some embodiments, gene expression is measured by measuring a polynucleotide, e.g., an mRNA polynucleotide, expressed by the gene. In some embodiments, the gene expression is measured by measuring a cDNA generated from an mRNA polynucleotide expressed by the gene.

In particular embodiments, the amount or level of a polynucleotide may be assessed, measured, determined, and/or quantified as a matter of routine. For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR methods (including, e.g., TAQMAN®, molecular beacon, LIGHTUP™, SCORPION™, SIMPLEPROBES®; see, e.g., U.S. Pat. Nos. 5,538,848; 5,925,517; 6,174,670; 6,329,144; 6,326,145 and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art, such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing. In particular embodiments, the levels of nucleic acid gene products are measured by qRT-PCR.

In particular embodiments, the expression of two or more of the genes are measured or assessed simultaneously. In certain embodiments, a multiplex PCR, e.g., a multiplex rt-PCR assessing, measuring, determining, and/or quantifying the level or amount of two or more gene products. In some embodiments, microarrays (e.g., AFFYMETRIX®, AGILENT® and ILLUMINA®-style arrays) are used for assessing, measuring, determining, and/or quantifying the level or amount of two or more gene products. In some embodiments, microarrays are used for assessing, measuring, determining, and/or quantifying the level or amount of a cDNA polynucleotide that is derived from an RNA gene product.

In particular embodiments, the expression of one or more polynucleotides are measured, determined, and/or quantified by RNA-Seq. RNA sequencing methods have been adapted for the most common DNA sequencing platforms [HiSeq systems (Illumina), 454 Genome Sequencer FLX System (Roche), Applied Biosystems SOLiD (Life Technologies), IonTorrent (Life Technologies)]. These platforms require initial reverse transcription of RNA into cDNA.

In certain embodiments, gene expression is assessed by measuring protein encoded by the gene. Suitable methods for assessing, measuring, determining, and/or quantifying the level or amount or more or more proteins include, but are not limited to quantitative immunocytochemistry or immunohistochemistry, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), RIA (radioimmunoassay), SPR (surface plasmon resonance), nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays).

In certain embodiments, the identification of genes that are differentially expressed in the mouse model may be used to identify potential targets for one or more test agents, for example to identify candidate agents to treat or ameliorate toxicity.

In certain embodiments, assessing, measuring, and/or quantifying one or more signs, symptoms, and/or outcomes of the mouse model is or includes assessing, measuring, and/or quantifying brain edema. In particular embodiments, the assessment, measurement, and/or quantification of brain edema is compared to another mouse, e.g., a mouse that receives a test agent and/or immunotherapy and/or a mouse that did not receive the immunotherapy. In some embodiments, the brain edema is assessed, measured, and/or quantified by detecting the brain water content.

In certain embodiments, the brain water content is measured, determined, and/or quantified by measuring the wet and dry weight of the brain. In some embodiments, the brain water content is the ratio of wet brain weight minus dry brain weight over wet brain weight multiplied by 100. In particular embodiments, the brain water content is [(wet brain weight−dry brain weight)/wet brain weight]*100. In particular embodiments, brain water content is determined as a matter of routine, and can be performed, for example, by any of the methods described in Kimbler et al. PLoS ONE 7(7): e41229 (2001); and in Wang et al. Annals of Clinical and Laboratory science 1(42):14-20 (2012).

In some embodiments, assessing, measuring, and/or quantifying one or more signs, symptoms, and/or outcomes of the mouse model is or includes assessing, measuring, and/or quantifying an aspect of blood and/or blood chemistry. In certain embodiments, the assessment, measurement, and/or quantification of the aspect of blood and/or blood chemistry is compared to another mouse, e.g., a mouse that receives a test agent and/or immunotherapy and/or a mouse that did not receive the immunotherapy. In certain embodiments, one or more aspects of blood chemistry are detected. In some embodiments, aspects of blood chemistry include, but are not limited to, levels, amounts, or concentrations of electrolytes, acid and base levels, blood iron content, hormone levels, markers of cardiovascular function, and proteins, e.g., serum albumin. In certain embodiments, aspects of blood and/or blood chemistry can be assessed by routine means, including by routine medical laboratory analysis, including with veterinary diagnostic and/or standard blood chemistry panels. In certain embodiments, aspects of blood chemistry may be measured, quantified, and/or assessed by standard research laboratory techniques such as immunoassays, ELISA, western blots, high performance liquid chromatography, and mass spectrometry.

In some embodiments, assessing, measuring, and/or quantifying one or more signs, symptoms, and/or outcomes of the mouse model is or includes assessing, measuring, and/or quantifying an aspect of tissue damage. In certain embodiments, the assessment, measurement, and/or quantification of the tissue damage is compared to another mouse, e.g., a mouse that receives a test agent and/or immunotherapy and/or a mouse that did not receive the immunotherapy. In certain embodiments, markers of tissue damage are quantified. In particular embodiments, markers of tissue damage include, but are not limited to, fibrosis, extramedullary hematopoiesis, infiltration, e.g., histiocytic granulomatous infiltration, lesions, and necrosis. In certain embodiments, tissue damage is assessed, measured, and/or quantified by pathophysiologic techniques, such as histological staining to sections obtained from tissue and/or an organ. In certain embodiments, the sections are frozen sections, semithin sections, paraffin fixed and/or embedded, or sections that are fixed in formalin, formaldehyde, and/or paraformaldehyde. In some embodiments, the tissue is stained with one or more histological stains, including but not limited to, hematoxylin and eosin (H&E), methyl green, methylene blue, pyronin G, toluidine blue, acid fushin, aniline blue, and/or orange G, Masson's trichrome, Periodic acid-Schiff reaction, alcian blue, van Gieson stain, reticulin stain, giemsa, Nissl and methylene blue, and/or Sudan Black and osmium. Methods of identifying damage to tissue is routine, and is described, for example, in Scudamore, “A Practical Guide to Histology in the Mouse”, John Wiley & Sons Inc., New York (2014); Treuting et al. “Comparative Anatomy and Histology”, Elsevier, Amsterdam (2012); Conti et al. “Atlas of Laboratory Mouse Histology”, Texas Histopages (2004); and Gude, “Histological Atlas of the Laboratory Mouse”, Springer, New York, (1982).

In some embodiments, assessing, measuring, and/or quantifying one or more signs, symptoms, and/or outcomes of the mouse model is or includes assessing, measuring, and/or quantifying one or more aspects of mouse physiology and/or behavior. In certain embodiments, the assessment, measurement, and/or quantification of physiology and/or behavior is compared to another mouse, e.g., a mouse that receives a test agent and/or immunotherapy and/or a mouse that did not receive the immunotherapy. In certain embodiments, the aspect of mouse physiology and/or behavior is or includes signs of disease or stress. In some embodiments, signs of disease or stress may include but are not limited to behavior such as avoidance of cage mates, reduced locomotor activity, increased vocalization, altered gait, hunched posture, reduced and/or a lack of grooming behavior, rough hair coat, decreased food and/or water consumption, decreased fecal or urine output, dehydration, labored breathing, squinted/sunken eyes, increase of self-mutilation behavior, alopecia, dermatitis, e.g., ulcerative dermatitis, and/or secretion of porphyrin, i.e., chromadacryorrhea. In some embodiments, the aspect of mouse behavior is food intake, e.g., reduced food intake. In particular embodiments, the aspect of mouse behavior is or includes reduced food intake, reduced water intake, reduced grooming, and/or reduced locomotor activity. In some embodiments, the aspect of mouse physiology is body weight, e.g., weight loss, or body temperature, e.g., reduced body temperature.

In particular embodiments, aspects of mouse physiology and/or behavior are assessed, measured, and/or quantified as a matter of routine. Methods for assessing and/or measuring aspects of mouse physiology and behavior are reviewed by Hedrich, “The Laboratory Mouse, Second Edition”, Elsevier, Amsterdam (2012); Crawly, “What's Wrong with My Mouse?”, Wiley (2007), and Bogdanske et al., “Laboratory Mouse Procedural Techniques”, CRC Press (2010)

In some embodiments, assessing, measuring, and/or quantifying one or more signs, symptoms, and/or outcomes of the mouse model is or includes assessing morbidity or death and/or the probability of death. In certain embodiments assessing morbidity or death is or includes assessing the severity of any signs or symptoms of toxicity, for example such as including reduced body temperature and or body weight. In some embodiments, morbidity or death is assessed by evaluating the mouse to determine if the mouse requires an intervention, e.g., treatment with subcutaneous injection of fluids, exposure to soft chow, and/or contact with heating pad. In some embodiments, morbidity or death is assessed by evaluating the mouse to determine if the mouse requires euthanasia as a human intervention to prevent undue suffering. In particular embodiments, the probability of morbidity or death is or includes determining the probability that the mouse will require an intervention or euthanasia within a given amount of time.

1. Compositions and Formulations

In some embodiments, the immunotherapy, such as cells genetically engineered with a recombinant receptor (e.g. CAR−T cells), the lymphodepleting agent or therapy, the antigen-expressing cells, and/or any test agents, test lymphodepletion agents, and/or test immunotherapies, are provided as a composition, including pharmaceutical compositions and/or pharmaceutical formulations. In some embodiments, the immunotherapy is provided in compositions such as unit dose form compositions including the amount of the T cell engaging therapy or the number of cells of a cell composition for administration in a given dose or fraction thereof. The pharmaceutical compositions and formulations generally include one or more optional pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the immunotherapy in amounts effective to create, stimulate, or trigger one or more signs, symptoms, and/or outcomes associated with toxicity in the mouse. The desired dosage can be delivered by a single bolus administration of the immunotherapy, by multiple bolus administrations of the immunotherapy, or by continuous infusion administration of the immunotherapy.

The cells and compositions may be administered using standard administration techniques, formulations, and/or devices. Administration of the cells can be autologous or heterologous. For example, immunoresponsive cells or progenitors can be obtained from one mouse, e.g., a donor mouse, and administered to the same mouse or a different, compatible mouse, e.g., a mouse with of the same strain, substrain, and/or genetic makeup. Immunoresponsive cells derived from a donor mouse or their progeny (e.g., in vivo, ex vivo or in vitro derived) can be administered via localized injection, including catheter administration, systemic injection, localized injection, intravenous injection, or parenteral administration.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells are administered in a single pharmaceutical composition.

In some embodiments, the cells are administered in a plurality of compositions, collectively containing the cells of a single dose.

Thus, one or more of the doses in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, multiple doses are given, in some aspects using the same timing guidelines as those with respect to the timing between the first and second doses, e.g., by administering a first and multiple subsequent doses, with each subsequent dose given at a point in time that is greater than about 28 days after the administration of the first or prior dose.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

As used herein, “percent (%) sequence identity” and “percent identity” when used with respect to a nucleotide sequence (reference nucleotide sequence) or amino acid sequence (reference amino acid sequence) is defined as the percentage of nucleotide residues or amino acid residues, respectively, in a candidate sequence that are identical with the residues in the reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Exemplary substitutions are shown in Table 1. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe

Non-conservative amino acid substitutions will involve exchanging a member of one of these classes for another class.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” refers to within ±25%, ±20%, ±15%, ±10%, ±5%, or ±1% of the value or parameter.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### V. EXEMPLARY EMBODIMENTS

Among the provided embodiments are:

1. A method for generating a mouse model of an immunotherapy-associated toxicity or an immunotherapy-associated toxic outcome, comprising:

i) administering a lymphodepleting agent or therapy to an immunocompetent mouse, wherein the lymphodepleting agent or therapy does not comprise total body radiation and/or does not comprise complete or substantially complete immune ablation; and

ii) subsequently administering to the mouse an immunotherapy, wherein the immunotherapy binds to and/or recognizes an antigen that is expressed on or in a cell or tissue of the immunocompetent mouse.

2. The method of embodiment 1, wherein the antigen is an antigen naturally expressed on murine cell and/or the antigen is a cell surface antigen and/or the immunotherapy binds to or recognizes an extracellular epitope of the antigen.

3. The method of embodiment 1 or embodiment 2, wherein the cell is a murine cell.

4. The method of any of embodiments 1-3, wherein the antigen is expressed on the surface of a circulating cell or the cell is a circulating cell.

5. The method of any of embodiments 1-4, wherein the antigen is a B cell antigen or is expressed on the surface of a B cell or wherein the cell is a murine B cell.

6. The method of any of embodiments 1-5, wherein the immunotherapy is an agent that stimulates or activates immune cells.

7. The method of embodiment 6, wherein the immunotherapy is a T cell-engaging therapy, optionally wherein the T-cell engaging therapy comprises a bispecific antibody, wherein at least one binding portion specifically binds to a T cell antigen, optionally CD3.

8. The method of embodiment 6 or embodiment 7, wherein the amino acid sequence of the T cell-engaging therapy comprises a murine sequence and/or is not immunogenic to the mouse.

9. The method of any of embodiments 1-5, wherein the immunotherapy comprises a cell therapy, said cell therapy optionally comprising a dose or composition of genetically engineered cells expressing a recombinant receptor.

10. The method of embodiment 9, wherein the engineered cells comprise cells obtained from a biological sample from the immunocompetent mouse or from a mouse that is of the same strain or substrain as the immunocompetent mouse.

11. The method of embodiment 10, wherein the biological sample comprises splenocytes.

12. The method of any of embodiments 9-11, wherein the engineered cells comprise NK cells or T cells, optionally wherein the T cells are CD4+ and/or CD8+ T cells.

13. A method for generating a mouse model of an immunotherapy-associated toxicity or an immunotherapy-associated toxic outcome, comprising:

i) administering a lymphodepleting agent or therapy to an immunocompetent mouse, wherein the lymphodepleting agent or therapy does not comprise total body radiation and/or does not comprise complete or substantially complete immune ablation; and

ii) subsequently administering to the mouse a cell therapy comprising murine T cells expressing a recombinant receptor that binds to and/or recognizes a murine antigen that is expressed on a B cell of the immunocompetent mouse.

14. The method of any of embodiments 9-13, wherein the recombinant receptor is a T cell receptor or a functional non-T cell receptor.

15. The method of any of embodiments 9-14, wherein the recombinant receptor is a chimeric receptor, optionally a chimeric antigen receptor (CAR).

16. The method of any of embodiments 9-15, wherein:

the amino acid sequence of the recombinant receptor is murine; and/or the individual regions or domains of the chimeric receptor comprise regions or domains of a natural murine protein and/or comprises a murine sequence; and/or the individual regions or domains of the chimeric receptor are not immunogenic to the mouse.

17. The method of embodiment 15 or embodiment 16, wherein the recombinant receptor is a chimeric antigen receptor (CAR) and the CAR comprises an extracellular antigen-binding domain that specifically binds to the antigen.

18. The method of embodiment 17, wherein the antigen-binding domain is an antibody or an antigen-binding fragment, wherein the antigen-binding fragment is optionally a single chain fragment, optionally an scFv.

19. The method of any of embodiments 15-18, wherein the CAR comprises an intracellular signaling domain comprising an ITAM, wherein optionally, the intracellular signaling domain comprises an intracellular domain of a CD3-zeta (CD3ζ) chain, optionally murine CD3-zeta.

20. The method of embodiment 19, wherein the intracellular signaling domain further comprises a costimulatory signaling region, which optionally comprises a signaling domain of CD28 or 4-1BB, optionally murine CD28 or murine 4-1BB.

21. The method of any of embodiments 1-20, wherein the antigen is or comprises ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen or; wherein the antigen is or comprises αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

22. The method of any of embodiments 1-21, wherein the antigen is B cell maturation antigen (BCMA), CD19, CD20, CD22, CD24, CD30, and/or CD38.

23. The method of any of embodiments 1-22, wherein the antigen is CD19.

24. The method of any of embodiments 1-23, wherein:

the antigen is expressed on cells administered to the mouse; and/or

the method comprises administering to the immunocompetent mouse one or more cells expressing the antigen, optionally wherein the antigen-expressing cells are administered prior to administering of the lymphodepleting agent or therapy.

25. The method of any of embodiments 1-24, wherein the antigen is expressed on or in tumor and/or cancer cells and/or the antigen-expressing cells are tumor and/or cancer cells, and wherein:

the immunocompetent mouse comprises the tumor and/or cancer cells; and/or

the method further comprises administering to the immunocompetent mouse one or more cancer cells and/or a tumor or tumor tissue, optionally prior to the administering of the lymphodepleting agent or therapy.

26. The method of embodiment 25, wherein the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor.

27. The method of embodiment 25 or embodiment 26, wherein the one or more cancer cells and/or the tumor comprise cancerous B cells, optionally mouse B cells and/or are B cell-derived.

28. The method of embodiment any of embodiments 1-27, wherein the mouse contains and/or the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

29. The method of any of embodiments 1-28, wherein the mouse contains and/or the one or more cancer cells or tumor cells comprise A20 cells.

30. The method of any of embodiments 1-29, wherein the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R.

31. The method of any of embodiments 1-30, wherein the immunocompetent mouse is not a C57BL/6 mouse or a substrain thereof.

32. The method of any of embodiments 1-31, wherein the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

33. The method of any of embodiments 1-32, wherein the immunocompetent mouse, following challenge with an antigen and optionally an adjuvant, has an increase in one or more cytokines compared to an immunocompetent C57BL/6mouse administered the same antigen, optionally wherein the one or more cytokine is an inflammatory cytokine.

34. The method of any of embodiments 1-33, wherein the immunocompetent mouse is a BALB/c mouse or is of a substrain thereof.

35. The method of embodiment 34, wherein the BALB/c mouse or substrain thereof is a BALB/cJ mouse or a BALB/cByJ mouse.

36. The method of any of embodiments 1-35, wherein at or about or within 24 hours after the administering the lymphodepleting agent or therapy the mouse comprises:

i) a depletion of a percentage of total circulating lymphocytes of between 10% and 95%, between 30% and 85%, or between about 50% and 75% compared to prior to initiation of the lymphodepleting agent or therapy; and/or

ii) a depletion of a percentage of circulating T cells of between 10% and 95%, between 30% and 85%, or between about 50% and 75% compared to prior to initiation of the lymphodepleting agent or therapy; and/or

iii) a depletion of a percentage of circulating B cells of between 50% and 99%, 75% and 99%, or 75% and 95% compared to prior to initiation of the lymphodepleting agent or therapy.

37. The method of any of embodiments 1-36, wherein the lymphodepleting agent or therapy comprises a chemotherapeutic agent.

38. The method of embodiment 37, wherein the chemotherapeutic agent comprises one or more a toxin, an alkylating agent, a DNA strand-breakage agent, a topoisomerase II inhibitors, a DNA minor groove binding agents, an antimetabolite, a tubulin interactive agent, a progestin, an adrenal corticosteroid, a luteinizing hormone releasing agent antagonist, a gonadotropin-releasing hormone antagonist, or an antihormonal antigen.

39. The method of embodiment 37 or embodiment 38, wherein chemotherapeutic agent comprises one or more of cyclophosphamide, chlorambucil, bendamustine, ifosfamide, prednisone, dexamethasone, cisplatin, carboplatin, oxaliplatin, fludarabine, pentostatin, clardribine, cytarabine, gemcitabine, methotrexate, pralatrexate, vincristine, doxorubicin, mitoxantrone, etoposide, bleomycin or combinations thereof.

40. The method of any of embodiments 37-39, wherein the chemotherapeutic agent is or comprises cyclophosphamide.

41. The method of any of embodiments 1-40, wherein:

the lymphodepleting agent or therapy comprises administering cyclophosphamide at a dose of at least or at least about 50 mg/kg, at least or at least about 100 mg/kg, at least or at least about 200 mg/kg, at least at least about 250 mg/kg, at least or at least about 300 mg/kg, at least or at least about 400 mg/kg, at least or at least about 500 mg/kg or at least or at least about 750 mg/kg or a range between any of the foregoing; or

the lymphodepleting agent or therapy comprises administering cyclophosphamide at a dose between or between about 50 mg/kg and 750 mg/kg, 50 mg/kg and 500 mg/kg, 50 mg/kg and 250 mg/kg, 50 mg/kg and 100 mg/kg, 100 mg/kg and 750 mg/kg, 100 mg/kg and 500 mg/kg, 100 mg/kg and 250 mg/kg, 250 mg/kg and 750 mg/kg, 250 mg/kg and 500 mg/kg or 500 mg/kg and 750 mg/kg, each inclusive.

42. The method of any of embodiments 1-41, wherein the lymphodepleting agent or therapy comprises administering cyclophosphamide at a dose of 250 mg/kg or about 250 mg/kg.

43. The method of embodiment 41 or embodiment 42, wherein the dose of cyclophosphamide is administered one time prior to initiation of administration of the immunotherapy.

44. The method of any of embodiments 40-43, wherein the cyclophosphamide is administered intraperitoneally.

45. The method of any of embodiments 1-5 and 9-44, wherein the cell therapy has not previously been cryofrozen.

46. The method of any of embodiments 1-45, wherein initiation of administration of the immunotherapy is between 0.5 hours and 120 hours after administering the lymphodepleting agent or therapy.

47. The method of any of embodiments 1-46, wherein initiation of administration of the immunotherapy is between 12 hours and 48 hours after administering the lymphodepleting agent or therapy.

48. The method of any of embodiments 1-47, wherein initiation of administration of the immunotherapy is 24 hours or about 24 hours after administering the lymphodepleting agent or therapy.

49. The method of any of embodiments 1-5 and 9-48, wherein the cell therapy comprises the administration of from or from about 1×10⁶ to 1×10⁸ total recombinant receptor-expressing cells or total T cells.

50. The method of any of any of embodiments 1-5 and 9-49, wherein a the cell therapy comprises the administration of at least or about at least or at or about 5×10⁶ total recombinant receptor-expressing cells or total T cells, 1×10⁷ total recombinant receptor-expressing cells or total T cells, or 5×10⁷ total recombinant receptor-expressing cells or total T cells.

51. The method of any of embodiments 1-50, wherein the method results in a toxicity comprising one or more signs, symptoms or outcomes associated with or selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior.

52. The method of embodiment 51, wherein the one or more signs, symptoms or outcomes is or is associated with inflammation, wherein the inflammation comprises histiocytic granulomatous infiltration, optionally of the liver, lung, spleen, or brain.

53. The method of embodiment 51, wherein the one or more signs, symptoms or outcomes is or is associated with altered level, amount or expression or ratio thereof of one or more molecules in the serum, wherein the one or more molecules is a cytokine, chemokine or growth factor.

54. The method of embodiment 51 or embodiment 53, wherein the altered level, amount or expression or ratio thereof of the molecule comprises an increased level, amount or expression compared to the level, amount or expression of the molecule in the mouse prior to administering the lymphodepleting therapy and/or immunotherapy and/or compared to the level, amount or expression of the molecule, on average, in a naïve mouse of the same strain and/or compared to the level, amount or expression of the molecule, on average, in a naïve mouse of the same strain and/or compared to the level, amount or expression of the molecule in a mouse administered a non-target immunotherapy.

55. The method of embodiment 54, wherein the level, amount or expression is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

56. The method of any of embodiments 53-55, wherein the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2.

57. The method of any of embodiments 54-56, wherein the increased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

58. The method of embodiment 51, wherein the altered level, amount or expression or ratio thereof is or comprises an altered ratio of angiopoetin-2 to angiopoetin-1 (Ang2:Ang1 ratio) in the serum, optionally wherein the altered ratio is an increased ratio.

59. The method of embodiment 58, wherein the Ang2:Ang1 ratio is increased by at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold compared to the Ang2:Ang1 ratio in the mouse prior to administering the lymphodepleting therapy and/or immunotherapy and/or compared to the Ang2:Ang1 ratio, on average, in a naïve mouse of the same strain and/or compared to the Ang2:Ang1 ratio in a mouse administered a non-target immunotherapy.

60. The method of embodiment 51, wherein the altered level, amount or expression or ratio thereof is or comprises a ratio of angiopoetin-2 to angiopoetin-1 (Ang2:Ang1 ratio) in the serum of at least 1, at least 2, at least 3, at least 4, at least 5, at least 10, at least 15, at least 20, at least 25, at least 30, at least 40, at least 50, at least 100, at least 500, at least 1,000, or at least 5,000 or higher.

61. The method of embodiment 60, wherein the Ang2:Ang1 ratio is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

62. The method of embodiment 51 or embodiment 53, wherein the altered level, amount or expression or ratio thereof of the molecule comprises a decreased level, amount or expression compared to the level, amount or expression of the molecule in the mouse prior to administering the lymphodepleting therapy and/or immunotherapy and/or compared to the level, amount or expression of the molecule, on average, in a naïve mouse of the same strain and/or compared to the level, amount or expression of the molecule, on average, in a naïve mouse of the same strain and/or compared to the level, amount or expression of the molecule in a mouse administered a non-target immunotherapy.

63. The method of embodiment 62, wherein the level, amount or expression is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

64. The method of embodiment 53, embodiment 62 or embodiment 63, wherein the one or more molecules is selected from among IL-9, VEGF, IL-17E/IL-25, IL-15, IL-22, MIP-3a and IL-12/IL-23p40.

65. The method of embodiment 63 or embodiment 64, wherein the decreased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

66. The method of embodiment 51, wherein the one or more signs, symptoms or outcomes is or is associated with altered expression or ratio thereof of one or more gene products in a tissue, wherein the tissue is brain.

67. The method of embodiment 51 or embodiment 66, wherein the one or more gene products is or comprises a polynucleotide or portion thereof, optionally wherein the portion is a partial transcript of the polynucleotide.

68. The method of embodiment 67, wherein the polynucleotide is an RNA, optionally wherein the RNA is a messenger RNA (mRNA).

69. The method of embodiment 51, embodiment 67 or embodiment 68, wherein expression of the one or more gene products or portions thereof is measured by polymerase chain reaction (PCR), northern blotting, Southern blotting, microarray, and/or a sequencing technique.

70. The method of any of embodiments 51 and 67-69, wherein expression of one or more gene products or portions thereof is assessed by reverse transcriptase PCR (rtPCR) and/or real-time or quantitative PCR (qPCR).

71. The method of any of embodiments 51 and 67-70, wherein the expression of the one or more gene products or portions thereof is assessed by microarray.

72. The method of any of embodiments 51 and 67-71, wherein the expression of the one or more gene products or portions thereof is assessed by a sequencing technique, optionally a non-Sanger sequencing technique and/or a next generation sequencing technique.

73. The method of any of embodiments 51 and 67-72, wherein the expression of the one or more gene products or portions thereof is assessed by massively parallel signature sequencing (MPSS), ion semiconductor sequencing, pyrosequencing, SOLiD sequencing, single molecule real time (SMRT) sequencing, and/or nanopore DNA sequencing.

74. The method of any of embodiments 51 and 67-73, wherein the expression of the one or more gene products or portions thereof is assessed by RNA sequencing (RNA-seq).

75. The method of any of embodiments 51 and 67-74, wherein the expression of the one or more gene products is increased, optionally is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

76. The method of any of embodiments 51 and 67-75, wherein the one or more gene products is associated with or involved in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

77. The method of any of embodiments 51 and 67-76, wherein the one or more gene products is associated with or involved in immune response, angiogenesis, sterol metabolic processes, oxidative stress, antioxidant defense, nitric oxide signaling pathway, cell adhesion or a combination of any of the foregoing.

78. The method of any of embodiments 51 and 67-77, wherein the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31.

79. The method of any of embodiments 51 and 67-74, wherein the expression of the one or more gene products is decreased, optionally is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

80. The method of embodiment 51, wherein the one or more signs, symptoms or outcomes associated is or is associated with altered blood chemistry and the altered blood chemistry comprises a decrease in serum glucose, serum albumin, total serum protein and/or serum levels of calcium.

81. The method of embodiment 51, wherein the one or more signs, symptoms or outcomes is or is associated with tissue damage, optionally wherein the tissue damage comprises histiocytic granulomatous infiltration of the tissue, necrosis, vascular damage and/or vascular leakage.

82. The method of embodiment 51, wherein the one or more signs, symptoms or outcomes is or is associated with altered behavior, optionally wherein the altered behavior comprises reduced food intake, reduced water intake, reduced grooming, and/or reduced locomotor activity.

83. A mouse model, comprising a mouse produced by the method of any of embodiments 1-82.

84. A mouse model, comprising an immunocompetent mouse comprising:

a partial depletion in number of one or more populations of lymphocytes compared to the number of the one or more populations of lymphocytes, on average, in a naïve mouse of the same strain; and

an immunotherapy, wherein the immunotherapy binds to and/or recognizes an antigen that is expressed on or in a cell or tissue of the immunocompetent mouse, optionally wherein the immunotherapy is exogenous to the immunocompetent mouse, optionally wherein the immunotherapy is recombinant or chimeric.

85. The mouse model of embodiment 84, wherein the partial depletion is not permanent or is transient, optionally wherein the partial depletion is present for greater than 14 days, 28 days, 45 days, 60 days, 3 months, 6 months, 1 year or more following administration of a lymphodepleting therapy or agent, optionally wherein the lymphodepleting agent or therapy comprises cyclophosphamide.

86. The mouse model of embodiment 84 or embodiment 85, wherein the mouse comprises:

i) a depletion of a percentage of total circulating lymphocytes of between 10% and 95%, between 30% and 85%, or between about 50% and 75%; and/or

ii) a depletion of a percentage of circulating T cells of between 10% and 95%, between 30% and 85%, or between about 50% and 75%; and/or

iii) a depletion of a percentage of circulating B cells of between 50% and 99%, 75% and 99%, or 75% and 95%.

87. The mouse model of any of embodiments 84-86, wherein the number of the one or more populations of lymphocytes comprises:

between or between about 0.1 and 1,000 lymphocytes per μl of blood;

between 0.1 and 1,000 B cells per μl of blood; and/or

between 0.1 and 100 T cells per μl of blood.

88. The mouse model of any of embodiments 84-87, wherein the antigen is an antigen naturally expressed on murine cell and/or the antigen is a cell surface antigen and/or the immunotherapy binds to or recognizes an extracellular epitope of the antigen.

89. The mouse model of any of embodiments 84-88, wherein the cell is a murine cell.

90. The mouse model of any of embodiments 84-89, wherein the antigen is expressed on the surface of a circulating cell or the cell is a circulating cell.

91. The mouse model of any of embodiments 84-90, wherein the antigen is a B cell antigen or is expressed on the surface of a B cell or wherein the cell is a murine B cell.

92. The mouse model of any of embodiments 84-91, wherein the immunotherapy is an agent that stimulates or activates immune cells.

93. The mouse of embodiment 92, wherein the immunotherapy is a T cell-engaging therapy, optionally wherein the T-cell engaging therapy comprises a bispecific antibody, wherein at least one binding portion specifically binds to a T cell antigen, optionally CD3.

94. The mouse model of embodiment 92 or embodiment 93, wherein the amino acid sequence of the T cell-engaging therapy comprises a murine sequence and/or is not immunogenic to the mouse.

95. The mouse model of any of embodiments 84-91, wherein the immunotherapy comprises a cell therapy, said cell therapy optionally comprising a dose or composition of genetically engineered cells expressing a recombinant receptor.

96. The mouse model of embodiment 95, wherein the engineered cells comprise cells obtained from a biological sample from the immunocompetent mouse or from a mouse that is of the same strain or substrain as the immunocompetent mouse.

97. The mouse model of embodiment 96, wherein the biological sample comprises splenocytes.

98. The mouse model of any of embodiments 95-97, wherein the engineered cells comprise NK cells or T cells, optionally wherein the T cells are CD4+ and/or CD8+ T cells.

99. The mouse model of any of embodiments 95-98, wherein the recombinant receptor is a T cell receptor or a functional non-T cell receptor.

100. The mouse model of any of embodiments 95-99, wherein the recombinant receptor is a chimeric receptor, optionally a chimeric antigen receptor (CAR).

101. The mouse model of any of embodiments 95-100, wherein:

the amino acid sequence of the recombinant receptor is murine; and/or

the individual regions or domains of the chimeric receptor comprise regions or domains of a natural murine protein and/or comprises a murine sequence; and/or

the individual regions or domains of the chimeric receptor are not immunogenic to the mouse.

102. The mouse model of embodiment 100 or embodiment 101, wherein the recombinant receptor is a chimeric antigen receptor (CAR) and the CAR comprises an extracellular antigen-binding domain that specifically binds to the antigen.

103. The mouse model of embodiment 102, wherein the antigen-binding domain is an antibody or an antigen-binding fragment, wherein the antigen-binding fragment is optionally a single chain fragment, optionally an scFv.

104. The mouse model of any of embodiments 100-103, wherein the CAR comprises an intracellular signaling domain comprising an ITAM, wherein optionally, the intracellular signaling domain comprises an intracellular domain of a CD3-zeta (CD3ζ) chain, optionally murine CD3-zeta.

105. The mouse model of embodiment 104, wherein the intracellular signaling domain further comprises a costimulatory signaling region, which optionally comprises a signaling domain of CD28 or 4-1BB, optionally murine CD28 or murine 4-1BB.

106. The mouse model of any of embodiments 84-105, wherein the antigen is or comprises ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen.

107. The mouse model of any of embodiments 84-106, wherein the antigen is B cell maturation antigen (BCMA), CD19, CD20, CD22, CD24, CD30, and/or CD38.

108. The mouse model of any of embodiments 84-107, wherein the antigen is CD19.

109. The mouse model of any of embodiments 84-108, wherein the mouse comprises one or more exogenous cells expressing the antigen.

110. The mouse model of embodiment 109, wherein the exogenous antigen-expressing cells comprise tumor and/or cancer cells.

111. The mouse model of embodiment 110, wherein the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor.

112. The mouse model of embodiment 110 or embodiment 111, wherein the one or more cancer cells and/or the tumor cells comprise cancerous B cells, optionally mouse B cells and/or are B cell-derived.

113. The mouse model of any of embodiments 110-112, wherein the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

114. The mouse model of any of embodiments 110-113, wherein the one or more cancer cells and/or tumor cells comprise A20 cells.

115. The mouse model of any of embodiments 84-114, wherein the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R.

116. The mouse model of any of embodiments 84-115, wherein the immunocompetent mouse is not a C57BL/6 mouse or a substrain thereof.

117. The mouse model of any of embodiments 84-116, wherein the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

118. The mouse model of any of embodiments 84-117, wherein the immunocompetent mouse, following challenge with an antigen and optionally an adjuvant, has an increase in one or more cytokines compared to an immunocompetent C57BL/6mouse administered the same antigen, optionally wherein the one or more cytokine is an inflammatory cytokine.

119. The mouse model of any of embodiments 84-118, wherein the immunocompetent mouse is a BALB/c mouse or is of a substrain thereof.

120. The mouse model of embodiment 119, wherein the BALB/c mouse or substrain thereof is a BALB/cJ mouse or a BALB/cByJ mouse.

121. The mouse model of any of embodiments 84-120, wherein the immunocompetent mouse exhibits one or more signs, symptoms or outcomes associated with a toxicity and/or selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior.

122. The mouse model of embodiment 121, wherein the toxicity and/or the one or more signs, symptoms or outcomes is or is associated with inflammation, wherein the inflammation comprises histiocytic granulomatous infiltration, optionally of the liver, lung, spleen, or brain.

123. The mouse model of embodiment 121, wherein the toxicity and/or the one or more signs, symptoms or outcomes is or is associated with altered level, amount or expression or ratio thereof of one or more molecules in the serum, wherein the one or more molecules is a cytokine, chemokine or growth factor.

124. The mouse model of embodiment 121 or embodiment 123, wherein the altered level, amount or expression or ratio thereof of the molecule comprises an increased level, amount or expression compared to the level, amount or expression of the molecule, on average, in a naïve mouse of the same strain and/or compared to the level, amount or expression of the molecule in a mouse administered a non-target immunotherapy.

125. The mouse model of embodiment 124, wherein the level, amount or expression is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

126. The mouse model of any of embodiments 123-125, wherein the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2.

127. The mouse model of any of embodiments 124-126, wherein the increased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

128. The mouse model of embodiment 121, wherein the altered level, amount or expression or ratio thereof is or comprises an altered ratio of angiopoetin-2 to angiopoetin-1 (Ang2:Ang1 ratio) in the serum, optionally wherein the altered ratio is an increased ratio.

129. The mouse model of embodiment 128, wherein the Ang2:Ang1 ratio is increased by at least 2-fold, at least 3-fold, at least 4-fold, at least 5-fold, at least 10-fold, at least 15-fold, at least 20-fold, at least 25-fold, at least 30-fold, at least 40-fold, at least 50-fold, at least 100-fold, at least 500-fold, at least 1,000-fold, or at least 5,000-fold compared to the Ang2:Ang1 ratio in the mouse prior to administering the lymphodepleting therapy and/or immunotherapy and/or compared to the Ang2:Ang1 ratio, on average, in a naïve mouse of the same strain and/or compared to the Ang2:Ang1 ratio in a mouse administered a non-target immunotherapy.

130. The mouse model of embodiment 121, wherein the altered level, amount or expression or ratio thereof is or comprises a ratio of angiopoetin-2 to angiopoetin-1 (Ang2:Ang1 ratio) in the serum of at least 1, at least 2, at least 3, at least 4, at least 5, at least 10, at least 15, at least 20, at least 25, at least 30, at least 40, at least 50, at least 100, at least 500, at least 1,000, or at least 5,000 or higher.

131. The mouse model of embodiment 130, wherein the Ang2:Ang1 ratio is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

132. The mouse model of embodiment 121 or embodiment 123, wherein the altered level, amount or expression or ratio thereof of the molecule comprises a decreased level, amount or expression compared to the level, amount or expression of the molecule, on average, in a naïve mouse of the same strain and/or compared to the level, amount or expression of the molecule in a mouse administered a non-target immunotherapy.

133. The mouse model of embodiment 132, wherein the level, amount or expression is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

134. The mouse model of embodiment 121, embodiment 132 or embodiment 133, wherein the one or more molecules is selected from among IL-9, VEGF, IL-17E/IL-25, IL-15, IL-22, MIP-3a and IL-12/IL-23p40.

135. The mouse model of embodiment 133 or embodiment 134, wherein the decreased level, amount or expression is observed about or at least about 1 hour, 2 hours, 3 hours, 4 hours, 5 hours, 6 hours, 9 hours, 12 hours, 24 hours, 36 hours, 48 hours, 60 hours, 72 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks after administering the immunotherapy.

136. The mouse model of embodiment 121, wherein the toxicity and/or the one or more signs, symptoms or outcomes is or is associated with altered expression or ratio thereof of one or more gene products in a tissue, wherein the tissue is brain.

137. The mouse model of embodiment 121 or embodiment 136, wherein the one or more gene products is or comprises a polynucleotide or portion thereof, optionally wherein the portion is a partial transcript of the polynucleotide.

138. The mouse model of embodiment 137, wherein the polynucleotide is an RNA, optionally wherein the RNA is a messenger RNA (mRNA).

139. The mouse model of embodiment 121, embodiment 137 or embodiment 138, wherein expression of the one or more gene products or portions thereof is determined by polymerase chain reaction (PCR), northern blotting, Southern blotting, microarray, and/or a sequencing technique.

140. The mouse model of any of embodiments 121 and 137-139, wherein expression of one or more gene products or portions thereof is determined by reverse transcriptase PCR (rtPCR) and/or real-time or quantitative PCR (qPCR).

141. The mouse model of any of embodiments 121 and 137-140, wherein the expression of the one or more gene products or portions thereof is determined by microarray.

142. The mouse model of any of embodiments 121 and 137-141, wherein the expression of the one or more gene products or portions thereof is determined by a sequencing technique, optionally a non-Sanger sequencing technique and/or a next generation sequencing technique.

143. The mouse model of any of embodiments 121 and 137-142, wherein the expression of the one or more gene products or portions thereof is assessed by massively parallel signature sequencing (MPSS), ion semiconductor sequencing, pyrosequencing, SOLiD sequencing, single molecule real time (SMRT) sequencing, and/or nanopore DNA sequencing.

144. The mouse model of any of embodiments 121 and 137-143, wherein the expression of the one or more gene products or portions thereof is assessed by RNA sequencing (RNA-seq).

145. The mouse model of any of embodiments 121 and 137-144, wherein the expression of the one or more gene products is increased, optionally is increased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

146. The mouse model of any of embodiments 121 and 137-145, wherein the one or more gene products is associated with or involved in response in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

147. The mouse model of any of embodiments 121 and 137-146, wherein the one or more gene products is associated with or involved in immune response, angiogenesis, sterol metabolic processes, oxidative stress, antioxidant defense, nitric oxide signaling pathway, cell adhesion or a combination of any of the foregoing.

148. The mouse model of any of embodiments 121 and 137-147, wherein the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31.

149. The mouse model of any of embodiments 121 and 137-148, wherein the expression of the one or more gene products is decreased, optionally is decreased at least 1.2-fold, 1.5-fold, 2.0-fold, 3.0-fold, 4.0-fold, 5.0-fold, 10.0-fold, 20.0-fold, 30.0-fold, 40.0-fold, 50.0-fold, 60.0-fold, 70.0-fold, 80.0-fold, 90.0-fold, 100-fold, 125-fold, 150-fold, 200-fold or more.

150. The mouse model of any of embodiments 121-149, wherein the toxicity and/or the one or more signs, symptoms or outcomes is or is associated with altered blood chemistry and the altered blood chemistry comprises a decrease in serum glucose, serum albumin, total serum protein and/or serum levels of calcium.

151. The mouse model of any of embodiments 121-150, wherein the toxicity and/or the one or more signs, symptoms or outcomes is or is associated with tissue damage, optionally wherein the tissue damage comprises histiocytic granulomatous infiltration of the tissue, necrosis, vascular damage and/or vascular leakage.

152. The mouse model of any of embodiments 121-151, wherein the toxicity and/or the one or more signs, symptoms or outcomes is or is associated with altered behavior, optionally wherein the altered behavior comprises reduced food intake, reduced water intake, reduced grooming, and/or reduced locomotor activity.

153. A tissue sample obtained from a mouse produced by the methods of any of embodiments 1-82, or from the mouse model of any of embodiments 83-152.

154. The tissue of embodiment 153, wherein the tissue sample is or comprises blood, serum, brain tissue, liver tissue, lung tissue, kidney tissue, and/or spleen tissue.

155. The tissue of embodiment 153 or embodiment 154, wherein the tissue sample is or comprise brain tissue.

156. A method of identifying and/or assessing one or more effects of an agent, the method comprising:

i) administering a lymphodepleting agent or therapy and an immunotherapy to an immunocompetent mouse to generate a toxicity and/or one or more sign, symptom or outcome associated with or indicative of a toxic outcome or side effect;

ii) administering a test agent, optionally at a test dosage regimen or frequency of the test agent, to the immunocompetent mouse; and

iii) assessing the toxicity and/or one or more of the sign, symptom, or outcome in the mouse.

157. The method of embodiment 156, wherein the test agent is administered prior to, subsequent to, or concurrently and/or or simultaneously with initiation of administration of the lymphodepleting agent or therapy and/or initiation of administration of the immunotherapy.

158. The method of embodiment 157, wherein the test agent is administered prior to initiation of administration of the lymphodepleting agent or therapy and/or initiation of administration of the immunotherapy.

159. The method of any of embodiments 156-158, wherein the method further comprising:

iv) comparing the toxicity and/or the one or more of the sign, symptom, or outcome to a control mouse, the control mouse having been administered the lymphodepleting agent or therapy and the immunotherapy but not the test agent, wherein the control mouse is immunocompetent.

160. A method of identifying and/or assessing one or more effects of an agent, the method comprising:

i) administering a test agent, optionally at a test dosage regimen or frequency of the test agent, to an immunocompetent mouse, the immunocompetent mouse having been previously administered a lymphodepleting agent or therapy and an immunotherapy, wherein the immunocompetent mouse exhibits a toxicity and/or one or more sign, symptom or outcome associated with or indicative of a toxic outcome or side effect; and

ii) assessing the toxicity and/or the one or more sign, symptom, or outcome in the mouse.

161. The method of embodiment 160, wherein the immunocompetent mouse is a mouse produced by the methods of any of embodiments 1-82, or the mouse model of any of embodiments 83-152.

162. The method of any embodiment 160 or embodiment 161, wherein the method further comprises:

iii) comparing the toxicity and/or the one or more sign, symptom, or outcome to a control mouse, the control mouse having been administered the lymphodepleting agent or therapy and the immunotherapy but not the test agent, wherein the control mouse is immunocompetent.

163. The method of any of embodiments 156-162, wherein the test agent is administered subsequent to the administration of the lymphodepleting agent or therapy and/or the immunotherapy.

164. The method of any of embodiments 156-163, wherein the test dosage regimen of the test agent is for assessing if a particular or predetermined amount or concentration of the test agent for administration and/or the dosing frequency of the agent for administration alters the toxicity and/or one or more of the sign, symptom, or outcome in the mouse.

165. The method of any of embodiments 156-164, wherein the test agent comprises a small molecule, a small organic compound, a peptide, a polypeptide, an antibody or antigen binding fragment thereof, a non-peptide compounds, a synthetic compound, a fermentation product, a cell extract, a polynucleotide, an oligonucleotide, an RNAi, an siRNA, an shRNA, a multivalent siRNA, an miRNA, and/or a virus.

166. The method of any of embodiments 156-165, wherein the test agent, optionally the test dosage regimen of the test agent, is a candidate for ameliorating the toxicity and/or the sign, symptom, or outcome.

167. The method of any of embodiments 156-166, wherein, if the comparison indicates the toxicity and/or the sign, symptom, or outcome is altered, optionally reduced, in the presence of the test agent, optionally the test dosage regimen of the test agent, the test agent is identified as an agent for ameliorating toxicity to the immunotherapy or likely to or predicted to ameliorate toxicity to the immunotherapy.

168. The method of any of embodiments 156-165, wherein the test agent, optionally the test dosage regimen of the test agent, is an agent for use in combination with the cell therapy, optionally wherein the agent is or is likely or is a candidate to improve the activity, efficacy, survival and/or persistence of the cell therapy.

169. The method of any of embodiments 156-165 and 168, wherein, if the comparison indicates the toxicity and/or the sign, symptom, or outcome is altered, optionally increased, in the presence of the test agent, optionally the test dosage regimen of the test agent, the test agent or test dosage regimen is identified as exacerbating the toxicity to the immunotherapy or is likely to or predicted to exacerbate toxicity to the immunotherapy.

170. The method of any of embodiments 156-169, wherein the toxicity comprises and/or the one or more signs, symptoms or outcomes associated with the toxicity is selected from increased inflammation, optionally systemic inflammation or neuroinflammation; altered level, amount or expression or ratio thereof of one or more molecules, optionally a cytokine, chemokine or growth factor, optionally an inflammatory molecule, optionally wherein the molecule is a serum protein; altered expression or ratio thereof of one or more gene product, optionally in a tissue, optionally wherein the tissue is brain; altered blood chemistry; tissue damage, optionally damage of the brain; brain edema; weight loss; reduced body temperature; and/or altered behavior.

171. The method of any of embodiments 156-170, wherein the toxicity and/or the one or more signs, symptoms or outcomes is or is associated with inflammation, wherein the inflammation comprises histiocytic granulomatous infiltration, optionally of the liver, lung, spleen, or brain.

172. The method of any of embodiments 156-170, wherein the toxicity and/or the one or more signs, symptoms or outcomes is or is associated with altered level, amount or expression or ratio thereof of one or more molecules in the serum, wherein the one or more molecules is a cytokine, chemokine or growth factor.

173. The method of embodiment 172, wherein the one or more molecules is selected from among IL-2, IL-4, IL-5, IL-6, IL-10, IL-13, IL-21, IL-23, IP-10, KC/GRO, IL-16, IL-17A, EPO, IL-30, TNFα, IFNγ, MCP-1, MIP-1a, MIP-1b, GM-CSF, and Angiopoetin-2.

174. The method of embodiment 172, wherein the one or more molecules is selected from among IL-9, VEGF, IL-17E/IL-25, IL-15, IL-22, MIP-3a and IL-12/IL-23p40.

175. The method of any of embodiments 156-170, wherein the toxicity and/or the one or more signs, symptoms or outcomes is or is associated with altered expression or ratio thereof of one or more gene products in a tissue, wherein the tissue is brain.

176. The method of embodiment 170 or embodiment 175, wherein the one or more gene products is or comprises a polynucleotide or portion thereof, optionally wherein the portion is a partial transcript of the polynucleotide.

177. The method of embodiment 176, wherein the polynucleotide is an RNA, optionally wherein the RNA is a messenger RNA (mRNA).

178. The method of any of embodiments 170 and 175-177, wherein the one or more gene products is associated with or involved in response to a cytokine, response to interferon beta, cellular response to interferon beta, antigen processing and presentation, regulation of cell morphogenesis, cellular response to cytokine stimulation, innate immune response, response to interferon gamma, cell junction assembly, angiogenesis, regulation of cell projection organization, regulation of neuron projection development, blood vessel morphogenesis, regulation of protein modification, regulation of neurotransmitter receptor activity, regulation of cell shape, regulation of cellular component size, response to fluid shear stress, cell junction organization, actin filament organization, endocytosis, cellular response to interferon gamma, regulation of glutamate receptor signaling pathway, regulation of phosphorylation, response to peptide hormone, regulation of cellular component biogenesis, positive regulation of cell migration, viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound, or a combination of any of the foregoing.

179. The method of any of embodiments 170 and 175-178, wherein the one or more gene products is associated with or involved in immune response, angiogenesis, sterol metabolic processes, oxidative stress, antioxidant defense, nitric oxide signaling pathway, cell adhesion or a combination of any of the foregoing.

180. The method of any of embodiments 170 and 175-179, wherein the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin or CD31.

181. The method of any of embodiments 156-172, wherein the toxicity and/or the one or more signs, symptoms or outcomes is or is associated with altered blood chemistry and the altered blood chemistry comprises a decrease in serum glucose, serum albumin, total serum protein and/or serum levels of calcium.

182. The method of any of embodiments 156-170, wherein the toxicity and/or the one or more signs, symptoms or outcomes is or is associated with tissue damage, optionally wherein the tissue damage comprises histiocytic granulomatous infiltration of the tissue, necrosis, vascular damage and/or vascular leakage.

183. The method of any of embodiments 156-170, wherein the toxicity and/or the one or more signs, symptoms or outcomes is or is associated with altered behavior, optionally wherein the altered behavior comprises reduced food intake, reduced water intake, reduced grooming, and/or reduced locomotor activity.

184. The method of any of embodiments 156-183, wherein assessing the toxicity and/or the one or more sign, symptom, or outcome in the mouse is determined by polymerase chain reaction (PCR), northern blotting, Southern blotting, microarray, a sequencing technique, an immunoassay, flow cytometry, histochemistry, monitoring weight, monitoring temperature, and/or observing physical, phenotypic and/or behavioral changes or features.

185. The method of any of embodiments 156-184, wherein the expression of the one or more gene products or portions thereof is assessed by RNA sequencing (RNA-seq).

186. The method of any of embodiments 156-185, wherein the lymphodepleting agent or therapy does not comprise total body radiation and/or does not comprise complete or substantially complete immune ablation.

187. The method of any of embodiments 156-186, wherein the immunotherapy binds to and/or recognizes an antigen that is expressed on or in a cell or tissue of the immunocompetent mouse.

188. The method of embodiment 187, wherein the antigen is an antigen naturally expressed on murine cell and/or the antigen is a cell surface antigen and/or the immunotherapy binds to or recognizes an extracellular epitope of the antigen.

189. The method of embodiment 187 or embodiment 188, wherein the cell is a murine cell.

190. The method of any of embodiments 187-189, wherein the antigen is expressed on the surface of a circulating cell or the cell is a circulating cell.

191. The method of any of embodiments 187-190, wherein the antigen is a B cell antigen or is expressed on the surface of a B cell or wherein the cell is a murine B cell.

192. The method of any of embodiments 156-191, wherein the immunotherapy is an agent that stimulates or activates immune cells.

193. The method of embodiment 192, wherein the immunotherapy is a T cell-engaging therapy, optionally wherein the T-cell engaging therapy comprises a bispecific antibody, wherein at least one binding portion specifically binds to a T cell antigen, optionally CD3.

194. The method of embodiment 192 or embodiment 193, wherein the amino acid sequence of the T cell-engaging therapy comprises a murine sequence and/or is not immunogenic to the mouse.

195. The method of any of embodiments 156-191, wherein the immunotherapy comprises a cell therapy, said cell therapy optionally comprising a dose or composition of genetically engineered cells expressing a recombinant receptor.

196. The method of embodiment 195, wherein the engineered cells comprise cells obtained from a biological sample from the immunocompetent mouse or from a mouse that is of the same strain or substrain as the immunocompetent mouse.

197. The method of embodiment 196, wherein the biological sample comprises splenocytes.

198. The method of any of embodiments 195-197, wherein the engineered cells comprise NK cells or T cells, optionally wherein the T cells are CD4+ and/or CD8+ T cells.

199. The method of any of embodiments 195-198, wherein the recombinant receptor is a T cell receptor or a functional non-T cell receptor.

200. The method of any of embodiments 195-199, wherein the recombinant receptor is a chimeric receptor, optionally a chimeric antigen receptor (CAR).

201. The method of any of embodiments 195-200, wherein:

the amino acid sequence of the recombinant receptor is murine; and/or

the individual regions or domains of the chimeric receptor comprise regions or domains of a natural murine protein and/or comprises a murine sequence; and/or

the individual regions or domains of the chimeric receptor are not immunogenic to the mouse.

202. The method of embodiment 200 or embodiment 201, wherein the recombinant receptor is a chimeric antigen receptor (CAR) and the CAR comprises an extracellular antigen-binding domain that specifically binds to the antigen.

203. The method of embodiment 202, wherein the antigen-binding domain is an antibody or an antigen-binding fragment, wherein the antigen-binding fragment is optionally a single chain fragment, optionally an scFv.

204. The method of any of embodiments 200-203, wherein the CAR comprises an intracellular signaling domain comprising an ITAM, wherein optionally, the intracellular signaling domain comprises an intracellular domain of a CD3-zeta (CD3ζ) chain, optionally murine CD3-zeta.

205. The method of embodiment 204, wherein the intracellular signaling domain further comprises a costimulatory signaling region, which optionally comprises a signaling domain of CD28 or 4-1BB, optionally murine CD28 or murine 4-1BB.

206. The method of any of embodiments 187-205, wherein the antigen is or comprises ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen 3(MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or a pathogen-specific antigen.

207. The method of any of embodiments 187-206, wherein the antigen is B cell maturation antigen (BCMA), CD19, CD20, CD22, CD24, CD30, and/or CD38.

208. The method of any of embodiments 187-207, wherein the antigen is CD19.

209. The method of any of embodiments 187-208, wherein:

the antigen is expressed on cells administered to the mouse; and/or

the method comprises administering to the immunocompetent mouse one or more cells expressing the antigen, optionally wherein the antigen-expressing cells are administered prior to administering of the lymphodepleting agent or therapy.

210. The method of any of embodiments 187-209, wherein the antigen is expressed on or in tumor and/or cancer cells and/or the antigen-expressing cells are tumor and/or cancer cells, and wherein:

the immunocompetent mouse comprises the tumor and/or cancer cells; and/or

the method further comprises administering to the immunocompetent mouse one or more cancer cells and/or a tumor or tumor tissue, optionally prior to the administering of the lymphodepleting agent or therapy.

211. The method of embodiment 210, wherein the cancer cells and/or tumor are of the same species as the immunocompetent mouse and/or are mouse cells or a mouse tumor, optionally wherein the antigen is expressed on or in, optionally on the surface of, the one or more cancer cells and/or expressed on or in the tumor.

212. The method of embodiment 210 or embodiment 211, wherein the one or more cancer cells and/or the tumor comprise cancerous B cells, optionally mouse B cells and/or are B cell-derived.

213. The method of any of embodiments 210-212, wherein the mouse contains and/or the one or more cancer cells and/or tumor cells comprise L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

214. The method of any of embodiments 210-213, wherein the mouse contains and/or the one or more cancer cells or tumor cells comprise A20 cells.

215. The method of any of embodiments 156-214, wherein the immunocompetent mouse does not comprise or is not engineered to comprise a mutation that reduces cytokine response and/or does not comprise a mutation in, an NLRP12 gene, which mutation in the NLRP12 gene is optionally at lysine 1034, optionally K1034R.

216. The method of any of embodiments 156-215, wherein the immunocompetent mouse is not a C57BL/6 mouse or a substrain thereof.

217. The method of any of embodiments 156-216, wherein the immunocompetent mouse is not a C57BL/6J mouse, C57BL/6JJcl mouse, C57BL/6JJmsSlc mouse, C57BL/6NJcl mouse, C57BL/6NCrlCrlj mouse, C57BL/6NTac mouse, or a C57BL/6CrSlc mouse and/or is not of a substrain of any of the foregoing.

218. The method of any of embodiments 156-217, wherein the immunocompetent mouse, following challenge with an antigen and optionally an adjuvant, has an increase in one or more cytokines compared to an immunocompetent C57BL/6mouse administered the same antigen, optionally wherein the one or more cytokine is an inflammatory cytokine.

219. The method of any of embodiments 156-218, wherein the immunocompetent mouse is a BALB/c mouse or is of a substrain thereof.

220. The method of embodiment 219, wherein the BALB/c mouse or substrain thereof is a BALB/cJ mouse or a BALB/cByJ mouse.

221. The method of any of embodiments 156-220, wherein the lymphodepleting agent or therapy comprises a chemotherapeutic agent.

222. The method of embodiment 221, wherein the chemotherapeutic agent comprises one or more a toxin, an alkylating agent, a DNA strand-breakage agent, a topoisomerase II inhibitors, a DNA minor groove binding agents, an antimetabolite, a tubulin interactive agent, a progestin, an adrenal corticosteroid, a luteinizing hormone releasing agent antagonist, a gonadotropin-releasing hormone antagonist, or an antihormonal antigen.

223. The method of embodiment 221 or embodiment 222, wherein chemotherapeutic agent comprises one or more of cyclophosphamide, chlorambucil, bendamustine, ifosfamide, prednisone, dexamethasone, cisplatin, carboplatin, oxaliplatin, fludarabine, pentostatin, clardribine, cytarabine, gemcitabine, methotrexate, pralatrexate, vincristine, doxorubicin, mitoxantrone, etoposide, bleomycin or combinations thereof.

224. The method of any of embodiments 221-223, wherein the chemotherapeutic agent is or comprises cyclophosphamide.

225. The method of any of embodiments 156-224, wherein:

the lymphodepleting agent or therapy comprises administering cyclophosphamide at a dose of at least or at least about 50 mg/kg, at least or at least about 100 mg/kg, at least or at least about 200 mg/kg, at least at least about 250 mg/kg, at least or at least about 300 mg/kg, at least or at least about 400 mg/kg, at least or at least about 500 mg/kg or at least or at least about 750 mg/kg or a range between any of the foregoing; or

the lymphodepleting agent or therapy comprises administering cyclophosphamide at a dose between or between about 50 mg/kg and 750 mg/kg, 50 mg/kg and 500 mg/kg, 50 mg/kg and 250 mg/kg, 50 mg/kg and 100 mg/kg, 100 mg/kg and 750 mg/kg, 100 mg/kg and 500 mg/kg, 100 mg/kg and 250 mg/kg, 250 mg/kg and 750 mg/kg, 250 mg/kg and 500 mg/kg or 500 mg/kg and 750 mg/kg, each inclusive.

226. The method of any of embodiments 156-225, wherein the lymphodepleting agent or therapy comprises administering cyclophosphamide at a dose of 250 mg/kg or about 250 mg/kg.

227. The method of embodiment 225 or embodiment 226, wherein the dose of cyclophosphamide is administered one time prior to initiation of administration of the immunotherapy.

228. The method of any of embodiments 225-227, wherein the cyclophosphamide is administered intraperitoneally.

229. The method of any of embodiments 156-228, wherein initiation of administration of the immunotherapy is between 0.5 hours and 120 hours after administering the lymphodepleting agent or therapy.

230. The method of any of embodiments 156-229, wherein initiation of administration of the immunotherapy is between 12 hours and 48 hours after administering the lymphodepleting agent or therapy.

231. The method of any of embodiments 156-230, wherein initiation of administration of the immunotherapy is 24 hours or about 24 hours after administering the lymphodepleting agent or therapy.

232. The method of any of embodiments 156-231, wherein the cell therapy comprises the administration of from or from about 1×10⁶ to 1×10⁸ total recombinant receptor-expressing cells or total T cells.

233. The method of any of any of embodiments 156-232, wherein a the cell therapy comprises the administration of at least or about at least or at or about 5×10⁶ total recombinant receptor-expressing cells or total T cells, 1×10⁷ total recombinant receptor-expressing cells or total T cells, or 5×10⁷ total recombinant receptor-expressing cells or total T cells.

234. The method of any of embodiments 1-82, wherein the cells expressing the antigen are administered prior to initiating administration of the lymphodepleting agent or therapy or the immunotherapy.

235. The method of any of embodiments 1-82 or 234, wherein the cells expressing the antigen are administered prior to initiating administration of the lymphodepleting agent or therapy or the immunotherapy.

236. A method for generating a mouse model of an immunotherapy-associated toxicity or an immunotherapy-associated toxic outcome, comprising:

i) administering to an immunocompetent mouse tumor cells that express an antigen;

ii) after administering the tumor cells, administering a lymphodepleting agent or therapy to the immunocompetent mouse, wherein the lymphodepleting agent or therapy does not comprise total body radiation and/or does not comprise complete or substantially complete immune ablation; and

iii) subsequently administering to the mouse an immunotherapy, wherein the immunotherapy binds to and/or recognizes the antigen that is expressed on the tumor cells.

237. A method for generating a mouse model of an immunotherapy-associated toxicity or an immunotherapy-associated toxic outcome, comprising:

i) administering a lymphodepleting agent or therapy to an immunocompetent mouse comprising tumor cells that express an antigen, optionally wherein the tumor cells had been administered to the mouse prior to initiation of administration of the lymphodepleting agent or therapy, wherein the lymphodepleting agent or therapy does not comprise total body radiation and/or does not comprise complete or substantially complete immune ablation; and

ii) subsequently administering to the mouse an immunotherapy, wherein the immunotherapy binds to and/or recognizes the antigen that is expressed on the tumor cells.

238. The method of any of embodiments 1-82 or 234-237, wherein the tumor cells are administered in an amount sufficient to form a tumor in the mouse.

239. The method of any of embodiments 1-82 or 234-238, wherein the lymphodepleting agent or therapy and/or the immunotherapy is administered to the mouse at a time after tumor burden in the mouse comprises:

a tumor size greater than or greater than about or about 5 mm, greater than or greater than about or about 10 mm, greater than or greater than about or about 15 mm, optionally 5 mm to 15 mm or 10 mm to 15 mm in diameter; and/or

a tumor volume of greater than or greater than about or about 60 mm³, greater than or greater than about or about 70 mm³, greater than or greater than about or about 80 mm³, greater than or greater than about or about 90 mm³, or greater than or greater than about or about 100 mm³.

240. The method of any of embodiments 1-82 or 234-239, wherein the tumor cells are administered between or between about 7 days and 28 days, 14 days and 21 days, or 17 days and 19 days, each inclusive, prior to initiation of administration of the lymphodepleting agent or therapy or the immunotherapy.

241. The method of any of embodiments 1-82 or 234-240, wherein the tumor cell is a B cell cancer cell line.

242. The method of any of embodiments 1-82 or 234-241, wherein the B cell cancer cell line is selected from L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells or a combination thereof.

243. The method of any of embodiments 1-82 or 234-242, wherein the cell therapy comprises murine T cells expressing a recombinant receptor that binds to and/or recognizes a murine antigen that is expressed on a B cell of the immunocompetent mouse

244. The method of any of embodiments 1-82 or 234-243, wherein the tumor cells are administered at or about 17 days, 18 days, or 19 days prior to administration of the immunotherapy.

245. The method of any of embodiments 1-82 or 234-244, wherein the lymphodepleting agent or therapy comprises a dose of at least or at least about 100 mg/kg cyclophosphamide or between or between about 50 mg/kg and 500 mg/kg cyclophosphamide, each inclusive.

246. The method of any of embodiments 1-82 or 234-245, wherein the lymphodepleting agent or therapy comprises a dose of 250 mg/kg or about 250 mg/kg cyclophosphamide.

247. The method of any of embodiments 1-82 or 234-246, wherein the cell therapy comprises the administration of between or between about 5×10⁶ and about 5×10⁷ total recombinant receptor-expressing cells or total T cells.

248. The method of any of embodiments 1-82 or 234-246, wherein the one or more gene products is associated with or involved in wherein the one or more gene products is associated with or involved in viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound.

249. The method of any of embodiments 1-82 or 234-246, wherein the one or more gene product is selected from among Gbp4, Gbp5, Gbp2, Gbp8, Angpt2, Angpt14, Hif3a, Lrg1, Mmrn2, Xdh, Acer2, Atf3, Pdk4, Pla2g3, Sult1a1, CD274 (PD-L1), Tgtp1, Vwf, Ncf1, Aox1, Bnip3, Pxdn, Scara3, Mgst3, Ptgs2, Nos3, VCAM-1, ICAM-1, E-Selectin, P-Selectin, or CD31.

250. The method of any of embodiments 1-82 or 234-249, wherein the one or more gene product is selected from among Adipoq (Adiponectin), Aif1 (Allograft inflammatory factor 1), Aqp4 (Aquaporin-4), Ccl2 (C-C motif chemokine 2), CD68, Edn1 (Endothelin-1), Serpine 1, Tgfb1 (Transforming growth factor beta-1), Tgfb2 (transforming growth factor beta 2), Tgfb3 (transforming growth factor beta 3), Tlr2 (Toll-like receptor 2), Tlr4 (toll like receptor 4), IL2ra, IL-13, Gzmb (Granzyme B), TNF, CXCL10 (IP-10), CCL2 (MCP-1, C-C motif chemokine 2), CXCL11 (I-TAC, C-X-C motif chemokine 11), CXCL1 (KC, Growth-regulated alpha protein), CCL4 (MIP-1b, C-C motif chemokine 4), NLRC5 (class I transactivator), or CIITA (class II transactivator).

251. A mouse model, comprising an immunocompetent mouse comprising:

a partial depletion in number of one or more populations of lymphocytes compared to the number of the one or more populations of lymphocytes, on average, in a naïve mouse of the same strain; an immunotherapy, wherein the immunotherapy binds to and/or recognizes an antigen, wherein the immunotherapy is exogenous to the immunocompetent mouse, optionally wherein the immunotherapy is recombinant or chimeric; and tumor cells comprising the antigen, optionally wherein the antigen is expressed on the tumor cell surface.

252. The mouse model of any of embodiments 83-151 or 252, wherein the immunotherapy comprises a cell therapy, said cell therapy comprising genetically engineered cells expressing a recombinant receptor.

253. The mouse model of any of embodiments 83-151 or 252-253, wherein the engineered cells comprise cells obtained from a biological sample from the immunocompetent mouse or from a mouse that is of the same strain or substrain as the immunocompetent mouse.

254. The mouse model of any of embodiments 83-151 or 252, wherein the biological sample comprises splenocytes.

255. The mouse model of any of embodiments 83-151 or 252-253, wherein the cell therapy comprises murine T cells expressing a recombinant receptor that binds to and/or recognizes a murine antigen that is expressed on a B cell of the immunocompetent mouse.

256. The mouse model of any of embodiments 83-151 or 252-254, wherein the recombinant receptor is a T cell receptor or a functional non-T cell receptor.

257. The mouse model of any of embodiments 83-151 or 252-256, wherein the recombinant receptor is a chimeric receptor, optionally a chimeric antigen receptor (CAR).

258. The mouse model of any of embodiments 83-151 or 252, wherein:

the amino acid sequence of the recombinant receptor is murine; and/or

the individual regions or domains of the chimeric receptor comprise regions or domains of a natural murine protein and/or comprises a murine sequence; and/or

the individual regions or domains of the chimeric receptor are not immunogenic to the mouse.

259. The mouse model of any of embodiments 83-151 or 252-260, wherein the antigen is B cell maturation antigen (BCMA), CD19, CD20, CD22, CD24, CD30, and/or CD38.

260. The mouse model of any of embodiments 83-151 or 252-259, wherein the antigen is CD19.

261. The mouse model of any of embodiments 83-151 or 252-260, wherein the one or more gene products is associated with or involved in viral process, multi-organism cellular process, reactive oxygen species metabolic process, negative regulation of protein modification process, positive regulation of cell adhesion, adhesion of symbiont to host, cell-substrate adhesion, chaperone-mediated protein folding, peptidyl-tyrosine modification, taxis, defense response to other organism, sterol biosynthetic process, cellular response to nitrogen compound.

262. The mouse model of any of embodiments 83-151 or 252-261, wherein the one or more gene products is associated with or involved in immune response, angiogenesis, sterol metabolic processes, oxidative stress, antioxidant defense, nitric oxide signaling pathway, cell adhesion or a combination of any of the foregoing.

263. The mouse model of any of embodiments 83-151 or 252-262, wherein toxicity comprises brain tissue damage.

264. The mouse model of any of embodiments 83-151 or 252-261, wherein the brain tissue damage comprises hemorrhaging.

265. The method of any of embodiments 51, 67-77, or 79-82, wherein the one or more gene products is selected from among Acer2 (Alkaline ceramidase 2), Adipoq (Adiponectin), Aif1 (Allograft inflammatory factor 1), Angpt1 (angeopotein 1), Angpt14 (angiopoietin-like 4), Angpt2 (angiopotein 2), Aox1 (Aldehyde oxidase), Aqp4 (Aquaporin-4), Atf3 (cyclic AMP-dependent transcription factor ATF-3), Bnip3 (BCL2/adenovirus E1B 19 kDa protein-interacting protein 3), Ccl2 (C-C motif chemokine 2), CCL4 (MIP-1b, C-C motif chemokine 4), CD31 (PECAM-1), CD274, CD68, CIITA (class II transactivator), CXCL1 (KC, Growth-regulated alpha protein), CXCL10 (IP-10), CXCL11 (I-TAC, C-X-C motif chemokine 11), Edn1 (Endothelin-1), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), HIF3a (hypoxia inducible factor 3 alpha subunit), ICAM-1 (Intercellular adhesion molecule 1), IL2ra (Interleukin-2 receptor subunit alpha), IL-4, IL-6, IL-13, Lrg1 (leucine rich alpha-2-glycoprotien 1), Mgst3 (Microsomal glutathione S-transferase 3), Mmrn2, (Multimerin-2), Ncf1 (Neutrophil cytosol factor 1), NLRC5 (class I transactivator), Nos3 (Nitric oxide synthase, endothelial), Pdk4 (pyruvate dehydrogenase kinase, isozyme 4), Pla2g3 (group 3 secretory phospholipase A2 precursor), Ptgs2 (Prostaglandin G/H synthase 2), Pxdn (Peroxidasin homolog), Scara3 (Scavenger receptor class A member 3), Sele (E-selectin), Selp (P-selectin), IL2ra, IL-13, Serpine 1, Sult1a1 (Sulfotransferase 1A1), Tgfb1 (Transforming growth factor beta-1). Tgfb2 (transforming growth factor beta 2), Tgfb3 (transforming growth factor beta 3), Tgtp1 (T-cell-specific guanine nucleotide triphosphate-binding protein 1), Tlr2 (Toll-like receptor 2), Tlr4 (toll like receptor 4), TNF (tumor necrosis factor), VCAM-1 (Vascular cell adhesion protein 1), Vwf (von Willebrand factor), or Xdh (xanthine dehydrogenase).

266. The mouse model of any of embodiments 121, 137-147, or 149-152, wherein the one or more gene products is selected from among Acer2 (Alkaline ceramidase 2), Adipoq (Adiponectin), Aif1 (Allograft inflammatory factor 1), Angpt1 (angeopotein 1), Angpt14 (angiopoietin-like 4), Angpt2 (angiopotein 2), Aox1 (Aldehyde oxidase), Aqp4 (Aquaporin-4), Atf3 (cyclic AMP-dependent transcription factor ATF-3), Bnip3 (BCL2/adenovirus E1B 19 kDa protein-interacting protein 3), Ccl2 (C-C motif chemokine 2), CCL4 (MIP-1b, C-C motif chemokine 4), CD31 (PECAM-1), CD274, CD68, CIITA (class II transactivator), CXCL1 (KC, Growth-regulated alpha protein), CXCL10 (IP-10), CXCL11 (I-TAC, C-X-C motif chemokine 11), Edn1 (Endothelin-1), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), HIF3a (hypoxia inducible factor 3 alpha subunit), ICAM-1 (Intercellular adhesion molecule 1), IL2ra (Interleukin-2 receptor subunit alpha), IL-4, IL-6, IL-13, Lrg1 (leucine rich alpha-2-glycoprotien 1), Mgst3 (Microsomal glutathione S-transferase 3), Mmrn2, (Multimerin-2), Ncf1 (Neutrophil cytosol factor 1), NLRC5 (class I transactivator), Nos3 (Nitric oxide synthase, endothelial), Pdk4 (pyruvate dehydrogenase kinase, isozyme 4), Pla2g3 (group 3 secretory phospholipase A2 precursor), Ptgs2 (Prostaglandin G/H synthase 2), Pxdn (Peroxidasin homolog), Scara3 (Scavenger receptor class A member 3), Sele (E-selectin), Selp (P-selectin), IL2ra, IL-13, Serpine 1, Sult1a1 (Sulfotransferase 1A1), Tgfb1 (Transforming growth factor beta-1). Tgfb2 (transforming growth factor beta 2), Tgfb3 (transforming growth factor beta 3), Tgtp1 (T-cell-specific guanine nucleotide triphosphate-binding protein 1), Tlr2 (Toll-like receptor 2), Tlr4 (toll like receptor 4), TNF (tumor necrosis factor), VCAM-1 (Vascular cell adhesion protein 1), Vwf (von Willebrand factor), or Xdh (xanthine dehydrogenase).

267. The method of any of embodiments 170, 175-179, or 181-233, wherein the one or more gene products is selected from among Acer2 (Alkaline ceramidase 2), Adipoq (Adiponectin), Aif1 (Allograft inflammatory factor 1), Angpt1 (angeopotein 1), Angpt14 (angiopoietin-like 4), Angpt2 (angiopotein 2), Aox1 (Aldehyde oxidase), Aqp4 (Aquaporin-4), Atf3 (cyclic AMP-dependent transcription factor ATF-3), Bnip3 (BCL2/adenovirus E1B 19 kDa protein-interacting protein 3), Ccl2 (C-C motif chemokine 2), CCL4 (MIP-1b, C-C motif chemokine 4), CD31 (PECAM-1), CD274, CD68, CIITA (class II transactivator), CXCL1 (KC, Growth-regulated alpha protein), CXCL10 (IP-10), CXCL11 (I-TAC, C-X-C motif chemokine 11), Edn1 (Endothelin-1), Gbp2 (guanylate-binding protein 2), Gbp4(guanylate-binding protein 4), Gdp5 (guanylate-binding protein 5), Gdp9 (guanylate-binding protein 9), GM-CSF, Gzmb (Granzyme B), HIF3a (hypoxia inducible factor 3 alpha subunit), ICAM-1 (Intercellular adhesion molecule 1), IL2ra (Interleukin-2 receptor subunit alpha), IL-4, IL-6, IL-13, Lrg1 (leucine rich alpha-2-glycoprotien 1), Mgst3 (Microsomal glutathione 5-transferase 3), Mmrn2 (Multimerin-2), Ncf1 (Neutrophil cytosol factor 1), NLRC5 (class I transactivator), Nos3 (Nitric oxide synthase, endothelial), Pdk4 (pyruvate dehydrogenase kinase, isozyme 4), Pla2g3 (group 3 secretory phospholipase A2 precursor), Ptgs2 (Prostaglandin G/H synthase 2), Pxdn (Peroxidasin homolog), Scara3 (Scavenger receptor class A member 3), Sele (E-selectin), Selp (P-selectin), IL2ra, IL-13, Serpine 1, Sult1a1 (Sulfotransferase 1A1), Tgfb1 (Transforming growth factor beta-1). Tgfb2 (transforming growth factor beta 2), Tgfb3 (transforming growth factor beta 3), Tgtp1 (T-cell-specific guanine nucleotide triphosphate-binding protein 1), Tlr2 (Toll-like receptor 2), Tlr4 (toll like receptor 4), TNF (tumor necrosis factor), VCAM-1 (Vascular cell adhesion protein 1), Vwf (von Willebrand factor), or Xdh (xanthine dehydrogenase).

### VI. EXAMPLES

The following examples are included for illustrative purposes only and are not intended to limit the scope of the invention.

### Example 1: Generation of Anti-Murine CD19 CAR−T Cells

A nucleic acid molecule was generated encoding an anti-murine CD19 chimeric antigen receptor (CAR), in which the encoded CAR contained an N-terminal CD8 alpha signal peptide (SEQ ID NO: 1); an anti-murine CD19 scFv containing a variable heavy (VH; SEQ ID NO: 2) and a variable light (VL; SEQ ID NO: 3) chain derived from the 1D3 rat monoclonal anti-murine CD19 antibody (ATCC NO. HB-305); a murine IgG3 hinge region (SEQ ID NO: 4); a transmembrane region derived from murine CD28 (SEQ ID NO: 5); an intracellular signaling region containing an intracellular signaling domain of murine 41BB (SEQ ID NO: 6); and an intracellular signaling domain of murine CD3 (SEQ ID NO: 7). The nucleic acid molecule also included a mouse Thy1.1 sequence (SEQ ID NO: 8) for use as a surrogate marker of CAR expression in BALB/c mice, which was separated from the CAR sequence by a self-cleaving T2A sequence. As a non-target control, a similar CAR construct was generated except containing an anti-human CD19 scFv containing a VH (SEQ ID NO: 9) and a VL (SEQ ID NO: 10) chain derived from FMC63.

The nucleic acid molecules were individually cloned into a retroviral vector for introduction into murine T cells. Lymph nodes and spleens were collected from BALB/c donor mice, processed into a single-cell suspension, and total T cells were positively selected using a pan-T cell isolation kit (Miltenyi Biotec). The T cells were cultured with 80 IU/ml murine IL-2, and on days 2 and 3 of cell culture were serially transduced with virus encoding the CAR. As a mock control, cells were incubated with media only. On day 4 of cell culture, fresh media containing 10 IU/ml IL-15 was added and cells were collected on day 5. The generated mouse anti-CD19-CAR-expressing T cells were administered into mice as described in the studies below. In some cases, the generated mouse CAR-expressing cells (muCAR−T) were cryofrozen and stored at −80C prior to use.

### Example 2: Development of a Mouse Model of Toxicity to an Immunotherapy

A single dose of either 100 mg/kg or 250 mg/kg cyclophosphamide (CPA) was injected into naïve BALB/c mice (tumor-free) by intraperitoneal (i.p.) injection. Twenty-four hours later, about 5×10⁶ mouse CAR-expressing T cells or mock (control) cells, generated as described in Example 1, were transferred into mice by intravenous injection into the lateral tail vein. As a control, mice were injected with CPA alone or were left untreated. Table E1 sets forth the treatment groups.

TABLE E1
Treatment Groups

| | | | Number |
|---|---|---|---|
| Group | CPA | muCAR-T or mock | of mice |
| 1 | — | — | 5 |
| 2 | 100 mg/kg | — | 5 |
| 3 | 100 mg/kg | 5 × 10⁶ muCAR-T | 5 |
| 4 | 250 mg/kg | — | 5 |
| 5 | 250 mg/kg | 5 × 10⁶ mock T | 5 |
| 6 | 250 mg/kg | 5 × 10⁶ muCAR-T | 5 |

In all groups, mice were administered fluids subcutaneously on the third and fourth day following cell infusion and were transferred to a soft diet if severe symptoms were observed. Survival was 100% in mice that received 250 mg/kg CPA alone, 250 mg/kg CPA and mock cell therapy, and 100 mg/kg CPA and muCAR−T cells. In the group of mice administered 250 mg/kg CPA and 5×10⁶ muCAR−T, one half ( 4/8) mice died or required euthanasia between 3 to 4 days after the cell infusion.

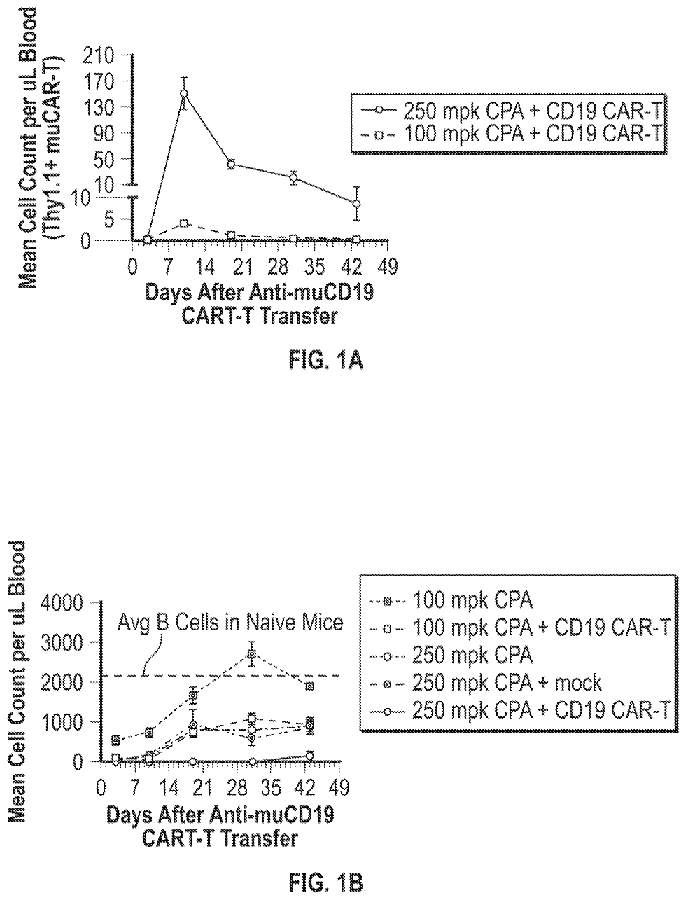

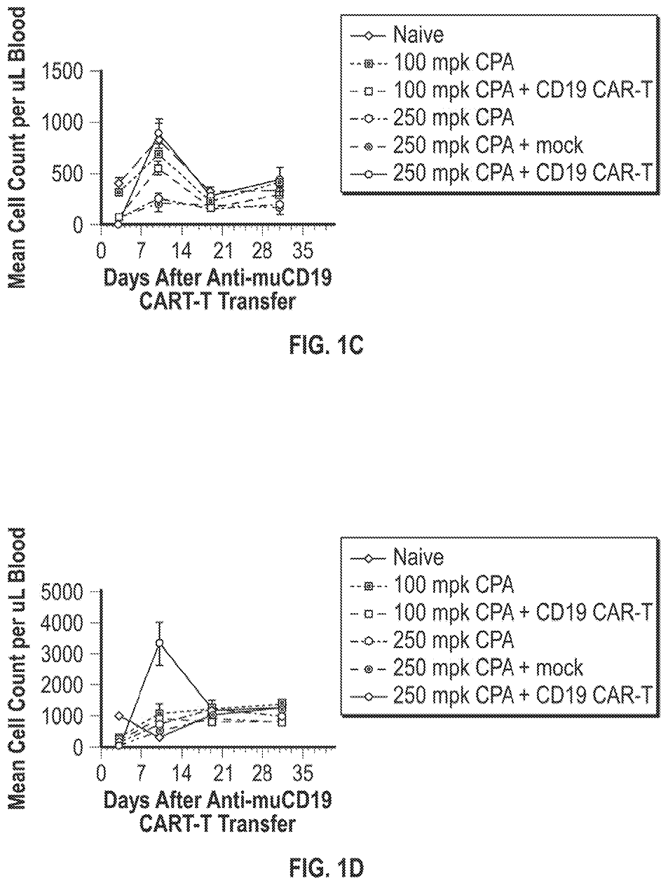

Blood samples were collected from treated mice at days 3, 10, 17, 31 and, in some mice at day 43, following cell infusion. The pharmacokinetics (PK) in the blood of administered cells was assessed by detection of the surrogate marker Thy 1.1. The number of circulating B cells, T cells and CD11b+ cells in the blood at the time points following treatment also was assessed. As shown in FIG. 1A, Thy 1.1+muCAR+ T cells peaked at about day 10 and cell numbers in the blood began to decline by day about day 17. An approximately 40-fold greater expansion of circulating Thy 1.1+CAR+ cells were observed in samples obtained from mice treated with 250 mg/kg CPA as compared to 100 mg/kg CPA. Treatment with CPA depleted circulating B cells (FIG. 1B), T cells (FIG. 1C), and CD11b+ cells (FIG. 1D). As shown in FIG. 1B, treatment with 250 mg/kg CPA reduced levels of circulating B cells to a greater degree than treatment with 100 mg/kg CPA, and for the group of mice administered 250 mg/kg CPA and muCAR−T cells, ongoing B cell aplasia was observed at day 43 days after administration of the cell composition. An early spike in levels of circulating CD11b+ cells was observed in mice treated with 250 mg/kg CPA and muCAR−T cells compared to the other groups (FIG. 1C).

The severe side effects in mice observed after B cell aplasia following treatment with 250 mg/kg CPA and anti-mouse CD19 CART cells demonstrates the use as a mouse model for evaluating toxicity associated with administration of CAR−T cells. Further, the mouse model may also be used to evaluate CAR T cell persistence and activity.

### Example 3: Cytokine Response in a Mouse Model of Toxicity

The levels of circulating cytokines and chemokines, and the changes in such levels over time, was assessed in the mouse model of toxicity described in Example 2.

### A. Circulating Cytokine and Chemokine Levels

The effects of administering CPA and CAR−T cell compositions on levels of circulating cytokines and chemokines were examined. Cryofrozen anti-mouse CD19 CAR−T cell and mock T cell compositions, prepared as described in Example 1, were thawed and administered to mice in the presence of 250 mg/kg CPA substantially as described in Example 2. Specifically, BALB/c mice were administered 250 mg/kg CPA (CPA alone), 250 mg/kg CPA and 5×10⁶ cells mock cells (CPA+mock), or 250 mg/kg CPA and 5×10⁶ cells muCAR−T cells (CPA+CAR−T). Naïve mice that did not receive any CPA or cell treatments were also used as a control.

Serum samples were collected from mice 72 hours after infusion of CAR T cells and levels of cytokines were analyzed by Meso Scale Discovery (MSD). The analysis included measurements of IL-2, IL-4, IL-5, IL-6, IL-10, TNF-alpha, IFN-gamma, MCP-1, MIP-1b, and GM-CSF, which are cytokines that have been observed to be increased in some human subjects experiencing toxicity, including neurotoxicity, following administration of CAR+ T cells. Table E2 depicts the results from two exemplary experiments. For each cytokine, the fold increase in serum samples from mice administered muCAR−T cells versus mock T cells is shown. IL-12p70, IL-1b, IL-17A/F, IL-17C, IL-17F, IL-31 and IL-33 also were assessed but were not detected.

TABLE E2
Fold Increase of serum cytokines in CPA + CAR-T
samples as compared to CPA + mock serum

| Cytokine | Experiment 1 | Experiment 2 |
|---|---|---|
| IL-4 | 352.66 | 300.71 |
| GM-CSF | 151.08 | 217.66 |
| IFN-gamma | 149.85 | 110.69 |
| IL-5 | 133.24 | 86.92 |
| IL-13 | 31.17 | 44.98 |
| MCP-1 | 21.08 | 10.60 |
| IL-10 | 18.68 | 7.09 |
| IL-2 | 15.01 | 42.44 |
| MIP-1a | 12.26 | 5.04 |
| IL-6 | 7.63 | 63.68 |
| IL-21 | 6.44 | 6.31 |
| MIP-1B | 6.31 | 2.41 |
| IL-23 | 6.24 | |
| IP-10 | 5.94 | 10.88 |
| TNF-alpha | 4.00 | 11.52 |
| KC/GRO | 2.26 | 6.69 |
| IL-16 | 1.49 | |
| IL-17A | 1.30 | 1.54 |
| EPO | 1.25 | 1.79 |
| IL-30 | 1.20 | |
| IL-9 | 0.87 | 1.36 |
| VEGF | 0.84 | 0.9 |
| IL-17E/IL-25 | 0.81 | 1.07 |
| IL-15 | 0.74 | 0.84 |
| IL-22 | 0.54 | 2.02 |
| MIP-3a | 0.38 | 0.71 |
| IL-12/IL-23p40 | 0.36 | 0.47 |

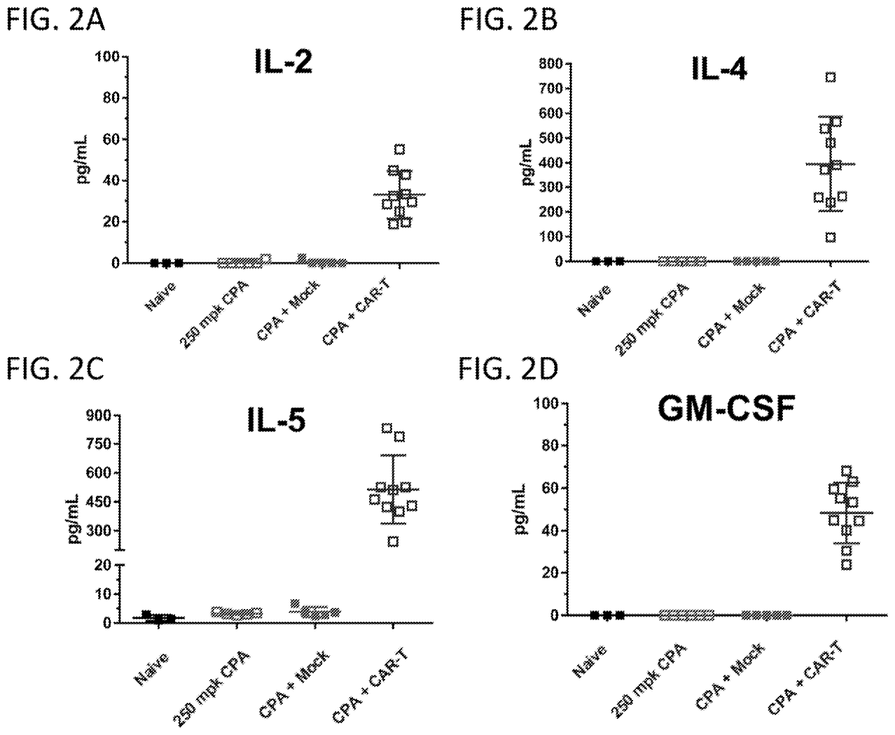

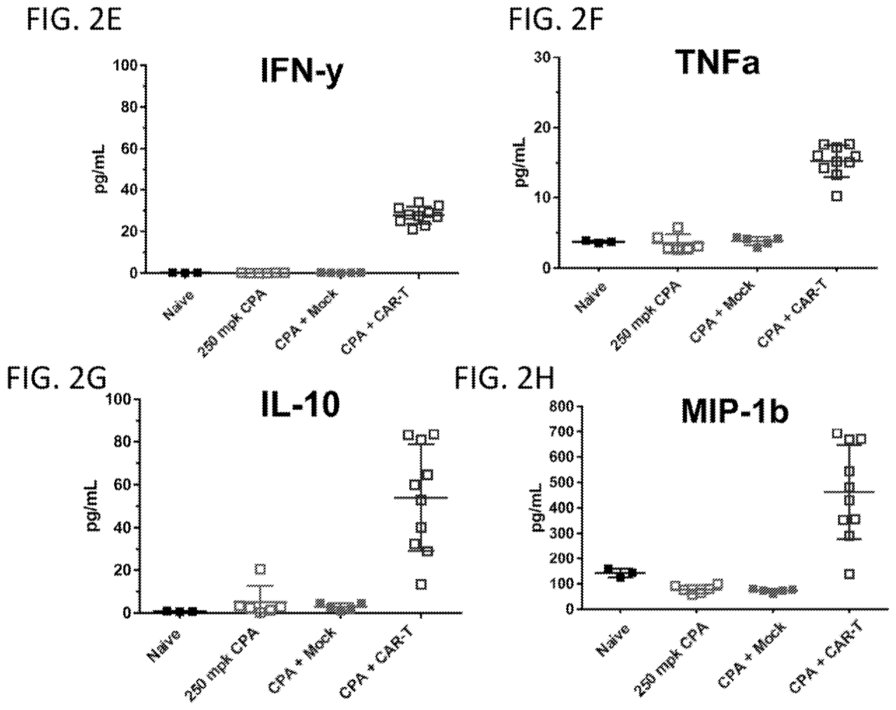

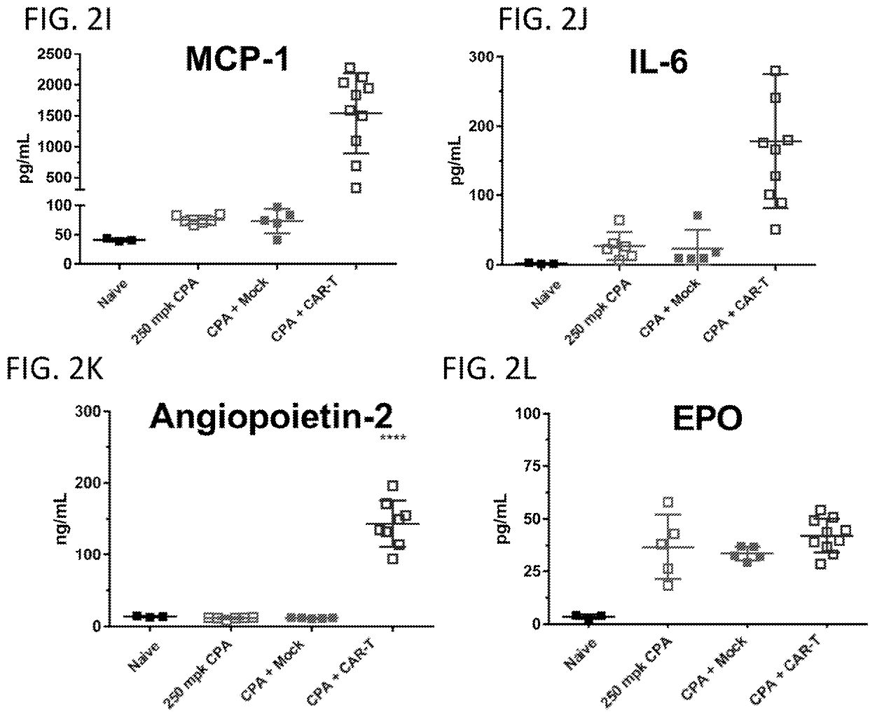

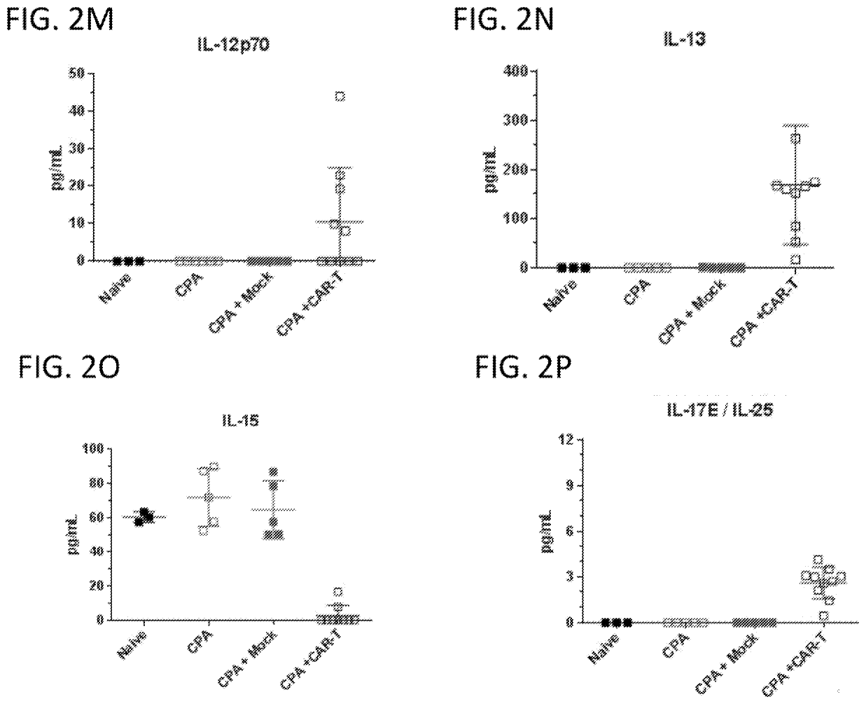

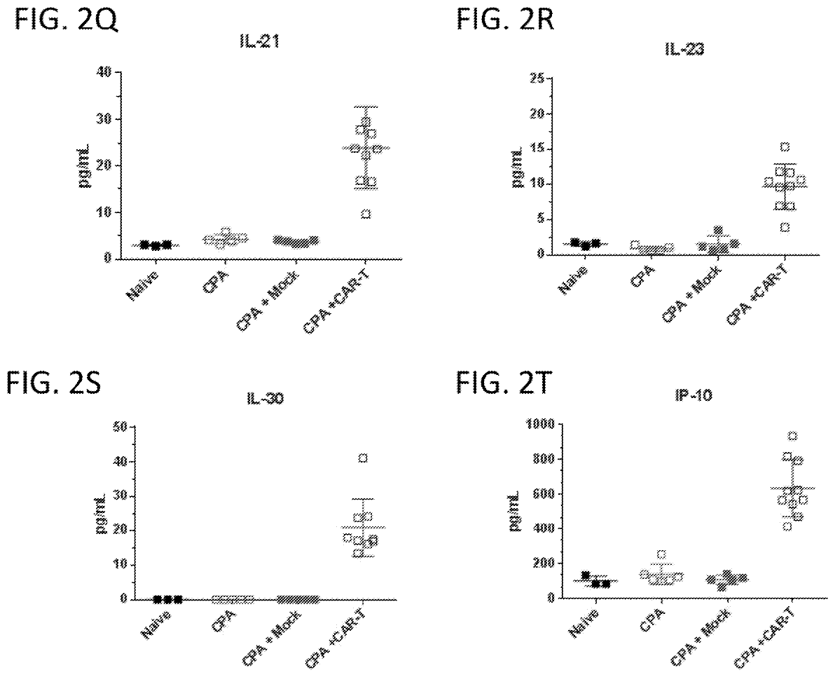

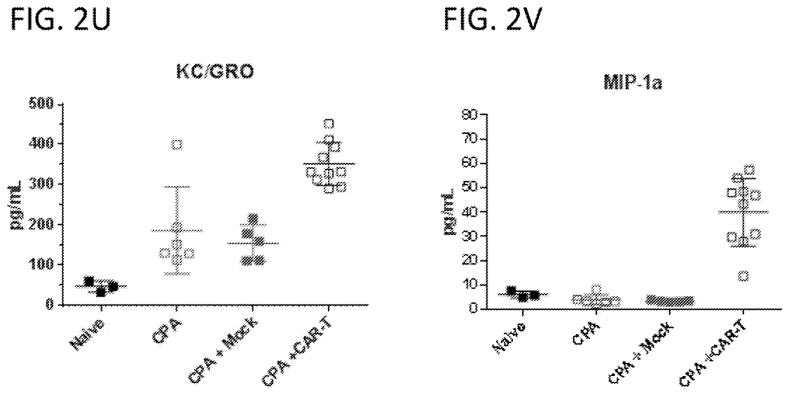

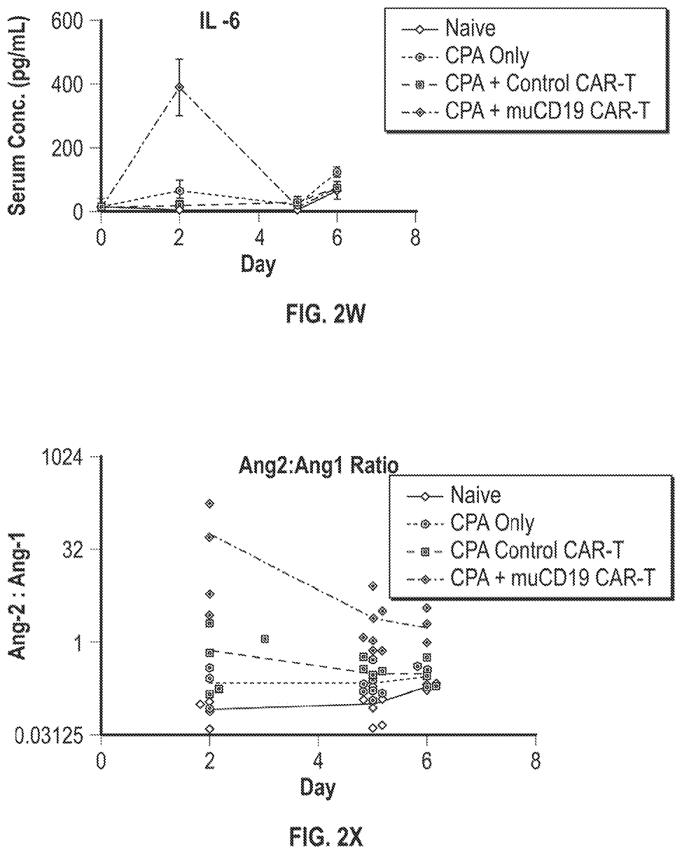

FIG. 2A-V depicts the level of exemplary cytokines detected in the serum of mice 72 hours post-CAR+ T cell administration in mice groups: naive, 250 mg/kg CPA, 250 mg/kg CPA+mock T cells or 250 mg/kg CPA+muCAR+ T cells. Circulating levels of IL-2 (FIG. 2A), IL-4 (FIG. 2B), IL-5 (FIG. 2C), GM-CSF (FIG. 2D), IFN-gamma (FIG. 2E), TNF-alpha (FIG. 2F), IL-10 (FIG. 2G), MIP-1b (FIG. 2H), MCP-1 (FIG. 2I), IL-6 (FIG. 2J), Angiopoietin-2 (FIG. 2K) EPO (FIG. 2L), IL-12p70 (FIG. 2M), IL-13 (FIG. 2N), IL-15 (FIG. 2O), IL-17E/IL25 (FIG. 2P), IL-21 (FIG. 2Q), IL-23 (FIG. 2R), IL-30 (FIG. 2S), IP-10 (FIG. 2T), KC/GRO (FIG. 2U), and MIP-1a (FIG. 2V) are shown.

The elevated levels of serum cytokines seen in samples obtained from CPA+CAR−T mice are consistent with a systemic inflammatory response in mice administered 250 mg/kg CPA+muCAR+ T cells compared to the other treatment groups. The observation that these cytokines are elevated in mice indicate that the mouse model, following administration into BALB/c mice 250 mg/kg CPA+muCAR+ T cells, may exhibit features indicative of toxicity similar to those observed in human subjects.

### B. Changes in Circulating Cytokine Levels Over Time

The changes over time in the level of cytokines after administration of CPA and CAR−T cell were assessed. Mice were administered mouse CAR-expressing cells or control cells substantially as described in Example 2. Specifically, BALB/c mice were injected with 250 mg/kg cyclophosphamide (CPA) i.p and twenty-four hours later were administered anti-mouse CD19 CAR-expressing T cells (muCD19 CAR−T) or non-target control anti-human CD19 CAR+ T cells (control CAR−T) cells, generated as described in Example 1. Naïve mice that did not receive any CPA or cell treatments were also used as a control. Serum samples were obtained days 2, 5 and 6 after infusion of CAR T cells.

FIG. 2W depicts the changes in serum IL-6 levels over time, on days 2, 5 and 6 after infusion of CART cells. As shown, the CPA+anti-muCD19 CAR−T mice exhibited elevated serum IL-6 levels on day 2 after CAR−T administration, with a decrease at day 5, compared to control mice that generally exhibited very low or no increase in serum IL-6 levels. FIG. 2X depicts the changes in the Angiopoietin-2: Angiopoietin-1 ratio (Ang2:Ang1 ratio) over time, on days 2, 5 and 6 after infusion of CAR T cells. The results showed that the CPA+CAR−T mice exhibited elevated Ang2:Ang1 ratio at all time points, with the highest Ang2:Ang1 ratio of approximately 32 on day 2. In comparison, the control mice generally had a low Ang2:Ang1 ratio at all time points. A similar elevation in Ang2: Ang1 ratio is observed in human subjects with severe CRS.

Thus, the results are consistent with the finding that the mouse model receiving CPA+CAR−T may exhibit features indicative of toxicity similar to those observed in human subjects.

### Example 4: Immune Cell and Expression Profile in a Mouse Model of Toxicity to an Immunotherapy

Various parameters relating to toxicity were assessed in BALB/c mice produced to develop B cell aplasia following administration of an immunotherapy involving anti-CD19 CAR T cells substantially as described in Example 2. BALB/c mice either received no treatment (naïve) or were administered 250 mg/kg CPA (CPA alone), CPA and 5×10⁶ cells (CPA+mock), or CPA and 5×10⁶ mouse anti-CD19 CAR+ T cells (CPA+CAR−T).

At 24, 48, and 72 hours after infusion with cell compositions, the three healthiest mice from each group were anesthetized, transcardially perfused with phosphate buffered saline (PBS) to flush brain blood vessels, and sacrificed. Blood and tissue samples (spleen, kidney, liver and brain) were collected for analysis of immune cell populations, gene expression, and blood chemistry. The blood and tissue samples were either processed for flow cytometry or were snap-frozen for RNA sequencing (RNA-Seq) analysis. For flow cytometry, tissue samples were digested and cell suspensions were layered over a density gradient to enrich immune cell populations.

A. Immune Cells

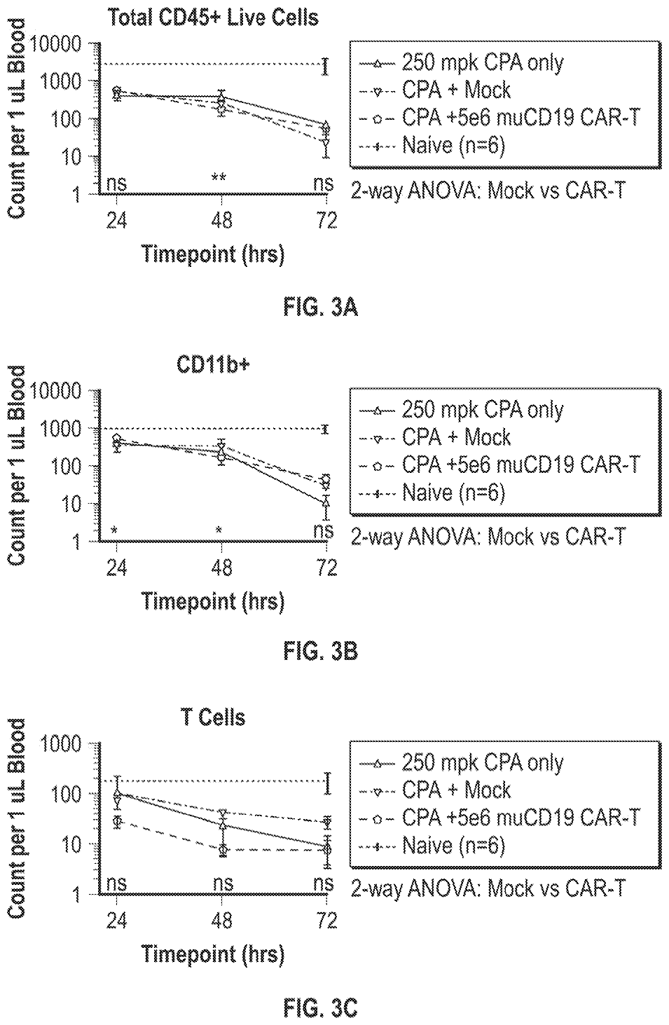

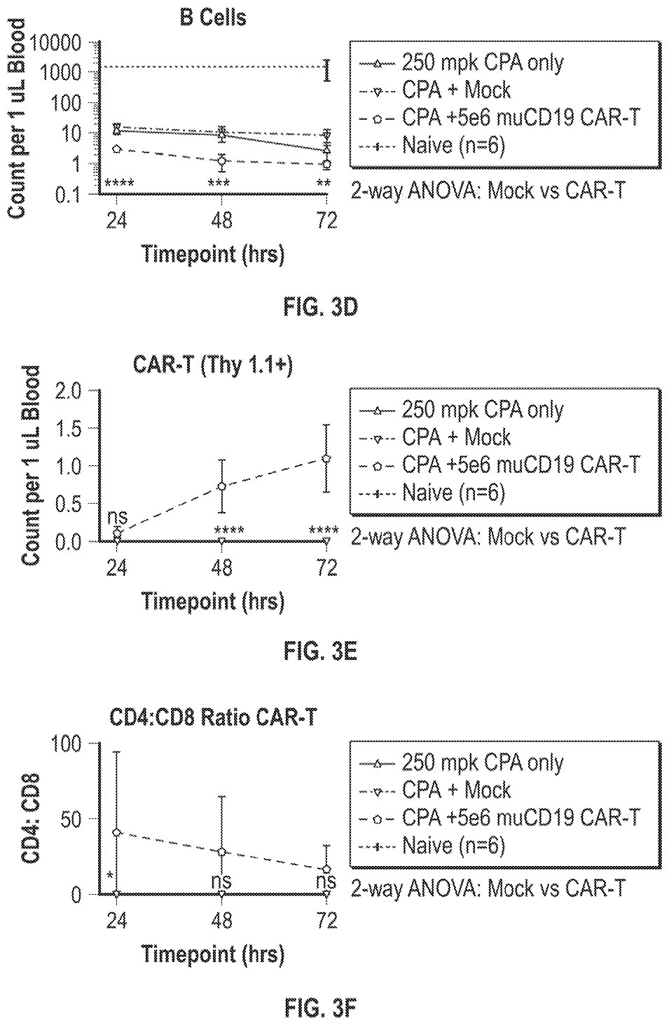

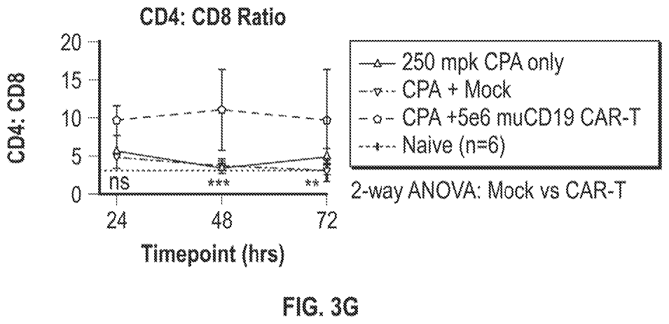

Levels of CAR+ T cells and immune cells in blood, as assessed by flow cytometry, are shown in FIGS. 3A-3D. CPA pre-conditioning resulted in a depletion of circulating CD45+ live cells (FIG. 3A), CD11b+ cells (FIG. 3B), endogenous T cells (FIG. 3C) and endogenous B cells (FIG. 3D) in blood, which continued to drop at least for 72 hours after administration of CAR+ T cells. While CPA alone depleted endogenous circulating B cells, B cell aplasia was significantly increased within 24 hours in mice that were additionally administered the anti-murine CD19 CAR+ T cells (FIG. 3D). Levels of circulating CAR+ T cells were detected at 48 hours after cell infusion and continued to expand up to 72 hours (FIG. 3E). As indicated by the CD4:CD8 ratio of CAR+ T cells after administration of mouse anti-CD19 CAR−T cells, nearly all circulating CAR+ T cells were CD4+ cells, particularly at early time points (FIG. 3F). Among non-transduced T cells, the CAR+ T cells the ratio of CD4:CD8 cells in the blood was altered after administration of muCAR−T cells (FIG. 3G).

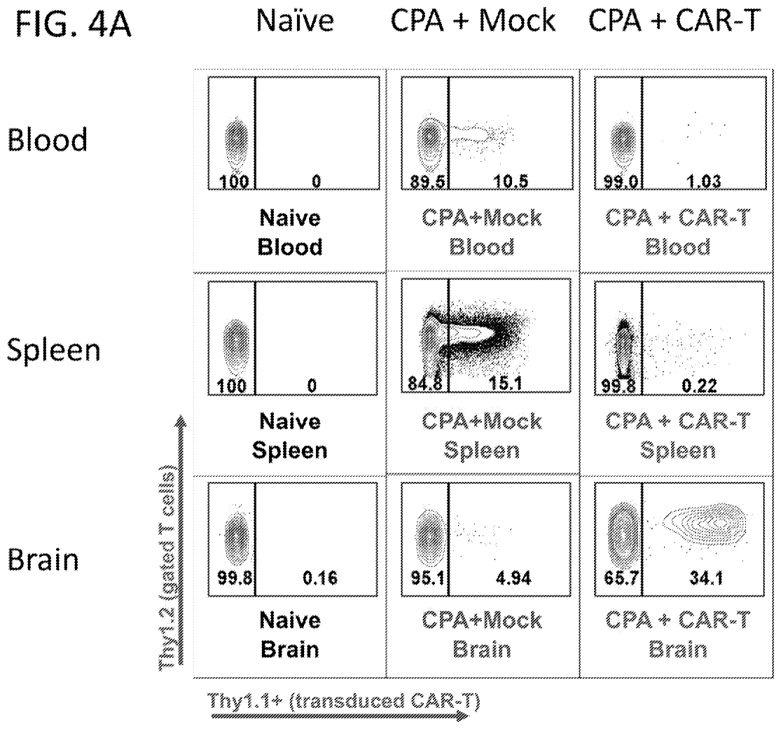

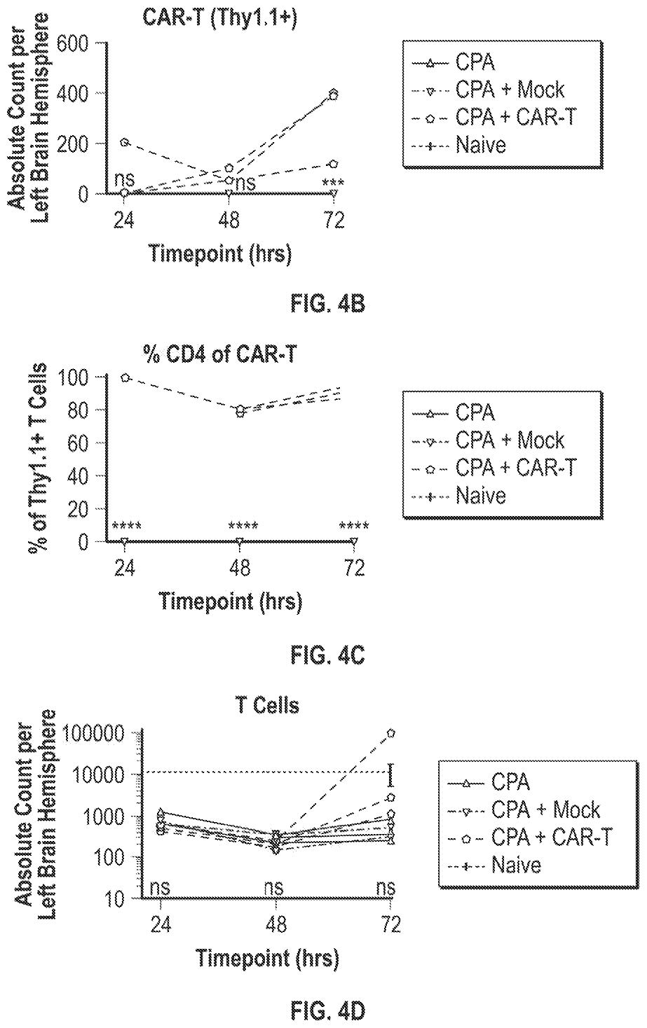

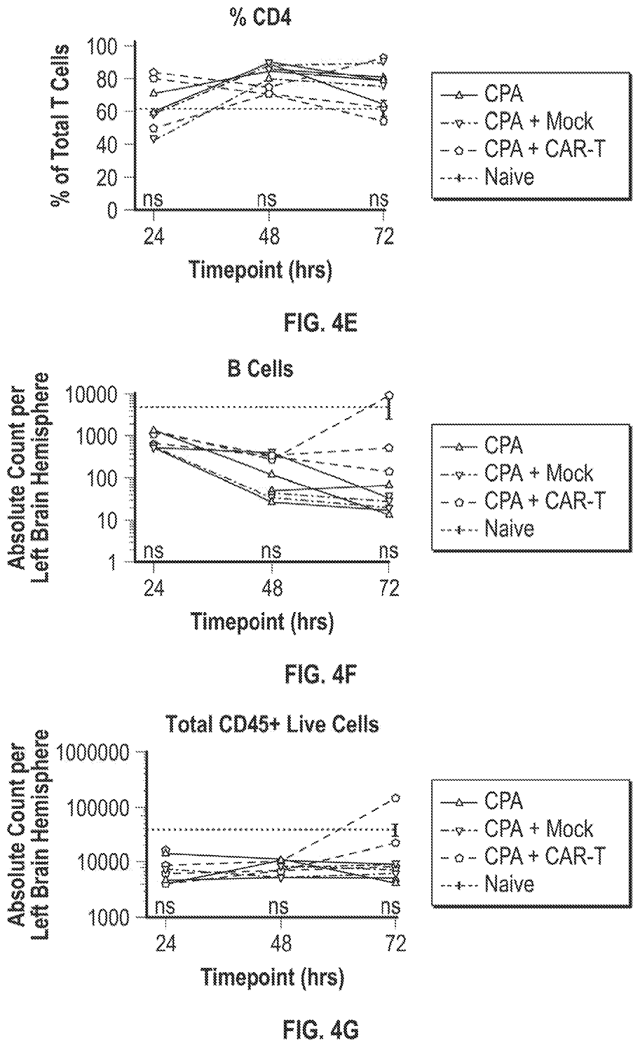

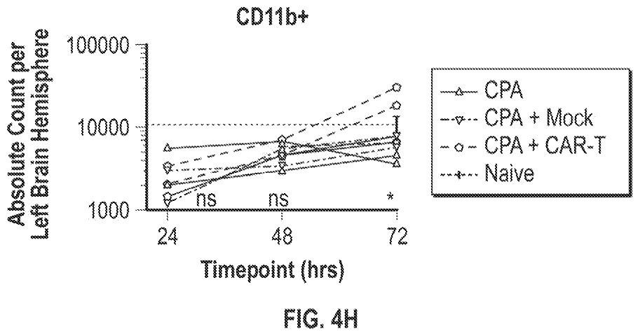

The presence of CAR+ T cells and endogenous T cells was assessed by flow cytometry based on surface markers Thy1.1 and Thy 1.2, respectively, 48 hours after administration of CAR+ T cells. FIG. 4A depicts exemplary flow cytometry results at 48 hours post-CAR−T administration in blood, spleen and brain. As shown, control (mock) T cells were detectable in blood and spleen, while mouse anti-CD19 CAR−T cells were detected in the brain. This result is consistent with CAR−T cell-specific transmigration into the brain. As described below, RNA-Seq analysis showing upregulated expression of ICAM-1, VCAM-1, E-Selectin and P-Selectin in samples from mice administered CPA+muCD19 CAR−T cells may support the hypothesis that the infiltration of CAR−T cells into the brain may indicate the presence of neuroinflammation and/or upregulation of endothelial cell adhesion molecules.

As shown in FIG. 4B, the presence of muCD19 CAR−T cells in the brain in mice was observed at all assessed time points, with a higher absolute number observed 72 hours after infusion of CAR−T cells compared to earlier time points. Of the CAR+ T cells in the brain, nearly all were CD4+ T cells (FIG. 4C). CPA pre-conditioning depleted immune cells in brain tissue, including endogenous T cells (FIG. 4D) and endogenous B cells (FIG. 4F). After 72 hours following administration of CPA+muCD19 CAR−T cells, there was a greater absolute number of endogenous T cells (FIG. 4D), endogenous B cells (FIG. 4F), CD45+ cells (FIG. 4G) and CD11b+ cells (FIG. 4H) in the brain compared to mice administered CPA alone control or the CPA+mock T cells. This result is consistent with an increased level of immune cell infiltrate into the brain over time after administration of CPA+mouse anti-CD19 CAR−T cells. FIG. 4E shows that as a percent of total T cells, the percentage of CD4+ cells was similar in mice among the treatment groups.

There was an increase in mouse anti-CD19 CAR−T cells, as detected by Thy 1.1+ expression, in liver within 24 hours, and in the spleen and kidney within 48 hours, following administration of CAR+ T cells. Similar to what was observed in brain tissue, the majority of CAR+ T cells in spleen, kidney and liver were CD4+. Treatment with CPA resulted in depletion of immune cells, including endogenous T cells, B cells, CD45+ cells, and CD11b+ cells in these tissues. In addition, a small but significant reduction in the percentage of T cells that were CD4+ was observed in liver, spleen, and kidney 72 hours following cell administration collected from CPA+CAR−T treated mice as compared to CPA-mock treated mice. Compared to the other treatment groups, administration of mouse anti-CD19 CAR+ T cells increased the percentage of immune cells, particularly CD11b+ cells, in spleen within 24 hours post-administration and increased the absolute number of B cells in the kidney by 72 hours post-administration. In liver, significantly less T cells were observed in tissue obtained from CPA+CAR−T mice at 48 and 72 hours following T cell administration as compared to liver of CPA-mock treated mice.

B. RNA Sequencing (RNA-Seq) of Brain Tissue

For transcriptome analysis by RNA-Seq, RNA from each brain tissue sample was obtained, fragmented and used to generate complementary DNA (cDNA) libraries for sequencing. Reads were processed and counts were normalized and log-transformed (log 2).

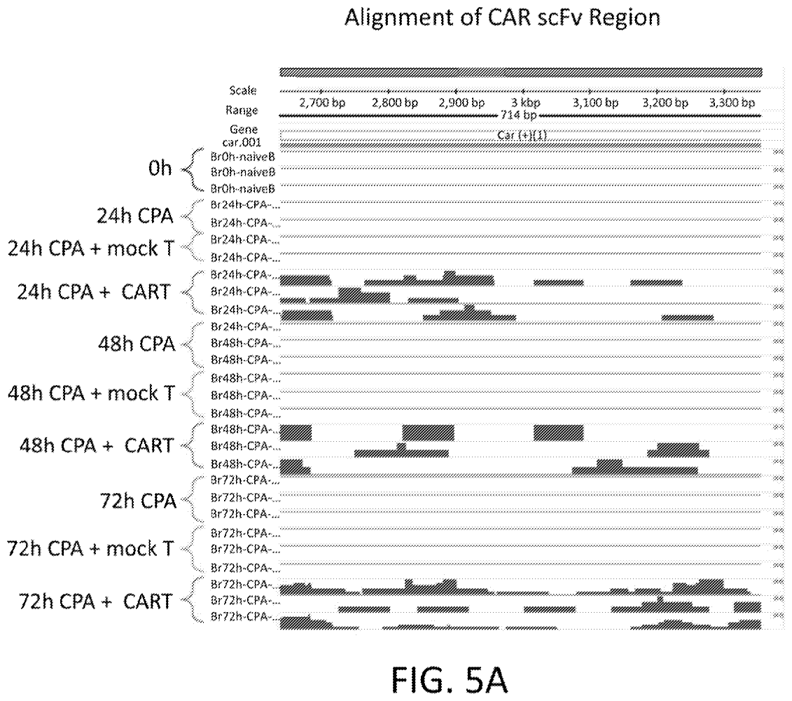

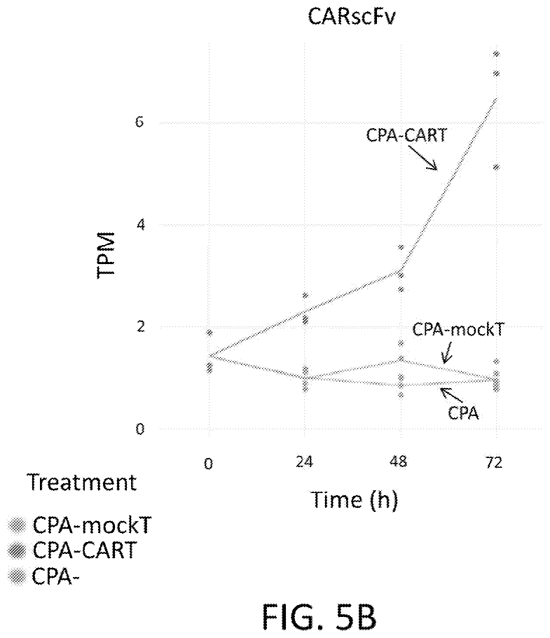

As shown in FIG. 5A, the anti-mouse CD19 CAR expression was detected by quantifying read counts of sequences that aligned to the scFv fragment of the anti-mouse CD19 CAR construct. Transcripts encoding the anti-mouse CD19 scFv were detected in brain tissue collected from the CPA+CAR−T group, and the levels of these transcripts increased in brain during the time points examined (FIG. 5A). Results from RNA-sequencing indicate possible infiltration of anti-mouse CD19 CAR−T cells into the brain.

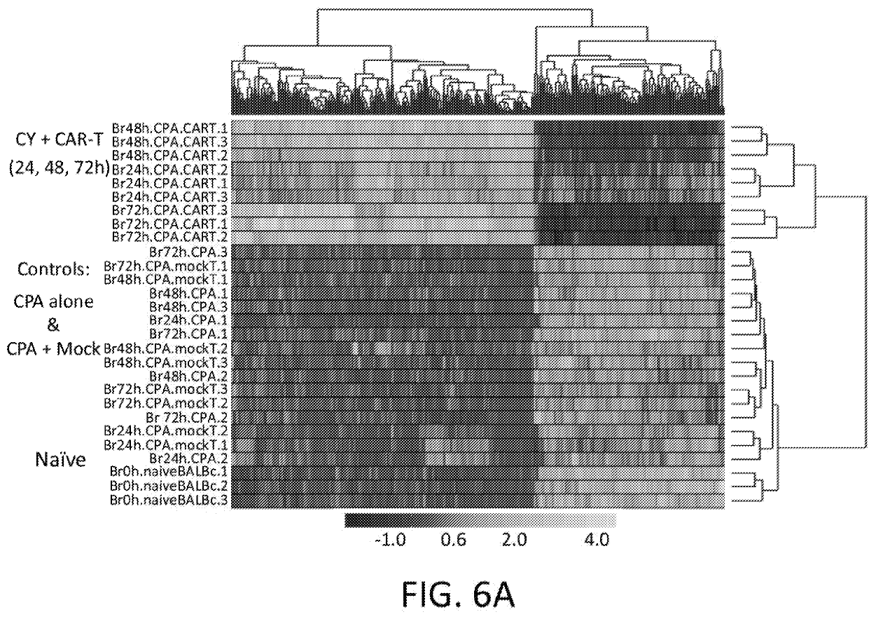

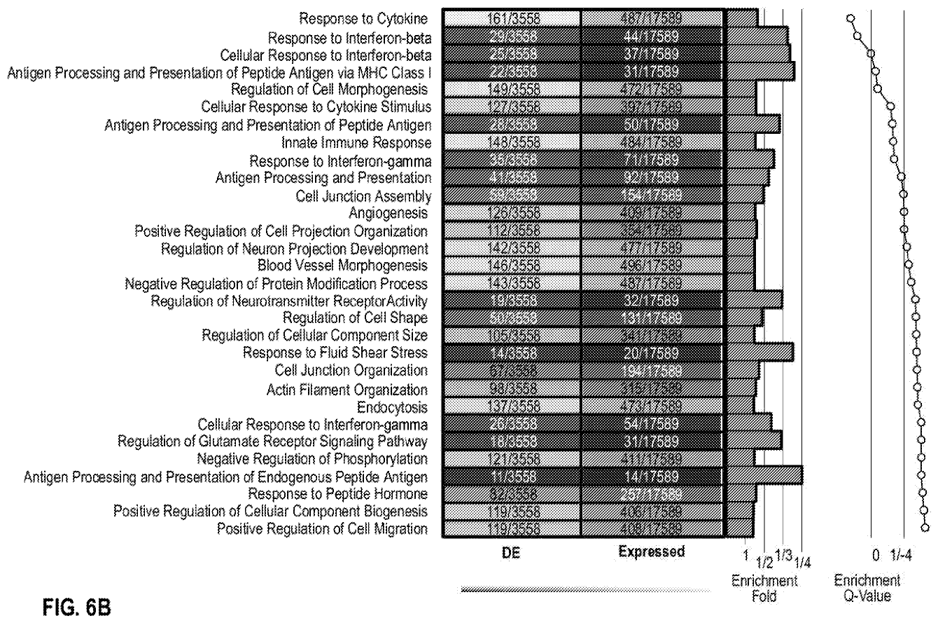

Genome-wide expression analysis by RNA-Seq identified approximately 17,589 genes that were expressed in brains of mice and, of these genes, 3,558 genes were differentially expressed in brains from mice treated with CPA+CAR−T cells as compared to brains from naïve mice (p<0.05 and log 2 fold-change >0.5 in CPA+CART vs 0; p>0.05 in all other treatment groups). The expression of 307 genes had a log 2 fold change of greater than 1.4 and a statistical significance of p<0.05 in at least one condition. Brains from CPA+CAR−T treated mice had a greater number of differentially expressed genes, i.e., genes with a significantly different expression of as compared to the naïve group, than brains from CPA alone or CPA-mock treated mice. The number of genes that were differentially expressed increased in brain samples obtained from at increasing times from 24 hours to 72 hours after administration of muCD19CAR−T cells. Hierachical clustering analysis was performed. As shown in FIG. 6A, the clustering analysis confirmed differential gene expression profiles in brains of mice treated with anti-mouse CD19 CAR T cells as compared to control groups.

As shown in FIG. 6B, ontological enrichment analysis demonstrated that, of the 3,558 differentially expressed genes that were identified in brains from CPA+CAR−T treated mice, thirty gene ontology (GO) categories had the greatest representation among the differentially expressed genes in brains from CPA+CAR−T treated mice. These included GO categories relating to cytokine activity, responses to interferons, antigen processing via MHC class I, innate immunity, and blood vessel morphogenesis. These results are consistent with involvement of inflammation in the observed transcriptional response in the brain following treatment with CPA and anti-mouse CD19 CAR−T cells.

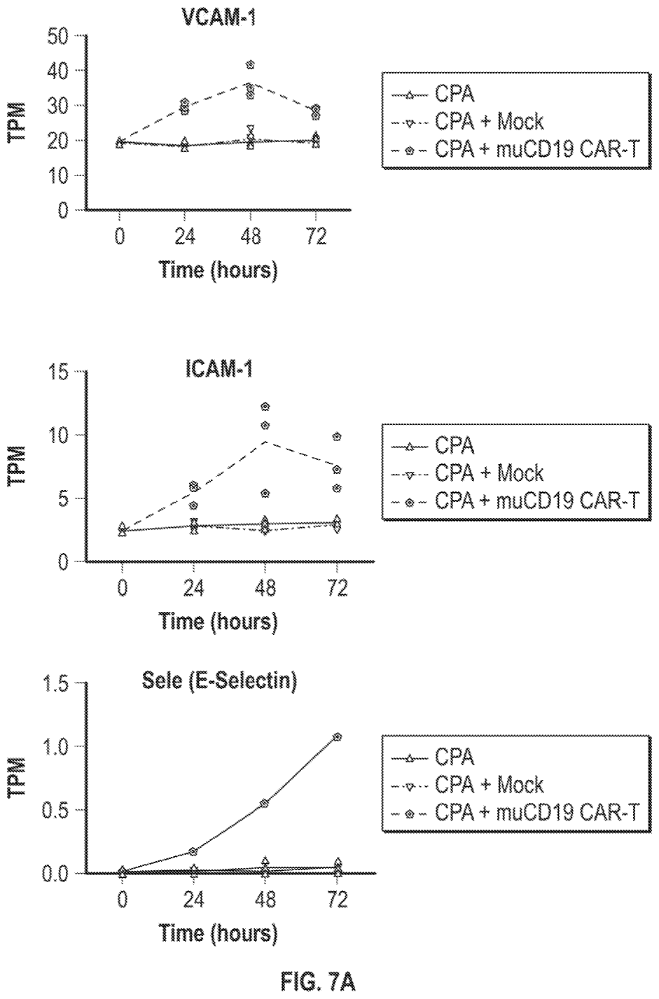

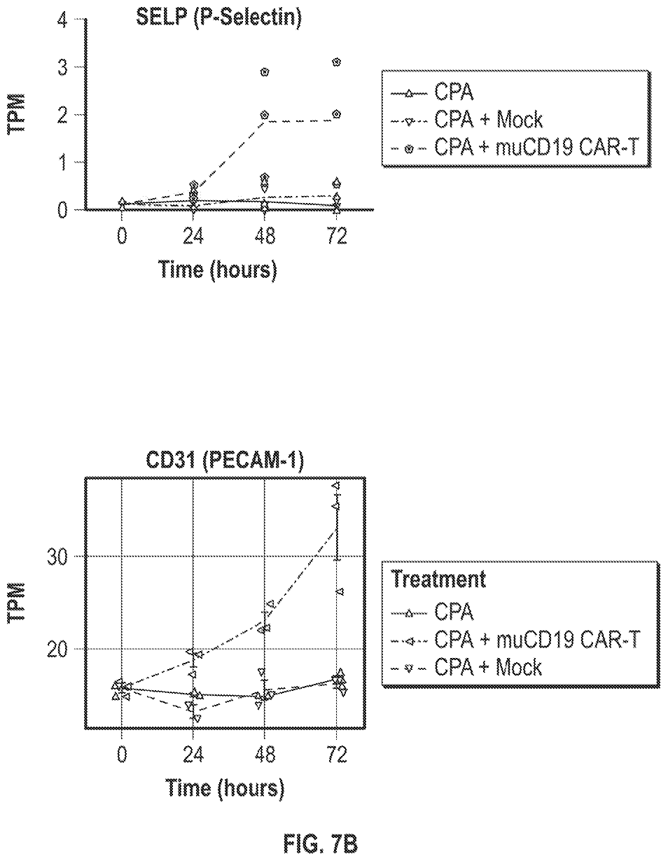

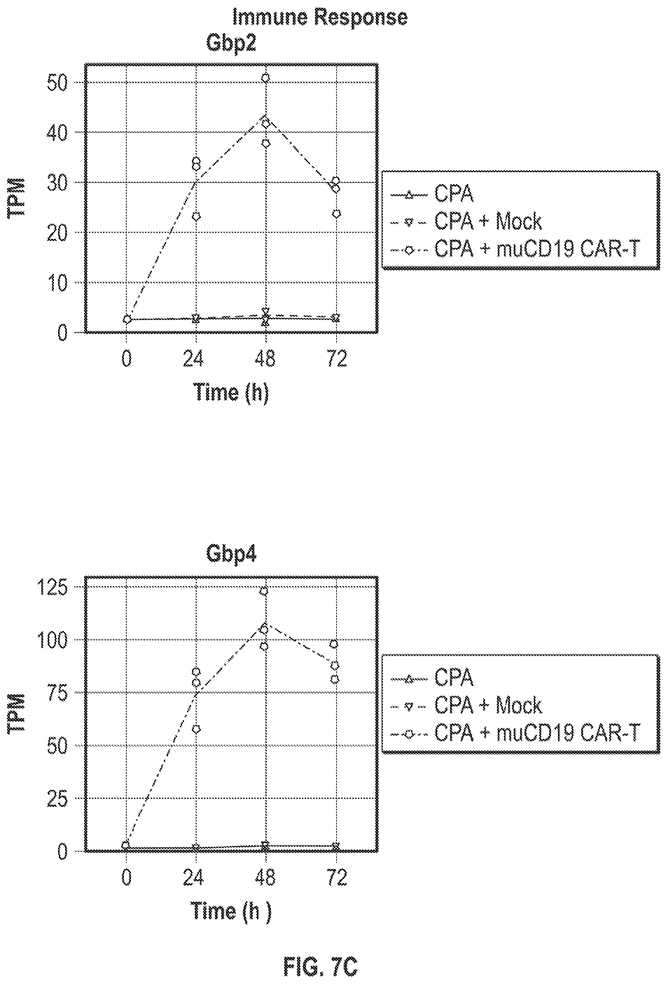

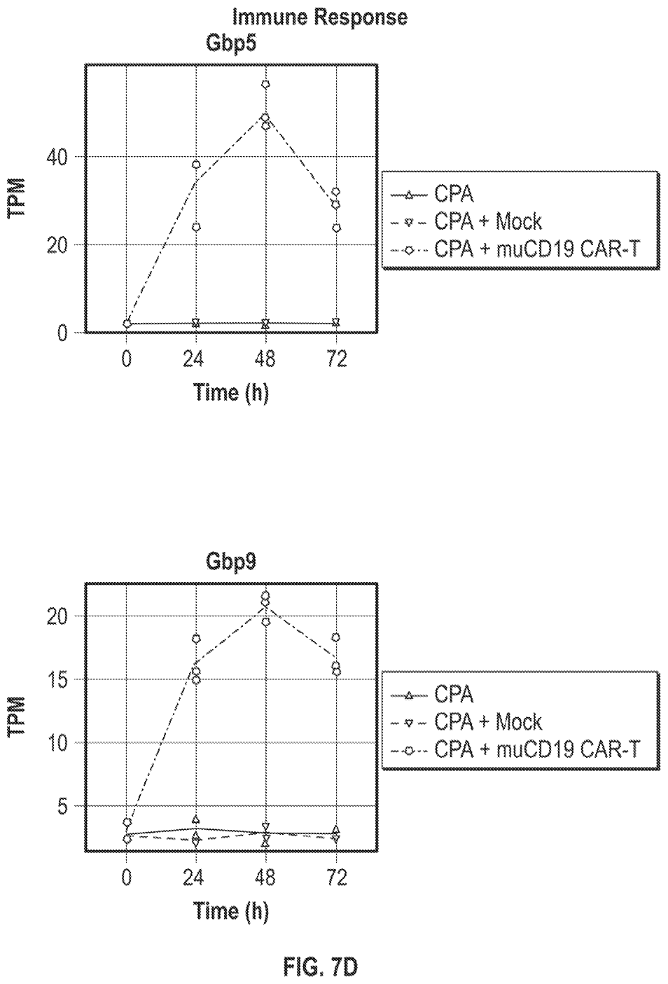

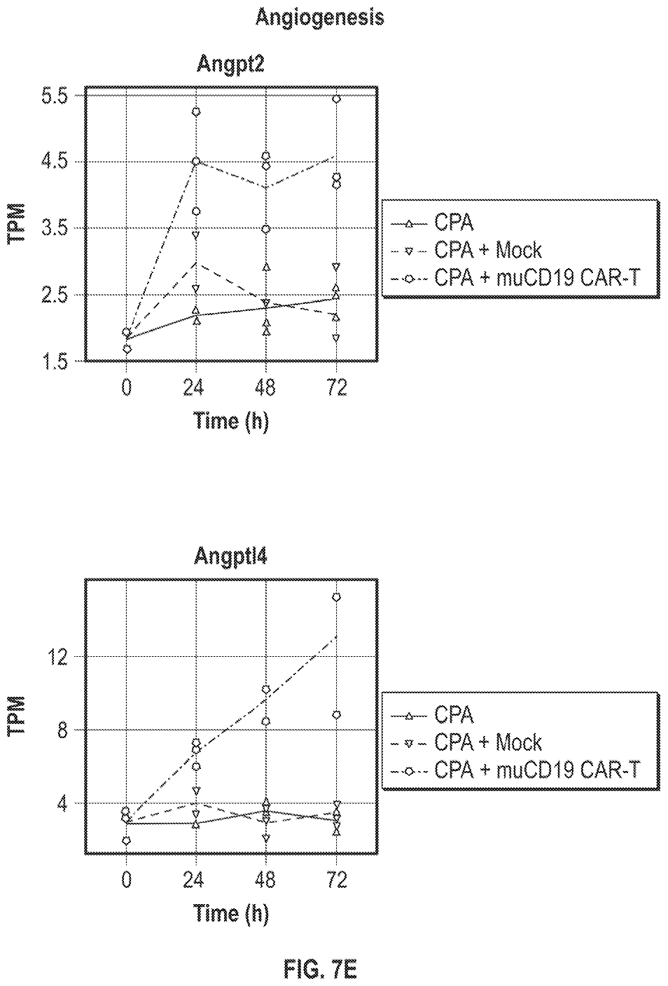

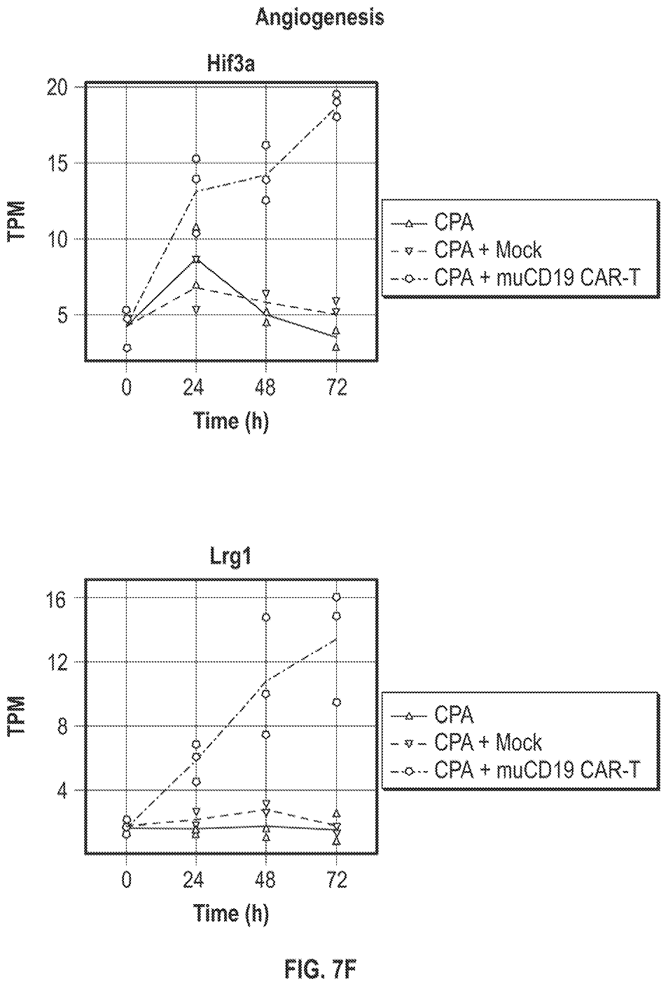

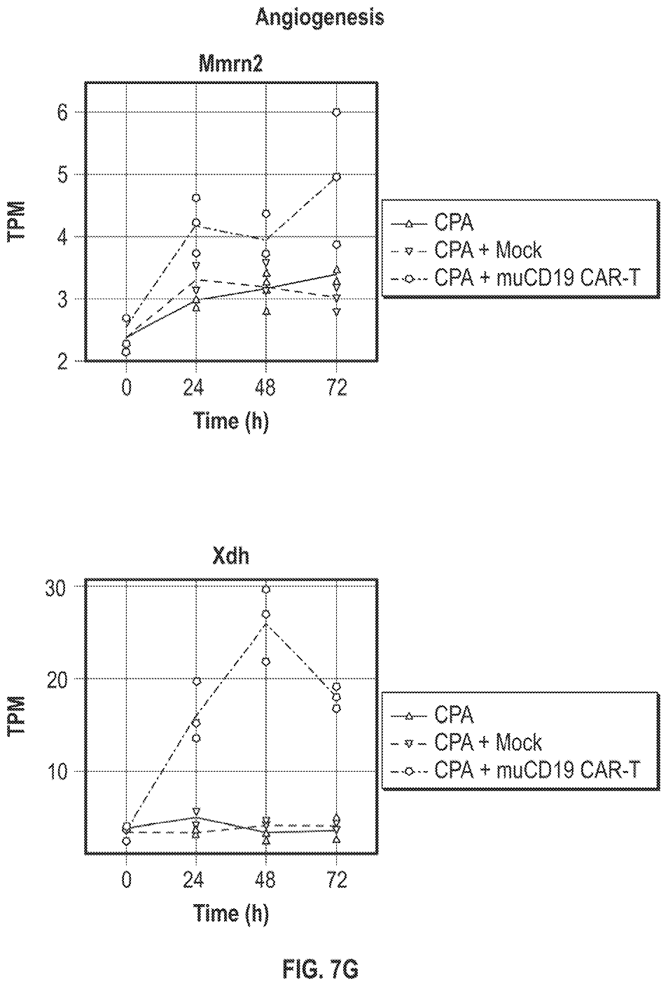

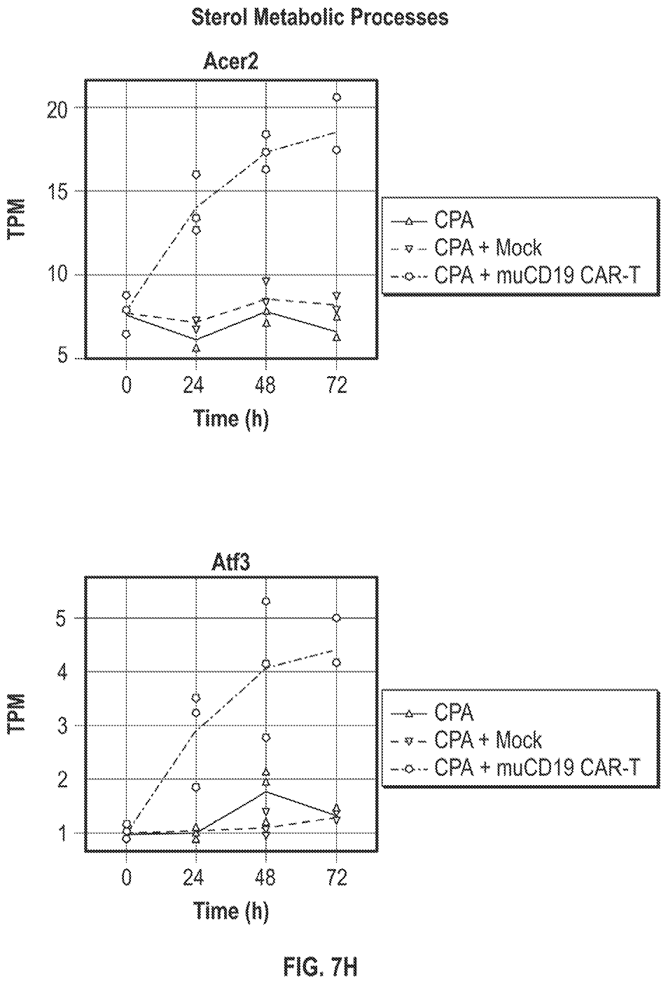

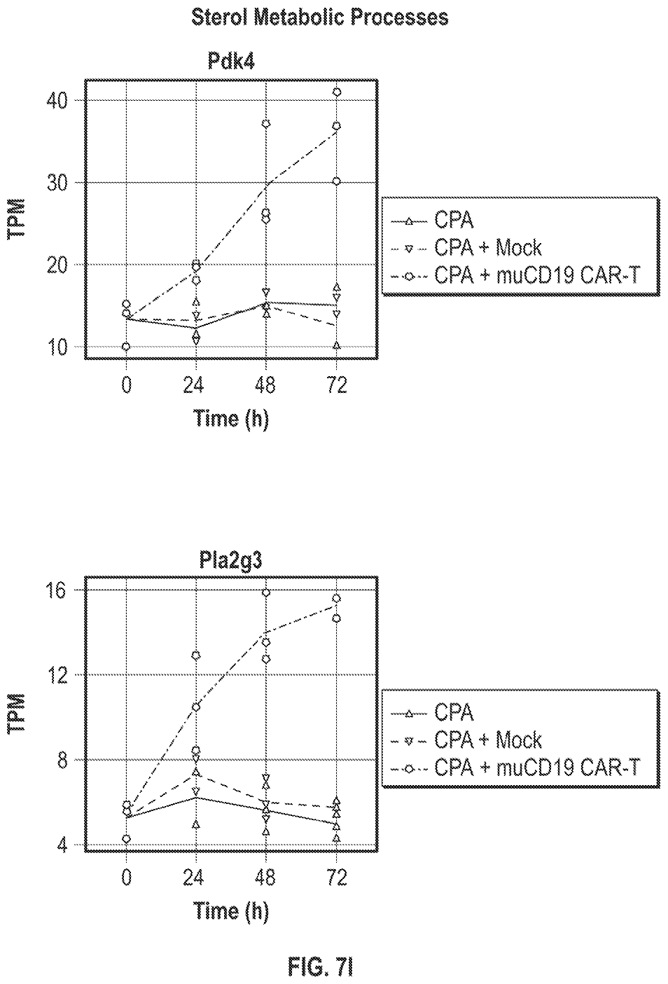

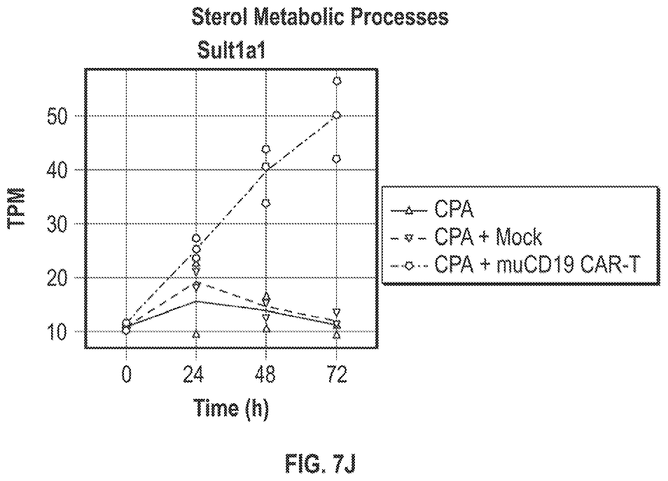

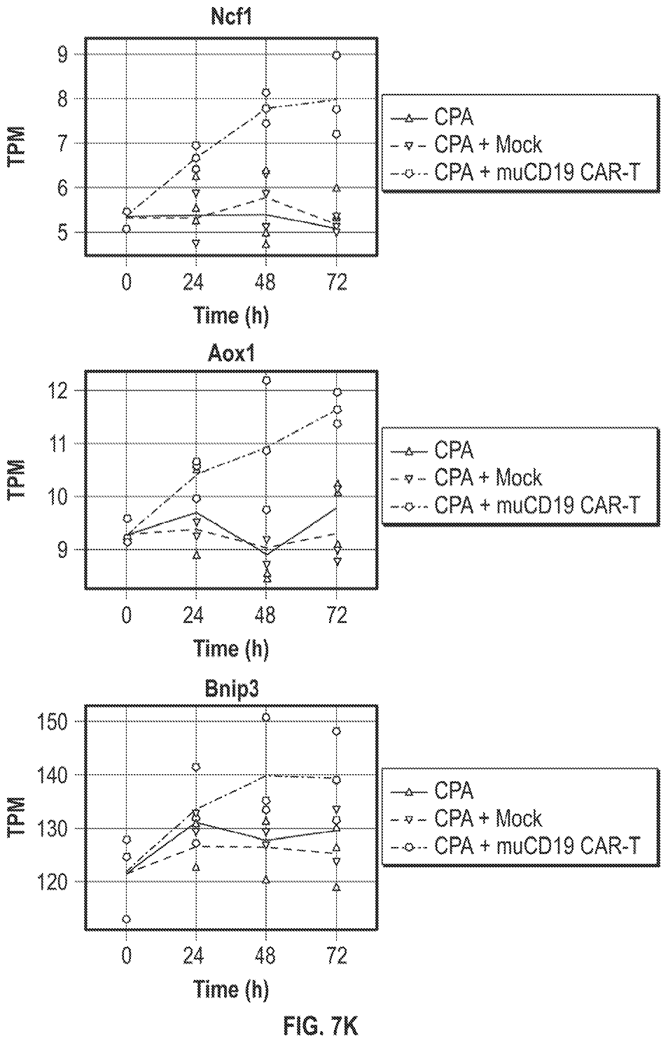

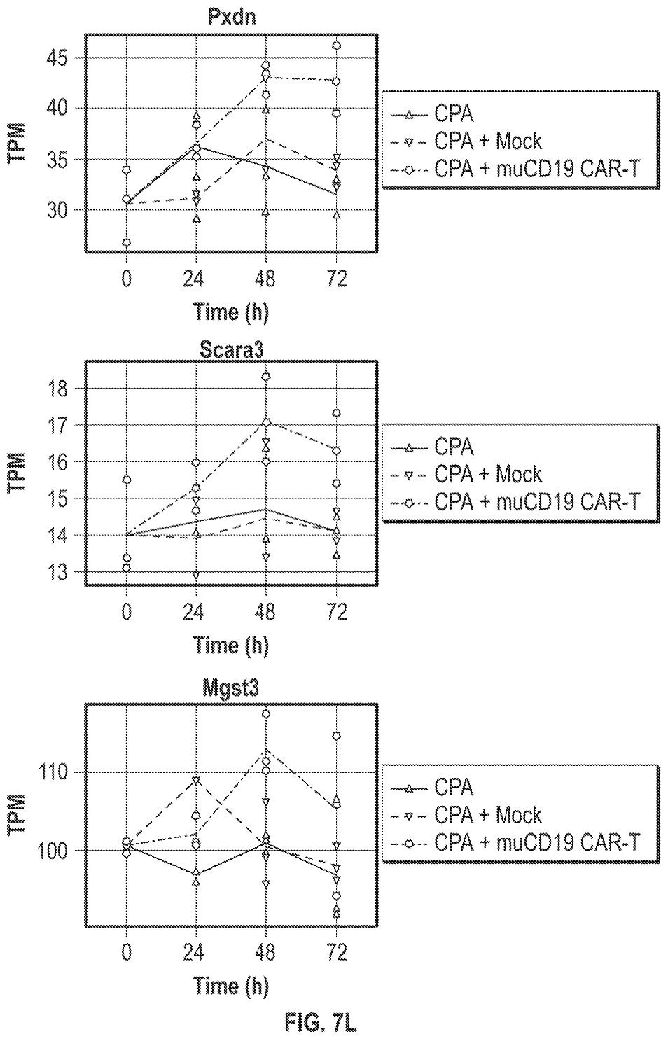

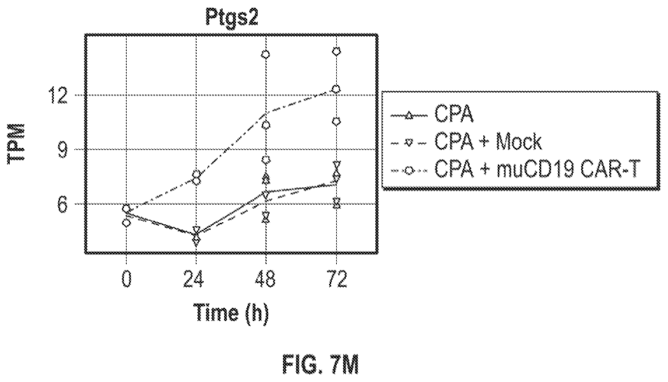

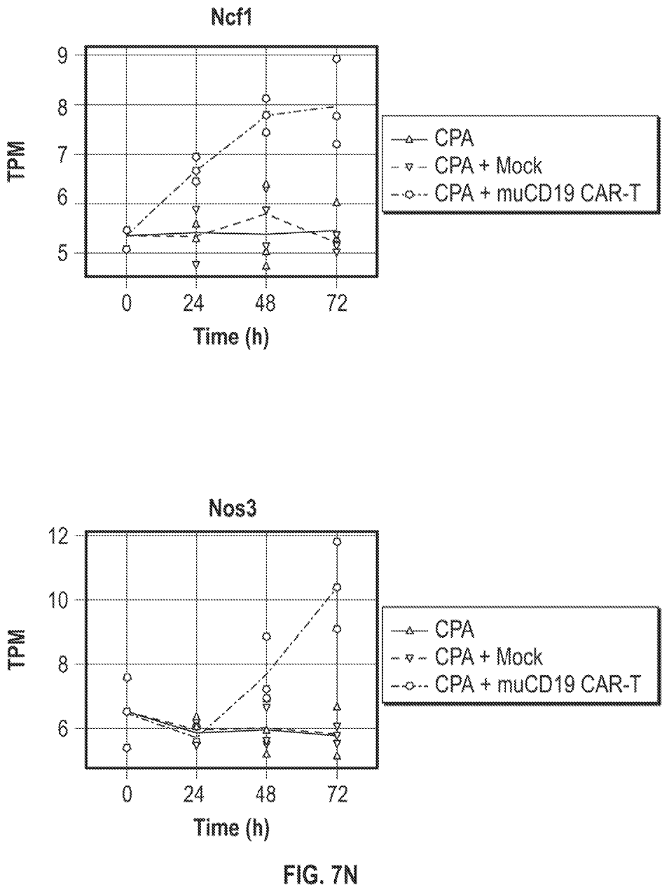

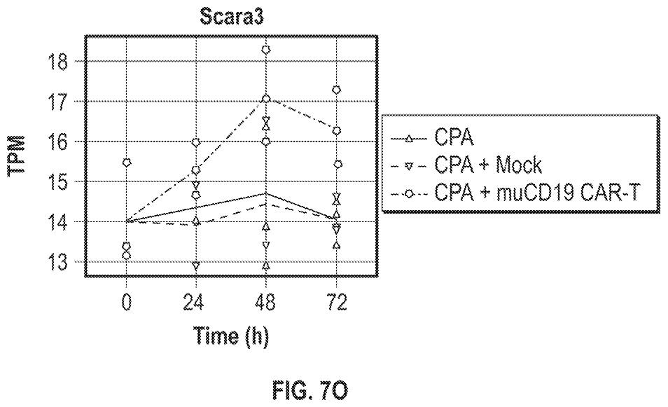

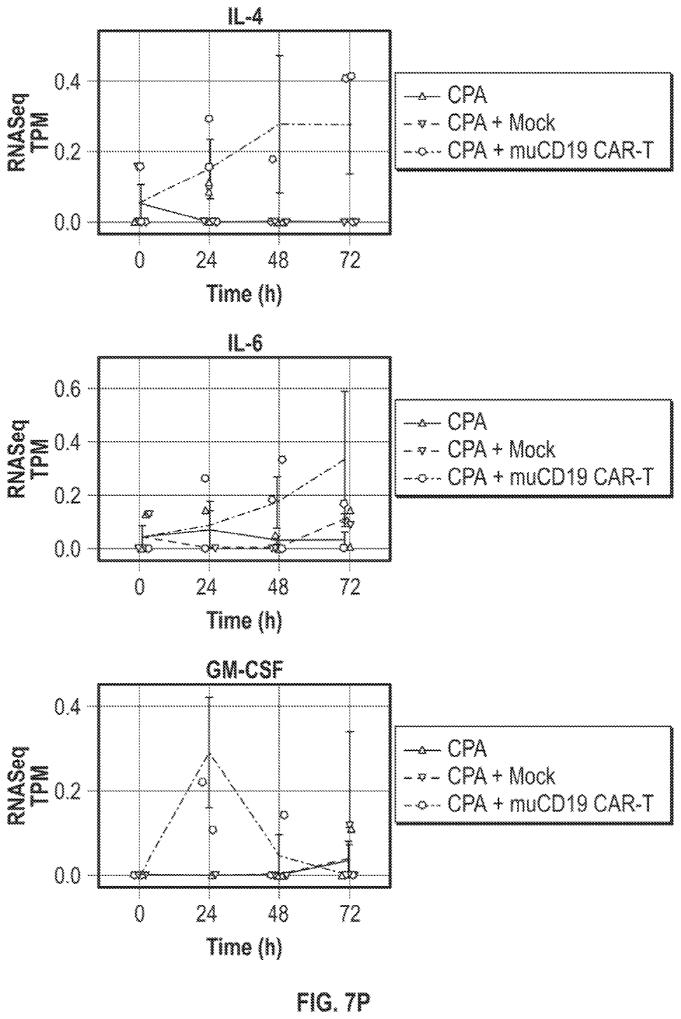

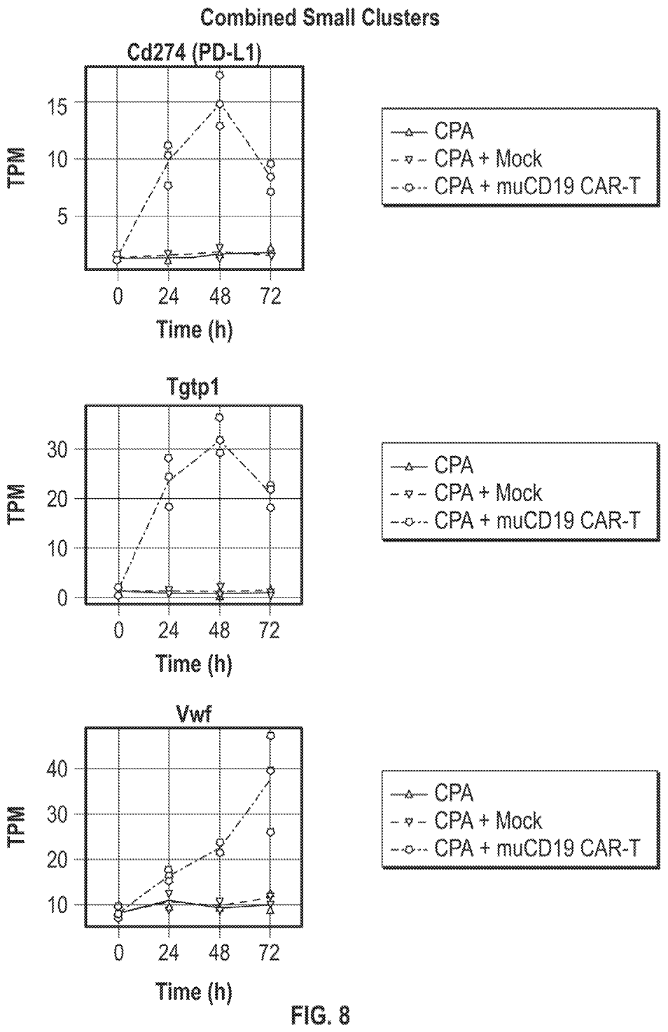

Exemplary genes in various GO classes that were differentially unregulated in brain following administration of CPA+CAR−T, but not following administration of CPA alone or CPA+mock, included: adhesion molecule genes (including VCAM-1, ICAM-1, E-Selectin, P-Selectin and CD31(PECAM-1) shown in FIGS. 7A and 7B); genes related to immune response (including Gbp2, Gbp4, Gbp5 and Gbp9 as shown in FIGS. 7C and 7D); genes related to angiogenesis (including Angpt2, Angpt14, Hif3a, Lrg1, Mmrn2 and Xdh as shown in FIGS. 7E-7G); genes related to sterol metabolic process (including Acer2, Atf3, Pdk4, Pla2g3, Sult1a1 as shown in FIGS. 7H-7J); genes involved in oxidative stress and antioxidant defense (including Ncf1, Aox1, Bnip3, Pxdn, Scara3, Mgst3, Ptgs2 as shown in FIGS. 7K-7M); and genes involved in nitric oxide signaling pathway (including Ncf1, Nos3, Scara3 as shown in FIGS. 7N and 7O). Genes encoding cytokines (including IL-4, IL-6, and GM-CSF as shown in FIG. 7P) were observed to be differentially expressed. Other exemplary genes that also were differentially expressed included those in other GO categories (including CD274 (also known as PD-L1), Tgtp1 and Vwf as shown in FIG. 8).

In sum, the results from the genome-wide expression analysis by RNA-seq demonstrated that treatment with CPA and mouse anti-CD19 CAR−T cells resulted in a transcriptional response in hundreds of genes in the brains of CPA+CD19 CAR−T-treated mice compared to controls. The gene ontology enrichment analysis was consistent with an inflammatory and vascular response following administration with CPA and mouse anti-CD19 CAR−T cells in the mouse model, including significant upregulation of several gene markers of inflammation, angiogenesis, endothelial activation and oxidative stress.

C. Blood Analysis

Serum chemistry profiles were analyzed in serum samples collected from naïve, CPA alone, CPA+mock, and CPA+CAR−T treated mice at 24 hours, 48 hours, and 72 hours, and 5 days post-CAR+ T cell administration. The serum was stored at −20° C. prior to analysis for complete serum chemistry panels, including for levels of serum glucose, albumin, albumin to globulin (A/G) ratio, globulin, total protein, calcium, phosphorus, ALT, AST, and BUN enzymes.

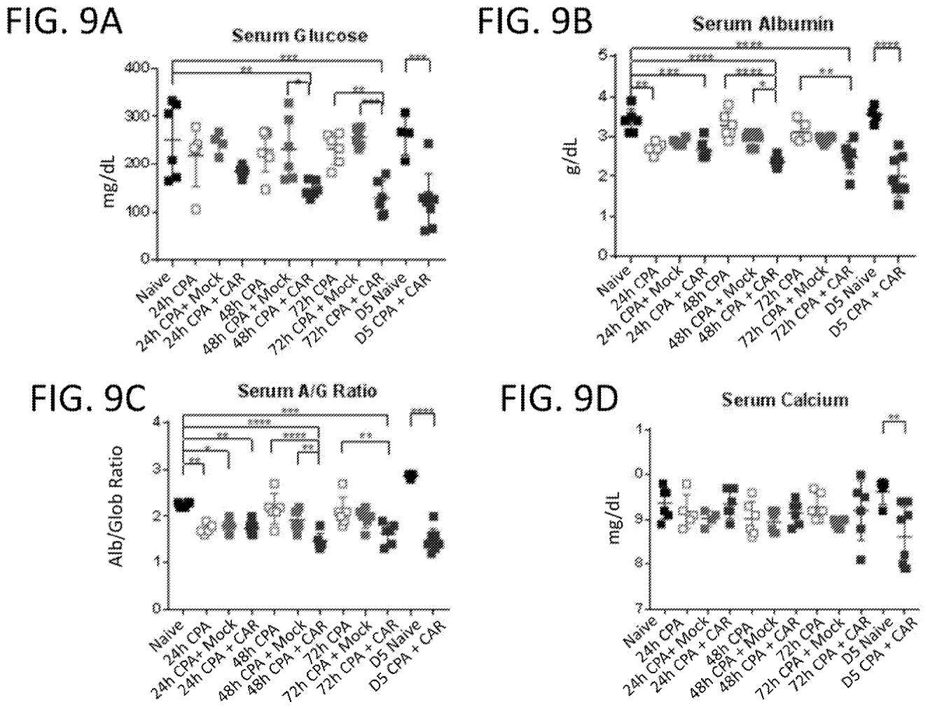

Levels of serum glucose (FIG. 9A) and serum albumin (FIG. 9B) were altered in mice following treatment with CPA and anti-mouse CD18 CAR−T cells as compared to other treatment groups. No significant differences in serum globulin levels were observed due to any treatments as compared to levels of samples from naïve mice, however, mice treated with CPA alone, CPA+mock T cells and CPA+CAR−T cells exhibited significantly reduced serum Albumin to Globulin ratio (A/G ratio) at various time points following cell treatment as shown in FIG. 9C. Reduced serum levels of calcium were observed in serum samples collected from CPA+CAR−T treated mice five days after cell treatment (FIG. 9D). No significant changes to serum phosphorus, ALT, AST, or BUN levels were observed in any treatment groups. The reductions in serum albumin and glucose levels are consistent with inflammatory responses seen in some human subjects experiencing toxicity to CAR+ T cell therapy.

### Example 5: Assessment of Body Weight and Pathology Associated with a Mouse Model of Toxicity

Mice were administered CPA alone, CPA and mock cell compositions (CPA+mock) and CPA and anti-mouse CD19 CAR−T cell compositions (CPA+CAR−T) substantially as described in Example 2. Naïve mice were also used as a control group. In this study, a total of 5×10⁶ cells of both mock and CAR−T cell compositions were administered to each mouse. Mice were anesthetized, transcardially perfused with PBS, and sacrificed. Tissues were collected for analysis by histology.

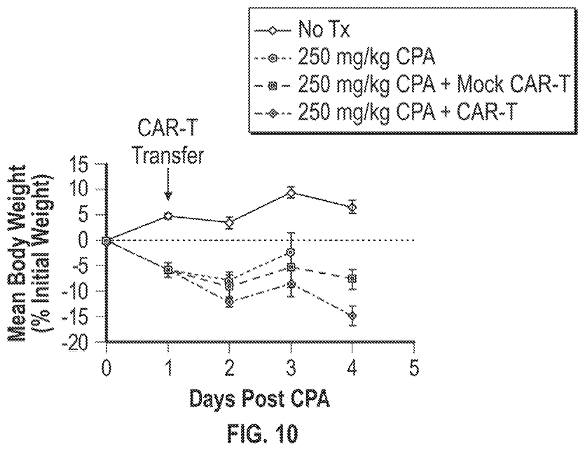

Body weights were measured in all groups at 24, 48, and 72 hours after treatment with the cell compositions. As shown in FIG. 10, all groups of mice treated with CPA exhibited weight loss that was detectable within 24 hours, and further weight loss was observed in mice treated with CPA+CAR−T at 96 hours as compared to mice treated with CPA+mock T cells.

To assess effects of treatment on the vasculature, mice from each treatment group were injected with approximately 70,000 MW albumin bound to Evans blue dye at 72 hours after cell infusion. No evidence of vascular leakage or breakdown of the blood brain barrier was observed by Evans blue staining in brains collected from any treatment groups.

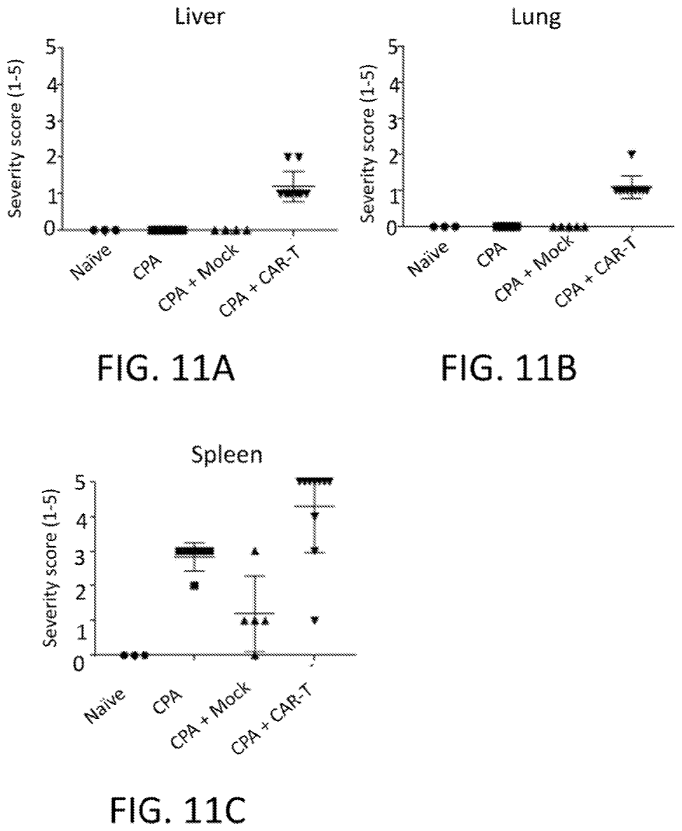

Tissues from brain, spleen, liver, kidney, and lung were examined for histopathology. As a measure of pathology, histiocytic granulomatous infiltration was evaluated and rated on a severity score of 1-5, with a score of 5 indicating the most severe pathology and a score of 0 indicating that no pathology was detected in the tissue. Scoring of histiocytic granulomatous infiltration indicated that significant pathology was present in liver and lung tissue from CPA+CAR−T treated mice (FIGS. 11A and B). Pathology was observed in spleens from all CPA treated groups, with spleens from CPA+CAR−T treated mice showing the highest degree of severity (FIG. 11C). In addition, minor necrosis was observed in the spleen and liver of CPA+CAR−T treated mice. No significant pathology was observed in brain tissues from any group.

### Example 6: Evaluation of Effects of Antigen-Expressing Tumor Burden on Mouse Model of Toxicity

To examine the effects of tumor burden on the toxicity following B cell aplasia in mice, BALB/c mice were injected with CD19-expressing A20 cells prior to treatment with CPA and mouse anti-CD19 CAR−T cells. A20 cells are a cell line of B lymphocytes derived from a spontaneous reticulum cell neoplasm in a BALB/cAnN mouse. As the cell line was originally derived from cells of an individual of a BALB/c mouse substrain, the cells are syngenic to the BALB/c mice and can be administered into an immunocompetent BALB/c mouse without triggering an immune response.

BALB/c mice were injected intravenously with A20 cells. After 26 days, mice with no tumor or mice having received A20 cells were injected with 250 mg/kg CPA i.p. and, for some groups, 24 hours later were administered 5×10⁶ anti-mouse CD19 CAR+ T cells or mock T cells, each generated as described in Example 1, following procedures substantially as described in Example 2. Table E3 sets forth the treatment groups.

TABLE E3
A20-Tumor Bearing Treatment Groups

| Group | Description | A20 cells | CPA | muCAR-T or mock |
|---|---|---|---|---|
| 1 | naive | No tumor | — | — |
| 2 | CPA + mock | No tumor | 250 mg/kg | 5 × 10⁶ mock T |
| 3 | CPA + CAR-T | No tumor | 250 mg/kg | 5 × 10⁶ muCAR-T |
| 4 | A20 alone | A20 (i.v.) | — | — |
| 5 | A20 + CPA | A20 (i.v.) | 250 mg/kg | — |
| 6 | A20 + CPA + mock | A20 (i.v.) | 250 mg/kg | 5 × 10⁶ mock T |
| 7 | A20 + CPA + CAR-T | A20 (i.v.) | 250 mg/kg | 5 × 10⁶ muCAR-T |

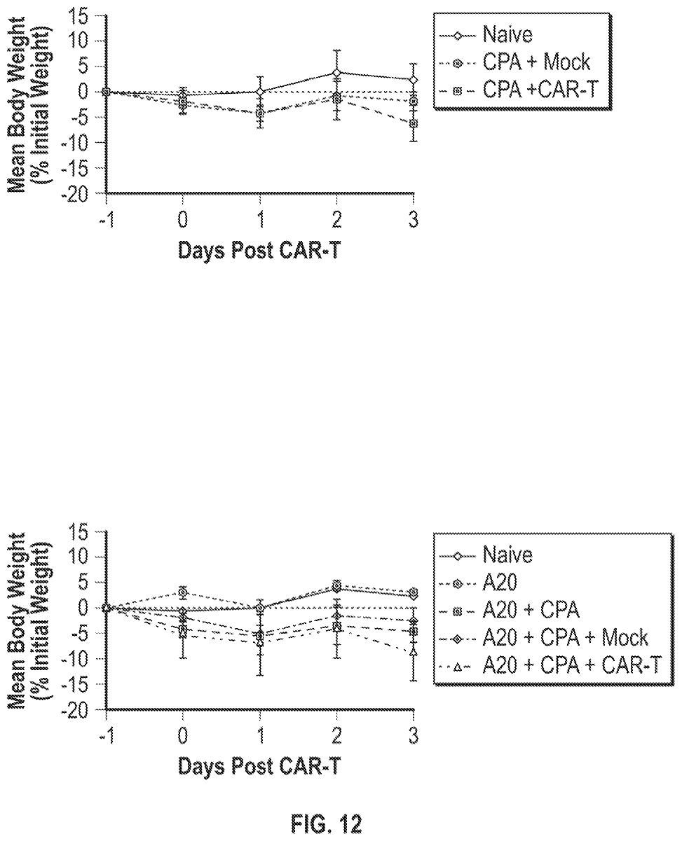

Weight measurements were taken 24 hours prior to infusion, at the time of infusion, and 24, 48, and 72 hours after infusion of mock and CAR−T cell compositions. As shown in FIG. 12, there was a slightly greater weight loss in mice administered CPA+CAR−T cells, including in the A20+CPA+CAR−T treated mice, as compared to other treatment groups. No difference in body temperature of mice was observed.

To test for potential vascular leakage and blood brain barrier disruption, three mice from each experimental group were injected 3 and 6 days following infusion of mock or CAR−T cell compositions with 3,000 MW dextran conjugated with Texas Red, 10,000 MW dextran conjugated with Fluorescein, and approximately 70,000 MW Albumin bound by Evans blue dye. Mice were anesthetized, perfused with PBS, and sacrificed. Tissues were collected for analysis by fluorescence microscopy and histology staining.

Texas red, fluorescein, and Evans blue dye were imaged in blood vessels in brain samples from naïve mice, CPA+CAR−T mice or A20+CPA+CAR−T by fluorescence microscopy for evidence of extravasation as an indicator of blood brain barrier disruption. Empty lumen and staining in the vessel wall in the pia on the surface of the brain and capillaries in the brain neuropil were observed with all stains from all samples. No signal from the stains was observed in extravascular spaces, indicating a lack of detectable extravasation.

For histology, mice from the different treatment groups were sacrificed at 3 and 6 days after infusion of the CAR−T or mock cell compositions, and liver and spleen tissues were collected and examined for extramedullary hematopoiesis and histiocytic/granulomatous infiltrates. Spleen was also examined for lymphoid depletion and fibrosis. These parameters were rated on a severity score of 1-5, with a score of 5 indicating the most severe pathology and a score of 0 indicating that no pathology was present.

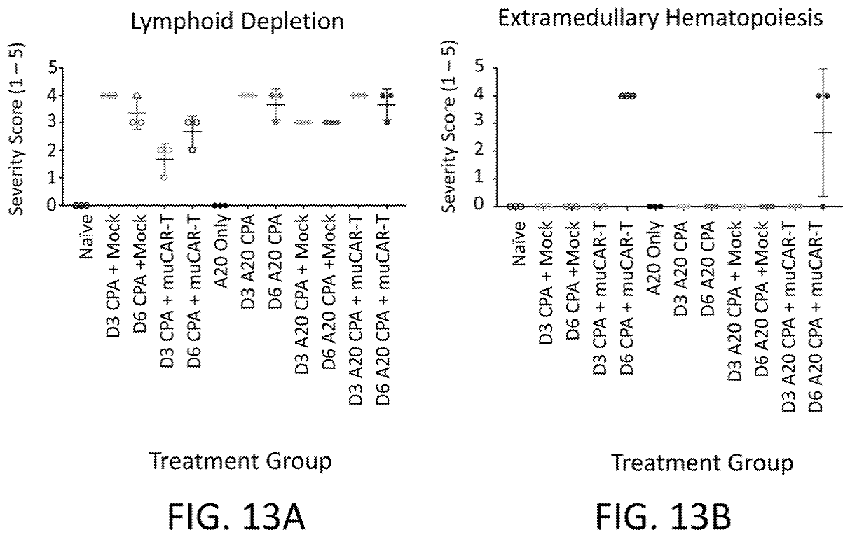

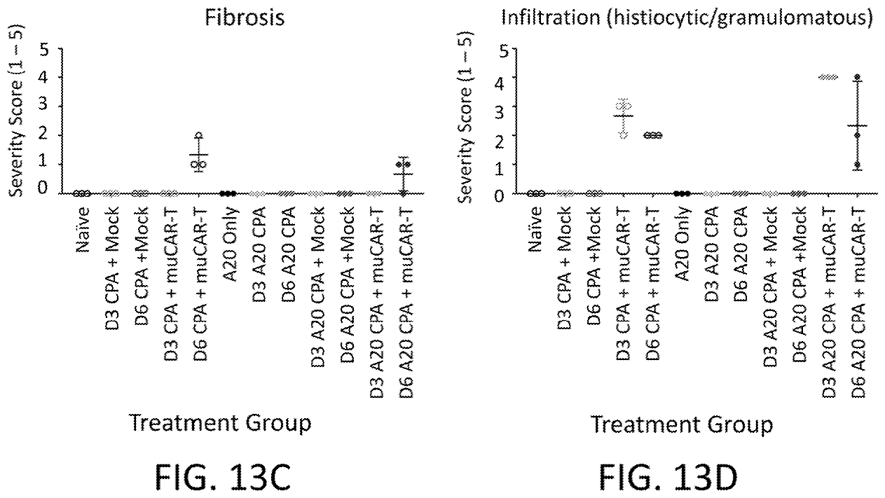

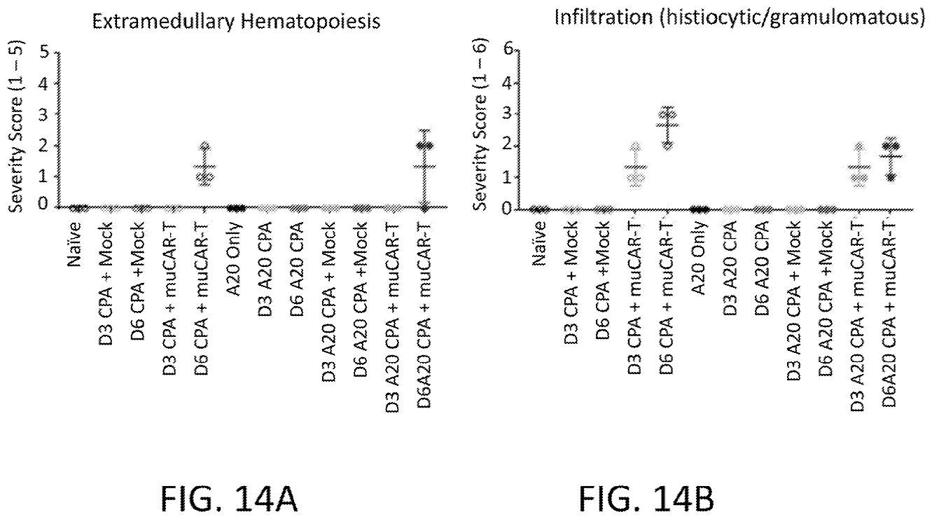

As shown in FIG. 13A, lymphoid depletion was observed in spleens collected from all groups that received treatment with CPA. Extramedullary hematopoiesis (FIG. 13B), which is indicative of bone marrow injury and regeneration, and fibrosis (FIG. 13C) were observed in spleens from CPA+CAR−T and A20+CPA+CAR−T treated mice collected 6 days after treatment with CAR−T cell compositions. Infiltration (FIG. 13D) was observed in spleens from CPA+CAR−T and A20+CPA+CAR−T treated mice collected 3 days and 6 days after treatment with CAR−T cell compositions. As shown in FIGS. 14A and 14B extramedullary hematopoiesis and histiocytic/granulomatous infiltration, respectively, were only observed in liver collected from mice at 3 and 6 days following administration of CAR−T or mock cell compositions. Similar to what was observed in spleen tissue, extramedullary hematopoiesis and infiltration were only observed in liver collected from CAR−T cell treated mice.

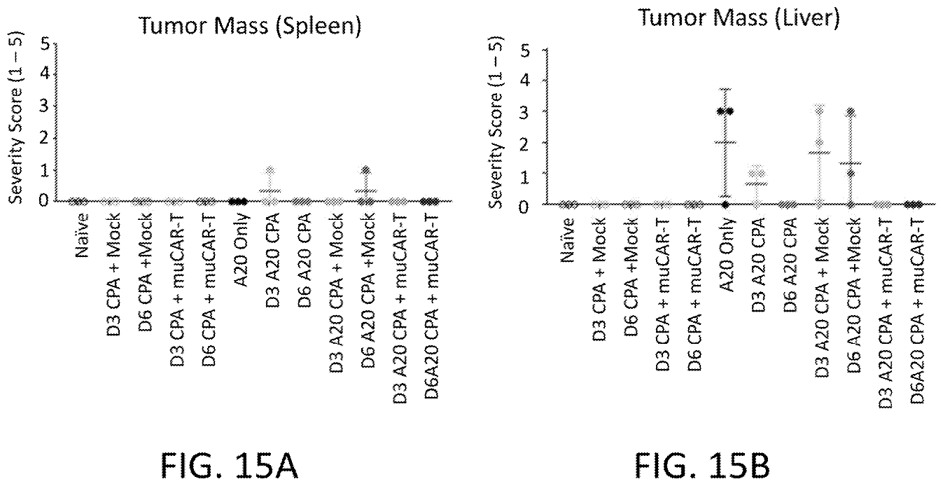

Tumor burden also was assessed by histology in treated mice. A20 tumor masses were observed in some liver (FIG. 15A) and spleen (FIG. 15B) tissues from A20 alone, A20+CPA, and A20+CPA+mock treatment groups, but not in the group treated with A20+CPA+CAR−T cell group. The tumors showed degeneration in the A20+mock and A20+CPA Treatment with anti-mouse CD19 CAR cells resulted in clearance of liver and spleen tumors.

### Example 7: Analysis of Effect of Dose of CAR−T Cells in Mouse Model of Toxicity

Mice were treated using methods substantially as described in Example 2, except that anti-mouse CD19 CAR+ T cells were administered at a dose of 10×10⁶ cells/mL. BALB/c mice were injected with 250 mg/kg cyclophosphamide (CPA) i.p and twenty-four hours later were administered 10×10⁶ mouse CAR-expressing T cells or non-target (control) cells, generated as described in Example 1.

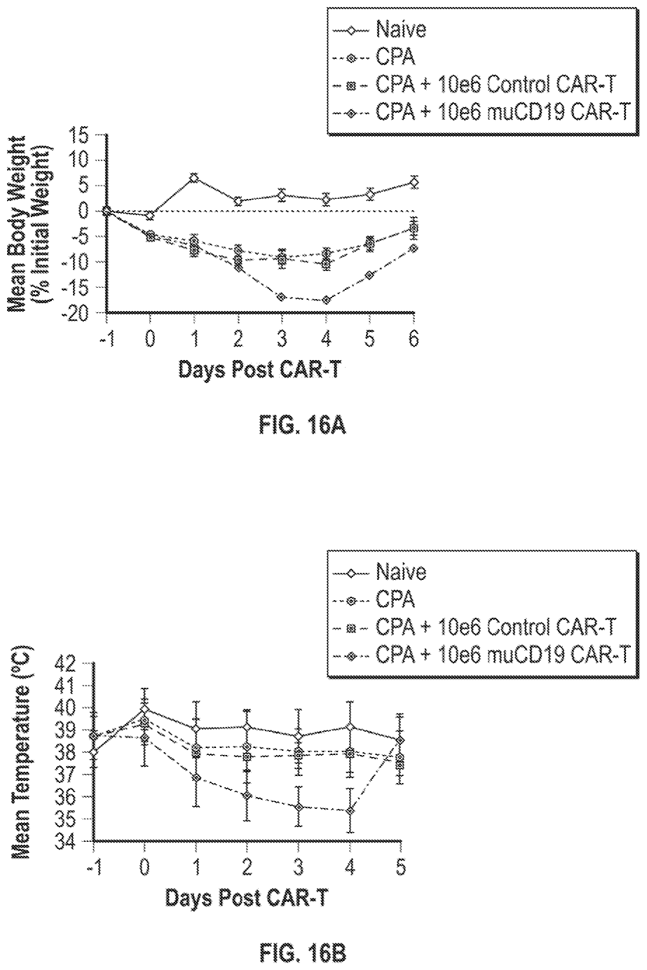

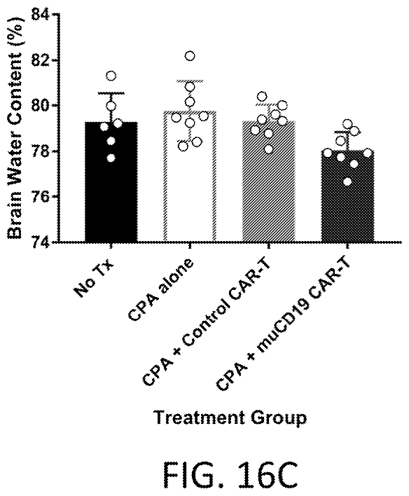

Body weight and temperature measurements were taken 24 hours prior to infusion, at the time of infusion, and daily for 5 days after infusion with the cell compositions. As shown in FIG. 16A, all mice that were administered CPA exhibited reduced body weight as compared to naïve controls. Treatment of mice with anti-mouse CD19 CAR+ T cells, but not with non-target control anti-human CD19 CAR+ T cells, exhibited a greater decrease in body weight compared to mice treated only with CPA. As shown in FIG. 16B, similar effects were seen on body temperature. The reduced body weight in the anti-mouse CD19 CAR treated mice recovered to similar levels as mice from the CPA alone and CPA+non-target control CAR−T treatment groups by 6 days following cell infusion. Body temperature recovered by day 5.

Mice were sacrificed at 5 days after cell infusion and brains were examined for brain water content using the wet-dry method. Brains were removed from mice and weighed immediately to obtain the wet brain weight. Brains were then dehydrated in an incubator oven at about 65° C. for 72 hours. The brains were weighed again at 72 hours to obtain the dry brain weight. Brain edema was estimated by comparing wet to dry weight ratios. The percent of tissue water content was calculated using the following formula: BWC=[(wet weight-dry weight)/wet weight]*100. The Brain water content of mice at day 5 in this study is shown in FIG. 16C.

Increased levels of the mRNA encoding these cytokines were also detected in brain tissue following treatment with anti-mouse CD19 CAR−T cells (FIG. 7G).

### Example 8: Effects of CPA and Anti-Mouse CD19 CAR−T Cell Treatment in A20 Tumor Bearing Mice

A20 tumor bearing mice were administered CPA and anti-mouse CD19 CAR−T cells and evaluated for various parameters. BALB/c mice were injected i.p. with 2×105 CD19-expressing A20 tumor cells similar to as described in Example 6. Eighteen days later, treatment with CPA and CAR−T cells was initiated. For treatment with CAR−T cells, mice were injected with 10×106 anti-mouse CD19 CAR−T cells or non-target anti-human CD19 CAR−T cells that were generated as described in Example 1. Control groups also included those injected with A20 tumor cells alone (A20 only) or with A20 tumor cells and CPA. Tissues and blood samples were collected from 4 to 8 mice at each time point.

### A. Serum Cytokine Levels

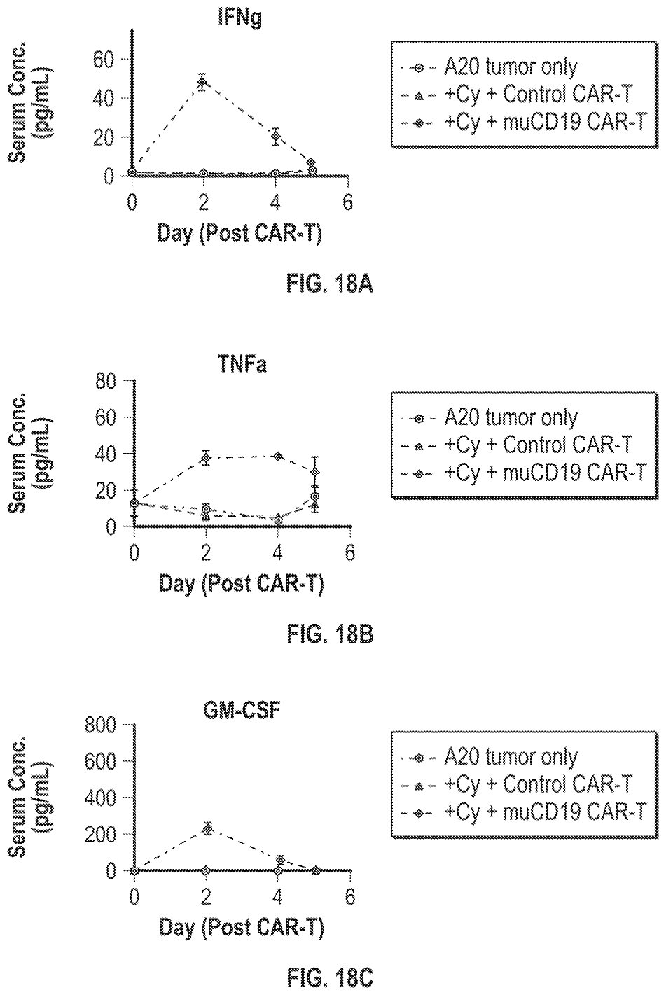

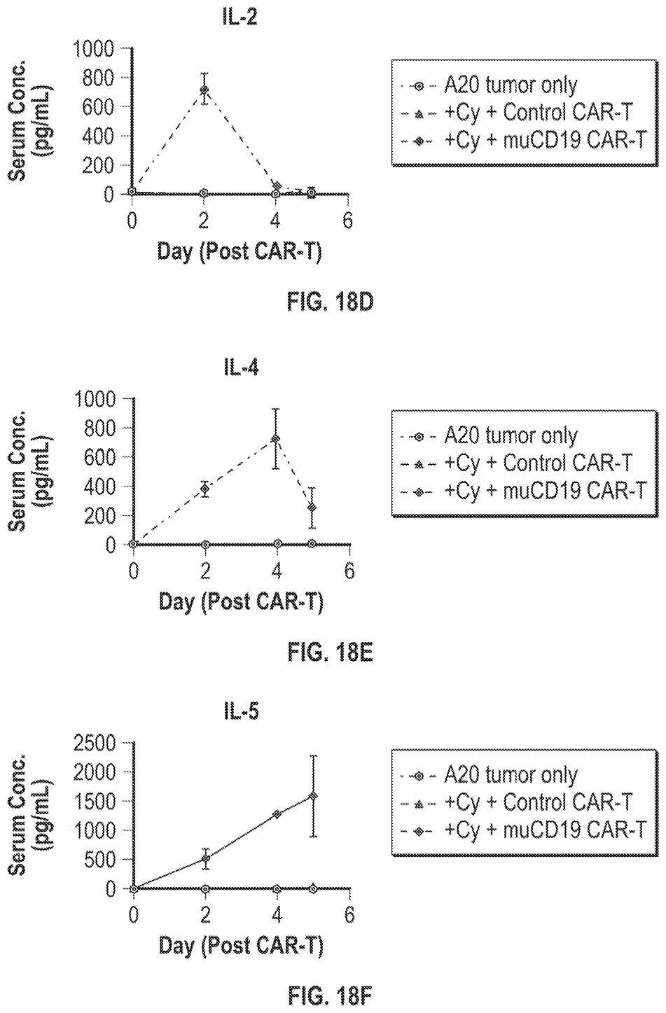

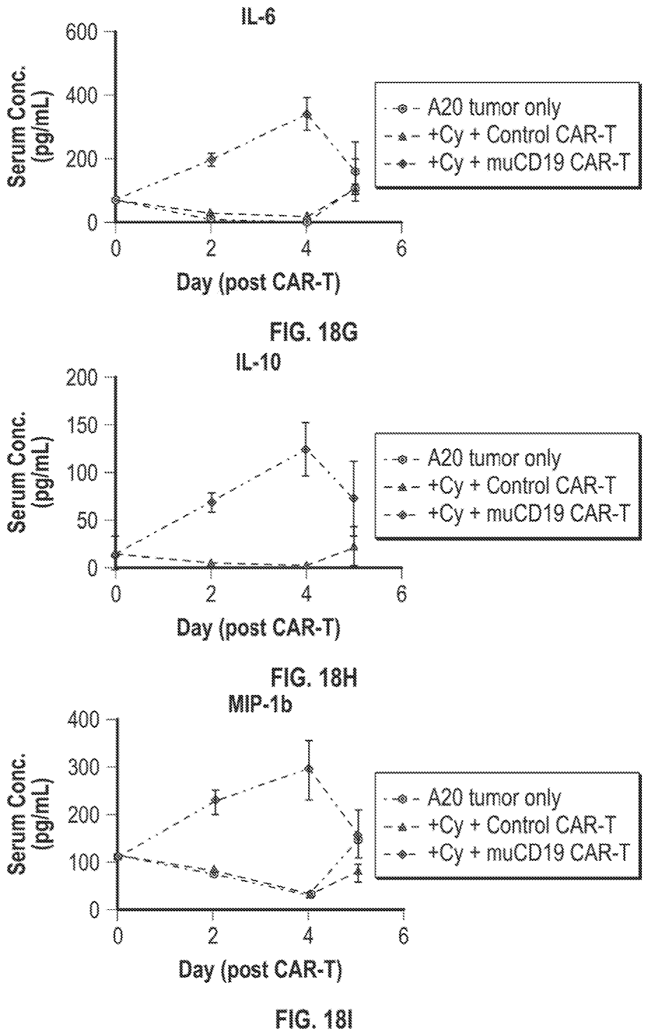

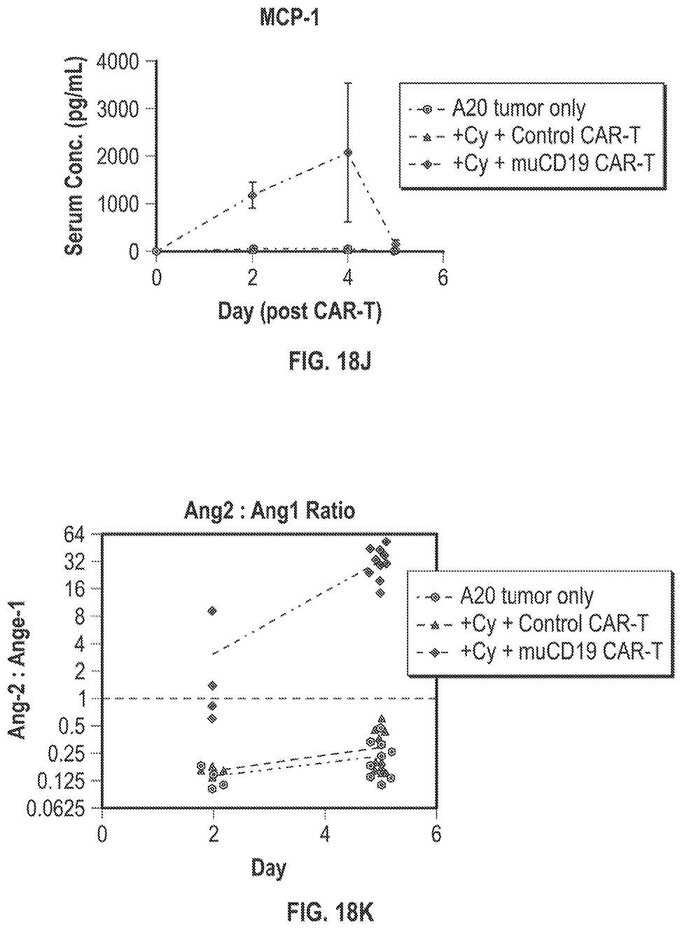

Serum samples were collected at 0, 2, 4, and 5 days following treatment with CAR−T cells and cytokines were measured substantially as described in Example 3. Animals treated with CPA and anti-mouse CD19 CAR−T cells were observed to have elevated levels of serum cytokines, as compared to controls. As shown in FIGS. 18A-18J, detectable increases of serum IFN-gamma (FIG. 18A), TNF-alpha (FIG. 18B), GM-CSF (FIG. 18C), IL-2(FIG. 18D), IL4 (FIG. 18E), IL-5(FIG. 18F), IL-6 (FIG. 18G), IL-10 (FIG. 18H), MIP-1b (FIG. 18I), and MCP-1 (FIG. 18J) levels were detected between 0 and 5 days after treatment with the anti-mouse CD19 CAR−T cells. Elevated levels of serum cytokines elevated in this study may correspond to those observed in human subjects that have been observed to exhibit toxicities following treatment with anti-CD19 CAR−T therapy.

Dysregulated Serum Levels of Angiopoietin-1 (ANG-1) and Angiopoietin-2 (ANG-2) also were observed following treatment with Cy+muCD19 CAR T. Following treatment with CPA (cyclophosphamide or Cy) and anti-mouse CD19 CAR−T cells, an elevated ratio of serum Angiopoietin2 to Angiopoietin 1 (Ang-2:Ang-1) was observed, as compared to treatment with anti-human (mock) CD19 CAR−T cells or no CAR−T cells (A20 cells alone) (FIG. 18K). Elevated Ang-2:Ang1 ratios may be correlated with poor outcomes in sepsis, cerebral edema, and blood brain barrier dysfunction, and elevated Ang-2 in some embodiments is a potential serum biomarker for severe cytokine release syndrome following treatment with CAR−T cells.

### B. T Cell Infiltration in Brain

Brain tissue was examined for T cell infiltration by immunohistochemistry (IHC). A20 tumor cell bearing mice injected with anti-mouse CD19 CAR−T cells or anti-human CD19 CAR−T cells were sacrificed and transcardially perfused 5 days after CAR−T cell injection. Brains were collected and bisected along the longitudinal fissure. One sagittal section was frozen for assessing BBB permeability by immunofluorescent microscopy, and the other half preserved for histology and IHC. Sections were stained with anti-CD3 antibody. Brains from mice treated with anti-mouse CD19 CAR−T cells had a greater number CD3+ cells detected in neuropil than mice anti-human CD19 CAR−T. These data are consistent with an anti-mouse CD19 CAR−T cell directed infiltration into brain.

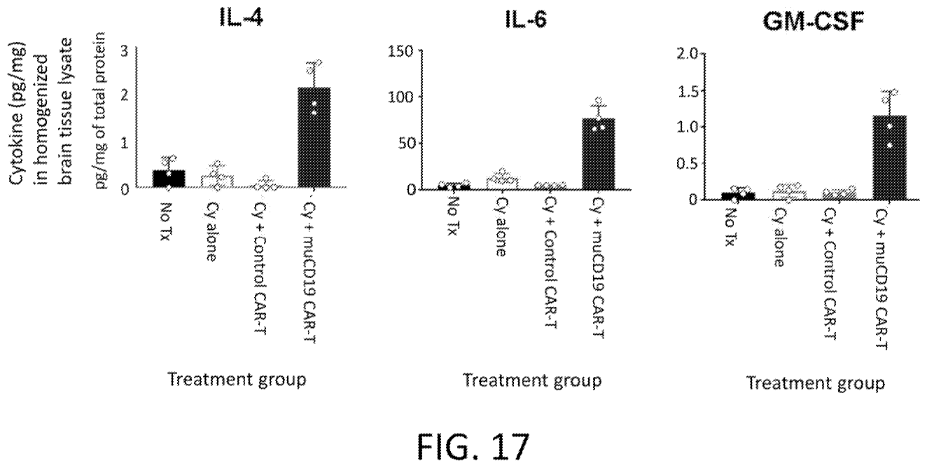

Brains were also analyzed for the presence of cytokines. Cytokines were measured in perfused brain tissue collected 48 hours after CAR−T injection similar to as described in Example 3. As shown in FIG. 17, the brain levels of IL-4, IL-6, and GM-CSF were increased following treatment with CPA and anti-mouse CD19 CAR−T cells as compared to other treatment groups.

### C. Gene Expression in Brain

Gene expression analysis was carried out by RNA-Seq on brains from mice sacrificed and transcardially perfused 48 hours following CAR−T cell injection and at which point brains were collected, flash frozen in liquid nitrogen and stored. RNA-Seq was performed substantially as described in Example 4. Genome-wide expression analysis identified 17,783 genes expressed above background in brains of the examined mice and 1,822 of the genes were deemed differentially expressed following administration of cyclophosphamide (Cy) and anti-mouse CD19 CAR−T cells, as compared controls.

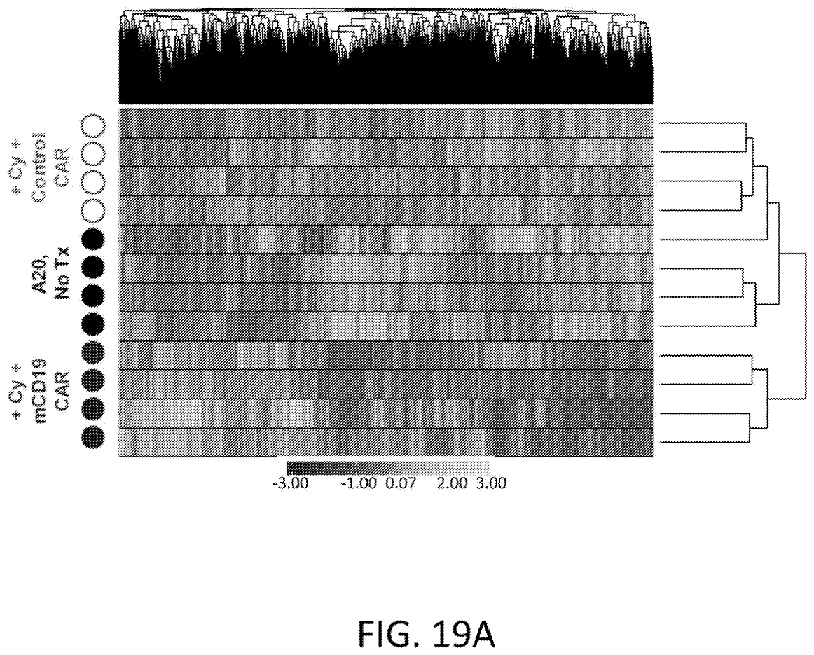

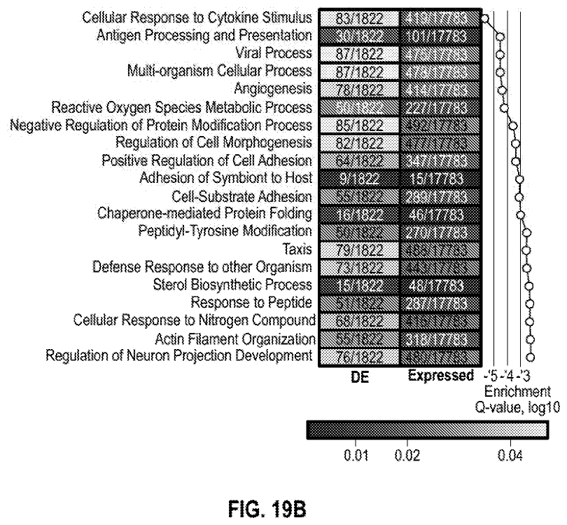

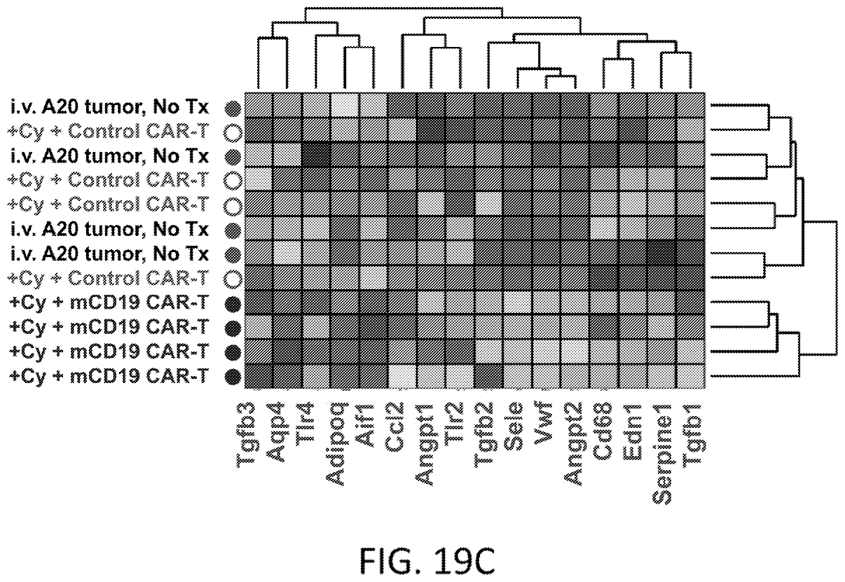

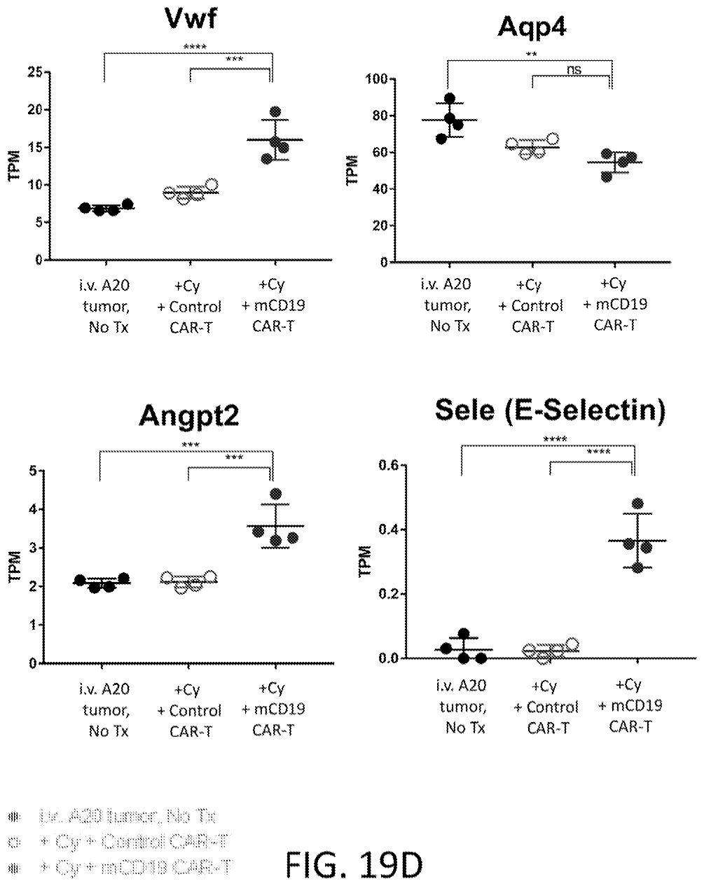

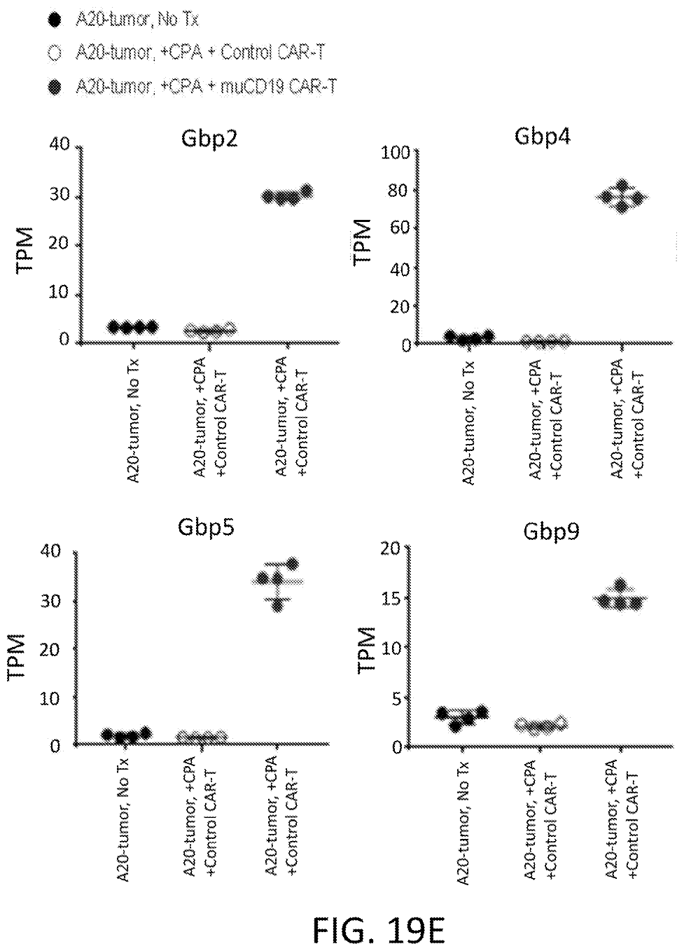

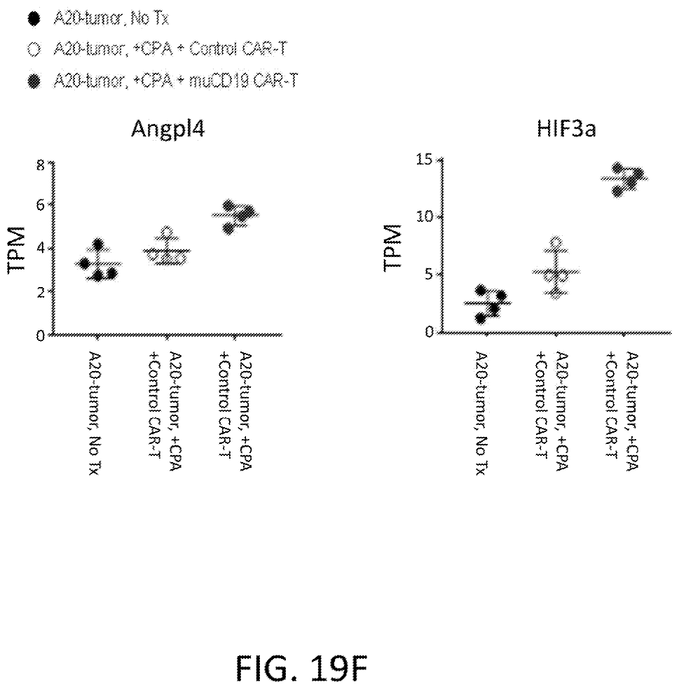

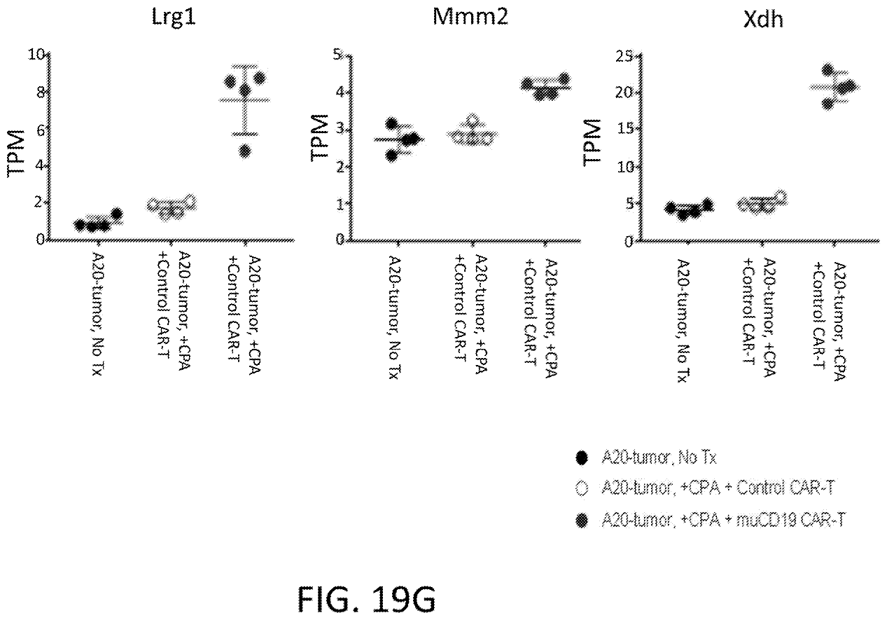

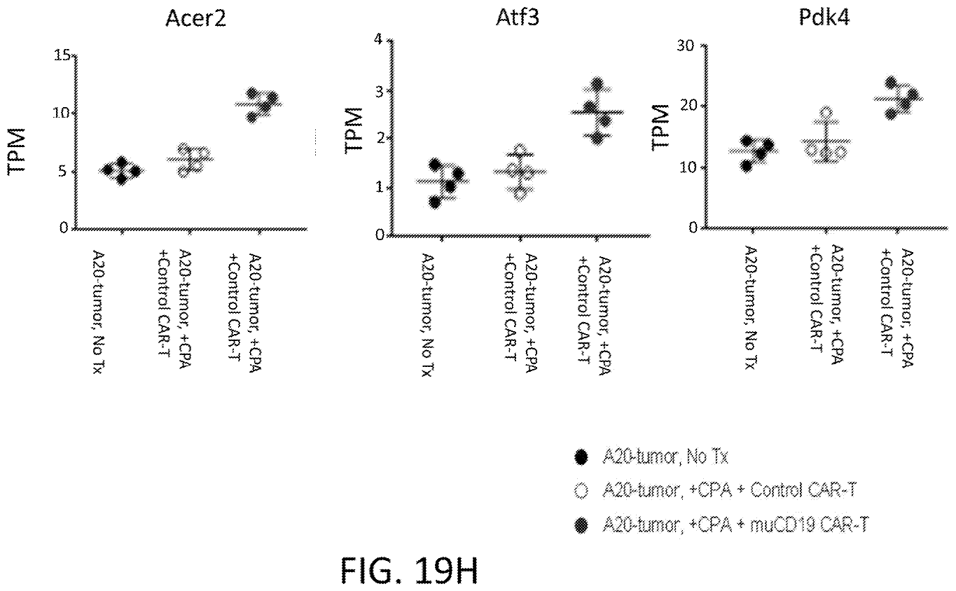

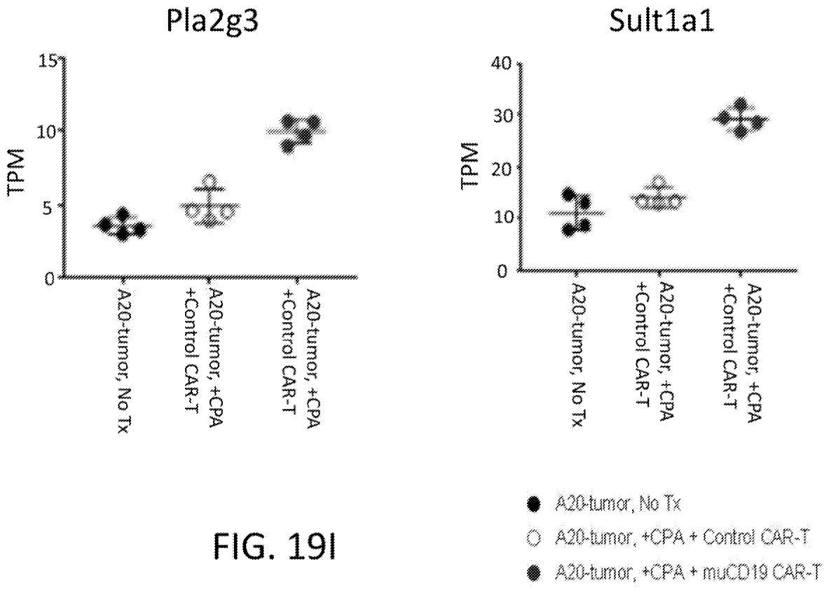

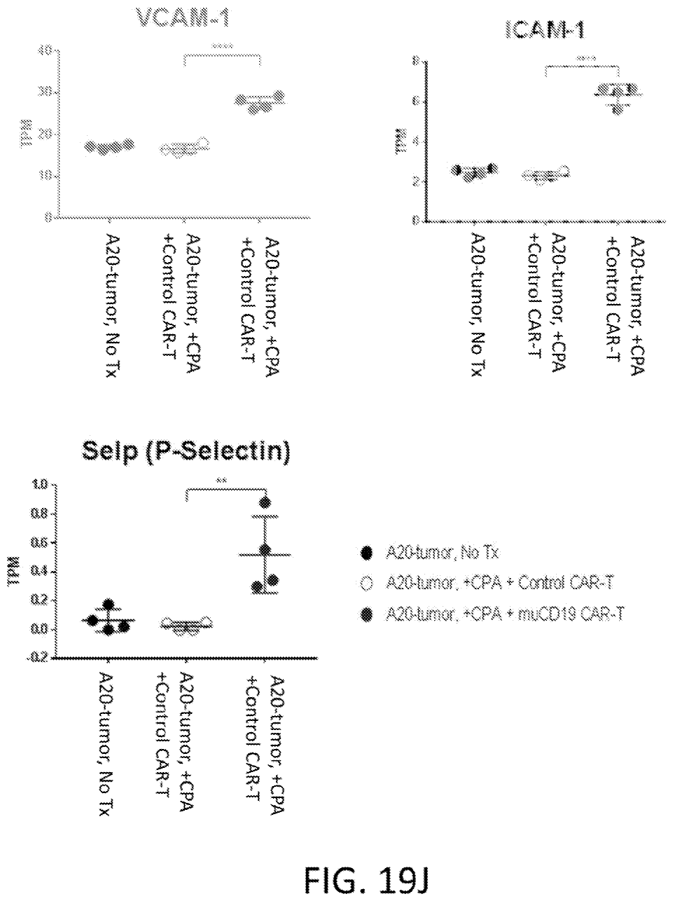

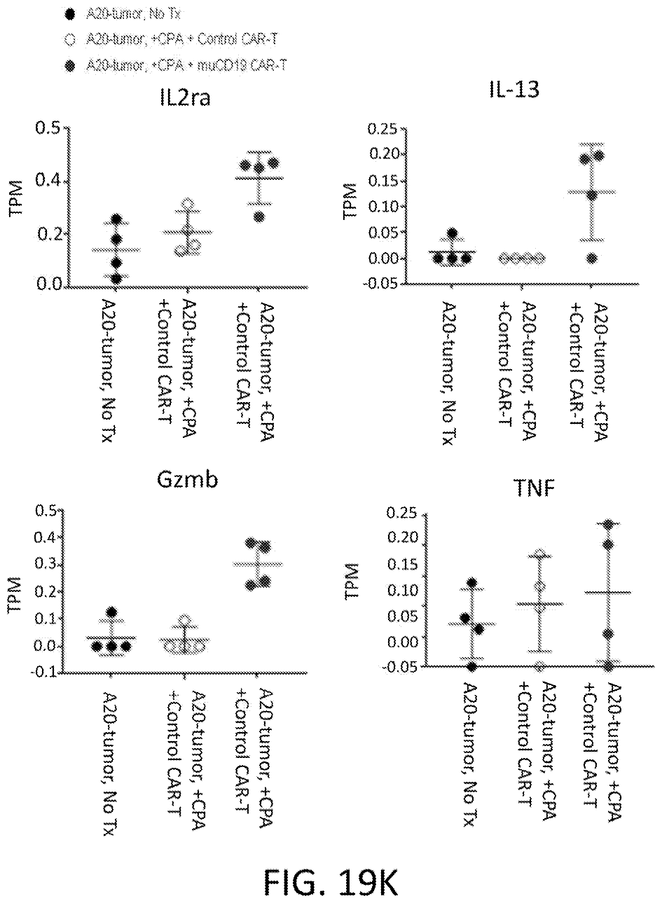

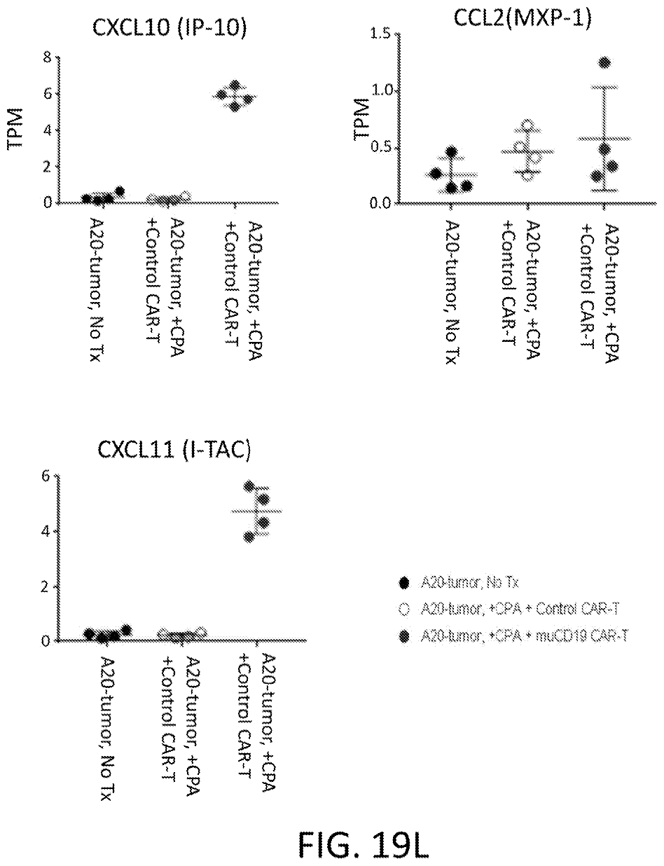

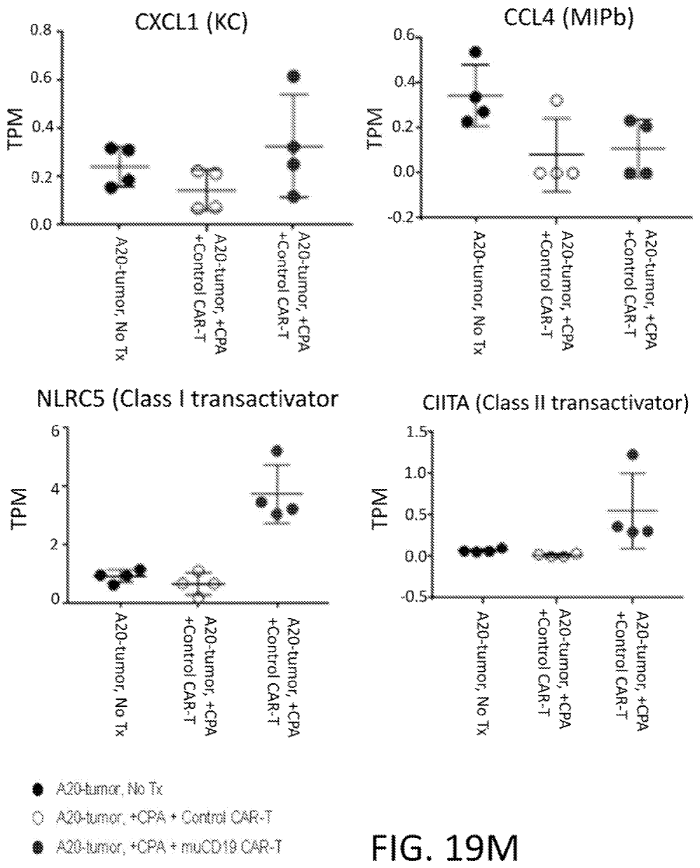

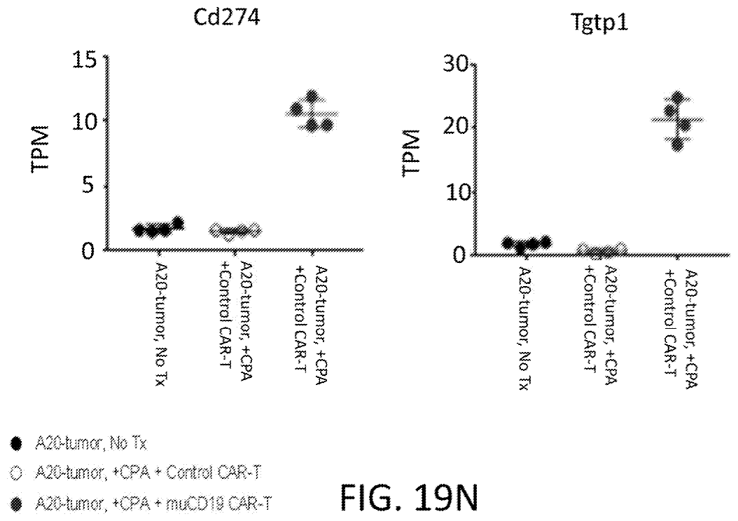

Hierachical clustering analysis was performed for genes deemed to be stably expressed (greater than 5 transcripts per million (TPM). As shown in FIG. 19A, the clustering analysis confirmed differential gene expression profiles in brains of A20 tumor bearing mice treated with anti-mouse CD19 CAR T cells, as compared to control groups.

Gene ontology enrichment analysis was performed for the 1822 genes deemed to be differentially expressed (DE) following treatment with cy and anti-mouse CD19 CAR−T, versus the 17,783 genes identified as expressed. FIG. 19B shows the top 20 gene ontology (GO) terms based on enrichment for the 1822 differentially expressed (DE) genes, with shading corresponding to Enrichment Q-value, log 10. These GO terms were cellular response to cytokine stimulus (83/1822 DE genes vs. 419/17783 expressed genes), antigen processing and presentation (30/1822 DE genes vs. 101/17783 expressed genes), viral process (87/1822 DE genes vs. 475/17783 expressed genes), multi-organism cellular process (75/1822 DE genes vs. 478/17783 expressed genes), angiogenesis (78/1822 DE genes vs. 414/17783 expressed genes), reactive oxygen species metabolic process (50/1822 DE genes vs. 227/17783 expressed genes), negative regulation of protein modification process (85/1822 DE genes vs. 492/17783 expressed genes), regulation of cell morphogenesis (82/1822 DE genes vs. 447/17783 expressed genes), positive regulation of cell adhesion (64/1822 DE genes vs. 347/17783 expressed genes), adhesion of symbiont to host (9/1822 DE genes vs. 15/17783 expressed genes), cell-substrate adhesion (55/1822 DE genes vs. 289/17783 expressed genes), chaperone-mediated protein folding (16/1822 DE genes vs. 46/17783 expressed genes), peptidyl-tyrosine modification (50/1822 DE genes vs. 270/17783 expressed genes), taxis (79/1822 DE genes vs. 488/17783 expressed genes), defense response to other organism (73/1822 DE genes vs. 443/17783 expressed genes), sterol biosynthetic process (15/1822 DE genes vs. 48/17783 expressed genes), response to peptide (51/1822 DE genes vs. 287/17783 expressed genes), cellular response to nitrogen compound (68/1822 DE genes vs. 416/17783 expressed genes), actin filament organization (55/1822 DE genes vs. 318/17783 expressed genes), and regulation of neuron projection development included categories relating to antigen processing and presentation, and angiogenesis.

Individual differentially expressed genes following CAR therapy in this model and categories of genes were identified in this study. Such genes and categories included genes that in some embodiments have involvement in neuroinflammation and neurotoxicity, including TGF-beta genes, angiopotein1 and 2, VWF, toll-like receptor genes and related categories, genes involved in adhesion or angiogenesis or vascular changes, for example, Tgfb3 (transforming growth factor beta 3), Aqp4 (Aquaporin-4), Tlr4 (toll like receptor 4), Adipoq (Adiponectin), Aif1 (Allograft inflammatory factor 1), Ccl2 (C-C motif chemokine 2), Angpt1 (angeopotein 1), Tlr2 (Toll-like receptor 2), Tgfb2 (transforming growth factor beta 2), Sele (E-selectin), Vwf (von Willebrand factor), Angpt2 (angiopotein 2), CD68, Edn1 (Endothelin-1), Serpine 1, Tgfb1 (Transforming growth factor beta-1). The differentially expressed genes and categories included those associated with inflammation and vascular changes (see, e.g., FIGS. 19C (Tgfb3, Aqp4, Tlr4, Adipoq, Aif1, Ccl2, Angpt1, Tlr2, Tgfb2, Sele, Vwf, Angpt2, CD68, Edn1, Serpine 1, Tgfb1) and 19D; immune response, including Gbp2 (guanylate-binding protein 2), Gbp4(guanylate-binding protein 4), Gdp5 (guanylate-binding protein 5), and Gdp9 (guanylate-binding protein 9; FIG. 19E); angiogenesis, including Angpt14 (angiopoietin-like 4), HIF3a (hypoxia inducible factor 3 alpha subunit), Lrg1 (leucine rich alpha-2-glycoprotien 1), Mmrn2, and Xdh (xanthine dehydrogenase; FIGS. 19F and 19G); sterol metabolic processes, including Acer2, Atf3 (cyclic AMP-dependent transcription factor ATF-3), Pdk4 (pyruvate dehydrogenase kinase, isozyme 4), Pla2g3 (group 3 secretory phospholipase A2 precursor), and Sult1a1 (Sulfotransferase 1A1; FIGS. 19H and 191); and adhesion molecules, including VCAM-1 (Vascular cell adhesion protein 1), ICAM-1 (Intercellular adhesion molecule 1), Selp (P-selectin), IL2ra, IL-13, Gzmb (Granzyme B), and TNF (FIGS. 19J and 19K). Mice treated with anti-mouse CD-19 CAR−T cells had altered expression of genes encoding cytokines, chemokines, and MHC proteins, including CXCL10 (IP-10), CCL2 (MCP-1, C-C motif chemokine 2), CXCL11 (I-TAC, C-X-C motif chemokine 11), CXCL1 (KC, Growth-regulated alpha protein), CCL4 (MIP-1b, C-C motif chemokine 4), NLRC5 (class I transactivator), and CIITA (class II transactivator; FIGS. 19L and 19M). Other exemplary genes that were differentially expressed included those such as CD274 and Tgtp (T-cell-specific guanine nucleotide triphosphate-binding protein 1; FIG. 19N).

### Example 9: Assessment of the Expression of Individual Genes in Brain Tissues from a Mouse Model of Toxicity

Exemplary genes that were identified as differentially expressed in mice treated with anti-mouse CD19 CAR expressing cells by the RNA-seq experiments described in Examples 3 and 8 were evaluated in brain tissue by in situ hybridization (ISH). Cryofrozen anti-mouse CD19 CAR−T cell (muCAR−T) and mock T cell (control) compositions, prepared as described in Example 1, were thawed and injected into mice that were previously injected with approximately 200,000 A20 cells 17 days prior and administered 250 mg/kg CPA 1 day prior to the T cell injection, similar to as described in Example 8. Mice were sacrificed either two or five days after treatment with the cells, and brains were harvested and in situ hybridization (ISH) was performed. Briefly, sections were subjected to ISH with probes that hybridized to certain genes identified as differentially expressed: Gbp5, Vwf, or Selp. Slides were washed to remove excess unhybridized probe, stained to visualize hybridized probes, and treated with a histological stain. Results days 2 and 5 are summarized in Table E4.

TABLE E4
Summary of ISH staining of exemplary differentially expressed genes
on brain tissue

| Gene | Description | Results |
|---|---|---|
| Gbp5 | IFN-gamma | At day 2: |
| | inducible | rare cells positive with low expression in controls |
| | gene | cells with high expression were common in all |
| | | regions of brain of muCAR-T treated mice |
| | | most endothelial cells were positive |
| | | Other cell types heterogenous for positive |
| | | staining, including microglia and |
| | | astrocytes |
| | | At day 5: |
| | | expression levels appeared lower than day 2, but |
| | | positive cells were still common in brains of |
| | | muCAR-T treated mice |
| Vwf | Role in | At day 5: |
| | coagulation | expression was increased in brains of muCAR-T |
| | | treated mice as compared to controls |
| Selp | Marker for | At day 2: |
| | endothelial | expression increased in endothelial cells of |
| | activation | meninges from muCAR-T treated mice as |
| | | compared to controls |
| | | At day 5: |
| | | some endothelial cells positive for Selp expression |
| | | were observed within brains from muCAR-T |
| | | treated mice |

The ISH results described in Table E4 were consistent with activation of endothelial cells following administration of the anti-CD19 mouse CAR−T cells to CPA treated mice. In some aspects, the endothelial activation may contribute, at least in part, to pathology or phenotypes observed in the mouse model, and may serve as a readout for testing candidate interventions that prevent or reduce toxicity following treatment with an immunotherapy. Further, the observation that high expression of Gbp5, a general marker of inflammation, was observed in mice treated with anti-CD19 mouse CAR−T cells in all regions of the brain, and not just in infiltrated cells around the muCAR−T cells, is consistent with an observation that the inflammation is due to systemic cytokines.

In addition to the differentially expressed genes described in Table E4, ISH was performed to examine expression of IFN-gamma, IL-6, and CD3 in brain tissue. Cells positive for IFN-gamma expression or IL-6 expression were observed in brains harvested at day 5 or at both 2 and 5 days, respectively, from mice injected with muCAR−T cells, although positively stained cells for either gene were very rare. This result is further consistent with a possible increase in systemic cytokines in the brain following administration of CAR−T cells, which may lead to general brain inflammation and endothelial cell activation. Occasional cells positive for CD3 were observed in brains from CPA treated mice injected with muCAR−T cells. The CD3 positive cells were consistent with the presence of T cells, and possibly CAR+ T cells, in the brain. A greater number of CD3 positive cells were observed at day 5 as compared to day 2, while Gbp5 staining was greater at day 2 compared to day 5, consistent with inflammation at day 2 being caused by systemic cytokines.

### Example 10: Observation of Brain Pathology in a Mouse Model of Neurotoxicity

Immunocompetent BALB/c mice were injected with approximately 2×10⁵ A20 cells by intravenous tail vein injection, and after 16 days, mice were injected with 250 mg/kg CPA i.p., followed 24 hours later by an injection of either anti-mouse CD19 CAR−T cell (muCAR−T; prepared as described in Example 1) or mock T cell (control) compositions, similar to as described in Example 8. Mice were sacrificed two and five days after T cell administration. A mouse that was injected with A20 cells only was sacrificed at the day 2 time point and served as a control. Liver, spleen, and brain tissues were sectioned, stained with hematoxylin and eosin, and examined for signs of pathology.

Brains collected from all groups at the day 2 time points generally did not display any notable signs of pathology However, at the day 5 time point, mice administered muCAR−T cells displayed minimal to mild multifocal parenchymal and lesser meningeal extravascular red blood cells (acute hemorrhage) in various regions of the brain, including diencephalon and cerebellum. These results are consistent with brain hemorrhage in these mice, further highlighting the utility of this model in investigating aspects of and potential interventions for severe neurotoxicity and/or cerebral edema following administration of CAR−T cells.

In livers collected from the A20 only and mock CAR−T administered mice, liver neoplasia was observed at the day 2 time point, with histological resolution of liver neoplasia observed in most mock CAR−T administered mice at the day 5 time point. In mice administered muCAR−T cells, moderate to regionally severe multifocal to coalescing perivascular, parenchymal and intrasinusoidal lymphocytic and histiocytic hepatitis was observed with occasional intravascular small to large round cells and rare mitoses. In addition, the mice administered muCAR−T cells displayed mild to moderate multifocal hepatic necrosis and generally mild cytoplasmic vacuolation of hepatocytes surrounding necrotic foci and to a lesser extent throughout the liver.

Spleens collected at the day 2 time point from the A20 only and mock CAR−T administered mice were generally characterized by mild red pulp hypoplasia with mild lymphoid/white pulp depletion. In spleens collected from mock CAR−T administered mice at the day 5 time point, there was mild to moderate EMH and hypercellular red pulp with occasional large/atypical cells but rare to no mitoses. Spleens collected from the muCAR−T administered mice at the day 2 time point were generally characterized with white pulp/lymphoid depletion (mild to moderate); mild apoptosis; marginal zone expansion by round cells with moderate cytoplasm and variably sized, ovoid nuclei (histiocytes, presumptive), which also expand the red pulp; mild eosinophilic inflammation and mild multifocal histiocytic (presumptive) cytoplasmic vacuolation and intracellular golden brown material (hemosiderin, presumptive).

The observations of acute hemorrhaging in brains collected from mice treated with muCAR−T cells are consistent with a use of the mouse model to investigate mechanisms and potential interventions for neurotoxicity associated with immunotherapies such as CAR−T cell therapy.

### Example 11: Assessment and Comparison of Neuropathology in Different Human Subjects

Autopsy assessments for neuropathology were performed in four subjects having Acute Lymphoblastic Leukemia (ALL) who developed severe neurotoxicity, including grade 4 or 5 neurotoxicity, and/or cerebral edema following the treatment with therapeutic compositions containing T cells engineered to express a chimeric antigen receptor (CAR). The results generally support the conclusion that brain involvement by B-ALL was not observed to be a factor. Further, in patients with cerebral edema, edema tended to be observed to be vasogenic, not cytotoxic. In patients with cerebral edema, perivascular fibrin and red blood cell extravasation suggested blood brain barrier breakdown. There appeared, however, to be an absence of any remarkable T-cell infiltration, consistent with a conclusion that cerebral edema had not developed as a result of CAR T cell infiltration and/or activation within the brain or CNS. Further, perivascular and more diffuse patterns of astrocytic and microglial damage/activation was observed in brains of subjects who had developed cerebral edema. The observation was consistent with a conclusion that microglia activation was a contributor to the development of cerebral edema in subjects administered a CAR−T cell therapy. In addition, irreversible damage to astrocytes (clasmatodendrosis) was observed in subjects who developed cerebral edema, contrasted by astrocytic proliferation observed in subjects who developed Grade 4 neurotoxicity. Complete breakdown of the BBB and resulting vasogenic edema was not observed in subjects who developed grade 4 neurotoxicity. In subjects who did not develop cerebral edema, diffuse CD8+ T-cell infiltration that was not consistent with simple reaction to focal injury was observed.

In another study involving the administration of engineered cells expressing an anti-CD19 CAR, among the subjects exhibiting complete responses were two subjects treated having DLBCL subjects with CNS involvement. In these subjects, the complete resolution of CNS lymphoma was observed without development of any grade of neurotoxicity. In other studies, among subjects having ALL treated with anti-CD19 CART cells, no clear correlation has been observed between incidence of neurotoxicity and the presence of CNS leukemia in the brain (which has been observed to respond to such CAR T cell therapy). Thus, whereas neurotoxicity can occur in some contexts following treatment with CAR−T therapies, such neurotoxicity may not necessarily be the result of target expression in the brain or activity of the CART cells in the CNS, and may not result from “on-target” toxicity by the CAR+ T cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | MASPLTRFLSLNLLLLGESIILGSGEA | Mouse N-terminal |
| | | CD8 alpha signal |
| | | peptide |
| 2 | lqqsgaelvrpgtsvklsckvsgdtitfyymhfvkqrpgqglewi | anti-murine CD19 |
| | gridpedestkysekfknkatltadtssntaylklssltsedtat | variable heavy |
| | yfciyggyyfdywgqgvmvtvs | chain derived |
| | | from the 1D3 rat |
| | | monoclonal anti- |
| | | CD19 antibody |
| 3 | iqmtqspaslstslgetvtiqcqasediysglawyqqkpgkspql | anti-murine CD19 |
| | liygasdlqdgvpsrfsgsgsgtqyslkitsmqtedegvyfcqqg | variable light |
| | ltyprtfgggtklel | chain derived |
| | | from the 1D3 rat |
| | | monoclonal anti- |
| | | CD19 antibody |
| 4 | PRIPKPSTPPGSSCPP | Murine IgG3 |
| | | hinge region |
| 5 | FWALVVVAGVLFCYGLLVTVALCVIWT | murine CD28 |
| | | transmembrane |
| | | region |
| 6 | SVLKWIRKKFPHIFKQPFKKTTGAAQEEDACSCRCPQEEEGGGGG | intracellular |
| | YEL | signaling domain |
| | | of murine 41BB |
| 7 | RAKFSRSAETAANLQDPNQLYNELNLGRREEYDVLEKKRARDPEM | Intracellular |
| | GGKQQRRRNPQEGVYNALQKDKMAEAYSEIGTKGERRRGKGHDGL | signaling domain |
| | YQGLSTATKDTYDALHMQTLAPR | of murine CD3 |
| 8 | MNPAISVALLLSVLQVSRGQKVTSLTACLVNQNLRLDCRHENNTK | Mouse Thy1.1 |
| | DNSIQHEFSLTREKRKHVLSGTLGIPEHTYRSRVTLSNQPYIKVL | |
| | TLANFTTKDEGDYFCELRVSGANPMSSNKSISVYRDKLVKCGGIS | |
| | LLVQNTSWMLLLLLSLSLLQALDFISL | |
| 9 | EVKLQESGPGLVAPSQSLSVTCTVSGVSLPDYGVSWIRQPPRKGL | anti-human CD19 |
| | EWLGVIWGSETTYYNSALKSRLTIIKDNSKSQVFLKMNSLQTDDT | variable heavy |
| | AIYYCAKHYYYGGSYAMDYWGQGTSVTVSS | chain derived |
| | | from the FMC63 |
| | | anti-CD19 |
| | | antibody |
| 10 | DIQMTQTTSSLSASLGDRVTISCRASQDISKYLNWYQQKPDGTVK | anti-murine CD19 |
| | LLIYHTSRLHSGVPSRFSGSGSGTDYSLTISNLEQEDIATYFCQQ | variable light |
| | GNTLPYTFGGGTKLEI | chain derived |
| | | from the FMC63 |
| | | anti-CD19 |
| | | antibody |

1. A method for generating a mouse model of an immunotherapy-associated toxicity or an immunotherapy-associated toxic outcome, comprising:
i) administering a lymphodepleting agent to an immunocompetent mouse, wherein the lymphodepleting agent or therapy does not comprise total body radiation and/or does not comprise complete or substantially complete immune ablation; and
ii) subsequently administering to the mouse an immunotherapy, wherein the immunotherapy binds to and/or recognizes an antigen that is expressed on or in a cell or tissue of the immunocompetent mouse.

2. (canceled)

3. The method of claim 1, wherein the antigen is a B cell antigen or is expressed on the surface of a B cell or wherein the cell is a murine B cell.

4. (canceled)

5. (canceled)

6. (canceled)

7. The method of claim 1, wherein the cells expressing the antigen are administered to the mouse prior to initiating administration of the lymphodepleting agent or therapy or the immunotherapy.

8. A method for generating a mouse model of an immunotherapy-associated toxicity or an immunotherapy-associated toxic outcome, comprising:
i) administering to an immunocompetent mouse tumor cells that express an antigen;
ii) after administering the tumor cells, administering a lymphodepleting agent or therapy to the immunocompetent mouse, wherein the lymphodepleting agent or therapy does not comprise total body radiation and/or does not comprise complete or substantially complete immune ablation; and
iii) subsequently administering to the mouse an immunotherapy, wherein the immunotherapy binds to and/or recognizes the antigen that is expressed on the tumor cells.

9. A method for generating a mouse model of an immunotherapy-associated toxicity or an immunotherapy-associated toxic outcome, comprising:
i) administering a lymphodepleting agent or therapy to an immunocompetent mouse comprising tumor cells that express an antigen, optionally wherein the tumor cells had been administered to the mouse prior to initiation of administration of the lymphodepleting agent or therapy, wherein the lymphodepleting agent or therapy does not comprise total body radiation and/or does not comprise complete or substantially complete immune ablation; and
ii) subsequently administering to the mouse an immunotherapy, wherein the immunotherapy binds to and/or recognizes the antigen that is expressed on the tumor cells.

10. (canceled)

11. (canceled)

12. The method of claim 8, wherein the tumor cells are administered between or between about 7 days and 28 days, 14 days and 21 days, or 17 days and 19 days, each inclusive, prior to initiation of administration of the lymphodepleting agent or therapy or the immunotherapy.

13. (canceled)

14. (canceled)

15. (canceled)

16. The method of claim 8, wherein the tumor cell is a B cell cancer cell line and is selected from L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells or a combination thereof.

17. (canceled)

18. The method of claim 8, wherein the immunotherapy comprises a cell therapy, said cell therapy comprising a dose of genetically engineered cells expressing a recombinant receptor.

19. The method of claim 18, wherein the engineered cells comprise cells obtained from a biological sample from the immunocompetent mouse or from a mouse that is of the same strain or substrain as the immunocompetent mouse.

20. (canceled)

21. The method of claim 18, wherein (i) the cell therapy comprises murine T cells expressing a recombinant receptor that binds to and/or recognizes a murine antigen that is expressed on a B cell of the immunocompetent mouse; (ii) the recombinant receptor is a T cell receptor or functional non-T cell receptor; and/or (iii) the recombinant receptor is a chimeric antigen receptor (CAR).

22-28. (canceled)

29. The method claim 8, wherein the antigen is B cell maturation antigen (BCMA), CD19, CD20, CD22, CD24, CD30, and/or CD38.

30-33. (canceled)

34. The method of claim 8, wherein the immunocompetent mouse is a BALB/c mouse or substrain thereof.

35. (canceled)

36. The method of claim 8, wherein the lymphodepleting agent comprises a chemotherapeutic agent.

37-39. (canceled)

40. The method of claim 36, wherein the chemotherapeutic agent is cyclophosphamide and is administered one time prior to initiation of administration of the immunotherapy.

41. (canceled)

42. (canceled)

43. The method of claim, wherein administration of the immunotherapy is initiated between 12 hours and 48 hours after administering the lymphodepleting agent or therapy.

44-46. (canceled)

47. The method of claim 8, wherein the method results in a toxicity comprising one or more signs, symptoms or outcomes associated with or selected from increased inflammation, altered level, amount or expression or ratio thereof of one or more molecules, selected from the group including a cytokine, chemokine or growth factor, optionally an inflammatory molecule, altered expression or ratio thereof of one or more gene product, altered blood chemistry; tissue damage, optionally damage of the brain; brain edema; weight loss; reduced body temperature; and/or altered behavior.

48-60. (canceled)

61. The method of claim 47, wherein toxicity comprises brain tissue damage.

62. (canceled)

63. A mouse model, comprising a mouse produced by the method of claim 1.

64. A mouse model, comprising an immunocompetent mouse comprising:
a partial depletion in number of one or more populations of lymphocytes compared to the number of the one or more populations of lymphocytes, on average, in a naïve mouse of the same strain; and
an immunotherapy, wherein the immunotherapy binds to and/or recognizes an antigen that is expressed on or in a cell or tissue of the immunocompetent mouse.

65. The mouse model of claim 64, wherein the partial depletion is not permanent or is transient.

66-68. (canceled)

69. The mouse model of claim 64, wherein the antigen is a B cell antigen or is expressed on the surface of a B cell or wherein the cell is a murine B cell.

70. (canceled)

71. The mouse model of claim 69, wherein the cells are tumor cells are exogenous to the mouse and are administered to the mouse prior to administration of the immunotherapy.

72. A mouse model, comprising an immunocompetent mouse comprising:
a partial depletion in number of one or more populations of lymphocytes compared to the number of the one or more populations of lymphocytes, on average, in a naïve mouse of the same strain;
an immunotherapy, wherein the immunotherapy binds to and/or recognizes an antigen, wherein the immunotherapy is exogenous to the immunocompetent mouse; and
tumor cells comprising the antigen, wherein the antigen is expressed on the tumor cell surface.

73. (canceled)

74. The mouse model of claim 72, wherein the tumor cells comprise a B cell cancer cell line that is selected from L1210 cells, 38C13 cells, BCL1 cells, A20 cells, 4TOO cells, B6 spontaneous model cells, CH44 cells, S11 cells, LY-ar cells, LY-as cells, Pi-BCL1 cells, 38C13 Her2/neu cells, Myc5-M5 cells, Mouse lymphosarcoma cell line cells, FL5.12 transfected by Bcl2 cells, 38C13 CD20+ cells, A20.IIA-GFP/IIA1.6-GFP cells, and/or LMycSN-p53null cells.

75. (canceled)

76. The mouse model of claim 72, wherein the immunotherapy comprises a cell therapy, said cell therapy comprising genetically engineered cells expressing a recombinant receptor.

77. (canceled)

78. (canceled)

79. The mouse model of claim 76, wherein the cell therapy comprises murine T cells expressing a recombinant receptor that binds to and/or recognizes a murine antigen that is expressed on a B cell of the immunocompetent mouse.

80. The mouse model of claim 79, wherein the recombinant receptor is (i) a T cell receptor, (ii) a functional non-T cell receptor, or (iii) the recombinant receptor is a chimeric antigen receptor (CAR).

81-86. (canceled)

87. The mouse model of claim 64, wherein the antigen is B cell maturation antigen (BCMA), CD19, CD20, CD22, CD24, CD30, and/or CD38.

88-91. (canceled)

92. The mouse model of claim 64, wherein the immunocompetent mouse is a BALB/c mouse or is of a substrain thereof.

93. The mouse model of claim 92, wherein the lymphodepleting agent comprises a chemotherapeutic agent.

94. (canceled)

95. The mouse model of claim 63, wherein the immunocompetent mouse exhibits one or more signs, symptoms or outcomes associated with a toxicity and/or selected from increased inflammation, altered level, amount or expression or ratio thereof of one or more molecules, selected from the group including a cytokine, chemokine or growth factor, optionally an inflammatory molecule, altered expression or ratio thereof of one or more gene product; altered blood chemistry; tissue damage; brain edema; weight loss; reduced body temperature; and/or altered behavior.

96-113. (canceled)

114. The mouse model of claim 95, wherein toxicity comprises brain tissue damage.

115. (canceled)

116. A tissue sample obtained from a mouse produced by the methods of claim 8.

117. (canceled)

118. (canceled)

119. A method of identifying and/or assessing one or more effects of an agent, the method comprising:
i) administering a lymphodepleting agent or therapy and an immunotherapy to an immunocompetent mouse to generate a toxicity and/or one or more sign, symptom or outcome associated with or indicative of a toxic outcome or side effect;
ii) administering a test agent, to the immunocompetent mouse; and
iii) assessing the toxicity and/or one or more of the sign, symptom, or outcome in the mouse.

120-122. (canceled)

123. A method of identifying and/or assessing one or more effects of an agent, the method comprising:
i) administering a test agent, to an immunocompetent mouse, the immunocompetent mouse having been previously administered a lymphodepleting agent or therapy and an immunotherapy, wherein the immunocompetent mouse exhibits a toxicity and/or one or more sign, symptom or outcome associated with or indicative of a toxic outcome or side effect; and
ii) assessing the toxicity and/or the one or more sign, symptom, or outcome in the mouse.

124. (canceled)

125. The method of claim 123, wherein the method further comprises:
iii) comparing the toxicity and/or the one or more sign, symptom, or outcome to a control mouse, the control mouse having been administered the lymphodepleting agent or therapy and the immunotherapy but not the test agent, wherein the control mouse is immunocompetent.

126-147. (canceled)

148. A mouse model, comprising a mouse produced by the method of claim 8.
